SEQUENCE LISTING (97 sequences)

SEQ ID NO: 1 - DNA, 1184 bp, Artificial Sequence
  source: /note=“Description of Artificial Sequence: Synthetic polynucleotide”
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 2 - protein, 21 aa, Artificial Sequence
  source: /note=“Description of Artificial Sequence: Synthetic peptide”
MALPVTALLLPLALLLHAARP

SEQ ID NO: 3 - DNA, 63 bp, Artificial Sequence
  source: /note=“Description of Artificial Sequence: Synthetic oligonucleotide”
atggccctgcctgtgacagccctgctgctgcctctggctctgctgctgcatgccgctagaccc

SEQ ID NO: 4 - protein, 45 aa, Artificial Sequence
  source: /note=“Description of Artificial Sequence: Synthetic polypeptide”
TTTPAPRPPTPAPTIASQPLSLRPEACRPAAGGAVHTRGLDFACD

SEQ ID NO: 5 - DNA, 135 bp, Artificial Sequence
  source: /note=“Description of Artificial Sequence: Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 6 - protein, 230 aa, Artificial Sequence
  source: /note=“Description of Artificial Sequence: Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 7 - DNA, 690 bp, Artificial Sequence
  source: /note=“Description of Artificial Sequence: Synthetic polynucleotide”
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 8 - protein, 282 aa, Artificial Sequence
  source: /note=“Description of Artificial Sequence: Synthetic polypeptide”
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 9 - DNA, 847 bp, Artificial Sequence
  source: /note=“Description of Artificial Sequence: Synthetic polynucleotide”
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 10 - protein, 10 aa, Artificial Sequence
  source: /note=“Description of Artificial Sequence: Synthetic peptide”
GGGGSGGGGS

SEQ ID NO: 11 - DNA, 30 bp, Artificial Sequence
  source: /note=“Description of Artificial Sequence: Synthetic oligonucleotide”
ggtggcggaggttctggaggtggaggttcc

SEQ ID NO: 12 - protein, 24 aa, Artificial Sequence
  source: /note=“Description of Artificial Sequence: Synthetic peptide”
IYIWAPLAGTCGVLLLSLVITLYC

SEQ ID NO: 13 - DNA, 72 bp, Artificial Sequence
  source: /note=“Description of Artificial Sequence: Synthetic oligonucleotide”
atctacatctgggcgcccttggccgggacttgtggggtccttctcctgtcactggttatcaccctttactgc

SEQ ID NO: 14 - protein, 42 aa, Artificial Sequence
  source: /note=“Description of Artificial Sequence: Synthetic polypeptide”
KRGRKKLLYIFKQPFMRPVQTTQEEDGCSCRFPEEEEGGCEL

SEQ ID NO: 15 - DNA, 126 bp, Artificial Sequence
  source: /note=“Description of Artificial Sequence: Synthetic polynucleotide”
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 16 - protein, 48 aa, Artificial Sequence
  source: /note=“Description of Artificial Sequence: Synthetic polypeptide”
QRRKYRSNKGESPVEPAEPCRYSCPREEEGSTIPIQEDYRKPEPACSP

SEQ ID NO: 17 - DNA, 123 bp, Artificial Sequence
  source: /note=“Description of Artificial Sequence: Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 18 - protein, 112 aa, Artificial Sequence
  source: /note=“Description of Artificial Sequence: Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 336 bp, Artificial Sequence
  source: /note=“Description of Artificial Sequence: Synthetic polynucleotide”
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 20 - protein, 112 aa, Artificial Sequence
  source: /note=“Description of Artificial Sequence: Synthetic polypeptide”
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 336 bp, Artificial Sequence
  source: /note=“Description of Artificial Sequence: Synthetic polynucleotide”
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - protein, 5 aa, Artificial Sequence
  source: /note=“Description of Artificial Sequence: Synthetic peptide”
  source: /note=“See specification as filed for detailed description of substitutions and preferred embodiments”
GGGGS

SEQ ID NO: 23 - DNA, 30 bp, Artificial Sequence; identical to SEQ ID NO: 11
  source: /note=“Description of Artificial Sequence: Synthetic oligonucleotide”

SEQ ID NO: 24 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 25 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 26 - protein, 242 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 27 - protein, 242 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 28 - protein, 40 aa, Artificial Sequence
  source: /note=“Description of Artificial Sequence: Synthetic polypeptide”
  SITE (1)..(40): /note=“This sequence may encompass 1-10 ′Gly Gly Gly Ser′ repeating units”
GGGSGGGSGGGSGGGSGGGSGGGSGGGSGGGSGGGSGGGS

SEQ ID NO: 29 - protein, 20 aa, Artificial Sequence
  source: /note=“Description of Artificial Sequence: Synthetic peptide”
GGGGSGGGGSGGGGSGGGGS

SEQ ID NO: 30 - protein, 15 aa, Artificial Sequence
  source: /note=“Description of Artificial Sequence: Synthetic peptide”
GGGGSGGGGSGGGGS

SEQ ID NO: 31 - protein, 4 aa, Artificial Sequence
  source: /note=“Description of Artificial Sequence: Synthetic peptide”
GGGS

SEQ ID NO: 32 - DNA, 2000 bp, Artificial Sequence
  source: /note=“Description of Artificial Sequence: Synthetic polynucleotide”
  misc_feature (1)..(2000): /note=“This sequence may encompass 50-2000 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 33 - DNA, 150 bp, Artificial Sequence
  source: /note=“Description of Artificial Sequence: Synthetic polynucleotide”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 34 - DNA, 5000 bp, Artificial Sequence
  source: /note=“Description of Artificial Sequence: Synthetic polynucleotide”
  misc_feature (1)..(5000): /note=“This sequence may encompass 50-5000 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - DNA, 100 bp, Artificial Sequence
  source: /note=“Description of Artificial Sequence: Synthetic polynucleotide”
tttttttttttttttttttttttttttttttttttttttttttttttttttttttttttttttttttttttttttttttttttttttttttttttttttt

SEQ ID NO: 36 - DNA, 5000 bp, Artificial Sequence
  source: /note=“Description of Artificial Sequence: Synthetic polynucleotide”
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 37 - DNA, 64 bp, Artificial Sequence
  source: /note=“Description of Artificial Sequence: Synthetic oligonucleotide”
aaaaaaaaaaaaaaaaaaaaaaaaaaaaaaaaaaaaaaaaaaaaaaaaaaaaaaaaaaaaaaaa

SEQ ID NO: 38 - DNA, 400 bp, Artificial Sequence
  source: /note=“Description of Artificial Sequence: Synthetic polynucleotide”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 39 - protein, 247 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 40-42 - skipped (3 entries)

SEQ ID NO: 43 - protein, 247 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 44 - skipped

SEQ ID NO: 45 - protein, 18 aa, Artificial Sequence
  source: /note=“Description of Artificial Sequence: Synthetic peptide”
GSTSGSGKPGSGEGSTKG

SEQ ID NO: 46 - protein, 247 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 47 - protein, 247 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 48 - protein, 247 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 49 - protein, 247 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 50 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 51 - protein, 242 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 52 - protein, 242 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 53 - protein, 119 aa, Artificial Sequence
  source: /note=“Description of Artificial Sequence: Synthetic polypeptide”
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVT

SEQ ID NO: 54 - protein, 111 aa, Artificial Sequence
  source: /note=“Description of Artificial Sequence: Synthetic polypeptide”
ELVLTQSPKFMSTSVGDRVSVTCKASQNVGTNVAWYQQKPGQSPKPLIYSATYRNSGVPDRFTGSGSGTDFTLTITNVQSKDLADYFYFCQYNRYPYTSGGGTKLEIKRRS

SEQ ID NO: 55 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactactcttcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 56 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 57 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 58 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 59 - DNA, 1458 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactattcatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 60 - protein, 486 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - DNA, 1458 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactatcaatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 62 - protein, 486 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 247 aa, Artificial Sequence; identical to SEQ ID NO: 39
  source: /note=“Description of Artificial Sequence: Synthetic polypeptide”

SEQ ID NO: 64 - DNA, 1473 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggcggaggcgggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactactcttcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 65 - protein, 491 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 66 - DNA, 1473 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggaggcggagggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 67 - protein, 491 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - DNA, 1473 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactattcatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggtggcggaagcgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 69 - protein, 491 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - DNA, 1473 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactatcaatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggcggtgggtcagaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 71 - protein, 491 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - DNA, 1473 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggaggcggtgggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaactcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 73 - protein, 491 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - DNA, 1473 bp, Artificial Sequence; identical to SEQ ID NO: 72
  source: /note=“Description of Artificial Sequence: Synthetic polynucleotide”

SEQ ID NO: 75 - protein, 491 aa, Artificial Sequence; identical to SEQ ID NO: 73
  source: /note=“Description of Artificial Sequence: Synthetic polypeptide”

SEQ ID NO: 76 - DNA, 1473 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactataactcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggtggcggaagcgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 77 - protein, 491 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaactcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 79 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - DNA, 1458 bp, Artificial Sequence
  source: /note=“Description of Artificial Sequence: Synthetic polynucleotide”
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 81 - protein, 486 aa, Artificial Sequence
  source: /note=“Description of Artificial Sequence: Synthetic polypeptide”
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82-83 - skipped (2 entries)

SEQ ID NO: 84 - protein, 244 aa, Artificial Sequence
  source: /note=“Description of Artificial Sequence: Synthetic polypeptide”
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVTGGGSGGGSGGGSGGGSELVLTQSPKFMSTSVGDRVSVTCKASQNVGTNVAWYQQKPGQSPKPLIYSATYRNSGVPDRFTGSGSGTDFTLTITNVQSKDLADYFCQYNRYPYTSFFFTKLEIKRRS

SEQ ID NO: 85 - protein, 464 aa, Artificial Sequence
  source: /note=“Description of Artificial Sequence: Synthetic polypeptide”
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVTGGGSGGGSGGGSGGGSELVLTQSPKFMSTSVGDRVSVTCKASQNVGTNVAWYQQKPGQSPKPLIYSATYRNSGVPDRFTGSGSGTDFTLTITNVQSKDLADYFCQYNRYPYTSFFFTKLEIKRRSKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 246 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSE

SEQ ID NO: 87 - protein, 439 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRL

SEQ ID NO: 88 - protein, 819 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 89 - protein, 245 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 90 - protein, 467 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - protein, 4 aa, Artificial Sequence
  source: /note=“Description of Artificial Sequence: Synthetic peptide”
RGDS

SEQ ID NO: 92 - protein, 150 aa, Artificial Sequence [sequence omitted]

SEQ ID NO: 93 - DNA, 450 bp, Artificial Sequence
  source: /note=“Description of Artificial Sequence: Synthetic polynucleotide”
cccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtc

SEQ ID NO: 94 - protein, 394 aa, Artificial Sequence
  source: /note=“Description of Artificial Sequence: Synthetic polypeptide”
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - DNA, 1182 bp, Artificial Sequence
  source: /note=“Description of Artificial Sequence: Synthetic polynucleotide”
atggccctccctgtcactgccctgcttctccccctcgcactcctgctccacgccgctagaccacccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtcacgaccactccggcgccgcgcccaccgactccggccccaactatcgcgagccagcccctgtcgctgaggccggaagcatgccgccctgccgccggaggtgctgtgcatacccggggattggacttcgcatgcgacatctacatttgggctcctctcgccggaacttgtggcgtgctccttctgtccctggtcatcaccctgtactgcaagcggggtcggaaaaagcttctgtacattttcaagcagcccttcatgaggcccgtgcaaaccacccaggaggaggacggttgctcctgccggttccccgaagaggaagaaggaggttgcgagctgcgcgtgaagttctcccggagcgccgacgcccccgcctataagcagggccagaaccagctgtacaacgaactgaacctgggacggcgggaagagtacgatgtgctggacaagcggcgcggccgggaccccgaaatgggcgggaagcctagaagaaagaaccctcaggaaggcctgtataacgagctgcagaaggacaagatggccgaggcctactccgaaattgggatgaagggagagcggcggaggggaaaggggcacgacggcctgtaccaaggactgtccaccgccaccaaggacacatacgatgccctgcacatgcaggcccttccccctcgc

SEQ ID NO: 96 - DNA, 5000 bp, Artificial Sequence; identical to SEQ ID NO: 34
  source: /note=“Description of Artificial Sequence: Synthetic polynucleotide”

SEQ ID NO: 97 - protein, 373 aa, Artificial Sequence
  source: /note=“Description of Artificial Sequence: Synthetic polypeptide”
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2021/0396739 A1
(43) Pub. Date: Dec. 23, 2021

(54) BIOMARKERS FOR EVALUATING CAR-T CELLS TO PREDICT CLINICAL OUTCOME

(71) Applicants: Novartis AG, Basel (CH); The Trustees of the University of Pennsylvania, Philadelphia, PA (US)

(72) Inventors: Iulian Pruteanu-Malinici, Medford, MA (US); Jennifer Brogdon, Sudbury, MA (US); Elena Orlando, Somerville, MA (US); Jan J. Melenhorst, Cherry Hill, NJ (US); Joseph A. Fraietta, Williamstown, NJ (US)

(21) Appl. No.: 17/052,051

(22) PCT Filed: May 1, 2019
(86) PCT No.: PCT/US2019/030229

§ 371 Date: Oct. 30, 2020

Related U.S. Application Data
(60) Provisional application No. 62/740,631, filed on Oct. 3, 2018.
(60) Provisional application No. 62/678,617, filed on May 31, 2018.
(60) Provisional application No. 62/672,925, filed on May 17, 2018.
(60) Provisional application No. 62/665,333, filed on May 1, 2018.

(51) Int. Cl. G01N 33/50; G01N 33/574; G01N 33/68

(52) U.S. Cl. CPC G01N 33/5094; G01N 33/6872; G01N 33/574

(57) ABSTRACT

The invention provides biomarkers, e.g, cancer biomarkers, and methods of using said biomarkers. Specifically, the invention provides biomarkers for use, e.g., in evaluating CAR-T cell therapies and predicting clinical outcome.

### CROSS-REFERENCE TO RELATED APPLICATIONS

This application is a U.S. National Stage Application under 35 U.S.C. § 371 of International Application No. PCT/US2019/030229, filed May 1, 2019, which claims the benefit of U.S. Provisional Application 62/665,333 filed on May 1, 2018, U.S. Provisional Application 62/672,925 filed on May 17, 2018, U.S. Provisional Application 62/678,617 filed on May 31, 2018, U.S. Provisional Application 62/740,631 filed on Oct. 3, 2018, the entire contents of each of which are hereby incorporated by reference.

### FIELD OF THE INVENTION

The invention relates to cancer biomarkers and uses thereof.

### BACKGROUND OF THE INVENTION

Many patients with B cell malignancies are incurable with standard therapy. In addition, traditional treatment options often have serious side effects. Attempts have been made in cancer immunotherapy, however, several obstacles render the goal of clinical effectiveness difficult to achieve. Although hundreds of so-called tumor antigens have been identified, these are generally derived from self and thus are poorly immunogenic. Furthermore, tumors use several mechanisms to render themselves hostile to the initiation and propagation of immune attack.

Recent developments using chimeric antigen receptor (CAR) modified autologous T cell (CART) therapy, which relies on redirecting T cells to a suitable cell-surface molecule on cancer cells such as B cell malignancies, show promising results in harnessing the power of the immune system to treat B cell malignancies and other cancers (see, e.g., Sadelain et al., CANCER DISCOVERY 3:388-398 (2013)). For example, the clinical results of a CART that binds to CD19 (i.e., “CTL019”) have shown promise in establishing complete remissions in patients suffering with chronic lymphocytic leukemia (CLL), as well as in childhood acute lymphocytic leukemia (ALL) (see, e.g., Kalos et al., SCI TRANSL MED 3:95ra73 (2011), Porter et al., NEJM 365:725-733 (2011), Grupp et al., NEJM 368:1509-1518 (2013)).

Besides the ability for the chimeric antigen receptor on the genetically modified T cells to recognize and destroy the targeted cells, a successful therapeutic T cell therapy needs to have the ability to proliferate, to persist over time, and to further monitor for leukemic cell escapees. The variable phenotypic state of T cells, whether it is in a state of anergy, suppression or exhaustion, will have effects on CAR-transformed T cells' efficacy. To be effective, CAR transformed patient T cells need to persist and maintain the ability to proliferate in response to the CAR's antigen.

A need, therefore, exists for a method of using biomarkers for use in connection with the differential diagnosis and treatment of cancer with CAR-expressing cell (e.g., T cell, NK cell) therapy. In particular, there is an unmet need for effective predictors of therapeutic response in subjects having a hematological cancer, such as CLL and ALL, to a CAR-expressing cell therapy, e.g., with CTL019 or other CD19 CAR-expressing cells.

### SUMMARY OF THE INVENTION

The present disclosure relates to the identification and use of analytes, analyte profiles, or markers (e.g., gene expression, flow cytometry and/or protein expression profiles) with clinical relevance to cancer (e.g., a hematological cancer such as chronic lymphocytic leukemia (CLL)). In some embodiments, the disclosure provides the identity of genes, whose expression, at the transcriptional and protein levels, are correlated with disease progression, e.g., CLL progression, e.g., as a way of predicting a response to a Chimeric Antigen Receptor (CAR)-expressing cell therapy (e.g., a therapy comprising a cell (e.g., an immune effector cell or population of cells) that expresses a CAR, e.g., a CAR that binds to a tumor antigen, e.g., CD19 (also referred to herein as a “CAR19” or “CD19 CAR”-expressing cell). In certain embodiments, one or more of a CAR-expressing cell gene set signature, a CD27 biomarker, a CD45RO biomarker, a CCR7 biomarker, a HLA-DR biomarker, a CD95 biomarker, a CD127 biomarker, a CD4 biomarker, a CD8 biomarker, a TH1+ helper T cell gene set signature, a TH2+ helper T cell gene set signature, a memory T cell (e.g., a CD8+ memory T cell, e.g., a naïve T cell (T_{N}), e.g. a memory stem cell (T_{SCM}), e.g. a central memory T cell (T_{CM}), e.g. an effector memory T cell (T_{EM})) gene set signature, and combinations thereof), a PD-1 biomarker, a PD-L1 biomarker, a sCD30 biomarker, or a sTNFR1 biomarker are evaluated. These gene expression profiles may be applied to the diagnosis and/or prognosis of a cancer, e.g., a hematological cancer such as CLL, and are particularly useful in predicting whether a subject will respond favorably to a CAR therapy (e.g., a CD19 CAR therapy as described here, e.g., a CTL019 therapy) in a subject diagnosed with a cancer, e.g., a hematological cancer such as CLL. The biomarkers disclosed herein can also be used to predict responsiveness to a CAR therapy, e.g., as described herein, and to evaluate, e.g., predict, a subject's risk of developing cytokine release syndrome (CRS) or neurotoxicity. Compared to clinical parameters or biochemical markers used in existing prognosis methods, the expression profiles of the genes disclosed herein constitute a more robust signature of disease progression, e.g., hematological cancer progression (e.g., CLL progression) and provide a more reliable, non-subjective basis for the selection of appropriate therapeutic regimens.

Amongst other things, the present disclosure provides novel gene signatures, e.g., at the transcriptional and protein levels, and methods of use thereof, that predict subject response to a cell expressing a CAR, e.g., a CD19 CAR (e.g., a CD19 CAR-expressing cell, e.g., T cell, NK cell, described herein such as, e.g., CTL019) therapy in a cancer, e.g., a hematological cancer such as CLL.

The present disclosure demonstrates, at least in part, that expression profiles and gene signatures, e.g., at the transcriptional and protein levels, are useful to distinguish among a responder, a partial responder, a non-responder, a relapser or a non-relapser to a therapy comprising a CAR-expressing cell (e.g., a CAR-expressing immune effector cell, e.g., a T cell, or an NK cell), (also referred to herein as a “CAR-expressing cell therapy”), in a cancer (e.g., a hematological cancer such as CLL and ALL). In one embodiment, the CAR-expressing cell is a CD19 CAR-expressing cell. In one embodiment, the therapy is a CTL019 therapy. In embodiments, the expression profiles and gene signatures disclosed herein distinguish among a CAR (or CD19 CAR)-expressing cell responder, a CAR (or CD19 CAR)-expressing cell partial responder, or a CAR (or CD19 CAR)-expressing cell non-responder (e.g., a CTL019-responder, a CTL019-partial responder, and a CTL019-non-responder); or a CAR (or CD19 CAR)-expressing cell relapser, or a CAR (or CD19 CAR)-expressing cell non-relapser (e.g., a CTL019-relapser, or a CTL019-relapser), in a cancer (e.g., a hematological cancer such as CLL and ALL). The present disclosure encompasses the identification of novel gene signatures predictive of subject response to a CAR-expressing cell therapy, e.g., a CD19 CAR-expressing cell therapy such as CTL019.

Thus, disclosed herein are methods, compositions, and kits for the identification, assessment and/or treatment of a subject having cancer. Exemplary cancers include, but are not limited to, B-cell acute lymphocytic leukemia (B-ALL), T-cell acute lymphocytic leukemia (T-ALL), acute lymphocytic leukemia (ALL), chronic myelogenous leukemia (CIVIL), chronic lymphocytic leukemia (CLL), B cell promyelocytic leukemia, blastic plasmacytoid dendritic cell neoplasm, Burkitts lymphoma, diffuse large B cell lymphoma (DLBCL), follicular lymphoma (FL), hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin lymphoma, Hodgkin lymphoma (HL), plasmablastic lymphoma, plasmacytoid dendritic cell neoplasm, and Waldenstrom macroglobulinemia. In one embodiment, the cancer is ALL. In another embodiment, the cancer is CLL. In one embodiment, the cancer is DLBCL, e.g., relapsed or refractory DLBCL. In an embodiment, the cancer is FL, e.g., relapsed or refractory FL. In an embodiment, the cancer is associated with CD19 expression.

Accordingly, in one aspect, the invention features a method of evaluating a subject, e.g., evaluating or monitoring the effectiveness of a CAR-expressing cell therapy in a subject, having a cancer, comprising:

acquiring a value of responder status to a therapy comprising a CAR-expressing cell population (e.g., a CAR19-expressing cell population) for the subject, wherein said value of responder status comprises a determination of one, two, three, four, five, six or more (all), of the following:

(i) the level or activity of CD27 (e.g., CD27+) immune effector cells, e.g., in a CD4+ or a CD8+ T cell population, in a sample (e.g., an apheresis sample or a manufactured CAR-expressing cell product sample);

(ii) the level or activity of CD45RO (e.g., CD45RO−) immune effector cells, e.g., in a CD4+ or a CD8+ T cell population, in a sample (e.g., an apheresis sample or a manufactured CAR-expressing cell product sample);

(iii) the level or activity of CCR7 (e.g., CCR7+ or CCR7−) immune effector cells, e.g., in a CD4+ or a CD8+ T cell population, in a sample (e.g., an apheresis sample or a manufactured CAR-expressing cell product sample);

(iv) the level or activity of HLA-DR (e.g., HLADR+ or HLA-DR−) immune effector cells, e.g., in a CD4+ or a CD8+ T cell population, in a sample (e.g., an apheresis sample or a manufactured CAR-expressing cell product sample);

(v) the level or activity of CD95 (e.g., CD95+) immune effector cells, e.g., in a CD4+ or a CD8+ T cell population, in a sample (e.g., an apheresis sample or a manufactured CAR-expressing cell product sample);

(vi) the level or activity of CD127 (e.g., CD127+ or CD127−) immune effector cells, e.g., in a CD4+ or a CD8+ T cell population, in a sample (e.g., an apheresis sample or a manufactured CAR-expressing cell product sample); or

(vii) the level, e.g., number, of functional and/or activated T cells, e.g., as described herein, in a sample (e.g., an apheresis sample or a manufactured CAR-expressing cell product sample), and

wherein said value is indicative of the subject's responsiveness status to the CAR-expressing cell therapy, thereby evaluating the subject,

thereby evaluating the subject.

In some embodiments, the determination of a value of responder status comprises acquiring a measure of one, two, three, four, five, six, seven, eight, nine, ten or all of:

a. (i), (ii) and (iii) in the immune effector cells, wherein (iii) comprises a measure of the level or activity of CCR7+ immune effector cells, e.g., CD27+CD45RO− CCR7+ immune effector cells;

b. (iii) and (iv) in the immune effector cells, wherein (iii) comprises a measure of the level or activity of CCR7+ immune effector cells, e.g., CCR7+ HLA-DR− immune effector cells;

c. (i)(ii) and (vi) in the immune effector cells, wherein (vi) comprises a measure of the level or activity of CD127+ immune effector cells, e.g., CD27+CD45RO− CD127+ immune effector cells;

d. (i) (iii) (v) and (vi) in the immune effector cells, wherein (iii) comprises a measure of the level or activity of CCR7− and (vi) comprises a measure of the level or activity of CD127+ immune effector cells, e.g., CD27+ CCR7− CD95+CD127+ immune effector cells;

e. (i)(iii) and (v) in the immune effector cells, wherein (iii) comprises a measure of the level or activity of CCR7− immune effector cells, e.g., CD27+ CCR7− CD95+ immune effector cells;

f. (i)(ii) and (v) in the immune effector cells, e.g., CD27+CD45RO− CD95+;

g. (ii) and (iii) in the immune effector cells, wherein (iii) comprises a measure of the level or activity of CCR7+ immune effector cells, e.g., CD45RO− CCR7+ immune effector cells;

h. (ii) (iii) and (vi) in the immune effector cells, wherein (iii) comprises a measure of the level or activity of CCR7+ and (vi) comprises a measure of the level or activity of CD127-immune effector cells, e.g., CD45RO− CCR7+CD127− immune effector cells;

i. (i), (ii), (iii) and (vi) in the immune effector cells, wherein (iii) comprises a measure of the level or activity of CCR7+ and (vi) comprises a measure of the level or activity of CD127+ immune effector cells, e.g., CD27+CD45RO− CCR7+CD127− immune effector cells;

j. (ii) and (vi) in the immune effector cells, wherein (iii) comprises a measure of the level or activity of CD127+ immune effector cells, e.g., CD45RO− CD127+ immune effector cells; and/or

k. any one of (i)-(vii).

In some embodiments, the responder status is indicative of a complete response, a partial response, a non-response, or a relapse to the CAR-expressing cell therapy

In some embodiments, the method further comprises performing one, two, three, four, five, six, seven, or more (e.g., all) of:

identifying the subject as a complete responder, partial responder or non-responder, or a relapser or a non-relapser;

administering a CAR-expressing cell therapy;

administered an altered dosing of a CAR-expressing cell therapy;

altering the schedule or time course of a CAR-expressing cell therapy;

administering, e.g., to a non-responder or a partial responder, an additional agent in combination with a CAR-expressing cell therapy, e.g., a checkpoint inhibitor, e.g., a checkpoint inhibitor described herein;

administering to a non-responder or partial responder a therapy that increases the number of younger T cells in the subject prior to treatment with a CAR-expressing cell therapy;

modifying a manufacturing process of a CAR-expressing cell therapy, e.g., enriching for younger T cells prior to introducing a nucleic acid encoding a CAR, or increasing the transduction efficiency, e.g., for a subject identified as a non-responder or a partial responder;

modifying the CAR-expressing cell product prior to infusion into the patient;

adjusting the CAR-expressing cell infusion dose to achieve clinical efficacy; administering an alternative therapy, e.g., for a non-responder or partial responder or relapser; administering an alternative therapy, e.g., for a non-responder or partial responder, e.g., a standard of care for a particular cancer type; or

if the subject is, or is identified as, a non-responder or a relapser, decreasing the TREG cell population and/or TREG gene signature, e.g., by CD25 depletion, administration of cyclophosphamide, anti-GITR antibody, mTOR inhibitor, or a combination thereof.

In another aspect, disclosed herein is method of evaluating or predicting the responsiveness of a subject having a cancer (e.g., a cancer described herein), to treatment with a CAR-expressing cell therapy, comprising a determination of one, two, three, four, five, six or more (all), of the following:

(i) the level or activity of CD27 (e.g., CD27+) immune effector cells, e.g., in a CD4+ or a CD8+ T cell population, in a sample (e.g., an apheresis sample or a manufactured CAR-expressing cell product sample);

(ii) the level or activity of CD45RO (e.g., CD45RO−) immune effector cells, e.g., in a CD4+ or a CD8+ T cell population, in a sample (e.g., an apheresis sample or a manufactured CAR-expressing cell product sample);

(iii) the level or activity of CCR7 (e.g., CCR7+ or CCR7−) immune effector cells, e.g., in a CD4+ or a CD8+ T cell population, in a sample (e.g., an apheresis sample or a manufactured CAR-expressing cell product sample);

(iv) the level or activity of HLA-DR (e.g., HLADR+ or HLA-DR−) immune effector cells, e.g., in a CD4+ or a CD8+ T cell population, in a sample (e.g., an apheresis sample or a manufactured CAR-expressing cell product sample);

(v) the level or activity of CD95 (e.g., CD95+) immune effector cells, e.g., in a CD4+ or a CD8+ T cell population, in a sample (e.g., an apheresis sample or a manufactured CAR-expressing cell product sample);

(vi) the level or activity of CD127 (e.g., CD127+ or CD127−) immune effector cells, e.g., in a CD4+ or a CD8+ T cell population, in a sample (e.g., an apheresis sample or a manufactured CAR-expressing cell product sample); or

(vii) the level, e.g., number, of functional and/or activated T cells, e.g., as described herein, in a sample (e.g., an apheresis sample or a manufactured CAR-expressing cell product sample), and thereby evaluating the subject, or predicting the responsiveness of the subject to the CAR-expressing cell.

In an additional aspect, disclosed herein is a composition comprising a population of immune effector cells that expresses a CAR molecule (a “CAR-expressing cell”), e.g., a CD19 CAR, for use, in treating, or in providing anti-tumor immunity to, a subject having a cancer, e.g., a hematological cancer, who has been identified as being responsive (e.g., identified as a complete responder, partial responder or a non-relapser) to a therapy comprising a CAR-expressing cell population (e.g., a CAR19-expressing cell population), wherein said identifying comprises a determination of one, two, three, four, five, six or more (all), of the following:

(i) the level or activity of CD27 (e.g., CD27+) immune effector cells, e.g., in a CD4+ or a CD8+ T cell population, in a sample (e.g., an apheresis sample or a manufactured CAR-expressing cell product sample);

(ii) the level or activity of CD45RO (e.g., CD45RO−) immune effector cells, e.g., in a CD4+ or a CD8+ T cell population, in a sample (e.g., an apheresis sample or a manufactured CAR-expressing cell product sample);

(iii) the level or activity of CCR7 (e.g., CCR7+ or CCR7−) immune effector cells, e.g., in a CD4+ or a CD8+ T cell population, in a sample (e.g., an apheresis sample or a manufactured CAR-expressing cell product sample);

(iv) the level or activity of HLA-DR (e.g., HLADR+ or HLA-DR−) immune effector cells, e.g., in a CD4+ or a CD8+ T cell population, in a sample (e.g., an apheresis sample or a manufactured CAR-expressing cell product sample);

(v) the level or activity of CD95 (e.g., CD95+) immune effector cells, e.g., in a CD4+ or a CD8+ T cell population, in a sample (e.g., an apheresis sample or a manufactured CAR-expressing cell product sample);

(vi) the level or activity of CD127 (e.g., CD127+ or CD127−) immune effector cells, e.g., in a CD4+ or a CD8+ T cell population, in a sample (e.g., an apheresis sample or a manufactured CAR-expressing cell product sample); or

(vii) the level, e.g., number, of functional and/or activated T cells, e.g., as described herein, in a sample (e.g., an apheresis sample or a manufactured CAR-expressing cell product sample), and.

In an aspect, the disclosure provides a method of treating, or providing anti-tumor immunity, to a subject having a cancer, e.g., a hematological cancer, who has been identified as being responsive (e.g., identified as a complete responder, partial responder or a non-relapser) to a therapy comprising a population of immune effector cells that expresses a CAR molecule (a “CAR-expressing cell” or a “CAR therapy”), e.g., a CD19 CAR, comprising administering to the subject an effective amount of the CAR-expressing cell population, wherein said identifying comprises a determination of one, two, three, four, five, six or more (all), of the following:

(i) the level or activity of CD27 (e.g., CD27+) immune effector cells, e.g., in a CD4+ or a CD8+ T cell population, in a sample (e.g., an apheresis sample or a manufactured CAR-expressing cell product sample);

(ii) the level or activity of CD45RO (e.g., CD45RO−) immune effector cells, e.g., in a CD4+ or a CD8+ T cell population, in a sample (e.g., an apheresis sample or a manufactured CAR-expressing cell product sample);

(iii) the level or activity of CCR7 (e.g., CCR7+ or CCR7−) immune effector cells, e.g., in a CD4+ or a CD8+ T cell population, in a sample (e.g., an apheresis sample or a manufactured CAR-expressing cell product sample);

(iv) the level or activity of HLA-DR (e.g., HLADR+ or HLA-DR−) immune effector cells, e.g., in a CD4+ or a CD8+ T cell population, in a sample (e.g., an apheresis sample or a manufactured CAR-expressing cell product sample);

(v) the level or activity of CD95 (e.g., CD95+) immune effector cells, e.g., in a CD4+ or a CD8+ T cell population, in a sample (e.g., an apheresis sample or a manufactured CAR-expressing cell product sample);

(vi) the level or activity of CD127 (e.g., CD127+ or CD127−) immune effector cells, e.g., in a CD4+ or a CD8+ T cell population, in a sample (e.g., an apheresis sample or a manufactured CAR-expressing cell product sample); or

(vii) the level, e.g., number, of functional and/or activated T cells, e.g., as described herein, in a sample (e.g., an apheresis sample or a manufactured CAR-expressing cell product sample), and

thereby treating, or providing anti-tumor immunity to, the subject.

In some embodiments, the method further comprises performing one, two, three, four, five, six, seven, or more (e.g., all) of:

identifying the subject as a complete responder, partial responder or non-responder, or a relapser or a non-relapser;

administering a CAR-expressing cell therapy;

administered an altered dosing of a CAR-expressing cell therapy;

altering the schedule or time course of a CAR-expressing cell therapy;

administering, e.g., to a non-responder or a partial responder, an additional agent in combination with a CAR-expressing cell therapy, e.g., a checkpoint inhibitor, e.g., a checkpoint inhibitor described herein;

administering to a non-responder or partial responder a therapy that increases the number of younger T cells in the subject prior to treatment with a CAR-expressing cell therapy;

modifying a manufacturing process of a CAR-expressing cell therapy, e.g., enriching for younger T cells prior to introducing a nucleic acid encoding a CAR, or increasing the transduction efficiency, e.g., for a subject identified as a non-responder or a partial responder;

modifying the CAR-expressing cell product prior to infusion into the patient;

adjusting the CAR-expressing cell infusion dose to achieve clinical efficacy; administering an alternative therapy, e.g., for a non-responder or partial responder or relapser; administering an alternative therapy, e.g., for a non-responder or partial responder, e.g., a standard of care for a particular cancer type; or

if the subject is, or is identified as, a non-responder or a relapser, decreasing the TREG cell population and/or TREG gene signature, e.g., by CD25 depletion, administration of cyclophosphamide, anti-GITR antibody, mTOR inhibitor, or a combination thereof.

In embodiments of any of the methods or compositions for use disclosed herein, the determination comprises acquiring a measure of one, two, three, four, five, six, seven, eight, nine, ten or all of:

a. (i), (ii) and (iii) in the immune effector cells, wherein (iii) comprises a measure of the level or activity of CCR7+ immune effector cells, e.g., CD27+CD45RO− CCR7+ immune effector cells;

b. (iii) and (iv) in the immune effector cells, wherein (iii) comprises a measure of the level or activity of CCR7+ immune effector cells, e.g., CCR7+ HLA-DR− immune effector cells;

c. (i)(ii) and (vi) in the immune effector cells, wherein (vi) comprises a measure of the level or activity of CD127+ immune effector cells, e.g., CD27+CD45RO− CD127+ immune effector cells;

d. (i) (iii) (v) and (vi) in the immune effector cells, wherein (iii) comprises a measure of the level or activity of CCR7− and (vi) comprises a measure of the level or activity of CD127+ immune effector cells, e.g., CD27+ CCR7− CD95+CD127+ immune effector cells;

e. (i)(iii) and (v) in the immune effector cells, wherein (iii) comprises a measure of the level or activity of CCR7− immune effector cells, e.g., CD27+ CCR7− CD95+ immune effector cells;

f. (i)(ii) and (v) in the immune effector cells, e.g., CD27+CD45RO− CD95+;

g. (ii) and (iii) in the immune effector cells, wherein (iii) comprises a measure of the level or activity of CCR7+ immune effector cells, e.g., CD45RO− CCR7+ immune effector cells;

h. (ii) (iii) and (vi) in the immune effector cells, wherein (iii) comprises a measure of the level or activity of CCR7+ and (vi) comprises a measure of the level or activity of CD127− immune effector cells, e.g., CD45RO− CCR7+CD127− immune effector cells;

i. (i), (ii), (iii) and (vi) in the immune effector cells, wherein (iii) comprises a measure of the level or activity of CCR7+ and (vi) comprises a measure of the level or activity of CD127+ immune effector cells, e.g., CD27+CD45RO− CCR7+CD127− immune effector cells;

j. (ii) and (vi) in the immune effector cells, wherein (iii) comprises a measure of the level or activity of CD127+ immune effector cells, e.g., CD45RO− CD127+ immune effector cells; and/or

k. any one of (i)-(vii).

In some embodiments of a method or composition for use disclosed herein, the responder status is indicative of a complete response, a partial response, a non-response, or a relapse to the CAR-expressing cell therapy.

In some embodiments of a method or composition for use disclosed herein, the immune effector cell comprises T cells, e.g., CD4+ or CD8+ T cells.

In some embodiments, a method or composition for use disclosed herein further comprises identifying the subject as a responder (e.g., a complete or partial responder), a non-responder, a relapser or a non-relapser, based on a measure of one or more of (i)-(vii).

In some embodiments of a method or composition for use disclosed herein, the measure of one or more of (i)-(vii) comprises evaluating a profile for one or more of gene expression, flow cytometry or protein expression.

In some embodiments of a method or composition for use disclosed herein, the level or activity of one or more of (i)-(vii) in an immune effector cell population is evaluated using a profile or signature indicative of the percentage of one or more of (i)-(vii) in the immune effector cell population in the sample.

In an aspect, the disclosure provides a method of evaluating the potency of a CAR-expressing cell product comprising immune effector cells, e.g., CAR19-expressing cell product sample (e.g., CTL019), said method comprising a determination of one, two, three, four, five, six or more (all), of the following:

(i) the level or activity of CD27 (e.g., CD27+) immune effector cells, e.g., in a CD4+ or a CD8+ T cell population, in a sample (e.g., an apheresis sample or a manufactured CAR-expressing cell product sample);

(ii) the level or activity of CD45RO (e.g., CD45RO−) immune effector cells, e.g., in a CD4+ or a CD8+ T cell population, in a sample (e.g., an apheresis sample or a manufactured CAR-expressing cell product sample);

(iii) the level or activity of CCR7 (e.g., CCR7+ or CCR7−) immune effector cells, e.g., in a CD4+ or a CD8+ T cell population, in a sample (e.g., an apheresis sample or a manufactured CAR-expressing cell product sample);

(iv) the level or activity of HLA-DR (e.g., HLADR+ or HLA-DR−) immune effector cells, e.g., in a CD4+ or a CD8+ T cell population, in a sample (e.g., an apheresis sample or a manufactured CAR-expressing cell product sample);

(v) the level or activity of CD95 (e.g., CD95+) immune effector cells, e.g., in a CD4+ or a CD8+ T cell population, in a sample (e.g., an apheresis sample or a manufactured CAR-expressing cell product sample);

(vi) the level or activity of CD127 (e.g., CD127+ or CD127−) immune effector cells, e.g., in a CD4+ or a CD8+ T cell population, in a sample (e.g., an apheresis sample or a manufactured CAR-expressing cell product sample); or

(vii) the level, e.g., number, of functional and/or activated T cells, e.g., as described herein, in a sample (e.g., an apheresis sample or a manufactured CAR-expressing cell product sample), and

wherein the sample is acquired from a subject, e.g., as described herein, and

wherein an increase in (i), (ii), (iv), (v) or (vii) or any combination thereof; or an increase in CCR7+ of (iii) and (iv), is indicative of increased suitability for manufacturing, e.g., increased potency, of the CAR-expressing cell product, thereby evaluating the potency of the CAR-expressing cell product.

In some embodiments, the determination comprises acquiring a measure of of one, two, three, four, five, six, seven, eight, nine, ten or all of:

a. (i), (ii) and (iii) in the immune effector cells, wherein (iii) comprises a measure of the level or activity of CCR7+ immune effector cells, e.g., CD27+CD45RO− CCR7+ immune effector cells;

b. (iii) and (iv) in the immune effector cells, wherein (iii) comprises a measure of the level or activity of CCR7+ immune effector cells, e.g., CCR7+ HLA-DR− immune effector cells;

c. (i)(ii) and (vi) in the immune effector cells, wherein (vi) comprises a measure of the level or activity of CD127+ immune effector cells, e.g., CD27+CD45RO− CD127+ immune effector cells;

d. (i) (iii) (v) and (vi) in the immune effector cells, wherein (iii) comprises a measure of the level or activity of CCR7− and (vi) comprises a measure of the level or activity of CD127+ immune effector cells, e.g., CD27+ CCR7− CD95+CD127+ immune effector cells;

e. (i)(iii) and (v) in the immune effector cells, wherein (iii) comprises a measure of the level or activity of CCR7− immune effector cells, e.g., CD27+ CCR7− CD95+ immune effector cells;

f. (i)(ii) and (v) in the immune effector cells, e.g., CD27+CD45RO− CD95+;

g. (ii) and (iii) in the immune effector cells, wherein (iii) comprises a measure of the level or activity of CCR7+ immune effector cells, e.g., CD45RO− CCR7+ immune effector cells;

h. (ii) (iii) and (vi) in the immune effector cells, wherein (iii) comprises a measure of the level or activity of CCR7+ and (vi) comprises a measure of the level or activity of CD127− immune effector cells, e.g., CD45RO− CCR7+CD127− immune effector cells;

i. (i), (ii), (iii) and (vi) in the immune effector cells, wherein (iii) comprises a measure of the level or activity of CCR7+ and (vi) comprises a measure of the level or activity of CD127+ immune effector cells, e.g., CD27+CD45RO− CCR7+CD127− immune effector cells;

j. (ii) and (vi) in the immune effector cells, wherein (iii) comprises a measure of the level or activity of CD127+ immune effector cells, e.g., CD45RO− CD127+ immune effector cells; and/or

k. any one of (i)-(vii).

In another aspect, the invention features a method to identify a likely responder (e.g., a complete responder or a partial responder, a non-relapser) to a therapy comprising a CAR-expressing cell (e.g., a T cell, an NK cell) (e.g., a CD19 CAR-expressing cell therapy, e.g., described herein, e.g., a CTL019 therapy). In an embodiment, a responder status (e.g. a complete responder, a partial responder, a non-responder, a relapser or a non-relapser to a therapy comprising a CAR-expressing cell (e.g., a T cell, an NK cell)) is determined by measuring one or more of a CD19 CAR-expressing cell gene set signature, a biomarker chosen from CD27 biomakrer, a CD45RO biomarker, a CCR7 biomarker, a CD95 biomarker, a CD127 biomarker, a HLA-DR biomarker, a CD4 biomarker, a CD8 biomarker, a TH1+ helper T cell gene set signature, a TH2+ helper T cell gene set signature, a memory T cell (e.g., a CD8+ memory T cell, e.g., a naïve T cell (T_{N}), e.g. a memory stem cell (T_{SCM}), e.g. a central memory T cell (T_{CM}), e.g. an effector memory T cell (T_{EM})) gene set signature, and combinations thereof.

In yet another aspect, provided herein is a method for optimizing manufacturing of a CAR-expressing cell product comprising immune effector cells, e.g., CAR19-expressing cell product sample (e.g., CTL019), comprising:
(1) acquiring from a subject a sample comprising CAR-expressing cell (e.g., a population of CAR-expressing immune effector cells);
(2) activating the CAR-expressing cell in vitro;
(3) evaluating the potency of the potency of the activated CAR-expressing cell by determining a value of one, two, three, four, five, six or more (all), of the following:

(i) the level or activity of CD27 (e.g., CD27+) immune effector cells, e.g., in a CD4+ or a CD8+ T cell population, in a sample (e.g., an apheresis sample or a manufactured CAR-expressing cell product sample);

(ii) the level or activity of CD45RO (e.g., CD45RO−) immune effector cells, e.g., in a CD4+ or a CD8+ T cell population, in a sample (e.g., an apheresis sample or a manufactured CAR-expressing cell product sample);

(iii) the level or activity of CCR7 (e.g., CCR7+ or CCR7−) immune effector cells, e.g., in a CD4+ or a CD8+ T cell population, in a sample (e.g., an apheresis sample or a manufactured CAR-expressing cell product sample);

(iv) the level or activity of HLA-DR (e.g., HLADR+ or HLA-DR−) immune effector cells, e.g., in a CD4+ or a CD8+ T cell population, in a sample (e.g., an apheresis sample or a manufactured CAR-expressing cell product sample);

(v) the level or activity of CD95 (e.g., CD95+) immune effector cells, e.g., in a CD4+ or a CD8+ T cell population, in a sample (e.g., an apheresis sample or a manufactured CAR-expressing cell product sample);

(vi) the level or activity of CD127 (e.g., CD127+ or CD127−) immune effector cells, e.g., in a CD4+ or a CD8+ T cell population, in a sample (e.g., an apheresis sample or a manufactured CAR-expressing cell product sample); or

(vii) the level, e.g., number, of functional and/or activated T cells, e.g., as described herein, in a sample (e.g., an apheresis sample or a manufactured CAR-expressing cell product sample), and

wherein an increase in (i), (ii), (iv), (v) or (vii) or any combination thereof; or an increase in CCR7+ of (iii) and (iv), is indicative of increased potency of the CAR-expressing cell product, thereby optimizing manufacturing of the product.

In some embodiments, the determination comprises acquiring a measure of of one, two, three, four, five, six, seven, eight, nine, ten or all of:

a. (i), (ii) and (iii) in the immune effector cells, wherein (iii) comprises a measure of the level or activity of CCR7+ immune effector cells, e.g., CD27+CD45RO− CCR7+ immune effector cells;

b. (iii) and (iv) in the immune effector cells, wherein (iii) comprises a measure of the level or activity of CCR7+ immune effector cells, e.g., CCR7+ HLA-DR− immune effector cells;

c. (i)(ii) and (vi) in the immune effector cells, wherein (vi) comprises a measure of the level or activity of CD127+ immune effector cells, e.g., CD27+CD45RO− CD127+ immune effector cells;

d. (i) (iii) (v) and (vi) in the immune effector cells, wherein (iii) comprises a measure of the level or activity of CCR7− and (vi) comprises a measure of the level or activity of CD127+ immune effector cells, e.g., CD27+ CCR7− CD95+CD127+ immune effector cells;

e. (i)(iii) and (v) in the immune effector cells, wherein (iii) comprises a measure of the level or activity of CCR7− immune effector cells, e.g., CD27+ CCR7− CD95+ immune effector cells;

f. (i)(ii) and (v) in the immune effector cells, e.g., CD27+CD45RO− CD95+;

g. (ii) and (iii) in the immune effector cells, wherein (iii) comprises a measure of the level or activity of CCR7+ immune effector cells, e.g., CD45RO− CCR7+ immune effector cells;

h. (ii) (iii) and (vi) in the immune effector cells, wherein (iii) comprises a measure of the level or activity of CCR7+ and (vi) comprises a measure of the level or activity of CD127− immune effector cells, e.g., CD45RO− CCR7+CD127− immune effector cells;

i. (i), (ii), (iii) and (vi) in the immune effector cells, wherein (iii) comprises a measure of the level or activity of CCR7+ and (vi) comprises a measure of the level or activity of CD127+ immune effector cells, e.g., CD27+CD45RO− CCR7+CD127− immune effector cells;

j. (ii) and (vi) in the immune effector cells, wherein (iii) comprises a measure of the level or activity of CD127+ immune effector cells, e.g., CD45RO− CD127+ immune effector cells; and/or

k. any one of (i)-(vii).

In some embodiments, a method disclosed herein further comprises a step of enriching for, e.g., isolating, cells, the immune effector cell population, e.g., the CAR-expressing immune effector cell population.

In some embodiments of a method of evaluating the potency of a CAR-expressing cell product, or a method for optimizing manufacturing of a CAR-expressing cell product:

an increase in, e.g., a greater percentage of, CD27+CD45RO− CCR7+ immune effector cells in the CAR-expressing cell product, e.g., in the CD8+ population,

an increase in, e.g., a greater percentage of, CD27+CD45RO− CD127+ immune effector cells in the CAR-expressing cell product, e.g., in the CD8+ population,

an increase in, e.g., a greater percentage of, CD27+ CCR7− CD95+ immune effector cells in the CAR-expressing cell product, e.g., in the CD8+ population,

an increase in, e.g., a greater percentage of, CD27+CD45RO− CD95+ immune effector cells in the CAR-expressing cell product, e.g., in the CD8+ population;

an increase in, e.g., a greater percentage of, CCR7+CD45RO− immune effector cells in the CAR-expressing cell product, e.g., in the CD8+ population;

an increase in, e.g., a greater percentage of, CD45RO− CCR7+CD127− immune effector cells in the CAR-expressing cell product, e.g., in the CD8+ population;

an increase in, e.g., a greater percentage of, CD27+CD45RO− CCR7+CD127+ immune effector cells in the CAR-expressing cell product, e.g., in the CD8+ population;

an increase in, e.g., a greater percentage of CD45RO− CD127+ immune effector cells in the CAR-expressing cell product, e.g., in the CD8+ population;

an increase in, e.g., a greater percentage of, CCR7+ HLA-DR− immune effector cells in the CAR-expressing cell product, e.g., in the CD8+ population; or

an increase in, e.g., a greater percentage of, CD27+ CCR7− CD95+CD127+ immune effector cells in the CAR-expressing cell product, e.g., in the CD8+ population,

compared to an otherwise identical cell population is indicative of increased suitability for manufacturing, e.g., increased potency, of the CAR-expressing cell product.

In an aspect, provided herein is method of treating a cancer, e.g., a hematological cancer (e.g., CLL), in a subject, comprising administering to said subject a population of CAR-expressing cells, e.g., CAR19-expressing cells, in an amount effective to result in a cellular response comprising one or more phases of response, e.g., an early phase and/or a late phase, thereby treating the cancer. In some embodiment, the method further comprises identifying the subject as being in an early and/or late phase of response.

In another aspect, the disclosure provides a method of evaluating a subject, or evaluating responsiveness to a CAR therapy in a subject, comprising:

determining if the subject has an early phase response, a late phase response, or both an early phase response and a late phase response, wherein:

(i) a determination of an early phase response or a late phase response is indicative that the subject is less responsive to a CAR therapy (e.g., compared to a subject that is determined to have both an early phase response and a late phase response), e.g., is a partial responder or a relapser, to the CAR therapy;

(ii) a determination of both an early phase response and a late phase response is indicative that the subject is more responsive to a CAR therapy (e.g., compared to a subject that is determined to have only an early phase response or a late phase response), e.g., is a complete responder, to the CAR therapy.

In some embodiments, the early phase is characterized by, or is identified as having, a greater number of CD8+ T cells relative to CD4+ T cells. In some embodiments, the early phase occurs, e.g., about 1-30 days (e.g., about 1-5, 5-10, 10-15, 15-20, 20-25, 25-30 days) or about 1-6 months (e.g., about 1, 2, 3, 4, 5, or 6 months) after administration of the CAR-expressing cell therapy. In some embodiments, the late phase is characterized by, or identified as having, a greater number of CD4+ T cells relative to CD8+ T cells. In some embodiments, the late phase occurs, e.g., about e.g., at least about 1-60 days (e.g., about 1-10, 10-20, 20-30, 30-40, 40-50, or 50-60 days) or about 1-12 months (e.g., about 1, 2, 3, 4, 5, 6, 7, 8, 9, 10, 11, or 12 months) after administration of the CAR-expressing cell therapy. In some embodiments, the early phase occurs about e.g., about 1-30 days (e.g., about 1-5, 5-10, 10-15, 15-20, 20-25, 25-30 days) or about 1-6 months (e.g., about 1, 2, 3, 4, 5, or 6 months) before the commencement, e.g., start, of the late phase. In some embodiments, the hematological cancer is a relapsed or refractory hematological cancer. In some embodiments, hematological cancer is chosen from ALL, CLL, NHL, or DLBCL.

In some embodiments, the population of CAR-expressing cells comprises immune effector cells, e.g., comprising a T cell having an activated phenotype, e.g., expressing HLA-DR. In some embodiments, the CAR-expressing cells comprise CD4+ or CD8+ T cells having a T_{CM} or a T_{EM} phenotype.

In some embodiments, the response results in a reduction, e.g., elimination, of cancer cells in the subject. In some embodiments, the reduction is measured compared to a pre-treatment evaluation of the subject, e.g., using a method described herein, e.g., imaging.

In some embodiments, the response (e.g., early and/or late response) mediated by the CAR-expressing T cells results in a partial or complete response.

In some embodiments, the early and late response mediated by the CAR-expressing T cells results in a remission, e.g., a long-term remission, e.g., a remission lasting about 2-20 years, e.g., about 3-19, 4-18, 5-17, 6-16, 7-15, 8-14, 9-13, 10-12 years, or about 2-5, 5-10, 10-15, or 15-20 years, in the subject.

### PD-1/PD-L1 Interaction Score as a Biomarker

In an aspect, the disclosure provides, a method of evaluating a subject, e.g., evaluating or monitoring the effectiveness of a CAR-expressing cell therapy in a subject, having a cancer, comprising:

acquiring a value of responder status to a therapy comprising a CAR-expressing cell population (e.g., a CAR19-expressing cell population) for the subject, wherein said value of responder status comprises a measure of the level or activity of PD-1 and/or PD-L1, wherein the measure comprises an interaction score, e.g., an interaction score of PD1 and PDL1 (PD1/PDL1), thereby evaluating the subject.

In some embodiments, the responder status is indicative of a complete response, a partial response, a non-response, or a relapse to the CAR-expressing cell therapy.

In some embodiments, a low PD1/PD-L1 interaction score, e.g., an interaction score less than about 1800 (e.g., about 1700-1500, 1500-1300, 1300-1100, 1100-900, 900-700, 700-500, 500-400, 400-300, 300-200, 200-100, 100-0, or at least about 1700, 1500, 1300, 1100, 1000, 900, 800, 700, 600, 500, 400, 300, 200, 100, or lesser) is indicative of a subject's responsiveness, e.g., complete response or partial response, to a CAR-expressing cell therapy. In some embodiments, a low PD1/PD-L1 interaction score, e.g., as described herein, is indicative of a subject not having a non-response or a relapse to CAR-expressing cell therapy, e.g., CAR19 expressing cell therapy, in less than about 3 months, e.g., less than about 2 months or 1 month, after administration of the CAR-expressing cell therapy. In some embodiments, a low PD1/PD-L1 interaction score, e.g., an interaction score less than about 1800 (e.g., about 1700, 1500, 1300, 1100, 1000, 900, 800, 700, 600, 500, 400, 300, 200, 100, or lesser), is indicative of improved efficacy of a CAR-expressing cell therapy, e.g., a CAR19 expressing cell therapy.

In some embodiments, a high PD1/PD-L1 interaction score, e.g., an interaction score of at least about 1800 or higher (e.g., about 1800-6000, e.g., about 1800-2500, about 2500-3500, about 3500-5000, or about 5000-6000, or at least about 2000, 2200, 2400, 2600, 2800, 3000, 3200, 3400, 3600, 3800, 4000, 4200, 4400, 4600, 4800, 5000, 5200, 5400, 5600, 5800, or 6000 or higher), is indicative of a lack of a subject's responsiveness, e.g., a non-response or relapse, to a CAR-expressing cell therapy. In some embodiments, a high PD1/PD-L1 interaction score, e.g., as described herein, is indicative of a subject having a non-response or a relapse to CAR-expressing cell therapy, e.g., CAR19 expressing cell therapy, in less than about 3 months, e.g., less than about 2 months or 1 month, after administration of the CAR-expressing cell therapy. In some embodiments, a high PD1/PD-L1 interaction score, e.g., at least about 1800 or higher (e.g., about 1800-6000), is indicative of reduced efficacy of a CAR-expressing cell therapy, e.g., a CAR19 expressing cell therapy. In some embodiments, a subject with a high PD1/PD-L1 interaction score, e.g., at least about 1800 or higher (e.g., about 1800-6000), is administered an additional therapy, e.g., as described herein. In some embodiments, a subject with a high PD1/PD-L1 interaction score is administered: (i) an altered, e.g., higher, dose of the CAR-expressing cell therapy; (ii) a different dosing regimen, e.g., a different frequency of dosing, of the CAR-expressing cell therapy; or (iii) an additional agent, e.g., as described herein, in combination with the CAR-expressing cell therapy.

In some embodiments, the measure of the activity or level of PD1 or PDL1 is obtained from: an apheresis sample acquired from the subject, wherein optionally the apheresis sample is evaluated prior to infusion or re-infusion, or after infusion; a biopsy sample acquired from the subject; or a manufactured CAR-expressing cell product sample, e.g., CAR19-expressing cell product sample (e.g., CTL019), wherein optionally the manufactured CAR-expressing cell product is evaluated prior to infusion or re-infusion, or after infusion.

In some embodiments, the measure of the activity or level of PD1 is obtained from immune effector cells from an apheresis sample, e.g., T cells.

In some embodiments, the measure of the activity or level of PD1 is obtained from a manufactured CAR-expressing cell product sample, e.g., CAR-expressing T cells, e.g., CAR19-expressing T cells.

In some embodiments, the measure of the activity or level of PDL1 is obtained from cells from an apheresis sample or a biopsy sample, e.g., wherein the sample comprises cancer cells, e.g., CD19 expressing cancer cells.

In some embodiments, the measure of the activity or level of PDL1 is obtained from cancer cells, e.g., CD19 expressing cancer cells.

In some embodiments, the interaction score is measured using a method described herein, e.g., in Example 3. In some embodiments, the interaction score is measured using AQUA technology, e.g., as described herein, e.g., in Example 3. In some embodiments, the interaction score comprises a measure of PD-1 and PD-L1. In some embodiments, the measure of PD-1- and PD-L1 is used to determine, e.g., calculate, the interaction score. In some embodiments, the interaction score is defined as the proportion of PD1 positive cells co-localized with PD-L1 positive cells.

Methods of measuring a PD-1/PD-L1 interaction score, e.g., as described herein, e.g., in Example 3, are disclosed in International Application WO 2017/070582 filed on 21 Oct. 2016, herein incorporated by reference in its entirety. Additional methods of measuring the interaction score are also disclosed in International Applications WO 2017/070584 and WO 2017/070585, the entire contents of all of which are hereby incorporated by reference in their entireties.

### Biomarkers for Predicting Neurotoxicity

In an aspect, the disclosure provides a method of evaluating, e.g., predicting, a subject's risk for developing neurotoxicity, e.g., as described herein, comprising:

acquiring a neurotoxicity risk status for the subject, e.g., in response to a CAR-expressing cell therapy (e.g., a CAR19-expressing cell therapy), wherein said neurotoxicity risk status comprises a measure of one, two, three, four, five, six, seven, eight or more (all) of the following:

(a-i) the level or activity of soluble CD30 (sCD30);

(a-ii) the level or activity of soluble tumor necrosis factor receptor-1 (sTNFR-1);

(a-iii) the level or activity of interleukin 2 (IL-2);

(a-iv) the level or activity of soluble interleukin 4 receptor (sIL-4R),

(a-v) the level or activity of hepatocyte growth factor (HGF), or

(a-vi) the level or activity of interleukin 15 (IL-15);

(a-vii) the level or activity of sRAGE;

(a-viii) the level or activity of VEGFR-1; or

(a-ix) the level or activity of VEGFR-2,

wherein the measure is acquired from a sample, e.g., a serum sample, blood sample, CSF sample or brain parenchyma sample, from the subject, and wherein the neurotoxicity risk status is indicative of the subject's risk for developing neurotoxicity, e.g., severe neurotoxicity,

thereby evaluating the subject's risk for developing neurotoxicity.

In some embodiments, the measure of any one or all of (a-i)-(a-ix) is compared to a measure obtained from a subject not predicted to be at risk of developing neurotoxicity.

In some embodiments, the measure of any one or all of (a-i)-(a-ix) is acquired within 3 days, e.g., within 1 day, 2 days or 3 days, post CAR expressing cell infusion.

In some embodiments, a decrease in (a-i) is indicative of the subject's risk for developing neurotoxicity, e.g., severe neurotoxicity.

In some embodiments, an increase in (ii) is indicative of the subject's risk for developing neurotoxicity, e.g., severe neurotoxicity.

In some embodiments, a difference in any one or all of (a-i)-(a-ix) compared to a corresponding measure of any one or all of (a-i)-(a-ix) obtained from a subject not predicted to be at risk of developing neurotoxicity, is indicative of the subject's risk of developing neurotoxicity.

In some embodiments, the risk status comprises a measure of (a-i)

In some embodiments, the risk status comprises a measure of (a-i) and (a-ii). In some embodiments, a subject with a decrease in (a-i) and an increase in (a-ii), is predicted to be at risk of developing neurotoxicity. In some embodiments, a measure of (a-i) or (a-ii) is acquired within 3 days, e.g., within 1 day, 2 days or 3 days, post CAR expressing cell infusion. In some embodiments, the subject predicted to be at risk of developing neurotoxicity is administered an agent that targets the TNF pathway, e.g., an inhibitor of TNFα, e.g., an anti-TNFα antibody (e.g., infliximab) or a soluble TNFα receptor (e.g., etanercept).

In some embodiments, the subject predicted to be at risk of developing neurotoxicity is administered an agent other than a CRS therapy, e.g., as described herein.

In an aspect, disclosed herein is a method of treating, e.g., preventing, neurotoxicity in a subject comprising administering to the subject an agent that targets the TNF pathway, e.g., as described herein. In some embodiments, the subject has been administered a CAR-expressing cell therapy, e.g., CAR19 expressing cell therapy. In an embodiment, the subject is selected for administration of an agent that targets the TNF pathway by evaluating, e.g., predicting, the subject's risk for developing neurotoxicity, e.g., as described herein, comprising:

acquiring a neurotoxicity risk status for the subject, e.g., in response to a CAR-expressing cell therapy (e.g., a CAR19-expressing cell therapy), wherein said neurotoxicity risk status comprises a measure of one, two, three, four, five, six, seven, eight or more (all) of the following:

(a-i) the level or activity of soluble CD30 (sCD30);

(a-ii) the level or activity of soluble tumor necrosis factor receptor-1 (sTNFR-1);

(a-iii) the level or activity of interleukin 2 (IL-2);

(a-iv) the level or activity of soluble interleukin 4 receptor (sIL-4R),

(a-v) the level or activity of hepatocyte growth factor (HGF), or

(a-vi) the level or activity of interleukin 15 (IL-15);

(a-vii) the level or activity of sRAGE;

(a-viii) the level or activity of VEGFR-1; or

(a-ix) the level or activity of VEGFR-2,

In some embodiments, the subject is a pediatric subject or a young adult.

In some embodiments, the subject has previously been administered a CAR-expressing cell therapy, e.g., a CAR19 expressing cell therapy, e.g., CTL019.

In some embodiments, the neurotoxicity, e.g., as described herein, is associated with a CAR-expressing cell therapy. In some embodiments, the CRS is associated with a CAR-expressing cell therapy

In some embodiments, the subject was administered the CAR-expressing cell therapy, e.g., about 30 days, e.g., 29, 28, 27, 26, 25, 24, 23, 22, 21, 20, 19, 18, 17, 16, 15, 14, 13, 12, 11, 10, 9, 8, 7, 6, 5, 4, 3, 2, or 1 days, prior to development, e.g., appearance, of neurotoxicity. In some embodiments, the subject is evaluated, about 30 days, e.g., 29, 28, 27, 26, 25, 24, 23, 22, 21, 20, 19, 18, 17, 16, 15, 14, 13, 12, 11, 10, 9, 8, 7, 6, 5, 4, 3, 2, or 1 days after administration of CAR-expressing cell therapy.

In some embodiments, the subject at risk of developing neurotoxicity is also at risk of developing CRS. In some embodiments, development of neurotoxicity, e.g., severe neurotoxicity, in the subject is correlated with development of CRS, e.g., high grade CRS.

In some embodiments, the subject at risk of developing neurotoxicity is not at risk of developing CRS. In some embodiments, development of neurotoxicity in the subject is not correlated with development of CRS. In some embodiments, CRS therapies e.g., as described herein, do not, e.g., reduce the symptoms of neurotoxicity.

In some embodiments, neurotoxicity includes, but is not limited to encephalopathy (e.g., as described herein), focal deficit (e.g., as described herein), seizure (e.g., as described herein), or other clearly defined neurologic symptom. In some embodiments, neurotoxicity comprises severe neurotoxicity. In some embodiments, severe neurotoxicity comprises any one or all of focal neurotoxicity, encephalopathy, or seizure.

In some embodiments, neurotoxicity does not include headache or delirium.

In some embodiments, neurotoxicity in a pediatric subject or a young adult, can be associated with a predisposition to developing neurotoxicity, e.g., a vulnerability, e.g., a prior injury. In some embodiments, neurotoxicity can be associated with, e.g., neurologic deficit.

In some embodiments, neurotoxicity in a pediatric subject or a young adult is different from neurotoxicity in an adult. In some embodiments neurotoxicity in a pediatric subject or a young adult does not include, e.g., aphasia or cerebral edema. Without wishing to be bound by theory, it is believed that in some embodiments, there may be different causes for the development of neurotoxicity from CAR-therapies in the developing pediatric brain compared to adults.

In some embodiments, development of neurotoxicity following CAR therapy can depend on the age of the subject.

In some embodiments, the level and or activity of soluble tumor necrosis factor receptor-1 (sTNFR-1) is higher in a subject who develops neurotoxicity, e.g., encephalopathy, compared to a subject who does not develop neurotoxicity, e.g., encephalopathy. In some embodiments, subjects who develop neurotoxicity, e.g., encephalopathy, have a higher 35-day peak cytokine level compared to subjects who do not develop neurotoxicity.

In some embodiments, a subject who develops neurotoxicity, e.g., as described herein, has a higher level of one or more of the following cytokines: interleukin 2 (IL-2), soluble interleukin 4 receptor (sIL-4R), hepatocyte growth factor (HGF), and interleukin 15 (IL-15), as compared to a subject who does not develop neurotoxicity. In some embodiments, the subject does not develop CRS.

In an aspect the disclosure provides a method of evaluating, e.g., predicting, a subject's risk for developing CRS, e.g., as described herein, comprising acquiring a CRS risk status for the subject, e.g., in response to a CAR-expressing cell therapy (e.g., a CAR19-expressing cell therapy), wherein said CRS risk status comprises a measure of the level or activity of soluble CD30 (sCD30), wherein the measure is acquired from a sample, e.g., a serum sample, blood sample, CSF sample or brain parenchyma sample, from the subject, and wherein the CRS risk status is indicative of the subject's risk for developing CRS, e.g., severe CRS, thereby evaluating the subject's risk for developing CRS.

In an aspect the disclosure provides a method of evaluating, e.g., predicting, a subject's risk for developing CRS and neurotoxicity, e.g., as described herein, comprising

acquiring a CRS risk status for the subject, e.g., in response to a CAR-expressing cell therapy (e.g., a CAR19-expressing cell therapy), wherein said CRS risk status comprises a measure of the level or activity of VEGF or VEGFR, wherein the measure is acquired from a sample, e.g., a blood sample, from the subject, wherein the CRS risk status is indicative of the subject's risk for developing CRS, e.g., severe CRS, and

acquiring a neurotoxicity risk status for the subject, e.g., in response to a CAR-expressing cell therapy (e.g., a CAR19-expressing cell therapy), wherein said neurotoxicity risk status comprises a measure of the level or activity of VEGF or VEGFR, wherein the measure is acquired from a sample, e.g., a blood sample, from the subject, wherein the neurotoxicity risk status is indicative of the subject's risk for developing neurotoxicity, e.g., severe neurotoxicity,

thereby evaluating the subject's risk for developing neurotoxicity and CRS.

In an aspect, the disclosure provides a method for treating a subject having a cancer, e.g., follicular lymphoma (FL), e.g., relapsed or refractory FL. In some embodiments, the method comprises administering to the subject a CAR expressing cell therapy, e.g., CAR19 expressing cell therapy, thereby treating the subject.

In some embodiments, the subject is an adult.

In some embodiments, the subject is administered about 0.6-6.0×10⁸ CAR expressing cells (e.g., 0.6-6.0×10⁸ CAR expressing cells), e.g., CAR19 expressing cells, e.g., in a single infusion.

In some embodiments, the subject is administered a lymhodepleting chemotherapy, e.g., a lymphodepletion regimen as described herein, prior to administration of the CAR-expressing cell, e.g., CAR19 expressing cell.

In a related aspect, the disclosure provides a composition for use comprising a CAR expressing cell therapy, e.g., CAR19 expressing cell therapy, in the treatment of a subject having a cancer, e.g., follicular lymphoma (FL), e.g., relapsed or refractory FL.

In some embodiments, the subject is an adult.

In some embodiments, the subject is about 0.6-6.0×10⁸ CAR expressing cells (e.g., 0.6-6.0×10⁸ CAR expressing cells), e.g., CAR19 expressing cells, e.g., in a single infusion.

In some embodiments, the subject is administered a lymhodepleting chemotherapy, e.g., a lymphodepletion regimen as described herein, prior to administration of the CAR-expressing cell, e.g., CAR19 expressing cell.

Additional features and embodiments of the present invention include one or more of the following:

In some embodiments of any of the methods, compositions for use, and kits disclosed herein, the CAR-expressing cell therapy comprises a plurality (e.g., a population) of CAR-expressing immune effector cells, e.g., a plurality (e.g., a population) of T cells or NK cells, or a combination thereof. In one embodiment, the CAR-expressing cell therapy is a CAR19 therapy (e.g., CTL019 therapy). In an embodiment, the CAR-expressing cell therapy comprises or consists of CTL019. In an embodiment, the CAR-expressing cell is a CTL019 product. In an embodiment, the CAR-expressing cell is a T cell, e.g., CD4+ T cell or a CD8+ T cell. In an embodiment, the CAR-expressing cell is a NK cell.

In some embodiments of any of the methods and compositions for use disclosed herein, the measure of one or more of (i)-(vi) or (a-i)-(aix) is obtained from an apheresis sample acquired from the subject. The apheresis sample can be evaluated prior to infusion or re-infusion.

In some embodiments of any of the methods and compositions for use disclosed herein, the measure of one or more of (i)-(vi) or (a-i)-(aix) is obtained from a manufactured CAR-expressing cell product sample, e.g., CAR19-expressing cell product sample (e.g., CTL019). The manufactured CAR-expressing cell product can be evaluated prior to infusion or re-infusion.

In some embodiments of any of the methods and compositions for use disclosed herein, the subject is evaluated prior to receiving, during, or after receiving, the CAR-expressing cell therapy.

In some embodiments of any of the methods and compositions for use disclosed herein, the hematological cancer is an ALL, CLL, DLBCL, e.g., relapsed or refractory DLBCL, or FL, e.g., relapsed or refractory FL. The subject can be a human patient.

In some embodiments of any of the methods and compositions for use disclosed herein, the cell, e.g., the population of immune effector cells (e.g., cells expressing a CAR molecule described herein) is administered in combination with an inhibitor of an immune checkpoint molecule chosen from one or more of PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, TGF (e.g., TGF beta), or a combination thereof.

In some embodiments of any of the methods and compositions for use disclosed herein, the subject receives concurrent treatment with an agent, e.g., an mTOR inhibitor, and/or a checkpoint inhibitor. In some embodiments, the subject receives treatment with an agent, e.g., an mTOR inhibitor, and/or a checkpoint inhibitor, post-CAR-expressing cell therapy. In some embodiments, the subject receives a pre-treatment of with an agent, e.g., an mTOR inhibitor, and/or a checkpoint inhibitor, prior to the initiation of a CAR-expressing cell therapy.

In some embodiments of any of the methods and compositions for use disclosed herein, T_{REG} cell population and/or T_{REG} gene signature is decreased prior to collection of cells for manufacturing. In some embodiments, the T_{REG} cell population and/or T_{REG} gene signature is decreased prior to CAR-expressing cell (e.g., T cell, or NK cell) therapy. In some embodiments, the T_{REG} cell population and/or T_{REG} gene signature is decreased by administration of cyclophosphamide, anti-GITR antibody, an mTOR inhibitor, or a combination thereof.

In some embodiments of any of the methods and compositions for use disclosed herein the value of responder or relapser status comprises a measure of a combination of a gene signature and a biomarker. In some embodiments, the value of the responder or relapser status comprises a measure of a CD19 CAR-expressing cell gene set signature and a combination of one or more of: a biomarker including but not limited to CD27, CD45RO, CCR7, HLA-DR, CD127, or CD95.

In some embodiments of any of the methods and compositions for use disclosed herein, the method further comprises identifying the subject as a responder (e.g., a complete or partial responder), a non-responder, a relapser or a non-relapser, based on a measure of one or more of (i)-(vi) or (a-i)-(aix).

In some embodiments of any of the methods and compositions for use disclosed herein, the measure of one or more of (i)-(vi) or (a-i)-(aix) evaluates a profile for one or more of gene expression, flow cytometry or protein expression.

In some embodiments of any of the methods and compositions for use disclosed herein, the expression profile includes one or more gene signatures based on mRNA expression levels of selected genes obtained from the apheresis sample or a manufactured CD19 CAR-expressing cell product (e.g., CTL019). In one embodiment, the expression profile includes one, two, three, four, five, or more of a biomarker including but not limited to CD27, CD45RO, CCR7, HLA-DR, CD127, or CD95, or a CD19 CAR-expressing cell gene set signature.

In some embodiments of any of the methods and compositions for use disclosed herein, the level or activity of a CD8+ T cell is evaluated using a profile or signature indicative of the percentage of CD8+ T cell in the sample.

In some embodiments of any of the methods and compositions for use disclosed herein, the level or activity of a CD4+ T cell is evaluated using a profile or signature indicative of the percentage of CD4+ T cell in the sample.

In some embodiments of any of the methods and compositions for use disclosed herein, the level or activity of CD27+CD45RO− immune effector cells is evaluated using a profile or signature indicative of the percentage of CD27+CD45RO− immune effector cells in the sample.

In some embodiments of any of the methods and compositions for use disclosed herein, the level or activity of CD27+CD45RO− CCR7+ immune effector cells is evaluated using a profile or signature indicative of the percentage of CD27+CD45RO− CCR7+ immune effector cells in the sample.

In some embodiments of any of the methods and compositions for use disclosed herein, the level or activity of CCR7+ HLA-DR− immune effector cells is evaluated using a profile or signature indicative of the percentage of CCR7+ HLA-DR− immune effector cells in the sample.

In some embodiments of any of the methods and compositions for use disclosed herein, the level or activity of CD27+CD45RO− CD127+ immune effector cells is evaluated using a profile or signature indicative of the percentage of CD27+CD45RO− CD127+ immune effector cells in the sample.

In some embodiments of any of the methods and compositions for use disclosed herein, the level or activity of CD27+ CCR7− CD95+CD127+ immune effector cells is evaluated using a profile or signature indicative of the percentage of CD27+ CCR7− CD95+CD127+ immune effector cells in the sample.

In some embodiments of any of the methods and compositions for use disclosed herein, the level or activity of CD27+ CCR7− CD95+ immune effector cells is evaluated using a profile or signature indicative of the percentage of CD27+ CCR7− CD95+ immune effector cells in the sample.

In some embodiments of any of the methods and compositions for use disclosed herein, the level or activity of CD27+CD45RO− CD95+ immune effector cells is evaluated using a profile or signature indicative of the percentage of CD27+CD45RO− CD95+ immune effector cells in the sample.

In some embodiments of any of the methods and compositions for use disclosed herein, the level or activity of CCR7+CD45RO− immune effector cells is evaluated using a profile or signature indicative of the percentage of CCR7+CD45RO− immune effector cells in the sample.

In some embodiments of any of the methods and compositions for use disclosed herein, the level or activity of CD45RO− CCR7+CD127− immune effector cells is evaluated using a profile or signature indicative of the percentage of CD45RO− CCR7+CD127− immune effector cells in the sample.

In some embodiments of any of the methods and compositions for use disclosed herein, the level or activity of CD27+CD45RO− CCR7+CD127+ immune effector cells is evaluated using a profile or signature indicative of the percentage of CD27+CD45RO− CCR7+CD127+ immune effector cells in the sample.

In some embodiments of any of the methods and compositions for use disclosed herein, the level or activity of CD45RO− CD127+ immune effector cells is evaluated using a profile or signature indicative of the percentage of CD45RO− CD127+ immune effector cells in the sample.

In some embodiments of any of the methods and compositions for use disclosed herein, the level or activity of one, two, three, four, five, or more of a biomarker chosen from CD27, CD45RO, CCR7, HLA-DR, CD127, or CD95, predicts a subject's response to a CAR19+ cell product (e.g., CTL019).

In some embodiments of any of the methods and compositions for use disclosed herein, the value of responder or relapser status comprises a measure of the level or activity of one, two, three, four, or more (e.g., all) of the biomarkers disclosed herein having a given FDR p-value, listed herein, e.g., in a Figure herein, e.g., FIG. 1. In some embodiments, the FDR p-value is below 0.2, 0.1, 0.05, 0.02, 0.01, 0.005, 0.002, or 0.001. In some embodiments, the FDR p-value is below 0.1 or 0.01. In some embodiments, the biomarkers include but are not limited to CD27, CD45RO, CCR7, HLA-DR, CD127, or CD95, or a combination thereof. In some embodiments, the measure comprises a measure of all of the biomarkers, e.g., all of CD27, CD45RO, CCR7, HLA-DR, CD127, or CD95, that have a p-value below a threshold of 0.2, 0.1, 0.05, 0.02, 0.01, 0.005, 0.002, or 0.001. In some embodiments, the measure comprises a measure of all of the biomarkers having a p-value below the threshold. In some embodiments, the measure comprises a measure of one, two, three, four, five, or more biomarkers having a p-value below the threshold. In some embodiments, the measure comprises a measure of at least one, two, three, four, five, or more biomarkers having a p-value below the threshold.

In some embodiments of any of the methods and compositions for use disclosed herein, the biomarker is a secreted or a cell surface biomarker. For example the biomarker can be measured by flow cytometry.

In some embodiments of any of the methods and compositions for use disclosed herein, a responder (e.g., a complete responder) has, or is identified as having, a greater level or activity of one, two, or more (all) of biomarkers associated with naïve T cells, e.g., as described herein, as compared to a non-responder.

In some embodiments of any of the methods and compositions for use disclosed herein, a non-responder has, or is identified as having, a greater level or activity of one, two, three, four, five, six, seven, or more (e.g., all) of biomarkers associated with, effector T cells (e.g., as described herein), or regulatory T cells (e.g., as described herein), as compared to a responder.

In some embodiments of any of the methods and compositions for use disclosed herein, a complete responder has, or is identified as having, a greater, e.g., a statistically significant greater, percentage of CD8+ T cells compared to a reference value, e.g., a non-responder percentage of CD8+ T cells.

In some embodiments of any of the methods and compositions for use disclosed herein, a complete responder or a partial responder has, or is identified as having, a greater, e.g., a statistically significant greater, percentage of CD4+ T cells compared to a reference value, e.g., a non-responder percentage of CD4+ T cells.

In some embodiments of any of the methods and compositions for use disclosed herein, a complete responder has, or is identified as having, a greater percentage (e.g., 5%, 6%, 7%, 10%, 15%, 20%, 25%, 27%, 30%, 35%, or 40% or greater number) of CD27+CD45RO− immune effector cells, e.g., in the CD8+ population, compared to a reference value, e.g., a non-responder number of CD27+CD45RO− immune effector cells.

In some embodiments of any of the methods and compositions for use disclosed herein, a complete responder has, or is identified as having, a greater percentage (e.g., 5%, 6%, 7%, 10%, 15%, 20%, 25%, 27%, 30%, 35%, or 40% or greater number) of CD27+CD45RO− CCR7+ immune effector cells, e.g., in the CD8+ population, compared to a reference value, e.g., a non-responder number of CD27+CD45RO− CCR7+ immune effector cells.

In some embodiments of any of the methods and compositions for use disclosed herein, a complete responder has, or is identified as having, a greater percentage (e.g., 5%, 6%, 7%, 10%, 15%, 20%, 25%, 27%, 30%, 35%, or 40% or greater number) of CCR7+ HLA-DR− immune effector cells, e.g., in the CD8+ population, compared to a reference value, e.g., a non-responder number of CCR7+ HLA-DR− immune effector cells.

In some embodiments of any of the methods and compositions for use disclosed herein, a complete responder has, or is identified as having, a greater percentage (e.g., 5%, 6%, 7%, 10%, 15%, 20%, 25%, 27%, 30%, 35%, or 40% or greater number) of CD27+CD45RO− CD127+ immune effector cells, e.g., in the CD8+ population, compared to a reference value, e.g., a non-responder number of CD27+CD45RO− CD127+ immune effector cells.

In some embodiments of any of the methods and compositions for use disclosed herein, a complete responder has, or is identified as having, a greater percentage (e.g., 5%, 6%, 7%, 10%, 15%, 20%, 25%, 27%, 30%, 35%, or 40% or greater number) of CD27+ CCR7− CD95+CD127+ immune effector cells, e.g., in the CD8+ population, compared to a reference value, e.g., a non-responder number of CD27+ CCR7− CD95+CD127+ immune effector cells.

In some embodiments of any of the methods and compositions for use disclosed herein, a complete responder has, or is identified as having, a greater percentage (e.g., 5%, 6%, 7%, 10%, 15%, 20%, 25%, 27%, 30%, 35%, or 40% or greater number) of CD27+ CCR7− CD95+ immune effector cells, e.g., in the CD8+ population, compared to a reference value, e.g., a non-responder number of CD27+ CCR7− CD95+ immune effector cells.

In some embodiments of any of the methods and compositions for use disclosed herein, a complete responder has, or is identified as having, a greater percentage (e.g., 5%, 6%, 7%, 10%, 15%, 20%, 25%, 27%, 30%, 35%, or 40% or greater number) of CD27+CD45RO− CD95+ immune effector cells, e.g., in the CD8+ population, compared to a reference value, e.g., a non-responder number of CD27+CD45RO− CD95+ immune effector cells.

In some embodiments of any of the methods and compositions for use disclosed herein, a complete responder has, or is identified as having, a greater percentage (e.g., 5%, 6%, 7%, 10%, 15%, 20%, 25%, 27%, 30%, 35%, or 40% or greater number) of CCR7+CD45RO− immune effector cells, e.g., in the CD8+ population, compared to a reference value, e.g., a non-responder number of CCR7+CD45RO− immune effector cells.

In some embodiments of any of the methods and compositions for use disclosed herein, a complete responder has, or is identified as having, a greater percentage (e.g., 5%, 6%, 7%, 10%, 15%, 20%, 25%, 27%, 30%, 35%, or 40% or greater number) of CD45RO− CCR7+CD127− immune effector cells, e.g., in the CD8+ population, compared to a reference value, e.g., a non-responder number of CD45RO− CCR7+CD127− immune effector cells.

In some embodiments of any of the methods and compositions for use disclosed herein, a complete responder has, or is identified as having, a greater percentage (e.g., 5%, 6%, 7%, 10%, 15%, 20%, 25%, 27%, 30%, 35%, or 40% or greater number) of CD27+CD45RO− CCR7+CD127+ immune effector cells, e.g., in the CD8+ population, compared to a reference value, e.g., a non-responder number of CD27+CD45RO− CCR7+CD127+ immune effector cells.

In some embodiments of any of the methods and compositions for use disclosed herein, a complete responder has, or is identified as having, a greater percentage (e.g., 5%, 6%, 7%, 10%, 15%, 20%, 25%, 27%, 30%, 35%, or 40% or greater number) of CD45RO− CD127+ immune effector cells, e.g., in the CD8+ population, compared to a reference value, e.g., a non-responder number of CD45RO− CD127+ immune effector cells.

In some embodiments of any of the methods and compositions for use disclosed herein, a complete responder has, or is identified as having, a greater percentage (e.g., 5%, 6%, 7%, 10%, 15%, 20%, 25%, 27%, 30%, 35%, or 40% or greater number) of CCR7+ HLA-DR− immune effector cells, e.g., in the CD8+ population, compared to a reference value, e.g., a non-responder number of CCR7+ HLA-DR− immune effector cells.

In some embodiments of any of the methods and compositions for use disclosed herein, a complete responder has, or is identified as having, a greater percentage of one, two, three, or more (e.g., all) of resting T_{EFF} cells, resting T_{REG} cells, younger T cells (e.g., younger CD4 or CD8 cells, or gamma/delta T cells), or early memory T cells, or a combination thereof, compared to a reference value, e.g., a non-responder number of resting T_{EFF} cells, resting T_{REG} cells, younger T cells (e.g., younger CD4 or CD8 cells), or early memory T cells.

In some embodiments of any of the methods and compositions for use disclosed herein, a non-responder has, or is identified as having, a greater percentage of one, two, three, or more (e.g., all) of activated T_{EFF} cells, activated T_{REG} cells, older T cells (e.g., older CD4 or CD8 cells), or late memory T cells, or a combination thereof, compared to a reference value, e.g., a responder number of activated T_{EFF} cells, activated T_{REG} cells, older T cells (e.g., older CD4 or CD8 cells), or late memory T cells.

In some embodiments of any of the methods and compositions for use disclosed herein, a non-responder has, or is identified as having, a greater percentage of an immune cell exhaustion marker, e.g., one, two or more immune checkpoint inhibitors (e.g., PD-1, TIM-3 and/or LAG-3). In one embodiment, a non-responder has, or is identified as having, a greater percentage of PD-1 or LAG-3 expressing immune effector cells (e.g., CD4+ T cells and/or CD8+ T cells) (e.g., CAR-expressing CD4+ cells and/or CD8+ T cells) compared to the percentage of PD-1 or LAG-3 expressing immune effector cells from a responder.

In some embodiments of any of the methods and compositions for use disclosed herein, a non-responder has, or is identified as having, a greater percentage of immune cells having an exhausted phenotype, e.g., immune cells that co-express at least two exhaustion markers, e.g., co-expresses PD-1 and TIM-3. In other embodiments, a non-responder has, or is identified as having, a greater percentage of immune cells having an exhausted phenotype, e.g., immune cells that co-express at least two exhaustion markers, e.g., co-expresses PD-1 and LAG-3.

In some embodiments of any of the methods and compositions for use disclosed herein, a non-responder has, or is identified as having, a greater percentage of PD-1+/LAG-3+ cells in the CAR-expressing cell population (e.g., a CAR19+ cell population) compared to a responder (e.g., a complete responder) to the CAR-expressing cell therapy.

In some embodiments of any of the methods and compositions for use disclosed herein, the presence of:

CD27+CD45RO− CD8+ T cells,

CD27+CD45RO− CCR7+CD8+ T cells,

CD27+CD45RO− CD127+CD8+ T cells,

CD27+ CCR7− CD95+CD8+ T cells,

CD27+CD45RO− CD95+CD8+ T cells,

CCR7+CD45RO− CD8+ T cells,

CD45RO− CCR7+CD127-CD8+ T cells,

CD27+CD45RO− CCR7+CD127+CD8+ T cells,

CD45RO− CD127+CD8+ T cells,

CCR7+ HLA-DR− CD8+ T cells, and/or

CD27+ CCR7− CD95+CD127+CD8+ T cells,

in an apheresis sample; is a positive predictor of the subject response to a CAR-expressing cell therapy (e.g., a CAR19 therapy (e.g., CTL019 therapy)).

In an embodiment, any of the methods and compositions for use disclosed herein can be used prior to administration of a CAR-expressing cell therapy. In some embodiments, provided methods can be used before, at the same time, or during course of a CAR-expressing cell therapy.

In an embodiment, any of the methods and compositions for use disclosed herein can be used to identify a subject having cancer, e.g., a hematological cancer such as, e.g., CLL or ALL, as having an increased or a decreased likelihood to respond to a treatment that comprises a CAR-expressing cell (e.g., T cell, NK cell) therapy, e.g., a CD19 CAR-expressing cell therapy. The method comprises: (1) acquiring a sample from the subject (e.g., an apheresis sample obtained from the blood of the subject; and/or e.g., a manufactured product sample, e.g., genetically engineered T cells); (2) determining a level (e.g., amount or activity) of one or more biomarkers described herein in the sample; and (3) (optionally) comparing the determined level of the one or more markers to a reference level; and (4) identifying the subject as a complete responder, partial responder, non-responder, a relapser or non-relapser to the CAR-expressing cell therapy. In embodiments, a difference, e.g., a statistically significant difference, between the determined level compared to a reference level is predictive of the subjects responsiveness to the CAR-expressing cell therapy.

In some embodiments of any of the methods and compositions for use disclosed herein, the CAR-expressing cell therapy comprises CTL019.

In some embodiments of any of the methods and compositions for use disclosed herein, the CAR-expressing cell comprises a nucleic acid encoding a CAR, e.g., a CAR molecule described herein, e.g., a CD19 CAR described herein (e.g., CTL019).

In some embodiments, any of the methods and compositions for use disclosed herein, further comprise selecting the subject for the CAR-expressing therapy.

In some embodiments of any of the methods and compositions for use disclosed herein, the subject has a disease associated with expression of a tumor- or cancer associated-antigen. In some embodiments, the disease associated with expression of a tumor- or cancer associated-antigen is a hyperproliferative disorder, e.g., a cancer, e.g., a hematological cancer or a solid tumor. In some embodiments, the hematological cancer is chosen from one or more of: a B-cell acute lymphocytic leukemia (B-ALL), T-cell acute lymphocytic leukemia (T-ALL), acute lymphocytic leukemia (ALL), chronic myelogenous leukemia (CIVIL), chronic lymphocytic leukemia (CLL), B cell promyelocytic leukemia, blastic plasmacytoid dendritic cell neoplasm, Burkitt lymphoma, diffuse large B cell lymphoma (DLBCL), follicular lymphoma (FL), hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin's lymphoma (NHL), Hodgkin's lymphoma (HL), plasmablastic lymphoma, plasmacytoid dendritic cell neoplasm, and Waldenstrom macroglobulinemia. In some embodiments, the cancer is a relapsed or refractory cancer, e.g, a relapsed or refractory cancer described herein.

In some embodiments, the hematological cancer is a leukemia (e.g., CLL, or ALL), or a lymphoma (e.g., DLBCL, FL, MCL, NHL, or HL). In some embodiments, the hematological cancer is CLL. In some embodiments, the hematological cancer is DLBCL, e.g., relapsed or refractory DLBCL. In some embodiments, the hematological cancer is FL, e.g., relapsed or refractory FL.

In some embodiments of any of the methods and compositions for use disclosed herein, the immune effector cell population is acquired from a subject, e.g., wherein acquisition occurs prior to, or after administration of chemotherapy, e.g., a lymphodepleting regimen, to the subject.

In some embodiments of any of the methods and compositions for use disclosed herein, the chemotherapy, e.g., cycle of chemotherapy, comprises one or more of an induction, a consolidation, an interim maintenance, a delayed intensification, or a maintenance therapy cycle.

In some embodiments of any of the methods and compositions for use disclosed herein, the immune effector cell population is acquired from the subject before the subject has been administered a lymphodepleting regimen, e.g., cyclophosphamide, fludarabine, bendamustine, or a combination thereof.

In some embodiments of any of the methods and compositions for use disclosed herein, the CAR-expressing cell therapy comprises a plurality of CAR-expressing immune effector cells.

In some embodiments of any of the methods and compositions for use disclosed herein, the value of one or more of (i)-(vi) or (a-i)-(aix) is obtained from an apheresis sample acquired from the subject, wherein optionally the apheresis sample is evaluated prior to infusion or re-infusion, or after infusion.

In some embodiments of any of the methods and compositions for use disclosed herein, the value of one or more of (i)-(vi) or (a-i)-(aix) is obtained from a manufactured CAR-expressing cell product sample, e.g., CAR19-expressing cell product sample (e.g., CTL019), wherein optionally the manufactured CAR-expressing cell product is evaluated prior to infusion or re-infusion, or after infusion.

In some embodiments of any of the methods and compositions for use disclosed herein, the subject is evaluated prior to, during, or after receiving the CAR-expressing cell therapy.

In some embodiments of any of the methods and compositions for use disclosed herein, the immune effector cell population comprises a higher number of less differentiated T cells, e.g., a higher number of one or more of naïve T cells, stem central memory T cells, and/or central memory T cells, e.g., compared to a reference value (e.g., a sample from the subject at a later time point or after exposure to additional rounds of chemotherapy). IN some embodiments, the naïve T cells are identified based upon an expression pattern of CCR7+, CD62L+, CD45RO−, CD95−; the stem central memory T cells are identified based upon an expression pattern of CCR7+, CD62L+, CD45RO−, CD95+; and the central memory T cells are identified based upon an expression pattern of CCR7+, CD62L+, CD45RO+, CD95+.

In some embodiments of any of the methods and compositions for use disclosed herein, the immune effector cell population is selected based upon the expression of one or more markers, e.g., CCR7, CD62L, CD45RO, and CD95, e.g., the population of immune effector cells (e.g., T cells) are CCR7+ and CD62L+.

In some embodiments, any of the methods and compositions for use disclosed herein, further comprise removing T regulatory cells, e.g., CD25+ T cells, from the acquired immune cell population, to thereby provide a population of T regulatory-depleted cells, e.g., CD25+ depleted cells.

In some embodiments of any of the methods and compositions for use disclosed herein, the immune effector cell population has been selected based upon the expression of one or more markers, e.g., CD3, CD28, CD4, CD8, CD45RA, and CD45RO, e.g., the provided population of immune effector cells (e.g., T cells) are CD3+ and/or CD28+.

In other embodiments, the population of cells is expanded in an appropriate media that includes one or more interleukin that result in at least a 200-fold, 250-fold, 300-fold, or 350-fold increase in cells over a 14 day expansion period, as measured by flow cytometry.

In other embodiments, the population of cells is cultured, e.g., expanded, in the presence IL-2, IL-15, (e.g, hetIL-15), IL-7, or any combination thereof.

In certain embodiments, the method further includes cryopreserving the population of the cells after the appropriate expansion period.

In yet other embodiments, the method of making disclosed herein further comprises contacting the population of immune effector cells with a nucleic acid encoding a telomerase subunit, e.g., hTERT. The nucleic acid encoding the telomerase subunit can be DNA.

In yet other embodiments, the method of making disclosed herein further comprises culturing the population of immune effector cells in serum comprising 2% hAB serum.

In any of the methods, and kits described herein, the CD19 CAR can comprise an anti-CD19 binding domain described in Table 12, or CDRs, e.g., one or more (e.g., all) of HC CDR1, HC CDR2, HC CDR3, LC CDR1, LC CDR2, and LC CDR3 of an anti-CD19 binding domain described in Table 12. In an embodiment, the CAR can comprise one of more of: a leader sequence, e.g., a leader sequence described herein, e.g., in Table 1; an anti-CD19 binding domain, e.g., an anti-CD19 binding domain described herein, e.g., in Table 12; a hinge region, e.g., a hinge region described herein, e.g., a hinge region described in Table 1; a transmembrane domain, e.g., a transmembrane domain described herein, e.g., in Table 1; and an intracellular signaling domain (e.g., a costimulatory domain and/or a primary signaling domain, e.g., a costimulatory domain described herein, e.g., in Table 1 and/or a primary signaling domain described herein, e.g., in Table 1). In an embodiment, the CD19 CAR-expressing cell (e.g., T cell, NK cell) is CTL019 or a CD19 CAR described in Table 13.

Although methods and materials similar or equivalent to those described herein can be used in the practice or testing of the present invention, suitable methods and materials are described below. All publications, patent applications, patents, and other references (e.g., sequence database reference numbers) mentioned herein are incorporated by reference in their entirety. When one gene or protein references a plurality of sequence accession numbers, all of the sequence variants are encompassed.

In addition, the materials, methods, and examples are illustrative only and not intended to be limiting. Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the invention will be apparent from the description and drawings, and from the claims.

### BRIEF DESCRIPTION OF THE DRAWINGS

FIG. 1 depicts a table summarizing exemplary data obtained from biomarker assessment of 31 CLL patient samples who received at least one dose of CART therapy.

FIG. 2 depicts a boxplot showing percentage of CD27-CD45RO+ cells in complete responders (CR) vs non-responder (NR).

FIG. 3 depicts a boxplot showing percentage of CD27+CD45RO− cells in complete responders (CR) vs non-responder (NR).

FIG. 4 is a graph depicting principal component analysis results of biomarker data obtained from 31 CLL patient samples.

FIG. 5 depicts a schematic of Neurotoxicity Classification. Electronic medical records were abstracted for the following terms to define overall neurotoxicity and the specified subgroups. Severe neurotoxicity was defined by encephalopathy, aphasia or seizures

FIGS. 6A-6C show the correlation between CRS and Neurotoxicity. FIG. 6A shows that CRS was common in this pediatric population, occurring in 47/51 (92%) of subjects. Most cases of CRS were grades 2-4, no deaths were observed (CRS grade 5). FIG. 6B is a graph showing the distribution of CRS score by neurotoxicity. FIG. 6C shows the Incidence of Neurotoxicity as being Positively Correlated with Increasing CRS grade.

FIGS. 7A-7B show the 35-day Peak Cytokines and Neurotoxicity. FIG. 7A shows that select 35-day peak cytokines were significantly and specifically elevated in subjects with neurotoxicity. FIG. 7B shows that the cytokines were not elevated for all sub types of neurotoxicity.

FIG. 8 shows Cytokines predictive of neurotoxicity using 3 day peak data. The table shows cytokines that were confirmed in the forward selection predicting modeling.

FIG. 9 depicts a schematic for tree modeling for severe neurotoxicity.

FIG. 10 is a graph showing the percentage of PD-L1 cells (out of total cells) by response.

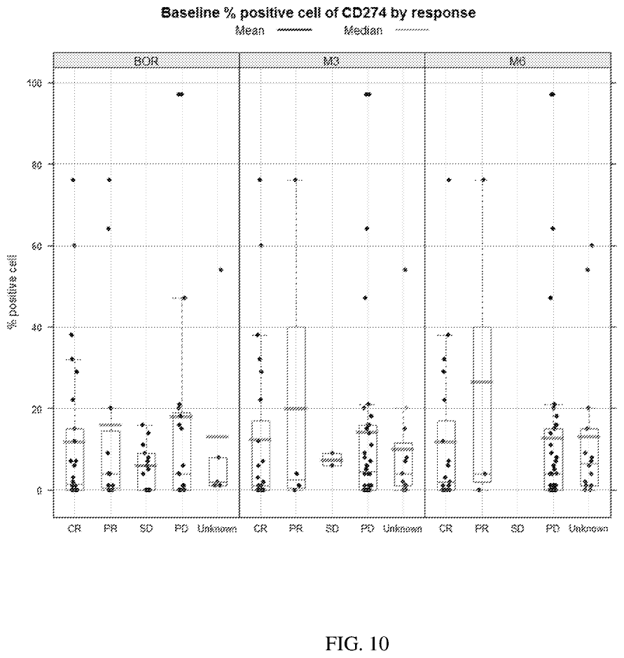

FIG. 11 is a graph showing percentage of PD1+ cells (out of total cells) by response. Areas analyzed were selected based on highest expression of PD-L1.

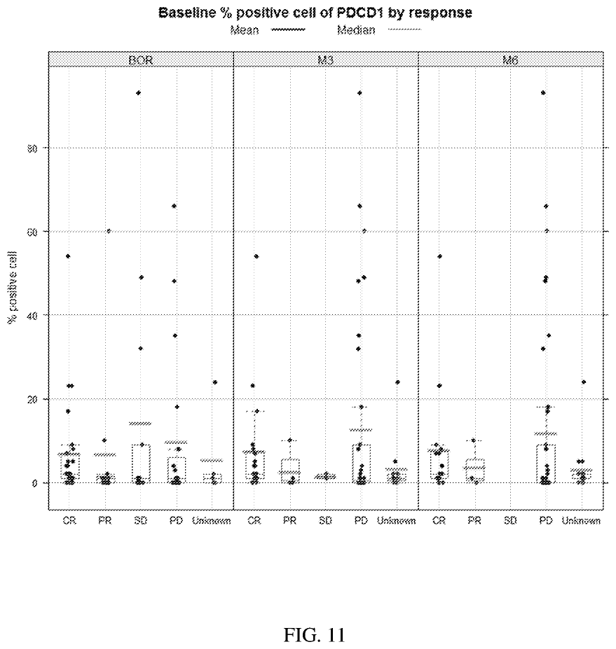

FIG. 12 is a graph showing the proportion (%) of T cells that are PD1+(in areas with highest expression of PD-L1.

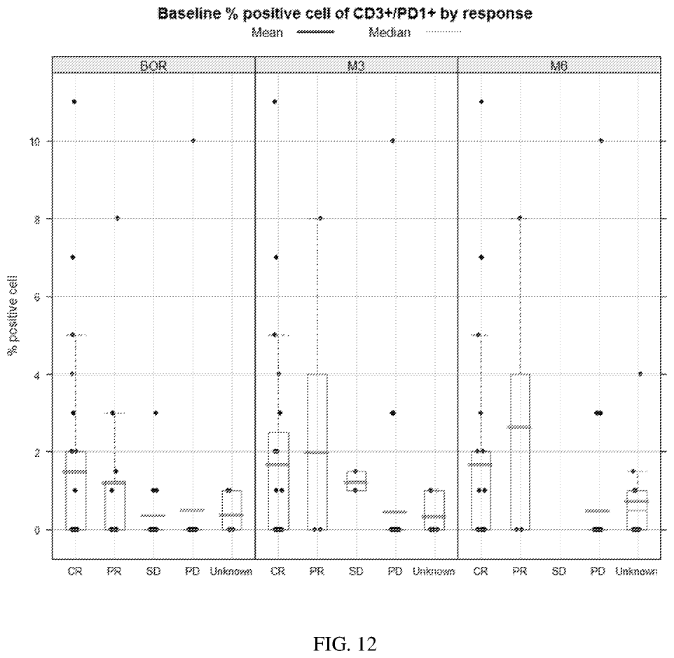

FIG. 13 is a graph depicting PD1/PD-L1 interaction scores by response. The data is plotted with AQUA scores on the y-axis and patient status, i.e., CR, PR, SD, PD or unknown, on the x-axis. The data points are divided by best overall response (BOR), response at 3 months (M3) and response at 6 months (M6).

FIG. 14 is a graph showing relative CD19 expression levels by best overall response (BOR). The data is plotted with AQUA scores on the y-axis and patient status, i.e., CR, PR, SD, PD or unknown, on the x-axis.

FIG. 15 is a schematic showing the study design for the clinical trial described in Example 4.

### DEFINITIONS

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

“Acquire” or “acquiring” as the terms are used herein, refer to obtaining possession of a physical entity (e.g., a sample, a polypeptide, a nucleic acid, or a sequence), or a value, e.g., a numerical value, by “directly acquiring” or “indirectly acquiring” the physical entity or value. “Directly acquiring” means performing a process (e.g., performing a synthetic or analytical method) to obtain the physical entity or value. “Indirectly acquiring” refers to receiving the physical entity or value from another party or source (e.g., a third party laboratory that directly acquired the physical entity or value). Directly acquiring a physical entity includes performing a process that includes a physical change in a physical substance, e.g., a starting material. Exemplary changes include making a physical entity from two or more starting materials, shearing or fragmenting a substance, separating or purifying a substance, combining two or more separate entities into a mixture, performing a chemical reaction that includes breaking or forming a covalent or non-covalent bond. Directly acquiring a value includes performing a process that includes a physical change in a sample or another substance, e.g., performing an analytical process which includes a physical change in a substance, e.g., a sample, analyte, or reagent (sometimes referred to herein as “physical analysis”), performing an analytical method, e.g., a method which includes one or more of the following: separating or purifying a substance, e.g., an analyte, or a fragment or other derivative thereof, from another substance; combining an analyte, or fragment or other derivative thereof, with another substance, e.g., a buffer, solvent, or reactant; or changing the structure of an analyte, or a fragment or other derivative thereof, e.g., by breaking or forming a covalent or non-covalent bond, between a first and a second atom of the analyte; or by changing the structure of a reagent, or a fragment or other derivative thereof, e.g., by breaking or forming a covalent or non-covalent bond, between a first and a second atom of the reagent.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “altered level of expression” of a biomarker as described herein (e.g., a biomarker including but not limited to CD27, CD45RO, CCR7, CD95, HLA-DR and CD127, and a CD19 CAR-expressing cell (e.g., T cell, NK cell) gene signature) refers to an increase (or decrease) in the expression level of a marker in a test sample, such as a sample derived from a patient suffering from cancer (e.g., a hematological cancer such as ALL and CLL) that is greater or less than the standard error of the assay employed to assess expression. In embodiments, the alteration can be at least twice, at least twice three, at least twice four, at least twice five, or at least twice ten or more times greater than or less than the expression level of the biomarkers in a control sample (e.g., a sample from a healthy subject not having the associated disease), or the average expression level in several control samples. An “altered level of expression” can be determined at the protein or nucleic acid (e.g., mRNA) level.

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab, F(ab)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3)(see U.S. Pat. No. 6,703,199, which describes fibronectin polypeptide minibodies). The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (□) and lambda (λ) light chains refer to the two major antibody light chain isotypes.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “apheresis” as used herein refers to an extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “autologous” refers to any material derived from the same individual to whom it is later to be re-introduced into the individual.

A “biomarker” or “marker” is a gene, mRNA, or protein that undergoes alterations in expression that are associated with progression of cancer (e.g., a hematological cancer such as ALL and CLL) or responsiveness to treatment. The alteration can be in amount and/or activity in a biological sample (e.g., a blood, plasma, or a serum sample) obtained from a subject having cancer, as compared to its amount and/or activity, in a sample obtained from a baseline or prior value for the subject, the subject at a different time interval, an average or median value for a cancer patient population, a healthy control, or a healthy subject population (e.g., a control); such alterations in expression and/or activity are associated with of the responsiveness of a subject having a cancer disease state (e.g., a hematological cancer such as ALL and CLL) to a CAR-expressing cell (e.g., a CAR-expressing immune effector cell (e.g., a CAR-expressing T cell, NK cell) therapy, e.g., a CD19 CAR-expressing cell therapy. For example, a marker of the invention which is predictive of responsiveness to therapeutics can have an altered expression level, protein level, or protein activity, in a biological sample obtained from a subject having, or suspected of having, cancer as compared to a biological sample obtained from a control subject.

The term “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Cancers include, but are not limited to, B-cell acute lymphocytic leukemia (B-ALL), T-cell acute lymphocytic leukemia (T-ALL), acute lymphocytic leukemia (ALL), chronic myelogenous leukemia (CML), chronic lymphocytic leukemia (CLL), B cell promyelocytic leukemia, blastic plasmacytoid dendritic cell neoplasm, Burkitt lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, and Waldenstrom macroglobulinemia. In an embodiment, the cancer is associated with CD19 expression. The terms “tumor” and “cancer” are used interchangeably herein, e.g., both terms encompass solid and liquid tumors. As used herein, the term “cancer” or “tumor” includes premalignant, as well as malignant cancers and tumors.

The terms “cancer associated antigen” or “tumor antigen” interchangeably refers to a molecule (typically protein, carbohydrate or lipid) that is preferentially expressed on the surface of a cancer cell, either entirely or as a fragment (e.g., MHC/peptide), in comparison to a normal cell, and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a cancer-associated antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a cancer-associated antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

As used herein, the term “CD19” refers to the Cluster of Differentiation 19 protein, which is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM_001178098. As used herein, “CD19” includes proteins comprising mutations, e.g., point mutations, fragments, insertions, deletions and splice variants of full length wild-type CD19. CD19 is expressed on most B lineage cancers, including, e.g., acute lymphoblastic leukemia, chronic lymphocyte leukemia and non-Hodgkin lymphoma. Other cells which express CD19 are provided below in the definition of “disease associated with expression of CD19.” It is also an early marker of B cell progenitors. See, e.g., Nicholson et al., MOL. IMMUN. 34 (16-17): 1157-1165 (1997). In one aspect the antigen-binding portion of the CAR-expressing cell (e.g., T cell, NK cell) recognizes and binds an antigen within the extracellular domain of the CD19 protein. In one aspect, the CD19 protein is expressed on a cancer cell. In one embodiment, the CD19 has a wild-type sequence, e.g., a wild-type human sequence. In another embodiment, the CD19 has a mutant sequence, e.g., a mutant human sequence.

The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain (e.g., comprise a chimeric fusion protein). In some embodiments, the set of polypeptides are not contiguous with each other, e.g., are in different polypeptide chains. In some aspects, the set of polypeptides include a dimerization switch that, upon the presence of a dimerization molecule, can couple the polypeptides to one another, e.g., can couple an antigen binding domain to an intracellular signaling domain. In one aspect, the stimulatory molecule of the CAR is the zeta chain associated with the T cell receptor complex. In one aspect, the cytoplasmic signaling domain comprises a primary signaling domain (e.g., a primary signaling domain of CD3-zeta). In one aspect, the cytoplasmic signaling domain further comprises one or more functional signaling domains derived from at least one costimulatory molecule as defined below. In one aspect, the costimulatory molecule is chosen from 4-1BB (i.e., CD137), CD27, ICOS, and/or CD28. In one aspect, the CAR comprises a chimeric fusion protein comprising an extracellular antigen recognition domain, a transmembrane domain and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., a scFv) during cellular processing and localization of the CAR to the cellular membrane. In an embodiment, the CAR is CTL019.

The portion of the CAR composition comprising an antibody or antibody fragment thereof may exist in a variety of forms where the antigen binding domain is expressed as part of a contiguous polypeptide chain including, for example, a single domain antibody fragment (sdAb), a single chain antibody (scFv) and a humanized antibody (Harlow et al., 1999, In: USING ANTIBODIES: A LABORATORY MANUAL, COLD SPRING HARBOR LABORATORY PRESS, NY; Harlow et al., 1989, In: ANTIBODIES: A LABORATORY MANUAL, Cold Spring Harbor, N.Y.; Houston et al., 1988, PROC. NATL. ACAD. SCI. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises a scFv.

As used herein, the term “binding domain” or “antibody molecule” refers to a protein, e.g., an immunoglobulin chain or fragment thereof, comprising at least one immunoglobulin variable domain sequence. The term “binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The portion of the CAR of the invention comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises a scFv.

The phrase “disease associated with expression of CD19” includes, but is not limited to, a disease associated with expression of CD19 (e.g., wild type or mutant CD19) or condition associated with cells which express, or at any time expressed, CD19 including, e.g., proliferative diseases such as a cancer or malignancy or a precancerous condition such as a myelodysplasia, a myelodysplastic syndrome or a preleukemia; or a noncancer related indication associated with cells which express CD19. For the avoidance of doubt, a disease associated with expression of CD19 may include a condition associated with cells which do not presently express CD19, e.g., because CD19 expression has been downregulated, e.g., due to treatment with a molecule targeting CD19, e.g., a CD19 CAR, but which at one time expressed CD19. In one aspect, a cancer associated with expression of CD19 is a hematological cancer. In one aspect, the hematological cancer is a leukemia or a lymphoma. In one aspect, a cancer associated with expression of CD19 includes cancers and malignancies including, but not limited to, e.g., one or more acute leukemias including but not limited to, e.g., acute myeloid leukemia (AML), B-cell acute lymphocytic leukemia (“B-ALL”), T-cell acute lymphocytic leukemia (“T-ALL”), acute lymphocytic leukemia (ALL); one or more chronic leukemias including but not limited to, e.g., chronic myelogenous leukemia (CIVIL), chronic lymphocytic leukemia (CLL). Additional cancers or hematologic conditions associated with expression of CD19 comprise, but are not limited to, e.g., B cell promyelocytic leukemia, blastic plasmacytoid dendritic cell neoplasm, Burkitts lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, myeloproliferative neoplasm; a histiocytic disorder (e.g., a mast cell disorder or a blastic plasmacytoid dendritic cell neoplasm); a mast cell disorder, e.g., systemic mastocytosis or mast cell leukemia; B-cell prolymphocytic leukemia, plasma cell myeloma, and “preleukemia” which are a diverse collection of hematological conditions united by ineffective production (or dysplasia) of myeloid blood cells, and the like. Further diseases associated with expression of CD19 expression include, but not limited to, e.g., atypical and/or non-classical cancers, malignancies, precancerous conditions or proliferative diseases associated with expression of CD19. Non-cancer related indications associated with expression of CD19 include, but are not limited to, e.g., autoimmune disease, (e.g., lupus), inflammatory disorders (allergy and asthma) and transplantation. In some embodiments, the tumor antigen-expressing cells express, or at any time expressed, mRNA encoding the tumor antigen. In an embodiment, the tumor antigen-expressing cells produce the tumor antigen protein (e.g., wild-type or mutant), and the tumor antigen protein may be present at normal levels or reduced levels. In an embodiment, the tumor antigen-expressing cells produced detectable levels of a tumor antigen protein at one point, and subsequently produced substantially no detectable tumor antigen protein. In other embodiments, the disease is a CD19-negative cancer, e.g., a CD19-negative relapsed cancer. In some embodiments, the tumor antigen (e.g., CD19)-expressing cell expresses, or at any time expressed, mRNA encoding the tumor antigen. In an embodiment, the tumor antigen (e.g., CD19)-expressing cell produces the tumor antigen protein (e.g., wild-type or mutant), and the tumor antigen protein may be present at normal levels or reduced levels. In an embodiment, the tumor antigen (e.g., CD19)-expressing cell produced detectable levels of a tumor antigen protein at one point, and subsequently produced substantially no detectable tumor antigen protein.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to MHC class I molecule, TNF receptor proteins, Immunoglobulin-like proteins, cytokine receptors, integrins, signalling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to an intracellular portion of a costimulatory molecule. The intracellular signaling domain can comprise the entire intracellular portion, or the entire native intracellular signaling domain, of the molecule from which it is derived, or a functional fragment thereof.

As used herein, a “value of responder or relapser status” includes a measure (e.g., level) predictive of responsiveness or relapse of a subject to a treatment (e.g., a treatment that comprises, or consists of, a CAR-expressing cell therapy as described herein). In some embodiments, the measure is qualitative or quantitative. In some embodiments, the value of responder or relapser status is complete responder, partial responder, non-responder, relapser or non-relapser. In some embodiments, the value of responder or relapser status is a probability of being a complete responder, a partial responder, a non-responder, a relapser or a non-relapser. In some embodiments, the value of responder or relapser status can be determined based on the measure of any of (i)-(viii) as described herein.

With respect to responsiveness, a subject responds to treatment if a parameter of a cancer (e.g., a hematological cancer, e.g., cancer cell growth, proliferation and/or survival) in the subject is retarded or reduced by a detectable amount, e.g., about 5%, 10%, 20%, 30%, 40%, 50%, 60%, 70%, 80%, 90% or more as determined by any appropriate measure, e.g., by mass, cell count or volume. In one example, a subject responds to treatment if the subject experiences a life expectancy extended by about 5%, 10%, 20%, 30%, 40%, 50% or more beyond the life expectancy predicted if no treatment is administered. In another example, a subject responds to treatment, if the subject has an increased disease-free survival, overall survival or increased time to progression.

Several methods can be used to determine if a patient responds to a treatment including, for example, criteria provided by NCCN Clinical Practice Guidelines in Oncology (NCCN) Guidelines®). For example, in the context of B-ALL, a complete response or complete responder, may involve one or more of: <5% BM blast, >1000 neutrophil/ANC (/□L). >100,000 platelets (/□L) with no circulating blasts or extramedullary disease (No lymphadenopathy, splenomegaly, skin/gum infiltration/testicular mass/CNS involvement), Trilineage hematopoiesis, and no recurrence for 4 weeks. A partial responder may involve one or more of ≥50% reduction in BM blast, >1000 neutrophil/ANC (/□L). >100,000 platelets (/□L). A non-responder can show disease progression, e.g., >25% in BM blasts.

A “complete responder” as used herein refers to a subject having a disease, e.g., a cancer, who exhibits a complete response, e.g., a complete remission, to a treatment. A complete response may be identified, e.g., using the NCCN Guidelines®, or Cheson et al, J Clin Oncol 17:1244 (1999) and Cheson et al., “Revised Response Criteria for Malignant Lymphoma”, J Clin Oncol 25:579-586 (2007) (both of which are incorporated by reference herein in their entireties), as described herein.

A “partial responder” as used herein refers to a subject having a disease, e.g., a cancer, who exhibits a partial response, e.g., a partial remission, to a treatment. A partial response may be identified, e.g., using the NCCN Guidelines®, or Cheson criteria as described herein.

A “non-responder” as used herein refers to a subject having a disease, e.g., a cancer, who does not exhibit a response to a treatment, e.g., the patient has stable disease or progressive disease. A non-responder may be identified, e.g., using the NCCN Guidelines®, or Cheson criteria as described herein.

The term “relapse” as used herein refers to reappearance of a disease (e.g., cancer) after an initial period of responsiveness, e.g., after prior treatment with a therapy, e.g., cancer therapy (e.g., complete response or partial response). The initial period of responsiveness may involve the level of cancer cells falling below a certain threshold, e.g., below 20%, 15%, 10%, 5%, 4%, 3%, 2%, or 1%. The reappearance may involve the level of cancer cells rising above a certain threshold, e.g., above 20%, 15%, 10%, 5%, 4%, 3%, 2%, or 1%. For example, e.g., in the context of B-ALL, the reappearance may involve, e.g., a reappearance of blasts in the blood, bone marrow (>5%), or any extramedullary site, after a complete response. A complete response, in this context, may involve <5% BM blast. More generally, in an embodiment, a response (e.g., complete response or partial response) can involve the absence of detectable MRD (minimal residual disease). In an embodiment, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

“Activated T cells” as used herein refer to T cells that show one or more characteristics of an immune response, including secretion of cytokines (e.g., secretion of IFN-gamma and/or TNFα) and target cell killing. An activated T cell can be a CD4+ or CD8+ T cell. In some embodiments, activated T cells include cytotoxic T lymphocytes.

“Cytotoxic T lymphocytes” (CTLs) as used herein refer to T cells that have the ability to kill a target cell. In embodiments, CTLs express CD8 on their cell surface.

A “clone” as used herein refers to a population of cells that are derived from the same ancestor cell. In embodiments, the cells within a clone of cells share the same phenotype(s) and/or genotype(s).

The term “clonality” as used herein refers to a characteristic of a clone, e.g., when progeny cells within a clone share the same phenotype(s) and/or genotype(s).

In some embodiments, a therapy that includes a CD19 inhibitor, e.g., a CD19 CAR therapy, may relapse or be refractory to treatment. The relapse or resistance can be caused by CD19 loss (e.g., an antigen loss mutation) or other CD19 alteration that reduces the level of CD19 (e.g., caused by clonal selection of CD19-negative clones). A cancer that harbors such CD19 loss or alteration is referred to herein as a “CD19-negative cancer” or a “CD19-negative relapsed cancer”). It shall be understood that a CD19-negative cancer need not have 100% loss of CD19, but a sufficient reduction to reduce the effectiveness of a CD19 therapy such that the cancer relapses or becomes refractory. In some embodiments, a CD19-negative cancer results from a CD19 CAR therapy. In some embodiments, a CD19-negative multiple myeloma can be treated with a CD19 CAR-expressing therapy, e.g., as described in PCT/US2015/024671, filed Apr. 7, 2015 (e.g., paragraphs 9 and 90, and Example 6 therein), which is incorporated by reference in its entirety. In some embodiments, a CD19-negative cancer can be treated with a CAR-expressing therapy, e.g., a CD123 CAR-expressing therapy, e.g., as described in PCT/US2015/045898 filed Aug. 19, 2015 (e.g., p. 26, p. 30, and Example 7 therein) which is incorporated by reference in its entirety.

The term “endogenous” refers to any material from or produced inside an organism, cell, tissue or system.

The term “effective amount” or “therapeutically effective amount” are used interchangeably herein, and refer to an amount of a compound, formulation, material, or composition, as described herein effective to achieve a particular biological result.

The term “exogenous” refers to any material introduced from or produced outside an organism, cell, tissue or system.

The term “expression” refers to the transcription and/or translation of a particular nucleotide sequence driven by a promoter.

The term “flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3, n=4, n=5, n=6, n=7, n=8, n=9 and n=10 (SEQ ID NO:28). In one embodiment, the flexible polypeptide linkers include, but are not limited to, (Gly₄Ser)₄ (SEQ ID NO:29) or (Gly₄ Ser)₃ (SEQ ID NO:30). In another embodiment, the linkers include multiple repeats of (Gly₂Ser), (GlySer) or (Gly₃Ser) (SEQ ID NO:31). Also included within the scope of the invention are linkers described in WO2012/138475, incorporated herein by reference.

The terms “homology” or “identity,” as used interchangeably herein, refer to sequence similarity between two polynucleotide sequences or between two polypeptide sequences, with identity being a more strict comparison. The phrases “percent identity or homology” and “% identity or homology” refer to the percentage of sequence similarity found in a comparison of two or more polynucleotide sequences or two or more polypeptide sequences. “Sequence similarity” refers to the percent similarity in base pair sequence (as determined by any suitable method) between two or more polynucleotide sequences. Two or more sequences can be anywhere from 0-100% similar, or any integer value there between. Identity or similarity can be determined by comparing a position in each sequence that can be aligned for purposes of comparison. When a position in the compared sequence is occupied by the same nucleotide base or amino acid, then the molecules are identical at that position. A degree of similarity or identity between polynucleotide sequences is a function of the number of identical or matching nucleotides at positions shared by the polynucleotide sequences. A degree of identity of polypeptide sequences is a function of the number of identical amino acids at positions shared by the polypeptide sequences. A degree of homology or similarity of polypeptide sequences is a function of the number of amino acids at positions shared by the polypeptide sequences. The term “substantial homology,” as used herein, refers to homology of at least 50%, at least 60%, at least 65%, at least 70%, at least 75%, at least 80%, at least 85%, at least 90%, at least 95% or more.

“Humanized” forms of non-human (e.g., murine) antibodies are chimeric immunoglobulins, immunoglobulin chains or fragments thereof (such as Fv, Fab, Fab, F(ab)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementarity-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., NATURE, 321: 522-525, 1986; Reichmann et al., NATURE, 332: 323-329, 1988; Presta, CURR. OP. STRUCT. BIOL., 2: 593-596, 1992.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NK-T) cells, mast cells, and myeloid-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc No. AAA62478.2, or the equivalent residues from a non-human species, e.g., mouse, rodent, monkey, ape and the like. In one aspect, the “4-1BB costimulatory domain” is the sequence provided as SEQ ID NO:14 or the equivalent residues from a non-human species, e.g., mouse, rodent, monkey, ape and the like.

An “intracellular signaling domain,” as the term is used herein, refers to an intracellular portion of a molecule. The intracellular signaling domain can generate a signal that promotes an immune effector function of the CAR containing cell, e.g., a CAR-expressing cell, e.g., a T cell or an NK cell. Examples of immune effector function, e.g., in a CAR-expressing cell include, cytolytic activity and helper activity, including the secretion of cytokines. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CAR-expressing cell (e.g., a T cell, an NK cell), a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (“ICOS”), FcεRI, CD66d, CD32, DAP10, and DAP12.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., MOL. THER. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “low, immune enhancing, dose” when used in conjunction with an mTOR inhibitor, e.g., an allosteric mTOR inhibitor, e.g., RAD001 or rapamycin, or a catalytic mTOR inhibitor, refers to a dose of mTOR inhibitor that partially, but not fully, inhibits mTOR activity, e.g., as measured by the inhibition of P70 S6 kinase activity. Methods for evaluating mTOR activity, e.g., by inhibition of P70 S6 kinase, are discussed herein. The dose is insufficient to result in complete immune suppression but is sufficient to enhance the immune response. In an embodiment, the low, immune enhancing, dose of mTOR inhibitor results in a decrease in the number of PD-1 positive immune effector cells, e.g., T cells or NK cells and/or an increase in the number of PD-1 negative immune effector cells, e.g., T cells or NK cells, or an increase in the ratio of PD-1 negative immune effector cells, e.g., T cells or NK cells/PD-1 positive immune effector cells, e.g., T cells or NK cells.

In general, the term “naïve T cell” refers to immune cells that comprise antigen-inexperienced cells, e.g., immune cells that are precursors of memory cells. In some embodiments, naïve T cells may be differentiated, but have not yet encountered their cognate antigen, and therefore are activated T cells or memory T cells. In some embodiments, naïve T cells may be characterized by expression of CD62L, CD27, CCR7, CD45RA, CD28, and CD127, and the absence of CD95, or CD45RO isoform. In certain embodiments, a naïve T cells is a type of younger T cell as described herein.

The term “less exhausted” or “less exhausted phenotype” refers to immune effector cells that have reduced (e.g., lack) expression of immune cell exhaustion markers, e.g. PD1, TIM3, and LAG3. In some embodiments, a less exhausted cell may be a younger T cell as described herein.

The term “nucleic acid” or “polynucleotide” refers to deoxyribonucleic acid (DNA) or ribonucleic acid (RNA), or a combination of a DNA or RNA thereof, and polymers thereof in either single- or double-stranded form. The term “nucleic acid” includes a gene, CDNA or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., NUCLEIC ACID RES. 19:5081 (1991); Ohtsuka et al., J. BIOL. CHEM. 260:2605-2608 (1985); and Rossolini et al., MOL. CELL. Probes 8:91-98 (1994)). In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

A “nucleic acid” “marker” or “biomarker” is a nucleic acid (e.g., DNA, mRNA, CDNA) encoded by or corresponding to a marker as described herein. For example, such marker nucleic acid molecules include DNA (e.g., genomic DNA and CDNA) comprising the entire or a partial sequence of any of the nucleic acid sequences set forth, or the complement or hybridizing fragment of such a sequence. The marker nucleic acid molecules also include RNA comprising the entire or a partial sequence of any of the nucleic acid sequences set forth herein, or the complement of such a sequence, wherein all thymidine residues are replaced with uridine residues. A “marker protein” is a protein encoded by or corresponding to a marker of the invention. A marker protein comprises the entire or a partial sequence of a protein encoded by any of the sequences set forth herein, or a fragment thereof. The terms “protein” and “polypeptide” are used interchangeably herein.

An “overexpression” or “significantly higher level of expression” of the gene products refers to an expression level or copy number in a test sample that is greater than the standard error of the assay employed to assess the level of expression. In embodiments, the overexpression can be at least two, at least three, at least four, at least five, or at least ten or more times the expression level of the gene in a control sample or the average expression level of gene products in several control samples.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

As used herein, a “poly(A)” is a series of adenosines attached by polyadenylation to the mRNA. In some embodiments of a construct for transient expression, the polyA is between 50 and 5000 (SEQ ID NO: 34) (e.g., 2000; SEQ ID NO: 32), e.g., 64 (SEQ ID NO: 37), e.g., greater than 100 (e.g., 150, SEQ ID NO: 33), e.g., greater than 400 (SEQ ID NO: 38). poly(A) sequences can be modified chemically or enzymatically to modulate mRNA functionality such as localization, stability or efficiency of translation.

The term “probe” refers to any molecule which is capable of selectively binding to a specifically intended target molecule, for example a marker of the invention. Probes can be either synthesized by one skilled in the art, or derived from appropriate biological preparations. For purposes of detection of the target molecule, probes can be specifically designed to be labeled, as described herein. Examples of molecules that can be utilized as probes include, but are not limited to, RNA, DNA, proteins, antibodies, and organic monomers.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “prophylaxis” as used herein means the prevention of or protective treatment for a disease or disease state.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

In embodiments, a reference or control level or activity is the level and/or activity in a subject, e.g., a sample obtained from one or more of: a baseline or prior value for the subject (e.g., prior to treatment with a CAR-expressing cell); the subject at a different time interval; an average or median value for a cancer patient population; a healthy control; or a healthy subject population (e.g., a control).

“Sample,” “tissue sample,” “patient sample,” “patient cell or tissue sample” or “specimen” each refers to a biological sample obtained from a tissue or bodily fluid of a subject or patient. The source of the tissue sample can be solid tissue as from a fresh, frozen and/or preserved organ, tissue sample, biopsy, or aspirate; blood or any blood constituents (e.g., serum, plasma); bodily fluids such as urine, cerebral spinal fluid, whole blood, plasma and serum. The sample can include a non-cellular fraction (e.g., urine, plasma, serum, or other non-cellular body fluid). In one embodiment, the sample is a urine sample. In other embodiments, the body fluid from which the sample is obtained from an individual comprises blood (e.g., whole blood). In an embodiment, the sample is a whole blood sample obtained from the subject. In certain embodiments, the blood can be further processed to obtain plasma or serum. In an embodiment, the sample is an apheresis sample obtained from the blood of the subject. In an embodiment, the sample is a manufactured product sample, e.g., genetically engineered T cells obtained from the blood of the subject, e.g., a manufactured CAR-expressing cell (e.g., T cell, NK cell) product, e.g., a manufactured CD19 CAR-expressing cell product. In another embodiment, the sample contains a tissue, cells (e.g., peripheral blood mononuclear cells (PBMC)). For example, the sample can be a fine needle biopsy sample, an archival sample (e.g., an archived sample with a known diagnosis and/or treatment history), a histological section (e.g., a frozen or formalin-fixed section, e.g., after long term storage), among others. The term sample includes any material obtained and/or derived from a biological sample, including a polypeptide, and nucleic acid (e.g., genomic DNA, CDNA, RNA) purified or processed from the sample. Purification and/or processing of the sample can involve one or more of extraction, concentration, antibody isolation, sorting, concentration, fixation, addition of reagents and the like. The sample can contain compounds that are not naturally intermixed with the tissue in nature such as preservatives, anticoagulants, buffers, fixatives, nutrients, antibiotics or the like.

The term “product” or “manufactured product” as used herein, refers to a manufactured composition comprising a genetically engineered cell (e.g., an immune effector cell), e.g., a population of cells in which a plurality of cells are engineered to express a CAR, e.g., a CAR described herein. A manufactured product can be any genetically engineered immune effector cell (e.g., T cell, NK cell), e.g., genetically engineered immune effector cells obtained from the blood of the subject, e.g., a manufactured CAR-expressing cell product, e.g., a manufactured CD19 CAR-expressing cell product. In an embodiment, a cell (e.g., an immune effector cell) engineered to express a CAR may be obtained from an activated cryopreserved expanded cell population (e.g., an expanded immune effector cell population).

The term “signaling domain” refers to the functional portion of a protein which acts by transmitting information within the cell to regulate cellular activity via defined signaling pathways by generating second messengers or functioning as effectors by responding to such messengers.

The amount of a biomarker, e.g., expression of gene products (e.g., one or more the biomarkers described herein), in a subject is “significantly” higher or lower than the normal amount of a marker, if the amount of the marker is greater or less, respectively, than the normal level by an amount greater than the standard error of the assay employed to assess amount, or at least two, three, four, five, ten or more times that amount. Alternatively, the amount of the marker in the subject can be considered “significantly” higher or lower than the normal amount if the amount is at least about 1.5, two, at least about three, at least about four, or at least about five times, higher or lower, respectively, than the normal amount of the marker.

The term “specifically binds,” refers to an antibody, or a ligand, which recognizes and binds with a cognate binding partner protein present in a sample, but which antibody or ligand does not substantially recognize or bind other molecules in the sample.

The term “stimulation,” refers to a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as down regulation of TGF-β, and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing cytoplasmic signaling sequence that is of particular use in the invention includes, but is not limited to, those derived from CD3 zeta, common FcR gamma (FCER1G), Fc gamma RIIa, FcR beta (Fc Epsilon Rib), CD3 gamma, CD3 delta, CD3 epsilon, CD79a, CD79b, DAP10, and DAP12. In a specific CAR of the invention, the intracellular signaling domain in any one or more CARS of the invention comprises an intracellular signaling sequence, e.g., a primary signaling sequence of CD3-zeta. In a specific CAR of the invention, the primary signaling sequence of CD3-zeta is the sequence provided as SEQ ID NO:18 (mutant CD3 zeta), or the equivalent residues from a non-human species, e.g., mouse, rodent, monkey, ape and the like. In a specific CAR of the invention, the primary signaling sequence of CD3-zeta is the sequence as provided in SEQ ID NO:20 (wild-type human CD3 zeta), or the equivalent residues from a non-human species, e.g., mouse, rodent, monkey, ape and the like.

The term “subject” is intended to include living organisms in which an immune response can be elicited (e.g., mammals, human). In an embodiment, a subject is a mammal. In an embodiment, a subject is a human. In an embodiment, a subject is a patient.

The term “therapeutic” as used herein means a treatment. A therapeutic effect is obtained by reduction, suppression, remission, or eradication of a disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “transmembrane domain,” refers to a polypeptide that spans the plasma membrane. In an embodiment, it links an extracellular sequence, e.g., a switch domain, an extracellular recognition element, e.g., an antigen binding domain, an inhibitory counter ligand binding domain, or costimulatory ECD domain, to an intracellular sequence, e.g., to a switch domain or an intracellular signaling domain. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). Examples of transmembrane domains are disclosed herein.

The terms “treat”, “treatment” and “treating” refer to the reduction or amelioration of the progression, severity and/or duration of a proliferative disorder, or the amelioration of one or more symptoms (e.g., one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count. In some embodiments, “treatment” refers to an approach for obtaining a beneficial or a desired result including, but not limited to: a therapeutic benefit; or prevention of a condition, e.g., a side effect, e.g., an unwanted effect as described herein. In some embodiments, a therapeutic benefit is obtained by eradication or amelioration of the underlying disorder being treated. In some embodiments, a therapeutic benefit is obtained by reduction of, eradication, or amelioration of one or more of the symptoms, e.g., physiological symptoms, associated with the underlying disorder such that an improvement, e.g., change, is observed in the patient. In some embodiments, the patient can still be afflicted with the underlying disorder. In some embodiments, treatment comprises prevention of a condition, e.g., a side effect, e.g., an unwanted side effect from a therapy. Treatment or prevention of a condition or a side effect need not be a complete treatment or prevention of the condition or side effect.

As used herein, unless otherwise specified, the terms “prevent,” “preventing” and “prevention” refer to an action that occurs before the subject begins to suffer from the condition, or relapse of the condition. Prevention need not result in a complete prevention of the condition; partial prevention or reduction of the condition or a symptom of the condition, or reduction of the risk of developing the condition, is encompassed by this term.

An “underexpression” or “significantly lower level of expression” of products (e.g., the markers set forth herein) refers to an expression level in a test sample that is greater than the standard error of the assay employed to assess expression, for example, at least 1.5, twice, at least three, at least four, at least five, or at least ten or more times less than the expression level of the gene in a control sample, or the average expression level of gene products in several control samples.

The term “xenogeneic” refers to a graft derived from an animal of a different species.

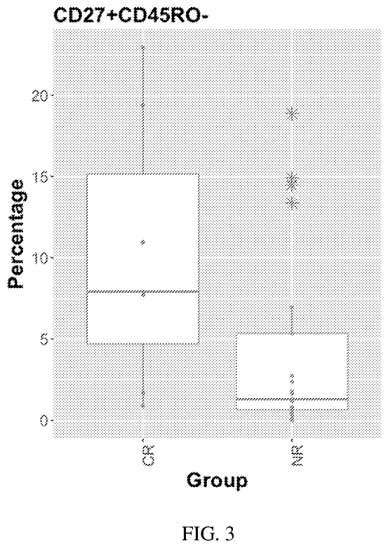

As used herein, the term “young T cell” or “younger T cell”, refers to an immune effector cell that comprises a less differentiated phenotype, e.g., a younger cell, e.g., a young T cell. In some embodiments, a younger T cell may be a naïve T cell (T_{N}). In some embodiments, a young T cell may be characterized by expression of CD62L, and the absence of CD25, CD44, or CD45RO isoform. In some embodiments, a younger T cell may be a memory stem cell (T_{SCM}). In some embodiments, a younger T cells may be a central memory T cell (T_{CM}). Phenotypic markers associated with T_{N}, T_{SCM} and T_{CM} are disclosed in, e.g., Maus, M. et al. (2014) Annu. Rev. Immunol. 32:189-225 (see for example, FIG. 3 therein), incorporated by reference herein. Exemplary phenotypes of T_{N} include one or more (or all) of the following: CD45RA+, CD45RO−, CD62L^{high}, CCR7^{high}, CD95−, CD122−, CD27^{high}, CD28+, CD57−, KLRG-1−, or long telomere length (or any combination of two, three, four, five, six, seven, eight, nine, or all of the aforesaid T_{N} markers). Exemplary phenotypes of T_{SCM} include one or more (or all) of the following: CD45RA+, CD45RO−, CD62L^{high}, CCR7^{high}, CD95+, CD122+, CD27^{high}, CD28^{high}, CD57−, KLRG-1−, or long telomere length (or any combination of two, three, four, five, six, seven, eight, nine, or all of the aforesaid T_{SCM} markers). Exemplary phenotypes of T_{CM} include one or more (or all) of the following: CD45RA−, CD45RO^{high}, CD62L^{high}, CCR7+, CD95+, CD122^{high}, CD27+, CD28^{high}, CD57−, KLRG-1−/+, or long/intermediate telomere length (or any combination of two, three, four, five, six, seven, eight, nine, or all of the aforesaid T_{CM} markers).

As used herein, the term “older T cell” refers to an immune effector cell that comprises a more exhausted phenotype. In some embodiments, an older T cell may be an effector memory T cell (T_{EM}). In other embodiments, an older T cell may be an effector T cell (T_{EFF}). In other embodiments, an older T cell has an exhausted phenotype. Phenotypic markers associated with T_{EM}, T_{EFF} and exhausted T cells are disclosed in, e.g., Maus, M. et al. (2014) Annu. Rev. Immunol. 32:189-225 (see for example, FIG. 3 therein), incorporated by reference herein. Exemplary phenotypes of T_{EM} include one or more (or all) of the following: CD45RA−/+, CD45RO^{high}, CD62L−, CCR7−, CD95−, CD122^{high}, CD27−/+, CD28−/+, CD57^{low}, KLRG-1+, or intermediate telomere length (or any combination of two, three, four, five, six, seven, eight, nine, or all of the aforesaid T_{EM} markers). Exemplary phenotypes of T_{EFF} include one or more (or all) of the following: CD45RA−/+, CD45RO+, CD62L−, CCR7−, CD95^{high}, CD122−/+, CD27−, CD28−, CD57+, KLRG-1^{high}, or short/intermediate telomere length (or any combination of two, three, four, five, six, seven, eight, nine, or all of the aforesaid T_{EFF} markers). Exemplary phenotypes of an exhausted T cell phenotype include one or more (or all) of the following: CD45RA−/+, CD45RO+, CD62L−, CCR7−, CD95^{high}, CD122^{low}, CD27−, CD28−, CD57^{high}, KLRG-1^{high}, or short telomere length (or any combination of two, three, four, five, six, seven, eight, nine, or all of the aforesaid markers).

The term “zeta” or alternatively “zeta chain”, “CD3-zeta” or “TCR-zeta” is defined as the protein provided as GenBank Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof. In one aspect, the “zeta stimulatory domain” or a “CD3-zeta stimulatory domain” is the sequence provided as SEQ ID NO:18. In one aspect, the “zeta stimulatory domain” or a “CD3-zeta stimulatory domain” is the sequence provided as SEQ ID NO:20.

Various aspects of the invention are described in further detail below. Additional definitions are set out throughout the specification.

### DETAILED DESCRIPTION OF CERTAIN EMBODIMENTS

Biomarkers predicting response to a therapy in subjects having cancer (e.g., a hematological cancer such as chronic lymphocytic leukemia (CLL)) are provided herein.

In one aspect, biomarkers predicting response to a cell expressing a CAR, e.g., a CD19 CAR-expressing cell (e.g., T cell, NK cell) (e.g., a CD19 CAR-expressing cell described herein such as, e.g., CTL019) in subjects having a cancer, e.g., CLL are provided herein.

Methods are provided for the diagnosis and monitoring of treatment of cancer (e.g., a hematological cancer such as CLL, ALL, DLBCL, e.g., relapsed or refractory DLBCL, or FL, e.g., relapsed or refractory FL) based on detection of certain biomarkers in samples from patients who have, or are suspected of having, cancer. Further, expression of one or more such biomarkers can be used to distinguish subjects that respond favorably to a CAR-expressing cell (e.g., T cell, NK cell) therapy (e.g., “complete responders” or “CR”) from subjects that don't respond to a CAR-expressing cell therapy (e.g., “non-responders” or “NR”) and from subjects that have a partial response to a CAR-expressing cell therapy (e.g., “partial responders” or “PR”).

### Use of Biomarkers to Evaluate Disease Progression and Predict Subject Response to CAR-Expressing Cell Therapy

In an embodiment, one or more genes encoding CD27, CD45RO, CCR7, HLA-DR, CD127, CD95 and/or a CD19 CAR-expressing cell (e.g., T cell, NK cell) gene signature can be used with methods of the present disclosure to acquire a disease progression value. The disease progression value can be used for, e.g., in evaluating the effectiveness of therapies in treating cancer (e.g., a hematological cancer such as ALL and CLL). In an embodiment, one or more genes encoding CD27, CD45RO, CCR7, HLA-DR, CD127, CD95 and/or a CD19 CAR-expressing cell gene signature are used to classify a subject as a complete responder, partial responder, or non-responder to CAR-expressing cell therapy (e.g., a CD19 CAR-expressing cell therapy described herein such as, e.g., CTL019). In an embodiment, one or more genes encoding CD27, CD45RO, CCR7, HLA-DR, CD127, CD95, and/or a CD19 CAR-expressing cell gene signature are used to predict a subject's responsiveness to a CAR-expressing cell therapy (e.g., a CD19 CAR-expressing cell therapy described herein such as, e.g., CTL019).

### Subjects

For any of the methods and kits disclosed herein, the subject treated, or the subject from which the value is obtained, is a subject having, or at risk of having, cancer at any stage of treatment. Exemplary cancers include, but are not limited to, B-cell acute lymphocytic leukemia (B-ALL), e.g., relapsed or refractory B-ALL, T-cell acute lymphocytic leukemia (T-ALL), acute lymphocytic leukemia (ALL), chronic myelogenous leukemia (CIVIL), chronic lymphocytic leukemia (CLL), B cell promyelocytic leukemia, blastic plasmacytoid dendritic cell neoplasm, Burkitt lymphoma, diffuse large B cell lymphoma, follicular lymphoma (FL) e.g., relapsed or refractory FL, Diffuse Large B cell lymphoma (DLBCL), e.g., relapsed or refractory DLBCL, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, and Waldenstrom macroglobulinemia.

In an embodiment, the cancer is a hematological cancer. In some embodiments, the cancer is a relapsed or refractory cancer, e.g., a relapsed or refractory cancer described herein, e.g., a relapsed or refractory hematological cancer. In an embodiment, the cancer is ALL. In another embodiment, the cancer is CLL. In some embodiments, the cancer is DLBCL, e.g., relapsed or refractory DLBCL. In some embodiments, the cancer is FL, e.g., relapsed or refractory FL. In an embodiment, the cancer is associated with CD19 expression.

In some embodiments, the subject is a pediatric subject or a young adult.

In some embodiments, the subject is an adult.

In an embodiment, the subject has received a pretreatment of an additional therapy, e.g., a subject that has been identified as a partial responder or non-responder and subsequently has been pretreated with an additional therapy. In an embodiment, the subject receives pretreatment with an mTOR inhibitor. In an embodiment, the mTOR inhibitor is administered at a dose or dosing schedule described herein. In one embodiment, a low, immune enhancing dose of an mTOR inhibitor is given to the subject prior to treatment with a CAR-expressing cell (e.g., a T cell, an NK cell). In an embodiment, administration of a low, immune enhancing, dose of an mTOR inhibitor, e.g., an allosteric inhibitor, e.g., RAD001, or a catalytic inhibitor, is initiated prior to administration of a CAR expressing cell described herein, e.g., T cells. In an embodiment, the CAR cells are administered after a sufficient time, or sufficient dosing, of an mTOR inhibitor, such that the level of PD1 negative immune effector cells, e.g., T cells, or the ratio of PD1 negative immune effector cells, e.g., T cells/PD1 positive immune effector cells, e.g., T cells, has been, at least transiently, increased. In an embodiment, the cell, e.g., T cell, to be engineered to express a CAR, is harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In one embodiment, the subject has received a pretreatment with a checkpoint inhibitor, e.g., a checkpoint inhibitor described herein. Examples of inhibitory molecules, e.g., checkpoint molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta). In embodiments, an inhibitory nucleic acid, e.g., an inhibitory nucleic acid, e.g., a dsRNA, e.g., an siRNA or shRNA; or e.g., an inhibitory protein or system, e.g., a clustered regularly interspaced short palindromic repeats (CRISPR), a transcription-activator like effector nuclease (TALEN), or a zinc finger endonuclease (ZFN), e.g., as described herein, can be used to inhibit expression of a molecule that modulates or regulates, e.g., inhibits, T-cell function in the CAR-expressing cell. In an embodiment the agent is an shRNA, e.g., an shRNA described herein. In one embodiment, the inhibitor of checkpoint molecule can be, e.g., an antibody or antibody fragment that binds to a checkpoint molecule. For example, the agent can be an antibody or antibody fragment that binds to PD1, PD-L1, PD-L2 (e.g., as described herein) or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206)). In an embodiment, the agent is an antibody or antibody fragment that binds to TIM3, e.g., as described herein. In an embodiment, the agent is an antibody or antibody fragment that binds to LAG3, e.g., as described herein. In an embodiment, the agent is an antibody or antibody fragment that binds to CEACAM, e.g., as described herein.

In an embodiment, the subject receives an additional therapy in combination with CAR-expressing cell (e.g., a T cell, an NK cell) therapy (e.g., a CD19 CAR-expressing cell therapy described herein such as, e.g., CTL019). In an embodiment, the subject receives an mTOR inhibitor, e.g., an mTOR inhibitor described herein, in combination with CAR-expressing cell therapy. In one embodiment, the mTOR inhibitor is administered at a dose and/or dosing schedule described herein. In one embodiment, the subject receives a checkpoint inhibitor, e.g., a checkpoint inhibitor described herein, in combination with CAR-expressing cell therapy. In one embodiment, the checkpoint inhibitor is administered at a dose and/or dosing schedule described herein. In an embodiment, the subject receives a kinase inhibitor, e.g., a kinase inhibitor described herein. In one embodiment, the kinase inhibitor is administered at a dose and/or dosing schedule described herein.

In an embodiment, the subject has been identified as a non-responder and the subject receives a therapy other than a CAR-expressing cell therapy, e.g., a standard of care therapy for the particular cancer type. In one embodiment, the subject receives one or more of an anti-CD20 antibody, or functional fragment thereof (e.g., ofatumumab, rituximab, obinutuzumab), an anti-CD52 antibody or functional fragment thereof (e.g., alemtuzumab), an alkylating agent (e.g., a nitrogen mustard alkylating agent such as, e.g., bendamustine HCl, chlorambucel, cyclophosphamide), a kinase inhibitor (e.g., a kinase inhibitor described herein such as, e.g., a BTK inhibitor described herein or a phosphonositide-3 kinase inhibitor described herein). In one embodiment, the subject receives a stem cell transplant.

Subject with Follicular Lymphoma

In some embodiments, the subject has a follicular lymphoma (FL). In some embodiments, the subject has a relapsed or refractory FL (r/r FL). In some embodiments, the subject having relapsed or refractory FL has not responded to, e.g., failed, e.g., at least 2 prior therapies, e.g., systemic therapies, e.g., as described herein. In some embodiments, the subject with relapsed or refractory FL meets one, two or more, e.g., all, of the following criteria: (i) refractory to, e.g., a second line, or later line therapy, e.g., systemic therapy, e.g., anti-CD20 therapy or alkylating agent; (ii) relapsed within, e.g., 6 months after completion of, e.g., a second line or later line therapy, e.g., systemic therapy; (iii) relapsed during or after an anti-CD20 antibody therapy (e.g., maintenance therapy, e.g., following at least two lines of therapy, e.g., systemic therapy) or within, e.g., 6 months after completion of anti-CD20 antibody therapy (e.g., maintenance therapy); (iv) relapsed after stem cell therapy (SCT), e.g., autologous or allogeneic SCT, e.g., autologous SCT, e.g., as described herein; or (v) previously treated with other FL-targeting agents, e.g., PI3K inhibitor, e.g., as described herein. In some embodiments, the subject with FL has a FL of grade 1, 2 or 3A. In some embodiments, the subject is an adult, e.g., is at least 18 years of age. In some embodiments, the subject with FL, e.g., relapsed or refractory FL, is administered a CAR-expressing cell therapy, e.g., a CAR19 expressing cell therapy, e.g., at a dosage regimen described herein. In some embodiments, the subject is administered about 0.6-6.0×10⁸ CAR-expressing cells, e.g., 0.6-6.0×10⁸ CAR19 expressing cells, in a single infusion. In some embodiments, the infusion, e.g., infusion bag, contains 5-100 ml of cells, e.g., 10-50 ml of cells, e.g., CAR expressing cells.

In some embodiments, prior to CAR19 expressing cell infusion, e.g., CTL019 infusion, a laboratory assessment, e.g., as described herein, can be performed. In some embodiments, exemplary laboratory assessments are described in Example 4. In some embodiments, the laboratory assessments include, but are not limited to one, two, three or all of the following:

(i) tumor clonal typing, e.g., by deep sequencing, of a sample from the subject, e.g., tumor biopsy, peripheral blood or circulating tumor DNA;

(ii) peripheral blood molecular characterization, e.g., by immunophenotyping or leukocyte gene expression profiling;

(iii) minimal residual disease assessment, e.g., by Ig deep sequencing; or

(iv) flow cytometry assessment on, e.g., pre-leukapheresis peripheral blood or leukapheresis product, e.g., ALC, absolute CD45⁺/CD3⁺, absolute CD45⁺/CD3⁺/CD28⁻/CD27⁻, absolute CD4⁺/CD25⁺, and/or absolute CD45⁺/CD14⁺.

### Biomarkers Assessment

### Analysis of CTL019 Biomarkers

Analysis of levels of expression and/or activity of gene products correlated with a subject's response to CAR-expressing cell (e.g., T cell, NK cell) therapy (e.g., a CD19 CAR-expressing cell therapy described herein such as, e.g., CTL019) and cancer disease progression (e.g., a hematological cancer such as ALL, CLL, DLBCL, e.g., relapsed or refractory DLBCL, or FL, e.g., relapsed or refractory FL) has led to the identification of novel CD19 CAR-expressing cell gene signatures. For example, the present invention provides methods for evaluation of expression level of one or more biomarkers including but not limited to CD27, CD45RO, CCR7, HLA-DR, CD127, and CD95 that, e.g., comprise a CD19 CAR-expressing cell gene signature.

In some embodiments, methods of the present disclosure can be used to determine the responsiveness of a subject to treatment with a CAR-expressing cell therapy (e.g., a CD19 CAR-expressing cell (e.g., T cell, NK cell) therapy described herein such as, e.g., CTL019), wherein a statistically significant difference in the amount, e.g., expression, and/or activity of a marker disclosed herein relative to a reference, e.g., a median value for a cancer patient population (e.g., a hematological cancer such as ALL, CLL, DLBCL, e.g., relapsed or refractory DLBCL, or FL, e.g., relapsed or refractory FL), a median value for a population of healthy, cancer-free subjects, a median value for a population of non-responders or partial responders, in a subjects sample, then the more likely the disease is to respond to CAR-expressing cell therapy.

In an embodiment, the disclosure provides a method of, or assay for, identifying a subject having cancer (e.g., a hematological cancer such as ALL, CLL, DLBCL, e.g., relapsed or refractory DLBCL, or FL, e.g., relapsed or refractory FL) as having an increased or decreased likelihood to respond to a treatment that comprises a CAR-expressing cell therapy (e.g., a CD19 CAR-expressing cell therapy described herein such as, e.g., CTL019), the method comprising:
(1) acquiring a sample from the subject;
(2) determining a level of one or more CD27, CD45RO, CCR7, HLA-DR, CD127, and CD95 in the sample; and
(3) comparing the determined level of one or more markers to a reference level; wherein a difference, e.g., statistically significant difference in the determined level to the reference level is predictive of the subjects responsiveness to the CAR-expressing cell therapy; and
(4) identifying the subject as a complete responder, partial responder or non-responder to the CAR-expressing cell therapy.

In one embodiment, the sample is a blood, plasma or a serum sample. In one embodiment, the sample is an apheresis sample, e.g., T cells obtained from the blood of the subject. In an embodiment, the sample is a manufactured product sample, e.g., genetically engineered T cells obtained from the blood of the subject, e.g., a manufactured CAR-expressing cell product, e.g., a manufactured CD19 CAR-expressing cell product.

In an embodiment, the disclosure provides a method of, or assay for, identifying a subject having a cancer including, but not limited to, B-cell acute lymphocytic leukemia (B-ALL), T-cell acute lymphocytic leukemia (T-ALL), acute lymphocytic leukemia (ALL), chronic myelogenous leukemia (CIVIL), chronic lymphocytic leukemia (CLL), B cell promyelocytic leukemia, blastic plasmacytoid dendritic cell neoplasm, Burkitt lymphoma, diffuse large B cell lymphoma (DLBCL), e.g., relapsed or refractory DLBCL, follicular lymphoma (FL), e.g., relapsed or refractory FL, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, and Waldenstrom macroglobulinemia. In an embodiment, the cancer is a hematological cancer. In an embodiment, the cancer is ALL. In another embodiment, the cancer is CLL. In an embodiment, the cancer is associated with CD19 expression.

In an embodiment, a CAR-expressing cell therapy comprises a CAR-expressing cell therapy described herein, e.g., CTL019.

In an embodiment, a CAR-expressing cell therapy consists of a CAR-expressing cell therapy described herein, e.g., CTL019.

In an embodiment, the disclosure provides a method of, or assay for, identifying a subject having cancer (e.g., a hematological cancer such as ALL, CLL, DLBCL, e.g., relapsed or refractory DLBCL, or FL, e.g., relapsed or refractory FL) as having an increased or decreased likelihood to respond to a treatment that comprises a CAR-expressing cell therapy (e.g., a CD19 CAR-expressing cell therapy described herein such as, e.g., CTL019), the method comprising:
(1) acquiring a sample from the subject;
(2) determining a gene signature of the sample; and
(3) comparing the determined gene signature to a reference gene signature;
   wherein a difference, e.g., statistically significant difference in expression level of one or more of the markers in the determined gene signature, e.g., as compared to a predetermined value, is predictive of the subjects responsiveness to the CAR-expressing cell therapy.

In an embodiment, the disclosure provides a method of, or assay for, determining the responsiveness of a subject having cancer (e.g., a hematological cancer such as ALL, CLL, DLBCL, e.g., relapsed or refractory DLBCL, or FL, e.g., relapsed or refractory FL) to a treatment comprising a cell expressing a CAR (e.g., a cell expressing a CD19 CAR, e.g., a CD19 CAR-expressing cell (e.g., T cell, NK cell) described herein such as, e.g., CTL019), the method comprising:

determining a level of one or more biomarkers CD27, CD45RO, CCR7, HLA-DR, CD127, and CD95 in a sample obtained prior to treatment;

wherein a statistically significant difference in expression level of one or more markers in the sample relative to a predetermined value is indicative of increased responsiveness to the CAR-expressing cell.

The methods provided herein are particularly useful for identifying subjects that are likely to respond to CAR-expressing cell (e.g., T cell, NK cell) therapy (e.g., a CD19 CAR-expressing cell therapy described herein such as, e.g., CTL019) prior to initiation of such treatment (e.g., pre-therapy) or early in the therapeutic regimen. In some embodiments, expression or activity of biomarkers is measured in a subject at least 2 weeks, at least 1 month, at least 3 months, at least 6 months, or at least 1 year prior to initiation of therapy. In some embodiments, expression or activity of biomarkers is measured less than 6 months prior to the initiation of therapy. Thus, in some embodiments, expression or activity of biomarkers is measured within 6 months, 5 months, 4 months, 3 months, 2 months, 1 month, 2 weeks, 1 week, 6 days, 5 days, 4 days, 3 days, 2 days, or 1 day prior to the initiation of therapy. In other embodiments, the expression or activity of biomarkers is determined after initiation of therapy (e.g., 1 month, 2 months, 3 months, 3.5 months, 4 months, 4.5 months, 5 months, 5.5 months, 6 months).

In an embodiment, the invention provides a method of evaluating a subject having cancer (e.g., a hematological cancer such as ALL, CLL, DLBCL, e.g., relapsed or refractory DLBCL, or FL, e.g., relapsed or refractory FL) comprising:

acquiring a value of responder status for the subject that comprises a measure of one or more of the following:

one or more of a CD27 biomarker, a CD45RO biomarker, a CCR7 biomarker, a CD27 biomakrer, a HLA-DR biomarker, a CD95 biomarker, a CD127 biomarker, a CD4 biomarker, a CD8 biomarker, a TH1+ helper T cell gene set signature, a TH2+ helper T cell gene set signature, a memory T cell (e.g., a CD8+ memory T cell, e.g., a naïve T cell (T_{N}), e.g. a memory stem cell (T_{SCM}), e.g. a central memory T cell (T_{CM}), e.g. an effector memory T cell (T_{EM})) gene set signature, and a CD19 CAR-expressing cell gene set signature, thereby evaluating the subject.

In an embodiment, the disclosure provides a method of evaluating a subject having cancer (e.g., a hematological cancer such as ALL, CLL, DLBCL, e.g., relapsed or refractory DLBCL, or FL, e.g., relapsed or refractory FL) comprising acquiring a value of responder status for the subject that comprises a measure of one or CD27, CD45RO, CCR7, HLA-DR, CD127, and CD95, and a CD19 CAR-expressing cell gene set signature, thereby evaluating the subject.

In an embodiment, the disclosure provides a method of evaluating or monitoring the effectiveness of a CAR-expressing cell therapy in a subject having cancer comprising:

acquiring a value of responder status for the subject that comprises a measure of one or more of the following:

one or more of a CD27 biomarker, a CD45RO biomarker, a CCR7 biomarker, a CD27 biomakrer, a HLA-DR biomarker, a CD95 biomarker, a CD127 biomarker, a CD4 biomarker, a CD8 biomarker, a TH1+ helper T cell gene set signature, a TH2+ helper T cell gene set signature, a memory T cell (e.g., a CD8+ memory T cell, e.g., a naïve T cell (T_{N}), e.g. a memory stem cell (T_{SCM}), e.g. a central memory T cell (T_{CM}), e.g. an effector memory T cell (T_{EM})) gene set signature, and a CD19 CAR-expressing cell gene set signature, thereby evaluating or monitoring the effectiveness of the CAR therapy in the subject.

In an embodiment, the disclosure provides a method of evaluating or monitoring the effectiveness of a CAR-expressing cell (e.g., T cell, NK cell) therapy (e.g., a CD19 CAR-expressing cell therapy described herein such as, e.g., CTL019) in a subject having cancer (e.g., a hematological cancer such as ALL, CLL, DLBCL, e.g., relapsed or refractory DLBCL, or FL, e.g., relapsed or refractory FL) comprising: acquiring a value of responder status for the subject that comprises a measure of one or more CD27, CD45RO, CCR7, HLA-DR, CD127, and CD95, and a CD19 CAR-expressing cell (e.g., T cell, NK cell) gene set signature, thereby evaluating or monitoring the effectiveness of the CAR-expressing cell (e.g., T cell, NK cell) therapy in the subject.

In an embodiment, the value of responder status comprises a measure of a combination of a gene signature and a biomarker.

In an embodiment, the value of the responder status comprises a measure of a CD19 CAR-expressing cell gene set signature and a combination of one or more of one or more of a CD27 biomarker, a CD45RO biomarker, a CCR7 biomarker, a CD27 biomakrer, a HLA-DR biomarker, a CD95 biomarker, a CD127 biomarker, a CD4 biomarker, a CD8 biomarker, a TH1+ helper T cell gene set signature, a TH2+ helper T cell gene set signature, a memory T cell (e.g., a CD8+ memory T cell, e.g., a naïve T cell (TN), e.g. a memory stem cell (TSCM), e.g. a central memory T cell (TCM), e.g. an effector memory T cell (TEM)) gene set signature, and a CD19 CAR-expressing cell gene set signature.

In an embodiment, the value for expression of the gene comprises a value for a transcriptional parameter, e.g., the level of an mRNA encoded by the gene.

In an embodiment, the value for expression of the protein comprises a value for a translational parameter, e.g., the level of a protein.

In an embodiment, provided methods further comprise obtaining a sample from the subject, wherein the sample comprises a cellular or tissue fraction. In an embodiment, the cellular fraction comprises blood.

In an embodiment, the measure of biomarker and/or gene signature is acquired before, at the same time, or during course of a CAR-expressing cell therapy (e.g., a CD19 CAR-expressing cell therapy described herein such as, e.g., CTL019).

In an embodiment, the measure of biomarker and/or gene signature is acquired less than 6 months, 5 months, 4 months, 3 months, 2 months, 1 month, 2 weeks, 1 week, 6 days, 5 days, 4 days, 3 days, 2 days prior to the initiation of a CAR-expressing cell therapy (e.g., a CD19 CAR-expressing cell therapy described herein such as, e.g., CTL019).

The methods described herein can also be used to monitor a positive response of a subject to CAR-expressing cell (e.g., T cell, NK cell) treatment (e.g., a CD19 CAR-expressing cell treatment described herein such as, e.g., CTL019). Such methods are useful for early detection of tolerance to therapy or to predict whether disease in a subject will progress. In such embodiments, the expression or activity of biomarkers is determined e.g., at least every week, at least every 2 weeks, at least every month, at least every 2 months, at least every 3 months, at least every 4 months, at least every 5 months, at least every 6 months, at least every 7 months, at least every 8 months, at least every 9 months, at least every 10 months, at least every 11 months, at least every year, at least every 18 months, at least every 2 years, at least every 3 years, at least every 5 years or more. It is also contemplated that expression or activity of the biomarkers is at irregular intervals e.g., biomarkers can be detected in a subject at 3 months of treatment, at 6 months of treatment, and at 7 months of treatment. Thus, in some embodiments, the expression or activity of the biomarkers is determined when deemed necessary by the skilled physician monitoring treatment of the subject.

The methods described herein can be used in treating any subject having cancer (e.g., a hematological cancer such as ALL, CLL, DLBCL, e.g., relapsed or refractory DLBCL, or FL, e.g., relapsed or refractory FL). In one aspect, the invention pertains to methods of treating cancer (e.g., a hematological cancer such as ALL, CLL, DLBCL, e.g., relapsed or refractory DLBCL, or FL, e.g., relapsed or refractory FL) in a subject.

In an embodiment, the disclosure provides methods for treating cancer including, but not limited to, B-cell acute lymphocytic leukemia (B-ALL), T-cell acute lymphocytic leukemia (T-ALL), acute lymphocytic leukemia (ALL), chronic myelogenous leukemia (CML), chronic lymphocytic leukemia (CLL), B cell promyelocytic leukemia, blastic plasmacytoid dendritic cell neoplasm, Burkitts lymphoma, diffuse large B cell lymphoma (DLBCL), e.g., relapsed or refractor DLBCL, follicular lymphoma (FL), e.g., relapsed or refractory FL, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, and Waldenstrom macroglobulinemia. In an embodiment, the invention provides methods for treating ALL. In another embodiment, the invention provides methods for treating CLL. In some embodiments, the invention provides methods for treating DLBCL, e.g., relapsed or refractory DLBCL. In some embodiments, the invention provides methods for treating FL, e.g., relapsed or refractory FL In an embodiment, the invention provides methods for treating cancer that is associated with CD19 expression.

In an embodiment, provided methods comprise administering to the subject a cell expressing a CAR, e.g. a CAR T cell, e.g. a CD19 CAR T cell, e.g., a CTL019 product, if the subject is identified as having a difference, e.g., statistically significant difference in expression level of one or more CD27, CD45RO, CCR7, HLA-DR, CD127, and CD95 relative to a reference level, such that the cancer (e.g., a hematological cancer such as ALL, CLL, DLBCL, e.g., relapsed or refractory DLBCL, or FL, e.g., relapsed or refractory FL) is treated in the subject.

As discussed above, an example of a cancer that is treatable by disclosed methods is a cancer associated with expression of CD19. In one aspect, the cancer associated with expression of CD19 is a hematological cancer. In one aspect, the hematological cancer is a leukemia or a lymphoma. In one aspect, a cancer associated with expression of CD19 includes cancers and malignancies including, but not limited to, e.g., one or more acute leukemias including but not limited to, e.g., B-cell acute lymphoid leukemia (“B-ALL”), T-cell acute lymphoid leukemia (“T-ALL”), acute lymphoid leukemia (ALL); one or more chronic leukemias including but not limited to, e.g., chronic myelogenous leukemia (CML), chronic lymphocytic leukemia (CLL). Additional cancers or hematologic conditions associated with expression of CD19 include, but are not limited to, e.g., B cell promyelocytic leukemia, blastic plasmacytoid dendritic cell neoplasm, Burkitts lymphoma, diffuse large B cell lymphoma (DLBCL), e.g., relapsed or refractory DLBCL, follicular lymphoma (FL), e.g., relapsed or refractory FL, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further, a disease associated with CD19 expression include, but not limited to, e.g., atypical and/or non-classical cancers, malignancies, precancerous conditions or proliferative diseases associated with expression of CD19.

In an embodiment, the disclosure provides a method for treating a subject having cancer (e.g., a hematological cancer such as ALL, CLL, DLBCL, e.g., relapsed or refractory DLBCL, or FL, e.g., relapsed or refractory FL) comprising:

determining if the subject has an increased likelihood to respond to a CAR-expressing cell (e.g., T cell, NK cell) therapy (e.g., a CD19 CAR-expressing cell therapy described herein such as, e.g., CTL019) by comparing the level of one or more markers including but not limited to CD27, CD45RO, CCR7, HLA-DR, CD127, and CD95

in a sample from the subject relative to a reference level, wherein a statistically significant difference in expression level of one or more maker genes relative to the reference level is indicative of an increased likelihood of response; and

administering to the subject a therapeutically effective dose of a CAR-expressing cell therapy.

In an embodiment, the disclosure provides a method for treating a subject having cancer (e.g., a hematological cancer such as ALL, CLL, DLBCL, e.g., relapsed or refractory DLBCL, or FL, e.g., relapsed or refractory FL), comprising:

obtaining a sample from the subject;

determining a level of one or more markers including but not limited to CD27, CD45RO, CCR7, HLA-DR, CD127, and CD95;

comparing the determined level of one or more markers including but not limited to CD27, CD45RO, CCR7, HLA-DR, CD127, and CD95; and

administering a therapeutically effective dose of a CAR-expressing cell (e.g., T cell, NK cell) therapy (e.g., a CD19 CAR-expressing cell therapy described herein such as, e.g., CTL019), if the subject is identified as having a statistically significant difference in the determined level of one or more markers including but not limited to CD27, CD45RO, CCR7, HLA-DR, CD127, and CD95 to a reference level, in the sample.

In an embodiment, the disclosure provides a method of treating cancer (e.g., a hematological cancer such as ALL, CLL, DLBCL, e.g., relapsed or refractory DLBCL, or FL, e.g., relapsed or refractory FL) in a subject, comprising:

acquiring a value of responder status for the subject that comprises a measure of one or more of the following:

one or more of a CD27 biomarker, a CD45RO biomarker, a CCR7 biomarker, a CD27 biomakrer, a HLA-DR biomarker, a CD95 biomarker, a CD127 biomarker, a CD4 biomarker, a CD8 biomarker, a TH1+ helper T cell gene set signature, a TH2+ helper T cell gene set signature, a memory T cell (e.g., a CD8+ memory T cell, e.g., a naïve T cell (TN), e.g. a memory stem cell (TSCM), e.g. a central memory T cell (TCM), e.g. an effector memory T cell (TEM)) gene set signature, and a CD19 CAR-expressing cell gene set signature, and

responsive to a determination of responder status, performing one, two, three, four or more of:

identifying the subject as a complete responder, partial responder or non-responder;

administering a CAR-expressing cell therapy (e.g., a CD19 CAR-expressing cell therapy described herein such as, e.g., CTL019);

selecting or altering a dosing of a CAR-expressing cell therapy;

selecting or altering the schedule or time course of a CAR-expressing cell therapy;

administering, e.g., to a non-responder or a partial responder, an additional agent in combination with a CAR-expressing cell therapy, e.g., a checkpoint inhibitor, e.g., a checkpoint inhibitor described herein;

administering to a non-responder or partial responder a therapy that increases the number of naïve T cells in the subject prior to treatment with a CAR-expressing cell therapy;

modifying a manufacturing process of a CAR-expressing cell therapy, e.g., enrich for naïve T cells prior to introducing a nucleic acid encoding a CAR, e.g., for a subject identified as a non-responder or a partial responder; or selecting an alternative therapy, e.g., for a non-responder or partial responder; or

selecting an alternative therapy, e.g., an alternative therapy described herein, e.g., a standard of care therapy for the cancer; thereby treating cancer in a subject.

In some embodiments, the amount of the biomarker determined in a sample from a subject is quantified as an absolute measurement (e.g., ng/mL). Absolute measurements can easily be compared to a reference value or cut-off value. For example, a cut-off value can be determined that represents a disease progressing status; any absolute values falling either above (i.e., for biomarkers that increase expression with progression of a cancer, e.g., a hematological cancer such as ALL, CLL, DLBCL, e.g., relapsed or refractory DLBCL, or FL, e.g., relapsed or refractory FL) or falling below (i.e., for biomarkers with decreased expression with progression of a cancer, e.g., a hematological cancer such as ALL, CLL, DLBCL, e.g., relapsed or refractory DLBCL, or FL, e.g., relapsed or refractory FL) the cut-off value are likely to be disease progressing.

Alternatively, the relative amount of a biomarker is determined. In one embodiment, the relative amount is determined by comparing the expression and/or activity of one or more biomarkers in a subject with cancer to the expression of the biomarkers in a reference parameter. In some embodiments, a reference parameter is obtained from one or more of: a baseline or prior value for the subject, the subject at a different time interval, an average or median value for a cancer subject (e.g., patient) population, a healthy control, or a healthy subject population.

The present disclosure also pertains to the field of predictive medicine in which diagnostic assays, pharmacogenomics, and monitoring clinical trials are used for predictive purposes to thereby treat an individual prophylactically. Accordingly, one aspect of the present disclosure relates to assays for determining the amount, structure, and/or activity of polypeptides or nucleic acids corresponding to one or more markers described herein, in order to determine whether an individual having cancer (e.g., a hematological cancer such as ALL, CLL, DLBCL, e.g., relapsed or refractory DLBCL, or FL, e.g., relapsed or refractory FL) or at risk of developing cancer (e.g., a hematological cancer such as ALL, CLL, DLBCL, e.g., relapsed or refractory DLBCL, or FL, e.g., relapsed or refractory FL) will be more likely to respond to CAR-expressing cell therapy (e.g., a CD19 CAR-expressing cell therapy described herein such as, e.g., CTL019).

Accordingly, in one aspect, the disclosure provides a method for determining whether a subject with cancer (e.g., a hematological cancer such as ALL, CLL, DLBCL, e.g., relapsed or refractory DLBCL, or FL, e.g., relapsed or refractory FL) is likely to respond to a cell expressing a CAR, e.g., a CD19 CAR-expressing cell described herein, such as CTL019. In another aspect, the disclosure is drawn to a method for predicting a time course of disease. In still another aspect, the method is drawn to a method for predicting a probability of a significant event in the time course of the disease (e.g., reoccurrence or remission). In certain embodiments, the method comprises detecting a combination of biomarkers associated with responsiveness to treatment as described herein and determining whether the subject is likely to respond to treatment.

In an aspect, the disclosure provides a method for providing a prognosis for success rate of a CAR-expressing cell therapy (e.g., a CD19 CAR-expressing cell therapy described herein such as, e.g., CTL019) in a subject having cancer (e.g., a hematological cancer such as ALL, CLL, DLBCL, e.g., relapsed or refractory DLBCL, or FL, e.g., relapsed or refractory FL), said method comprising steps of:

providing a biological sample from the subject;

determining the levels of expression of one or more biomarkers including but not limited to CD27, CD45RO, CCR7, HLA-DR, CD127, and CD95 to obtain a gene expression pattern for the sample; and

based on the gene expression pattern obtained, providing a prognosis to the subject.

In an embodiment, the step of determining the levels of expression of the set of genes further comprises detecting the expression of mRNA expressed from said genes. In an embodiment, provided methods further comprise a step wherein determining the expression of mRNA comprises exposing said mRNA to a nucleic acid probe complementary to said mRNA.

In an embodiment, the step of determining the levels of expression of the set of genes further comprises detecting the expression of a polypeptide encoded by said genes.

In an embodiment, provided methods comprise selecting a CAR-expressing cell (e.g., T cell, NK cell) therapy (e.g., a CD19 CAR-expressing cell therapy described herein such as, e.g., CTL019) for the subject, based on the prognosis provided.

In some embodiments, the methods involve evaluation of a biological sample, e.g., a sample from a subject, e.g., a patient who has been diagnosed with or is suspected of having cancer (e.g., a hematological cancer such as ALL, CLL, DLBCL, e.g., relapsed or refractory DLBCL, or FL, e.g., relapsed or refractory FL, e.g., presents with symptoms of ALL, CLL, DLBCL, e.g., relapsed or refractory DLBCL, or FL, e.g., relapsed or refractory FL) to detect changes in expression and/or activity of one or more biomarkers, including but not limited to CD27, CD45RO, CCR7, HLA-DR, CD127, and CD95, and/or a CD19 CAR-expressing cell (e.g., T cell, NK cell) gene signature.

The results of the screening method and the interpretation thereof are predictive of the patient disease progression (e.g., progression of a cancer, e.g., a hematological cancer such as ALL, CLL, DLBCL, e.g., relapsed or refractory DLBCL, or FL, e.g., relapsed or refractory FL). According to the present invention, alterations in expression or activity of one or more biomarkers, including but not limited to CD27, CD45RO, CCR7, HLA-DR, CD127, and CD95, and/or a CD19 CAR-expressing cell (e.g., T cell, NK cell) gene signature is indicative of cancer progression (e.g., a hematological cancer such as ALL, CLL, DLBCL, e.g., relapsed or refractory DLBCL, or FL, e.g., relapsed or refractory FL) relative to an average or median value for a cancer patient population or to an average median for a population of healthy, cancer free subjects.

In some embodiments, a subject having FL, e.g., relapsed or refractory FL, is evaluated for a biomarker in a sample from the subject, e.g., a tumor biopsy, peripheral blood or circulating tumor DNA. For example, a tumor biopsy can be evaluated for expression of, e.g., CD19, PD-1 or PD-L1, as described in Example 4. In some embodiments, soluble immune markers, the serum levels of inflammatory cytokines or other soluble factors can be assessed pre or post CAR expressing cell infusion, e.g., as described in Example 4. In some embodiments, effect of CAR expressing cell therapy, e.g., CAR19 expressing cell therapy, is evaluated in, e.g., peripheral blood, to assess, e.g., on target effect of CAR therapy on, e.g., levels of CD19 positive B cells. In some embodiments, peripheral blood characterization can include immunophenotyping, T cell subset frequency, transcriptome analysis or SNP analysis. In some embodiments, immunophenotyping of peripheral blood includes, e.g., deep sequencing, e.g., Ig deep sequencing. In some embodiments, a sample from the subject can be evaluated for minimal residual disease, e.g., as described in Example 4.

In yet another embodiment, the one or more alterations, e.g., alterations in biomarker expression are assessed at pre-determined intervals, e.g., a first point in time and at least at a subsequent point in time. In one embodiment, a time course is measured by determining the time between significant events in the course of a subject disease, wherein the measurement is predictive of whether a subject has a long time course. In another embodiment, the significant event is the progression from diagnosis to death. In another embodiment, the significant event is the progression from diagnosis to worsening disease.

### PD1/PD-L1 Interaction Score as a Biomarker

In an aspect, the disclosure provides a method of evaluating a subject, e.g., evaluating or monitoring the effectiveness of a CAR-expressing cell therapy in a subject, having a cancer, comprising: acquiring a value of responder status to a therapy comprising a CAR-expressing cell population (e.g., a CAR19-expressing cell population) for the subject, wherein said value of responder status comprises a measure of the level or activity of PD-1 and/or PD-L1, wherein the measure comprises an interaction score, e.g., an interaction score of PD1 and PDL1 (PD1/PDL1), thereby evaluating the subject. In some embodiments, the responder status is indicative of a complete response, a partial response, a non-response, or a relapse to the CAR-expressing cell therapy. Also provided herein are methods of measuring a PD1/PD-L1 interaction score, e.g., using AQUA technology, e.g., as described in Example 3. Detailed methods of measuring an interaction score, e.g., of PD1/PD-L1, are described in International Application WO 2017/070582 filed on 21 Oct. 2016, the entire contents of which are hereby incorporated by reference.

In some embodiments, the interaction score is obtained by, e.g., performing at least the following steps:

(i) generating image data based on detected electromagnetic radiation;

(ii) analyzing image data to determine a score representative of a nearness between a least one pair of cells, a first member of the least one pair of cells expressing a first biomarker, e.g., PD1 or PD-L1, and a second member of the at least one pair of cells expressing a second biomarker, e.g., PD1 or PD-L1 that is different from the first biomarker; and

(iii) recording the score, which score when compared to a threshold value is indicative of a likelihood that the subject will respond, e.g., positively to immune-therapy, e.g., immunotherapy, e.g., CAR-expressing cell therapy as described herein.

In some embodiments, the score is representative of a nearness between at least one pair cells is representative of an extent that the pair of cells are within a predetermined proximity of one another.

In some embodiments, the score is calculated by obtaining a proximity between the boundaries of the pair of cells.

In some embodiments, a low PD1/PD-L1 interaction score, e.g., an interaction score less than about 1800 (e.g., about 1700-1500, 1500-1300, 1300-1100, 1100-900, 900-700, 700-500, 500-400, 400-300, 300-200, 200-100, 100-0, or at least about 1700, 1500, 1300, 1100, 1000, 900, 800, 700, 600, 500, 400, 300, 200, 100, or lesser) is indicative of a subject's responsiveness, e.g., complete response or partial response, to a CAR-expressing cell therapy. In some embodiments, a low PD1/PD-L1 interaction score, e.g., as described herein, is indicative of a subject not having a non-response or a relapse to CAR-expressing cell therapy, e.g., CAR19 expressing cell therapy, in less than about 3 months, e.g., less than about 2 months or 1 month, after administration of the CAR-expressing cell therapy. In some embodiments, a low PD1/PD-L1 interaction score, e.g., an interaction score less than about 1800 (e.g., about 1700, 1500, 1300, 1100, 1000, 900, 800, 700, 600, 500, 400, 300, 200, 100, or lesser), is indicative of improved efficacy of a CAR-expressing cell therapy, e.g., a CAR19 expressing cell therapy.

In some embodiments, a high PD1/PD-L1 interaction score, e.g., an interaction score of at least about 1800 or higher (e.g., about 1800-6000, e.g., about 1800-2500, about 2500-3500, about 3500-5000, or about 5000-6000, or at least about 2000, 2200, 2400, 2600, 2800, 3000, 3200, 3400, 3600, 3800, 4000, 4200, 4400, 4600, 4800, 5000, 5200, 5400, 5600, 5800, or 6000 or higher), is indicative of a lack of a subject's responsiveness, e.g., a non-response or relapse, to a CAR-expressing cell therapy. In some embodiments, a high PD1/PD-L1 interaction score, e.g., as described herein, is indicative of a subject having a non-response or a relapse to CAR-expressing cell therapy, e.g., CAR19 expressing cell therapy, in less than about 3 months, e.g., less than about 2 months or 1 month, after administration of the CAR-expressing cell therapy. In some embodiments, a high PD1/PD-L1 interaction score, e.g., at least about 1800 or higher (e.g., about 1800-6000), is indicative of reduced efficacy of a CAR-expressing cell therapy, e.g., a CAR19 expressing cell therapy. In some embodiments, a subject with a high PD1/PD-L1 interaction score, e.g., at least about 1800 or higher (e.g., about 1800-6000), is administered an additional therapy, e.g., as described herein. In some embodiments, a subject with a high PD1/PD-L1 interaction score is administered: (i) an altered, e.g., higher, dose of the CAR-expressing cell therapy; (ii) a different dosing regimen, e.g., a different frequency of dosing, of the CAR-expressing cell therapy; or (iii) an additional agent, e.g., as described herein, in combination with the CAR-expressing cell therapy.

### Biomarkers for Predicting Neurotoxicity

The disclosure provides, inter alia, methods of evaluating, e.g., predicting, a subject's a subject's risk for developing neurotoxicity and/or CRS. In some embodiments, the method comprises acquiring a neurotoxicity risk status for the subject, e.g., in response to a CAR-expressing cell therapy (e.g., a CAR19-expressing cell therapy), wherein said neurotoxicity risk status comprises a measure of one, two, three, four, five, six, seven, eight or more (all) of the following:

(i) the level or activity of soluble CD30 (sCD30);

(ii) the level or activity of soluble tumor necrosis factor receptor-1 (sTNFR-1);

(iii) the level or activity of interleukin 2 (IL-2);

(iv) the level or activity of soluble interleukin 4 receptor (sIL-4R),

(v) the level or activity of hepatocyte growth factor (HGF), or

(vi) the level or activity of interleukin 15 (IL-15);

(vii) the level or activity of sRAGE;

(viii) the level or activity of VEGFR-1; or

(ix) the level or activity of VEGFR-2,

wherein the measure is acquired from a sample, e.g., a serum sample, blood sample, CSF sample or brain parenchyma sample, from the subject, and wherein the neurotoxicity risk status is indicative of the subject's risk for developing neurotoxicity, e.g., severe neurotoxicity, thereby evaluating the subject's risk for developing neurotoxicity.

In some embodiments, the measure of any one or all of (i)-(ix) is compared to a measure obtained from a subject not predicted to be at risk of developing neurotoxicity.

In some embodiments, the measure of any one or all of (i)-(ix) is acquired within 3 days, e.g., within 1 day, 2 days or 3 days, post CAR expressing cell infusion.

In some embodiments, a decrease in (i) is indicative of the subject's risk for developing neurotoxicity, e.g., severe neurotoxicity.

In some embodiments, an increase in (ii) is indicative of the subject's risk for developing neurotoxicity, e.g., severe neurotoxicity.

In some embodiments, a difference in any one or all of (i)-(ix) compared to a corresponding measure of any one or all of (i)-(ix) obtained from a subject not predicted to be at risk of developing neurotoxicity, is indicative of the subject's risk of developing neurotoxicity.

In some embodiments, the risk status comprises a measure of (i). Without wishing to be bound by theory, it is believed that, in some embodiments, low levels of SCD30 may indicate a shift towards a more inflammatory Th1 immune response, e.g., a response that is more neurotoxic.

In some embodiments, the risk status comprises a measure of (i) and (ii). In some embodiments, a subject with a decrease in (i) and an increase in (ii), is predicted to be at risk of developing neurotoxicity. In some embodiments, a measure of (i) or (ii) is acquired within 3 days, e.g., within 1 day, 2 days or 3 days, post CAR expressing cell infusion. In some embodiments, the subject predicted to be at risk of developing neurotoxicity is administered an agent that targets the TNF pathway. Without wishing to be bound by theory, it is believed that, in some embodiments, high levels of sTNFR-1 may indicate a shift towards a more inflammatory Th1 immune response, e.g., a response that is more neurotoxic. In some embodiments, the subject predicted to be at risk of developing neurotoxicity is administered an agent other than a CRS therapy, e.g., as described herein.

In an aspect, disclosed herein is a method of treating, e.g., preventing, neurotoxicity in a subject comprising administering to the subject an agent that targets the TNF pathway, e.g., as described herein. In some embodiments, the subject has been administered a CAR-expressing cell therapy, e.g., CAR19 expressing cell therapy. In an embodiment, the subject is selected for administration of an agent that targets the TNF pathway by evaluating, e.g., predicting, the subject's risk for developing neurotoxicity, e.g., as described herein, comprising:

acquiring a neurotoxicity risk status for the subject, e.g., in response to a CAR-expressing cell therapy (e.g., a CAR19-expressing cell therapy), wherein said neurotoxicity risk status comprises a measure of one, two, three, four, five, six, seven, eight or more (all) of the following:

(i) the level or activity of soluble CD30 (sCD30);

(ii) the level or activity of soluble tumor necrosis factor receptor-1 (sTNFR-1);

(iii) the level or activity of interleukin 2 (IL-2);

(iv) the level or activity of soluble interleukin 4 receptor (sIL-4R),

(v) the level or activity of hepatocyte growth factor (HGF), or

(vi) the level or activity of interleukin 15 (IL-15);

(vii) the level or activity of sRAGE;

(viii) the level or activity of VEGFR-1; or

(ix) the level or activity of VEGFR-2.

In some embodiments, the subject is a pediatric subject or a young adult.

In some embodiments, the subject has previously been administered a CAR-expressing cell therapy, e.g., a CAR19 expressing cell therapy, e.g., CTL019.

In some embodiments, the neurotoxicity, e.g., as described herein, is associated with a CAR-expressing cell therapy. In some embodiments, the CRS is associated with a CAR-expressing cell therapy

In some embodiments, the subject was administered the CAR-expressing cell therapy, e.g., about 30 days, e.g., 29, 28, 27, 26, 25, 24, 23, 22, 21, 20, 19, 18, 17, 16, 15, 14, 13, 12, 11, 10, 9, 8, 7, 6, 5, 4, 3, 2, or 1 days, prior to development, e.g., appearance, of neurotoxicity. In some embodiments, the subject is evaluated, about 30 days, e.g., 29, 28, 27, 26, 25, 24, 23, 22, 21, 20, 19, 18, 17, 16, 15, 14, 13, 12, 11, 10, 9, 8, 7, 6, 5, 4, 3, 2, or 1 days after administration of CAR-expressing cell therapy.

In some embodiments, the subject at risk of developing neurotoxicity is also at risk of developing CRS. In some embodiments, development of neurotoxicity, e.g., severe neurotoxicity, in the subject is correlated with development of CRS, e.g., high grade CRS.

In some embodiments, the subject at risk of developing neurotoxicity is not at risk of developing CRS. In some embodiments, development of neurotoxicity in the subject is not correlated with development of CRS. In some embodiments, CRS therapies e.g., as described herein, do not, e.g., reduce the symptoms of neurotoxicity. Without wishing to be bound by theory, it is believed that in some embodiments the mechanism of neurotoxicity development differs from the mechanism of CRS development. In some embodiments, neurotoxicity can be caused by, e.g., NK cell mediated inflammation. In some embodiments, a subject with neurotoxicity has increased levels of e.g., IL-2 or IL-15.

In some embodiments, neurotoxicity includes, but is not limited to encephalopathy (e.g., as described herein), focal deficit (e.g., as described herein), seizure (e.g., as described herein), or other clearly defined neurologic symptom. In some embodiments, neurotoxicity comprises severe neurotoxicity. In some embodiments, severe neurotoxicity comprises any one or all of focal neurotoxicity, encephalopathy, or seizure.

In some embodiments, encephalopathy includes Speech impairment, Blurred vision, Hallucination, Diplopia, Ataxia, Ophthalmoplegia, Pupil fixed and dilated, Dysarthria, Loss of vision, Somnolence, Mutism, Encephalopathy, Hypotonia, Productive aphasia, Aphasia, Akinetic mutism, Confusion, Mental status change, Tremor, Speech impairment, Mood alteration, Irritability, Dizziness, Facial droop, Insomnia, Fatigue, Photophobia, Double vision, Lip/jaw numbness, Delayed response to questions, Decreased vibration, Agitation/emergence delirium, “body shaking”, Memory problems, Encephalopathy, Seizure, Electrographic seizure, or Subclinical seizure.

In some embodiments, focal neurotoxicity includes, speech impairment, blurred vision, diplopia, ataxia, ophthalmoplegia, pupil fixed and dilated, dysarthria, loss of vision, mutism, productive aphasia, aphasia, akinetic mutics, speech impairment, facial droop, double vision, or lip/jaw numbness.

In some embodiments, encephalopathy includes symptoms reported or noted on history, impression, or physical examination, e.g., a diagnosis of “patient not arousable”.

In some embodiments, seizure includes electropaphic seizure, seizure, or subclinical seizure.

In some embodiments, severe neurotoxicity includes encephalopathy, seizure or aphasia.

In some embodiments, neurotoxicity does not include headache or delirium.

In some embodiments, neurotoxicity in a pediatric subject or a young adult, can be associated with a predisposition to developing neurotoxicity, e.g., a vulnerability, e.g., a prior injury. In some embodiments, neurotoxicity can be associated with, e.g., neurologic deficit.

In some embodiments, neurotoxicity in a pediatric subject or a young adult is different from neurotoxicity in an adult. In some embodiments neurotoxicity in a pediatric subject or a young adult does not include, e.g., aphasia or cerebral edema. Without wishing to be bound by theory, it is believed that in some embodiments, there may be different causes for the development of neurotoxicity from CAR-therapies in the developing pediatric brain compared to adults.

In some embodiments, development of neurotoxicity following CAR therapy can depend on the age of the subject.

In some embodiments, the level and or activity of soluble tumor necrosis factor receptor-1 (sTNFR-1) is higher in a subject who develops neurotoxicity, e.g., encephalopathy, compared to a subject who does not develop neurotoxicity, e.g., encephalopathy. In some embodiments, subjects who develop neurotoxicity, e.g., encephalopathy, have a higher 35-day peak cytokine level compared to subjects who do not develop neurotoxicity.

In some embodiments, a subject who develops neurotoxicity, e.g., as described herein, has a higher level of one or more of the following cytokines: interleukin 2 (IL-2), soluble interleukin 4 receptor (sIL-4R), hepatocyte growth factor (HGF), and interleukin 15 (IL-15), as compared to a subject who does not develop neurotoxicity. In some embodiments, the subject does not develop CRS.

In embodiments, CRS is graded according to Table 8:

TABLE 8
CRS grading

| | |
|---|---|
| Gr1 | Supportive care only |
| Gr2 | IV therapies +/− hospitalization. |
| Gr3 | Hypotension requiring IV fluids or low-dose vasoactives or |
| | hypoxemia requiring oxygen, CPAP, or BIPAP. |
| Gr4 | Hypotension requiring high-dose vasoactives or hypoxemia |
| | requiring mechanical ventilation. |
| Gr 5 | Death |

Therapies for CRS include IL-6 inhibitor or IL-6 receptor (IL-6R) inhibitors (e.g., tocilizumab or siltuximab), bazedoxifene, sgp130 blockers, vasoactive medications, corticosteroids, immunosuppressive agents, and mechanical ventilation. Exemplary therapies for CRS are described in International Application WO2014011984, which is hereby incorporated by reference.

Tocilizumab is a humanized, immunoglobulin G1kappa anti-human IL-6R monoclonal antibody. See, e.g., id. Tocilizumab blocks binding of IL-6 to soluble and membrane bound IL-6 receptors (IL-6Rs) and thus inhibitors classical and trans-IL-6 signaling. In embodiments, tocilizumab is administered at a dose of about 4-12 mg/kg, e.g., about 4-8 mg/kg for adults and about 8-12 mg/kg for pediatric subjects, e.g., administered over the course of 1 hour.

In some embodiments, the CRS therapeutic is an inhibitor of IL-6 signalling, e.g., an inhibitor of IL-6 or IL-6 receptor. In one embodiment, the inhibitor is an anti-IL-6 antibody, e.g., an anti-IL-6 chimeric monoclonal antibody such as siltuximab. In other embodiments, the inhibitor comprises a soluble gp130 (sgp130) or a fragment thereof that is capable of blocking IL-6 signalling. In some embodiments, the sgp130 or fragment thereof is fused to a heterologous domain, e.g., an Fc domain, e.g., is a gp130-Fc fusion protein such as FE301. In embodiments, the inhibitor of IL-6 signalling comprises an antibody, e.g., an antibody to the IL-6 receptor, such as sarilumab, olokizumab (CDP6038), elsilimomab, sirukumab (CNTO 136), ALD518/BMS-945429, ARGX-109, or FM101. In some embodiments, the inhibitor of IL-6 signalling comprises a small molecule such as CPSI-2364.

Exemplary vasoactive medications include but are not limited to angiotensin-11, endothelin-1, alpha adrenergic agonists, rostanoids, phosphodiesterase inhibitors, endothelin antagonists, inotropes (e.g., adrenaline, dobutamine, isoprenaline, ephedrine), vasopressors (e.g., noradrenaline, vasopressin, metaraminol, vasopressin, methylene blue), inodilators (e.g., milrinone, levosimendan), and dopamine.

Exemplary vasopressors include but are not limited to norepinephrine, dopamine, phenylephrine, epinephrine, and vasopressin. In some embodiments, a high-dose vasopressor includes one or more of the following: norepinephrine monotherapy at ≥20 ug/min, dopamine monotherapy at ≥10 ug/kg/min, phenylephrine monotherapy at ≥200 ug/min, and/or epinephrine monotherapy at ≥10 ug/min. In some embodiments, if the subject is on vasopressin, a high-dose vasopressor includes vasopressin+norepinephrine equivalent of ≥10 ug/min, where the norepinephrine equivalent dose=[norepinephrine (ug/min)]+[dopamine (ug/kg/min)/2]+[epinephrine (ug/min)]+[phenylephrine (ug/min)/10]. In some embodiments, if the subject is on combination vasopressors (not vasopressin), a high-dose vasopressor includes norepinephrine equivalent of ≥20 ug/min, where the norepinephrine equivalent dose=[norepinephrine (ug/min)]+[dopamine (ug/kg/min)/2]+[epinephrine (ug/min)]+[phenylephrine (ug/min)/10]. See e.g., Id.

In some embodiments, a low-dose vasopressor is a vasopressor administered at a dose less than one or more of the doses listed above for high-dose vasopressors.

Exemplary corticosteroids include but are not limited to dexamethasone, hydrocortisone, and methylprednisolone. In embodiments, a dose of dexamethasone of 0.5 mg/kg is used. In embodiments, a maximum dose of dexamethasone of 10 mg/dose is used. In embodiments, a dose of methylprednisolone of 2 mg/kg/day is used.

Exemplary immunosuppressive agents include but are not limited to an inhibitor of TNFα or an inhibitor of IL-1. In embodiments, an inhibitor of TNFα comprises an anti-TNFα antibody, e.g., monoclonal antibody, e.g., infliximab. In embodiments, an inhibitor of TNFα comprises a soluble TNFα receptor (e.g., etanercept). In embodiments, an IL-1 or IL-1R inhibitor comprises anakinra.

In some embodiments, the subject at risk of developing severe CRS is administered an anti-IFN-gamma or anti-sIL2Ra therapy, e.g., an antibody molecule directed against IFN-gamma or sIL2Ra.

In embodiments, for a subject who has received a therapeutic antibody molecule such as blinatumomab and who has CRS or is at risk of developing CRS, the therapeutic antibody molecule is administered at a lower dose and/or a lower frequency, or administration of the therapeutic antibody molecule is halted.

In embodiments, a subject who has CRS or is at risk of developing CRS is treated with a fever reducing medication such as acetaminophen.

In embodiments, a subject herein is administered or provided one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation, in any combination, e.g., in combination with a CAR-expressing cell described herein.

In embodiments, a subject at risk of developing CRS (e.g., severe CRS) (e.g., identified as having a high risk status for developing severe CRS) is administered one or more therapies for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation, in any combination, e.g., in combination with a CAR-expressing cell described herein.

In embodiments, a subject herein (e.g., a subject at risk of developing severe CRS or a subject identified as at risk of developing severe CRS) is transferred to an intensive care unit. In some embodiments, a subject herein (e.g., a subject at risk of developing severe CRS or a subject identified as at risk of developing severe CRS) is monitored for one ore more symptoms or conditions associated with CRS, such as fever, elevated heart rate, coagulopathy, MODS (multiple organ dysfunction syndrome), cardiovascular dysfunction, distributive shock, cardiomyopathy, hepatic dysfunction, renal dysfunction, encephalopathy, clinical seizures, respiratory failure, or tachycardia. In some embodiments, the methods herein comprise administering a therapy for one of the symptoms or conditions associated with CRS. For instance, in embodiments, e.g., if the subject develops coagulopathy, the method comprises administering cryoprecipitate. In some embodiments, e.g., if the subject develops cardiovascular dysfunction, the method comprises administering vasoactive infusion support. In some embodiments, e.g., if the subject develops distributive shock, the method comprises administering alpha-agonist therapy. In some embodiments, e.g., if the subject develops cardiomyopathy, the method comprises administering milrinone therapy. In some embodiments, e.g., if the subject develops respiratory failure, the method comprises performing mechanical ventilation (e.g., invasive mechanical ventilation or noninvasive mechanical ventilation). In some embodiments, e.g., if the subject develops shock, the method comprises administering crystalloid and/or colloid fluids.

In embodiments, the CAR-expressing cell is administered prior to, concurrently with, or subsequent to administration of one or more therapies for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation. In embodiments, the CAR-expressing cell is administered within 2 weeks (e.g., within 2 or 1 week, or within 14 days, e.g., within 14, 13, 12, 11, 10, 9, 8, 7, 6, 5, 4, 3, 2, 1 day or less) of administration of one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation. In embodiments, the CAR-expressing cell is administered at least 1 day (e.g., at least 1, 2, 3, 4, 5, 6, 7, 8, 9, 10, 11, 12, 13, 14, 15, 16, 17, 18, 19, 20, 1, week, 2 weeks, 3 weeks, 4 weeks, 1 month, 2 months, 3 months, 3 months, or more) before or after administration of one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation.

In embodiments, a subject herein (e.g., a subject at risk of developing severe CRS or a subject identified as at risk of developing severe CRS) is administered a single dose of an IL-6 inhibitor or IL-6 receptor (IL-6R) inhibitor (e.g., tocilizumab). In embodiments, the subject is administered a plurality of doses (e.g., 2, 3, 4, 5, 6, or more doses) of an IL-6 inhibitor or IL-6 receptor (IL-6R) inhibitor (e.g., tocilizumab).

In embodiments, a subject at low or no risk of developing CRS (e.g., severe CRS) (e.g., identified as having a low risk status for developing severe CRS) is not administered a therapy for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation.

In embodiments, a subject is determined to be at high risk of developing severe CRS by using an evaluation or prediction method described herein. In embodiments, a subject is determined to be at low risk of developing severe CRS by using an evaluation or prediction method described herein.

### Methods for Detection of Gene Expression

Biomarker expression level can also be assayed. Expression of a marker described herein can be assessed by any of a wide variety of known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods. Exemplary methods of detection of gene expression is disclosed in International Application WO 2016/057705 filed on 7 Oct. 2015, the entire contents of which are hereby incorporated by reference.

In certain embodiments, activity of a particular gene is characterized by a measure of gene transcript (e.g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, CDNA, mRNA, protein, or enzyme activity), or, alternatively, can be a qualitative assessment of the level of gene expression, in particular in comparison with a control level. The type of level being detected will be clear from the context.

Methods of detecting and/or quantifying the gene transcript (mRNA or CDNA made therefrom) using nucleic acid hybridization techniques are known to those of skill in the art (see e.g., Sambrook et al. supra), and are disclosed in International Application WO 2016/057705 filed on 7 Oct. 2015.

As an alternative to making determinations based on the absolute expression level of the marker, determinations can be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a healthy subject, or between samples from different sources.

Alternatively, the expression level can be provided as a relative expression level. To determine a relative expression level of a marker, the level of expression of the marker can be determined for 10 or more samples of normal versus cancer isolates, or even 50 or more samples, prior to the determination of the expression level for the sample in question. The mean expression level of each of the genes assayed in the larger number of samples can be determined and this can be used as a baseline expression level for the marker. The expression level of the marker determined for the test sample (absolute level of expression) then can be divided by the mean expression value obtained for that marker. This provides a relative expression level.

In certain embodiments, the samples used in the baseline determination will be from samples derived from a subject having cancer (e.g., a hematological cancer such as ALL and CLL) versus samples from a healthy subject of the same tissue type. The choice of the cell source is dependent on the use of the relative expression level. Using expression found in normal tissues as a mean expression score aids in validating whether the marker assayed is specific to the tissue from which the cell was derived (versus normal cells). In addition, as more data is accumulated, the mean expression value can be revised, providing improved relative expression values based on accumulated data. Expression data from normal cells provides a means for grading the severity of the cancer disease state.

In another embodiment, expression of a marker is assessed by preparing genomic DNA or mRNA/CDNA (i.e., a transcribed polynucleotide) from cells in a subject sample, and by hybridizing the genomic DNA or mRNA/CDNA with a reference polynucleotide which is a complement of a polynucleotide comprising the marker, and fragments thereof. CDNA can, optionally, be amplified using any of a variety of polymerase chain reaction methods prior to hybridization with the reference polynucleotide. Expression of one or more markers can likewise be detected using quantitative PCR (QPCR) to assess the level of expression of the marker(s). Alternatively, any of the many known methods of detecting mutations or variants (e.g., single nucleotide polymorphisms, deletions, etc.) of a marker of the invention can be used to detect occurrence of a mutated marker in a subject.

In another embodiment, a combination of methods to assess the expression of a marker is utilized.

Because the compositions, kits, and methods rely on detection of a difference in expression levels of one or more markers described herein, in certain embodiments the level of expression of the marker is significantly greater than the minimum detection limit of the method used to assess expression in at least one of a biological sample from a subject with cancer (e.g., a hematological cancer such as ALL and CLL) or a reference (e.g., a biological sample from a healthy subject, e.g., a subject without cancer).

### Nucleic Acid Molecules and Probes

One aspect of the disclosure pertains to isolated nucleic acid molecules that correspond to one or markers described herein, including nucleic acids which encode a polypeptide corresponding to one or more markers described herein or a portion of such a polypeptide. The nucleic acid molecules include those nucleic acid molecules which reside in genomic regions identified herein. Isolated nucleic acid molecules also include nucleic acid molecules sufficient for use as hybridization probes to identify nucleic acid molecules that correspond to a marker described herein, including nucleic acid molecules which encode a polypeptide corresponding to a marker described herein, and fragments of such nucleic acid molecules, e.g., those suitable for use as PCR primers for the amplification or mutation of nucleic acid molecules. Nucleic acid molecules can be DNA molecules (e.g., CDNA or genomic DNA) and RNA molecules (e.g., mRNA) and analogs of the DNA or RNA generated using nucleotide analogs. The nucleic acid molecule can be single-stranded or double-stranded; in certain embodiments the nucleic acid molecule is double-stranded DNA.

An “isolated” nucleic acid molecule is one which is separated from other nucleic acid molecules which are present in the natural source of the nucleic acid molecule. In certain embodiments, an “isolated” nucleic acid molecule is free of sequences (such as protein-encoding sequences) which naturally flank the nucleic acid (i.e., sequences located at the 5 and 3 ends of the nucleic acid) in the genomic DNA of the organism from which the nucleic acid is derived.

The language “substantially free of other cellular material or culture medium” includes preparations of nucleic acid molecule in which the molecule is separated from cellular components of the cells from which it is isolated or recombinantly produced. Thus, nucleic acid molecule that is substantially free of cellular material includes preparations of nucleic acid molecule having less than about 30%, less than about 20%, less than about 10%, or less than about 5% (by dry weight) of other cellular material or culture medium.

If so desired, a nucleic acid molecule, e.g., the marker gene products identified herein, can be isolated using standard molecular biology techniques and the sequence information in the database records described herein. Using all or a portion of such nucleic acid sequences, nucleic acid molecules can be isolated using standard hybridization and cloning techniques (e.g., as described in Sambrook et al., ed., MOLECULAR CLONING: A LABORATORY MANUAL, 2nd ed., Cold Spring Harbor Laboratory Press, Cold Spring Harbor, N.Y., 1989).

A nucleic acid molecule can be amplified using CDNA, mRNA, or genomic DNA as a template and appropriate oligonucleotide primers according to standard PCR amplification techniques. Additional details of nucleic acid amplification is provided in International Application WO 2016/057705 filed on 7 Oct. 2015, the contents of which is hereby incorporated by reference in its entirety.

### Polypeptide Detection

Methods to measure biomarkers described herein, include, but are not limited to: Western blot, immunoblot, enzyme-linked immunosorbant assay (ELISA), radioimmunoassay (MA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF) mass spectrometry, microcytometry, microarray, microscopy, fluorescence activated cell sorting (FACS), magnetic activated cell sorting (MACS), flow cytometry, time of flight mass spectrometry (CyTOF), laser scanning cytometry, hematology analyzer and assays based on a property of the protein including but not limited to DNA binding, ligand binding, or interaction with other protein partners.

The activity or level of a marker protein can also be detected and/or quantified by detecting or quantifying the expressed polypeptide. The polypeptide can be detected and quantified by any of a number of means well known to those of skill in the art. These can include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (MA), enzyme-linked immunosorbent assays (ELISAs), immunofluorescent assays, Western blotting, immunohistochemistry and the like. A skilled artisan can readily adapt known protein/antibody detection methods for use in determining the expression level of one or more biomarkers in a serum sample.

Another agent for detecting a polypeptide is an antibody capable of binding to a polypeptide corresponding to a marker described herein, e.g., an antibody with a detectable label. Antibodies can be polyclonal or monoclonal. An intact antibody, or a fragment thereof (e.g., Fab or F(ab)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In another embodiment, the polypeptide is detected and/or quantified using Luminex® assay technology. The Luminex® assay separates tiny color-coded beads into e.g., distinct sets that are each coated with a reagent for a particular bioassay, allowing the capture and detection of specific analytes from a sample in a multiplex manner. The Luminex® assay technology can be compared to a multiplex ELISA assay using bead-based fluorescence cytometry to detect analytes such as biomarkers.

Additional techniques of detecting polypeptides, including protein isolation from cells, are disclosed in International Application WO 2016/057705 filed on 7 Oct. 2015, the contents of which is hereby incorporated by reference in its entirety.

The disclosure also encompasses kits for detecting the presence of a polypeptide or nucleic acid corresponding to a marker described herein in a biological sample, e.g., a sample containing tissue, whole blood, serum, plasma, buccal scrape, saliva, cerebrospinal fluid, urine, stool, and bone marrow. Such kits can be used to determine if a subject is suffering from or is at increased risk of developing cancer (e.g., a hematological cancer such as CLL and ALL). For example, the kit can comprise a labeled compound or agent capable of detecting a polypeptide or an mRNA encoding a polypeptide corresponding to a marker described herein in a biological sample and means for determining the amount of the polypeptide or mRNA in the sample (e.g., an antibody which binds the polypeptide or an oligonucleotide probe which binds to DNA or mRNA encoding the polypeptide). Kits can also include instructions for interpreting the results obtained using the kit.

The disclosure thus includes a kit for assessing the disease progression of a subject having cancer (e.g., a hematological cancer such as CLL and ALL).

In an embodiment, a kit can be used to assess the disease progression of a cancer including, but not limited to, B-cell acute lymphocytic leukemia (B-ALL), T-cell acute lymphocytic leukemia (T-ALL), acute lymphocytic leukemia (ALL), chronic myelogenous leukemia (CIVIL), chronic lymphocytic leukemia (CLL), B cell promyelocytic leukemia, blastic plasmacytoid dendritic cell neoplasm, Burkitt lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, and Waldenstrom macroglobulinemia. In an embodiment, the disclosure provides a kit for assessing the disease progression of a subject having a hematological cancer. In an embodiment, the disclosure provides a kit for assessing the disease progression of a subject having ALL. In another embodiment, the disclosure provides a kit for assessing the disease progression of a subject having CLL. In an embodiment, the disclosure provides a kit for assessing the disease progression of a subject having cancer that is associated with CD19 expression.

In an embodiment, the disclosure provides a kit for assessing and characterizing responder status (e.g., compete responder, partial responder or non-responder) of a subject having a hematological cancer to a CAR-expressing cell (e.g., T cell, NK cell) therapy (e.g., a CD19 CAR-expressing cell therapy as described herein, such as e.g., CTL019). In an embodiment, the disclosure provides a kit for assessing and characterizing responder status (e.g., compete responder, partial responder or non-responder) of a subject having ALL to a CAR-expressing cell therapy (e.g., a CD19 CAR-expressing cell therapy as described herein, such as e.g., CTL019). In an embodiment, the disclosure provides a kit for assessing and characterizing responder status (e.g., compete responder, partial responder or non-responder) of a subject having CLL to a CAR-expressing cell therapy (e.g., a CD19 CAR-expressing cell therapy as described herein, such as e.g., CTL019).

Suitable reagents for binding with a polypeptide corresponding to a marker described herein include antibodies, antibody derivatives, antibody fragments, and the like. Suitable reagents for binding with a nucleic acid (e.g., a genomic DNA, an mRNA, a spliced mRNA, a CDNA, or the like) include complementary nucleic acids. For example, the nucleic acid reagents can include oligonucleotides (labeled or non-labeled) fixed to a substrate, labeled oligonucleotides not bound with a substrate, pairs of PCR primers, molecular beacon probes, and the like.

The kit can optionally comprise additional components useful for performing the methods described herein. By way of example, the kit can comprise fluids (e.g., SSC buffer) suitable for annealing complementary nucleic acids or for binding an antibody with a protein with which it specifically binds, one or more sample compartments, an instructional material which describes performance of a method of the invention, a reference sample for comparison of expression levels of the biomarkers described herein, and the like.

A kit of the invention can comprise a reagent useful for determining protein level or protein activity of a marker.

In an embodiment, a kit is provided for providing a prognosis for success rate of a CAR-expressing cell (e.g., T cell, NK cell) therapy (e.g., a CD19 CAR-expressing cell therapy as described herein, such as e.g., CTL019) in a subject having cancer (e.g., a hematological cancer such as CLL and ALL), said kit comprising:

a set of reagents that specifically detects expression levels of one or more (e.g., 2, 3, 4, or all) of CD27, CD45RO, CCR7, CD95, CD127 and HLA-DR, and a CD19 CAR-expressing cell gene set signature; and

instructions for using said kit;

wherein said instructions for use provide that if one or more of the detected expression levels is greater than a reference level, the subject is more likely to respond positively to a CAR-expressing cell therapy.

In an embodiment, the set of reagents detects the expression of mRNA expressed from said set of genes.

In an embodiment, the set of reagents comprises nucleic acid probes complementary to mRNA expressed from said set of genes.

In an embodiment, the nucleic acid probes complementary to mRNA are CDNA or oligonucleotides.

In an embodiment, the nucleic acid probes complementary to mRNA are immobilized on a substrate surface.

In an embodiment, the set of reagents detects the expression of polypeptides encoded by said set of genes.

### Therapeutic Agents, Compositions and Administration

The methods described herein can be used to assess a responder status to a cell expressing a CAR. In one embodiment, the cell expresses a CAR molecule comprising an antigen binding domain (e.g., an antibody or antibody fragment that specifically binds to a tumor antigen), a transmembrane domain, and an intracellular signaling domain (e.g., an intracellular signaling domain comprising a costimulatory domain and/or a primary signaling domain). In an embodiment, the antigen binding domain comprises any antibody, or a fragment thereof, e.g., an scFv, known in the art that targets or specifically binds to any of the tumor antigens described herein. For example, the tumor antigen is BCMA (also known as TNFRSF17, Tumor Necrosis Factor Receptor Superfamily, Member 17, or B Cell Maturation Antigen), CD33, CLL-1 (also known as C-type Lectin-Like domain family 1, or CLECL1) or claudin-6 (CLDN6). The antibody, or fragment thereof, can be a murine, humanized, or fully human antibody or fragment thereof, e.g., an scFv.

In one embodiment, the CAR comprises an antibody or antibody fragment which includes an anti-CD19 binding domain described herein (e.g., a murine or humanized antibody or antibody fragment that specifically binds to CD19 as described herein), a transmembrane domain described herein, and an intracellular signaling domain described herein (e.g., an intracellular signaling domain comprising a costimulatory domain and/or a primary signaling domain described herein).

Provided herein are compositions of matter and methods of use for the treatment of a disease such as cancer using immune effector cells (e.g., T cells, NK cells) engineered with CARs of the invention.

In one aspect, the invention provides a number of chimeric antigen receptors (CAR) comprising an antigen binding domain (e.g., antibody or antibody fragment, TCR or TCR fragment) engineered for specific binding to a tumor antigen, e.g., a tumor antigen described herein. In one aspect, the invention provides an immune effector cell (e.g., T cell, NK cell) engineered to express a CAR, wherein the engineered immune effector cell exhibits an anticancer property. In one aspect, a cell is transformed with the CAR and the CAR is expressed on the cell surface. In some embodiments, the cell (e.g., T cell, NK cell) is transduced with a viral vector encoding a CAR. In some embodiments, the viral vector is a retroviral vector. In some embodiments, the viral vector is a lentiviral vector. In some such embodiments, the cell may stably express the CAR. In another embodiment, the cell (e.g., T cell, NK cell) is transfected with a nucleic acid, e.g., mRNA, CDNA, DNA, encoding a CAR. In some such embodiments, the cell may transiently express the CAR.

In one aspect, the antigen binding domain of a CAR described herein is a scFv antibody fragment. In one aspect, such antibody fragments are functional in that they retain the equivalent binding affinity, e.g., they bind the same antigen with comparable affinity, as the IgG antibody from which it is derived. In other embodiments, the antibody fragment has a lower binding affinity, e.g., it binds the same antigen with a lower binding affinity than the antibody from which it is derived, but is functional in that it provides a biological response described herein. In one embodiment, the CAR molecule comprises an antibody fragment that has a binding affinity KD of 10⁻⁴ M to 10⁻⁸M, e.g., 10⁻⁵ M to 10⁻⁷ M, e.g., 10⁻⁶ M or 10⁻⁷ M, for the target antigen. In one embodiment, the antibody fragment has a binding affinity that is at least five-fold, 10-fold, 20-fold, 30-fold, 50-fold, 100-fold or 1,000-fold less than a reference antibody, e.g., an antibody described herein.

In one aspect such antibody fragments are functional in that they provide a biological response that can include, but is not limited to, activation of an immune response, inhibition of signal-transduction origination from its target antigen, inhibition of kinase activity, and the like, as will be understood by a skilled artisan.

In one aspect, the antigen binding domain of the CAR is a scFv antibody fragment that is humanized compared to the murine sequence of the scFv from which it is derived.

In one aspect, the antigen binding domain of a CAR of the invention (e.g., a scFv) is encoded by a nucleic acid molecule whose sequence has been codon optimized for expression in a mammalian cell. In one aspect, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

In one aspect, the CARs of the invention combine an antigen binding domain of a specific antibody with an intracellular signaling molecule. For example, in some aspects, the intracellular signaling molecule includes, but is not limited to, CD3-zeta chain, 4-1BB and CD28 signaling modules and combinations thereof. In one aspect, the antigen binding domain binds to a tumor antigen as described herein.

Furthermore, the present invention provides CARs and CAR-expressing cells and their use in medicaments or methods for treating, among other diseases, cancer or any malignancy or autoimmune diseases involving cells or tissues which express a tumor antigen as described herein.

In one aspect, the CAR of the invention can be used to eradicate a normal cell that express a tumor antigen as described herein, thereby applicable for use as a cellular conditioning therapy prior to cell transplantation. In one aspect, the normal cell that expresses a tumor antigen as described herein is a normal stem cell and the cell transplantation is a stem cell transplantation.

In one aspect, the invention provides an immune effector cell (e.g., T cell, NK cell) engineered to express a chimeric antigen receptor (CAR), wherein the engineered immune effector cell exhibits an antitumor property. A preferred antigen is a cancer associated antigen (i.e., tumor antigen) described herein. In one aspect, the antigen binding domain of the CAR comprises a partially humanized antibody fragment. In one aspect, the antigen binding domain of the CAR comprises a partially humanized scFv. Accordingly, the invention provides CARs that comprises a humanized antigen binding domain and is engineered into a cell, e.g., a T cell or a NK cell, and methods of their use for adoptive therapy.

In one aspect, the CARs of the invention comprise at least one intracellular domain selected from the group of a CD137 (4-1BB) signaling domain, a CD28 signaling domain, a CD27 signal domain, a CD3zeta signal domain, and any combination thereof. In one aspect, the CARs of the invention comprise at least one intracellular signaling domain is from one or more costimulatory molecule(s) other than a CD137 (4-1BB) or CD28.

Sequences of some examples of various components of CARs of the instant invention is listed in Table 1, where aa stands for amino acids, and na stands for nucleic acids that encode the corresponding peptide.

TABLE 1
Sequences of various components of CAR (aa—amino acids, na—nucleic acids that
encodes the corresponding protein)

| SEQ | | | Corresp. |
|---|---|---|---|
| ID | | | To |
| NO | description | Sequence | huCD19 |
| 1 | EF-1 | CGTGAGGCTCCGGTGCCCGTCAGTGGGCAGAGCGC | 100 |
| | promoter | ACATCGCCCACAGTCCCCGAGAAGTTGGGGGGAG | |
| | | GGGTCGGCAATTGAACCGGTGCCTAGAGAAGGTG | |
| | | GCGCGGGGTAAACTGGGAAAGTGATGTCGTGTACT | |
| | | GGCTCCGCCTTTTTCCCGAGGGTGGGGGAGAACCG | |
| | | TATATAAGTGCAGTAGTCGCCGTGAACGTTCTTTTT | |
| | | CGCAACGGGTTTGCCGCCAGAACACAGGTAAGTGC | |
| | | CGTGTGTGGTTCCCGCGGGCCTGGCCTCTTTACGG | |
| | | GTTATGGCCCTTGCGTGCCTTGAATTACTTCCACCT | |
| | | GGCTGCAGTACGTGATTCTTGATCCCGAGCTTCGG | |
| | | GTTGGAAGTGGGTGGGAGAGTTCGAGGCCTTGCGC | |
| | | TTAAGGAGCCCCTTCGCCTCGTGCTTGAGTTGAGG | |
| | | CCTGGCCTGGGCGCTGGGGCCGCCGCGTGCGAATC | |
| | | TGGTGGCACCTTCGCGCCTGTCTCGCTGCTTTCGAT | |
| | | AAGTCTCTAGCCATTTAAAATTTTTGATGACCTGCT | |
| | | GCGACGCTTTTTTTCTGGCAAGATAGTCTTGTAAAT | |
| | | GCGGGCCAAGATCTGCACACTGGTATTTCGGTTTTT | |
| | | GGGGCCGCGGGCGGCGACGGGGCCCGTGCGTCCC | |
| | | AGCGCACATGTTCGGCGAGGCGGGGCCTGCGAGC | |
| | | GCGGCCACCGAGAATCGGACGGGGGTAGTCTCAA | |
| | | GCTGGCCGGCCTGCTCTGGTGCCTGGCCTCGCGCC | |
| | | GCCGTGTATCGCCCCGCCCTGGGCGGCAAGGCTGG | |
| | | CCCGGTCGGCACCAGTTGCGTGAGCGGAAAGATGG | |
| | | CCGCTTCCCGGCCCTGCTGCAGGGAGCTCAAAATG | |
| | | GAGGACGCGGCGCTCGGGAGAGCGGGCGGGTGAG | |
| | | TCACCCACACAAAGGAAAAGGGCCTTTCCGTCCTC | |
| | | AGCCGTCGCTTCATGTGACTCCACGGAGTACCGGG | |
| | | CGCCGTCCAGGCACCTCGATTAGTTCTCGAGCTTTT | |
| | | GGAGTACGTCGTCTTTAGGTTGGGGGGAGGGGTTT | |
| | | TATGCGATGGAGTTTCCCCACACTGAGTGGGTGGA | |
| | | GACTGAAGTTAGGCCAGCTTGGCACTTGATGTAAT | |
| | | TCTCCTTGGAATTTGCCCTTTTTGAGTTTGGATCTT | |
| | | GGTTCATTCTCAAGCCTCAGACAGTGGTTCAAAGT | |
| | | TTTTTTCTTCCATTTCAGGTGTCGTGA | |
| 2 | Leader (aa) | MALPVTALLLPLALLLHAARP | 13 |
| 3 | Leader (na) | ATGGCCCTGCCTGTGACAGCCCTGCTGCTGCCTCTG | 54 |
| | | GCTCTGCTGCTGCATGCCGCTAGACCC | |
| 4 | CD 8 hinge | TTTPAPRPPTPAPTIASQPLSLRPEACRPAAGGAVHTR | 14 |
| | (aa) | GLDFACD | |
| 5 | CD8 hinge | ACCACGACGCCAGCGCCGCGACCACCAACACCGG | 55 |
| | (na) | CGCCCACCATCGCGTCGCAGCCCCTGTCCCTGCGC | |
| | | CCAGAGGCGTGCCGGCCAGCGGCGGGGGGCGCAG | |
| | | TGCACACGAGGGGGCTGGACTTCGCCTGTGAT | |
| 6 | Ig4 hinge | ESKYGPPCPPCPAPEFLGGPSVFLFPPKPKDTLMISRTP | 102 |
| | (aa) | EVTCVVVDVSQEDPEVQFNWYVDGVEVHNAKTKPR | |
| | | EEQFNSTYRVVSVLTVLHQDWLNGKEYKCKVSNKG | |
| | | LPSSIEKTISKAKGQPREPQVYTLPPSQEEMTKNQVSL | |
| | | TCLVKGFYPSDIAVEWESNGQPENNYKTTPPVLDSD | |
| | | GSFFLYSRLTVDKSRWQEGNVFSCSVMHEALHNHYT | |
| | | QKSLSLSLGKM | |
| 7 | Ig4 hinge | GAGAGCAAGTACGGCCCTCCCTGCCCCCCTTGCCC | 103 |
| | (na) | TGCCCCCGAGTTCCTGGGCGGACCCAGCGTGTTCC | |
| | | TGTTCCCCCCCAAGCCCAAGGACACCCTGATGATC | |
| | | AGCCGGACCCCCGAGGTGACCTGTGTGGTGGTGGA | |
| | | CGTGTCCCAGGAGGACCCCGAGGTCCAGTTCAACT | |
| | | GGTACGTGGACGGCGTGGAGGTGCACAACGCCAA | |
| | | GACCAAGCCCCGGGAGGAGCAGTTCAATAGCACCT | |
| | | ACCGGGTGGTGTCCGTGCTGACCGTGCTGCACCAG | |
| | | GACTGGCTGAACGGCAAGGAATACAAGTGTAAGG | |
| | | TGTCCAACAAGGGCCTGCCCAGCAGCATCGAGAAA | |
| | | ACCATCAGCAAGGCCAAGGGCCAGCCTCGGGAGC | |
| | | CCCAGGTGTACACCCTGCCCCCTAGCCAAGAGGAG | |
| | | ATGACCAAGAACCAGGTGTCCCTGACCTGCCTGGT | |
| | | GAAGGGCTTCTACCCCAGCGACATCGCCGTGGAGT | |
| | | GGGAGAGCAACGGCCAGCCCGAGAACAACTACAA | |
| | | GACCACCCCCCCTGTGCTGGACAGCGACGGCAGCT | |
| | | TCTTCCTGTACAGCCGGCTGACCGTGGACAAGAGC | |
| | | CGGTGGCAGGAGGGCAACGTCTTTAGCTGCTCCGT | |
| | | GATGCACGAGGCCCTGCACAACCACTACACCCAGA | |
| | | AGAGCCTGAGCCTGTCCCTGGGCAAGATG | |
| 8 | IgD hinge | RWPESPKAQASSVPTAQPQAEGSLAKATTAPATTRN | 47 |
| | (aa) | TGRGGEEKKKEKEKEEQEERETKTPECPSHTQPLGVY | |
| | | LLTPAVQDLWLRDKATFTCFVVGSDLKDAHLTWEV | |
| | | AGKVPTGGVEEGLLERHSNGSQSQHSRLTLPRSLWN | |
| | | AGTSVTCTLNHPSLPPQRLMALREPAAQAPVKLSLNL | |
| | | LASSDPPEAASWLLCEVSGFSPPNILLMWLEDQREVN | |
| | | TSGFAPARPPPQPGSTTFWAWSVLRVPAPPSPQPATY | |
| | | TCVVSHEDSRTLLNASRSLEVSYVTDH | |
| 9 | IgD hinge | AGGTGGCCCGAAAGTCCCAAGGCCCAGGCATCTAG | 48 |
| | (na) | TGTTCCTACTGCACAGCCCCAGGCAGAAGGCAGCC | |
| | | TAGCCAAAGCTACTACTGCACCTGCCACTACGCGC | |
| | | AATACTGGCCGTGGCGGGGAGGAGAAGAAAAAGG | |
| | | AGAAAGAGAAAGAAGAACAGGAAGAGAGGGAGA | |
| | | CCAAGACCCCTGAATGTCCATCCCATACCCAGCCG | |
| | | CTGGGCGTCTATCTCTTGACTCCCGCAGTACAGGA | |
| | | CTTGTGGCTTAGAGATAAGGCCACCTTTACATGTTT | |
| | | CGTCGTGGGCTCTGACCTGAAGGATGCCCATTTGA | |
| | | CTTGGGAGGTTGCCGGAAAGGTACCCACAGGGGG | |
| | | GGTTGAGGAAGGGTTGCTGGAGCGCCATTCCAATG | |
| | | GCTCTCAGAGCCAGCACTCAAGACTCACCCTTCCG | |
| | | AGATCCCTGTGGAACGCCGGGACCTCTGTCACATG | |
| | | TACTCTAAATCATCCTAGCCTGCCCCCACAGCGTCT | |
| | | GATGGCCCTTAGAGAGCCAGCCGCCCAGGCACCAG | |
| | | TTAAGCTTAGCCTGAATCTGCTCGCCAGTAGTGAT | |
| | | CCCCCAGAGGCCGCCAGCTGGCTCTTATGCGAAGT | |
| | | GTCCGGCTTTAGCCCGCCCAACATCTTGCTCATGTG | |
| | | GCTGGAGGACCAGCGAGAAGTGAACACCAGCGGC | |
| | | TTCGCTCCAGCCCGGCCCCCACCCCAGCCGGGTTC | |
| | | TACCACATTCTGGGCCTGGAGTGTCTTAAGGGTCC | |
| | | CAGCACCACCTAGCCCCCAGCCAGCCACATACACC | |
| | | TGTGTTGTGTCCCATGAAGATAGCAGGACCCTGCT | |
| | | AAATGCTTCTAGGAGTCTGGAGGTTTCCTACGTGA | |
| | | CTGACCATT | |
| 10 | GS | GGGGSGGGGS | 49 |
| | hinge/linker | | |
| | (aa) | | |
| 11 | GS | GGTGGCGGAGGTTCTGGAGGTGGAGGTTCC | 50 |
| | hinge/linker | | |
| | (na) | | |
| 12 | CD8TM | IYIWAPLAGTCGVLLLSLVITLYC | 15 |
| | (aa) | | |
| 13 | CD8 TM | ATCTACATCTGGGCGCCCTTGGCCGGGACTTGTGG | 56 |
| | (na) | GGTCCTTCTCCTGTCACTGGTTATCACCCTTTACTG | |
| | | C | |
| 14 | 4-1BB | KRGRKKLLYIFKQPFMRPVQTTQEEDGCSCRFPEEEE | 16 |
| | intracellular | GGCEL | |
| | domain | | |
| | (aa) | | |
| 15 | 4-1BB | AAACGGGGCAGAAAGAAACTCCTGTATATATTCAA | 60 |
| | intracellular | ACAACCATTTATGAGACCAGTACAAACTACTCAAG | |
| | domain | AGGAAGATGGCTGTAGCTGCCGATTTCCAGAAGAA | |
| | (na) | GAAGAAGGAGGATGTGAACTG | |
| 16 | CD27 (aa) | QRRKYRSNKGESPVEPAEPCRYSCPREEEGSTIPIQED | 51 |
| | | YRKPEPACSP | |
| 17 | CD27 (na) | AGGAGTAAGAGGAGCAGGCTCCTGCACAGTGACT | 52 |
| | | ACATGAACATGACTCCCCGCCGCCCCGGGCCCACC | |
| | | CGCAAGCATTACCAGCCCTATGCCCCACCACGCGA | |
| | | CTTCGCAGCCTATCGCTCC | |
| 18 | CD3-zeta | RVKFSRSADAPAYKQGQNQLYNELNLGRREEYDVL | 17 |
| | (aa) | DKRRGRDPEMGGKPRRKNPQEGLYNELQKDKMAEA | |
| | | YSEIGMKGERRRGKGHDGLYQGLSTATKDTYDALH | |
| | | MQALPPR | |
| 19 | CD3-zeta | AGAGTGAAGTTCAGCAGGAGCGCAGACGCCCCCG | 101 |
| | (na) | CGTACAAGCAGGGCCAGAACCAGCTCTATAACGA | |
| | | GCTCAATCTAGGACGAAGAGAGGAGTACGATGTTT | |
| | | TGGACAAGAGACGTGGCCGGGACCCTGAGATGGG | |
| | | GGGAAAGCCGAGAAGGAAGAACCCTCAGGAAGGC | |
| | | CTGTACAATGAACTGCAGAAAGATAAGATGGCGG | |
| | | AGGCCTACAGTGAGATTGGGATGAAAGGCGAGCG | |
| | | CCGGAGGGGCAAGGGGCACGATGGCCTTTACCAG | |
| | | GGTCTCAGTACAGCCACCAAGGACACCTACGACGC | |
| | | CCTTCACATGCAGGCCCTGCCCCCTCGC | |
| 20 | CD3-zeta | RVKFSRSADAPAYQQGQNQLYNELNLGRREEYDVL | 43 |
| | (aa) | DKRRGRDPEMGGKPRRKNPQEGLYNELQKDKMAEA | |
| | | YSEIGMKGERRRGKGHDGLYQGLSTATKDTYDALH | |
| | | MQALPPR | |
| 21 | CD3-zeta | AGAGTGAAGTTCAGCAGGAGCGCAGACGCCCCCG | 44 |
| | (na) | CGTACCAGCAGGGCCAG | |
| | | AACCAGCTCTATAACGAGCTCAATCTAGGACGAAG | |
| | | AGAGGAGTACGATGTTT | |
| | | TGGACAAGAGACGTGGCCGGGACCCTGAGATGGG | |
| | | GGGAAAGCCGAGAAGGA | |
| | | AGAACCCTCAGGAAGGCCTGTACAATGAACTGCAG | |
| | | AAAGATAAGATGGCGG | |
| | | AGGCCTACAGTGAGATTGGGATGAAAGGCGAGCG | |
| | | CCGGAGGGGCAAGGGGC | |
| | | ACGATGGCCTTTACCAGGGTCTCAGTACAGCCACC | |
| | | AAGGACACCTACGACGC | |
| | | CCTTCACATGCAGGCCCTGCCCCCTCGC | |
| 22 | linker | GGGGS | 18 |
| 23 | linker | GGTGGCGGAGGTTCTGGAGGTGGAGGTTCC | 50 |
| 24 | PD-1 | Pgwfldspdrpwnpptfspallvvtegdnatftcsfsntsesfylnwyrmspsnq | |
| | extracellular | tdklaafpedrsqpgqdcrfrvtqlpngrdfhmsvvrarrndsgtylcgaislapk | |
| | domain | aqikeslraelrvterraevptahpspsprpagqfqtlv | |
| | (aa) | | |
| 25 | PD-1 | Cccggatggtttctggactctccggatcgcccgtggaatcccccaaccttctcaccg | |
| | extracellular | gcactcttggttgtgactgagggcgataatgcgaccttcacgtgctcgttctccaaca | |
| | domain | cctccgaatcattcgtgctgaactggtaccgcatgagcccgtcaaaccagaccgac | |
| | (na) | aagctcgccgcgtttccggaagatcggtcgcaaccgggacaggattgtcggttccg | |
| | | cgtgactcaactgccgaatggcagagacttccacatgagcgtggtccgcgctaggc | |
| | | gaaacgactccgggacctacctgtgcggagccatctcgctggcgcctaaggccca | |
| | | aatcaaagagagcttgagggccgaactgagagtgaccgagcgcagagctgaggt | |
| | | gccaactgcacatccatccccatcgcctcggcctgcggggcagtttcagaccctgg | |
| | | tc | |
| 26 | PD-1 CAR | Malpvtalllplalllhaarppgwfldspdrpwnpptfspallvvtegdnatftcsf | |
| | (aa) with | sntsesfvlnwyrmspsnqtdklaafpedrsqpgqdcrfrvtqlpngrdfhmsv | |
| | signal | vrarrndsgtylcgaislapkaqikeslraelrvterraevptahpspsprpagqfqt | |
| | | lvtttpaprpptpaptiasqplslrpeacrpaaggavhtrgldfacdiyiwaplagtc | |
| | | gvlllslvitlyckrgrkkllyifkqpfmrpvqttqeedgcscrfpeeeeggcelrvk | |
| | | fsrsadapaykqgqnqlynelnlgrreeydvldkrrgrdpemggkprrknpqeg | |
| | | lynelqkdkmaeayseigmkgerrrgkghdglyqglstatkdtydalhmqalp | |
| | | pr | |
| 27 | PD-1 CAR | Atggccctccctgtcactgccctgcttctccccctcgcactcctgctccacgccgcta | |
| | (na) | gaccacccggatggtttctggactctccggatcgcccgtggaatcccccaaccttct | |
| | | caccggcactcttggttgtgactgagggcgataatgcgaccttcacgtgctcgttctc | |
| | | caacacctccgaatcattcgtgctgaactggtaccgcatgagcccgtcaaaccaga | |
| | | ccgacaagctcgccgcgtttccggaagatcggtcgcaaccgggacaggattgtcg | |
| | | gttccgcgtgactcaactgccgaatggcagagacttccacatgagcgtggtccgcg | |
| | | ctaggcgaaacgactccgggacctacctgtgcggagccatctcgctggcgcctaa | |
| | | ggcccaaatcaaagagagcttgagggccgaactgagagtgaccgagcgcagagc | |
| | | tgaggtgccaactgcacatccatccccatcgcctcggcctgcggggcagtttcaga | |
| | | ccctggtcacgaccactccggcgccgcgcccaccgactccggccccaactatcgc | |
| | | gagccagcccctgtcgctgaggccggaagcatgccgccctgccgccggaggtgc | |
| | | tgtgcatacccggggattggacttcgcatgcgacatctacatttgggctcctctcgcc | |
| | | ggaacttgtggcgtgctccttctgtccctggtcatcaccctgtactgcaagcggggtc | |
| | | ggaaaaagcttctgtacattttcaagcagcccttcatgaggcccgtgcaaaccaccc | |
| | | aggaggaggacggttgctcctgccggttccccgaagaggaagaaggaggttgcg | |
| | | agctgcgcgtgaagttctcccggagcgccgacgcccccgcctataagcagggcca | |
| | | gaaccagctgtacaacgaactgaacctgggacggcgggaagagtacgatgtgctg | |
| | | gacaagcggcgcggccgggaccccgaaatgggcgggaagcctagaagaaaga | |
| | | accctcaggaaggcctgtataacgagctgcagaaggacaagatggccgaggccta | |
| | | ctccgaaattgggatgaagggagagcggcggaggggaaaggggcacgacggc | |
| | | ctgtaccaaggactgtccaccgccaccaaggacacatacgatgccctgcacatgca | |
| | | ggcccttccccctcgc | |
| 28 | linker | (Gly-Gly-Gly-Ser)n, where n = 1-10 | 105 |
| 29 | linker | (Gly4 Ser)4 | 106 |
| 30 | linker | (Gly4 Ser)3 | 107 |
| 31 | linker | (Gly3Ser) | 108 |
| 32 | polyA | aaaaaaaaaa aaaaaaaaaa aaaaaaaaaa aaaaaaaaaa aaaaaaaaaa | 118 |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| 33 | polyA | aaaaaaaaaa aaaaaaaaaa aaaaaaaaaa aaaaaaaaaa aaaaaaaaaa | 104 |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| 34 | polyA | aaaaaaaaaa aaaaaaaaaa aaaaaaaaaa aaaaaaaaaa aaaaaaaaaa | 109 |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| 35 | polyA | tttttttttt tttttttttt tttttttttt tttttttttt tttttttttt | 110 |
| | | tttttttttt tttttttttt tttttttttt tttttttttt tttttttttt | |
| 36 | polyA | tttttttttt tttttttttt tttttttttt tttttttttt tttttttttt | 111 |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| | | tttttttttt tttttttttt tttttttttt tttttttttt tttttttttt | |
| 37 | polyA | aaaaaaaaaa aaaaaaaaaa aaaaaaaaaa aaaaaaaaaa aaaaaaaaaa | 112 |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| 38 | polyA | aaaaaaaaaa aaaaaaaaaa aaaaaaaaaa aaaaaaaaaa aaaaaaaaaa | 113 |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| | | aaaaaaaaaa aaaaaaaaaa aaaaaaaaaa aaaaaaaaaa aaaaaaaaaa | |
| 39 | PD1 CAR | Pgwfldspdrpwnpptfspallvvtegdnatftcsfsntsesfvlnwyrmspsne | |
| | (aa) | tdklaafpedrsqpgqdcrfrvtqlpngrdfhmsvvrarrndsgtylcgaislapk | |
| | | aqikeslraelrvterraevptahpspsprpagqfqtlvtttpaprpptpaptiasqpl | |
| | | slrpeacrpaaggavhtrgldfacdiyiwaplagtcgvlllslvitlyckrgrkkllyi | |
| | | fkqpfmrpvqttqeedgcscrfpeeeeggcelrvkfsrsadapaykqgqnqlyn | |
| | | elnlgrreeydvldkrrgrdpemggkprrknpqeglynelqkdkmaeayseig | |
| | | mkgerrrgkghdglyqglstatkdtydalhmqalppr | |

### Cancer Associated Antigens

The present invention provides immune effector cells (e.g., T cells, NK cells) that are engineered to contain one or more CARs that direct the immune effector cells to cancer. This is achieved through an antigen binding domain on the CAR that is specific for a cancer associated antigen. There are two classes of cancer associated antigens (tumor antigens) that can be targeted by the CARs of the instant invention: (1) cancer associated antigens that are expressed on the surface of cancer cells; and (2) cancer associated antigens that itself is intracelluar, however, a fragment of such antigen (peptide) is presented on the surface of the cancer cells by MHC (major histocompatibility complex).

Accordingly, the present invention provides CARs that target the following cancer associated antigens (tumor antigens): CD19, CD123, CD22, CD30, CD171, CS-1, CLL-1 (CLECL1), CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6, E7, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RUL RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

### Tumor-Supporting Antigens

A CAR described herein can comprise an antigen binding domain (e.g., antibody or antibody fragment, TCR or TCR fragment) that binds to a tumor-supporting antigen (e.g., a tumor-supporting antigen as described herein). In some embodiments, the tumor-supporting antigen is an antigen present on a stromal cell or a myeloid-derived suppressor cell (MDSC). Stromal cells can secrete growth factors to promote cell division in the microenvironment. MDSC cells can inhibit T cell proliferation and activation. Without wishing to be bound by theory, in some embodiments, the CAR-expressing cells destroy the tumor-supporting cells, thereby indirectly inhibiting tumor growth or survival.

In some embodiments, the stromal cell antigen is chosen from one or more of: bone marrow stromal cell antigen 2 (BST2), fibroblast activation protein (FAP) and tenascin. In an embodiment, the FAP-specific antibody is, competes for binding with, or has the same CDRs as, sibrotuzumab. In some embodiments, the MDSC antigen is chosen from one or more of: CD33, CD11b, C14, CD15, and CD66b. Accordingly, in some embodiments, the tumor-supporting antigen is chosen from one or more of: bone marrow stromal cell antigen 2 (BST2), fibroblast activation protein (FAP) or tenascin, CD33, CD11b, C14, CD15, and CD66b.

### Chimeric Antigen Receptor (CAR)

The present invention encompasses a recombinant DNA construct comprising sequences encoding a CAR, wherein the CAR comprises an antigen binding domain (e.g., antibody or antibody fragment, TCR or TCR fragment) that binds specifically to a cancer associated antigen described herein, wherein the sequence of the antigen binding domain is contiguous with and in the same reading frame as a nucleic acid sequence encoding an intracellular signaling domain. The intracellular signaling domain can comprise a costimulatory signaling domain and/or a primary signaling domain, e.g., a zeta chain. The costimulatory signaling domain refers to a portion of the CAR comprising at least a portion of the intracellular domain of a costimulatory molecule.

In specific aspects, a CAR construct of the invention comprises a scFv domain, wherein the scFv may be preceded by an optional leader sequence such as provided in SEQ ID NO: 2, and followed by an optional hinge sequence such as provided in SEQ ID NO:4 or SEQ ID NO:6 or SEQ ID NO:8 or SEQ ID NO:10, a transmembrane region such as provided in SEQ ID NO:12, an intracellular signalling domain that includes SEQ ID NO:14 or SEQ ID NO:16 and a CD3 zeta sequence that includes SEQ ID NO:18 or SEQ ID NO:20, e.g., wherein the domains are contiguous with and in the same reading frame to form a single fusion protein.

In one aspect, an exemplary CAR constructs comprise an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain described herein). In one aspect, an exemplary CAR construct comprises an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

An exemplary leader sequence is provided as SEQ ID NO: 2. An exemplary hinge/spacer sequence is provided as SEQ ID NO: 4 or SEQ ID NO:6 or SEQ ID NO:8 or SEQ ID NO:10. An exemplary transmembrane domain sequence is provided as SEQ ID NO:12. An exemplary sequence of the intracellular signaling domain of the 4-1BB protein is provided as SEQ ID NO: 14. An exemplary sequence of the intracellular signaling domain of CD27 is provided as SEQ ID NO:16. An exemplary CD3zeta domain sequence is provided as SEQ ID NO: 18 or SEQ ID NO:20.

In one aspect, the present invention encompasses a recombinant nucleic acid construct comprising a nucleic acid molecule encoding a CAR, wherein the nucleic acid molecule comprises the nucleic acid sequence encoding an antigen binding domain, e.g., described herein, that is contiguous with and in the same reading frame as a nucleic acid sequence encoding an intracellular signaling domain.

In one aspect, the present invention encompasses a recombinant nucleic acid construct comprising a nucleic acid molecule encoding a CAR, wherein the nucleic acid molecule comprises a nucleic acid sequence encoding an antigen binding domain, wherein the sequence is contiguous with and in the same reading frame as the nucleic acid sequence encoding an intracellular signaling domain. An exemplary intracellular signaling domain that can be used in the CAR includes, but is not limited to, one or more intracellular signaling domains of, e.g., CD3-zeta, CD28, CD27, 4-1BB, and the like. In some instances, the CAR can comprise any combination of CD3-zeta, CD28, 4-1BB, and the like.

The nucleic acid sequences coding for the desired molecules can be obtained using recombinant methods known in the art, such as, for example by screening libraries from cells expressing the nucleic acid molecule, by deriving the nucleic acid molecule from a vector known to include the same, or by isolating directly from cells and tissues containing the same, using standard techniques. Alternatively, the nucleic acid of interest can be produced synthetically, rather than cloned.

The present invention includes retroviral and lentiviral vector constructs expressing a CAR that can be directly transduced into a cell.

The present invention also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a polyA tail, typically 50-2000 bases in length (SEQ ID NO:32). RNA so produced can efficiently transfect different kinds of cells. In one embodiment, the template includes sequences for the CAR. In an embodiment, an RNA CAR vector is transduced into a cell, e.g., a T cell or a NK cell, by electroporation.

### Antigen Binding Domain

In one aspect, the CAR of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR of the invention include those associated with viral, bacterial and parasitic infections, autoimmune disease and cancer cells.

In one aspect, the CAR-mediated T-cell response can be directed to an antigen of interest by way of engineering an antigen binding domain that specifically binds a desired antigen into the CAR.

In one aspect, the portion of the CAR comprising the antigen binding domain comprises an antigen binding domain that targets a tumor antigen, e.g., a tumor antigen described herein.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

In one embodiment, the CD19 CAR is a CD19 CAR described in U.S. Pat. Nos. 8,399,645; 7,446,190; Xu et al., Leuk Lymphoma. 2013 54(2):255-260 (2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25): 4129-39 (2013); or 16th Annu Meet Am Soc Gen Cell Ther (ASGCT) (May 15-18, Salt Lake City) 2013, Abst 10 (each of which is herein incorporated by reference in their entirety). In one embodiment, an antigen binding domain against CD19 is an antigen binding portion, e.g., CDRs, of a CAR, antibody or antigen-binding fragment thereof described in, e.g., PCT publication WO2012/079000 (incorporated herein by reference in its entirety). In one embodiment, an antigen binding domain against CD19 is an antigen binding portion, e.g., CDRs, of a CAR, antibody or antigen-binding fragment thereof described in, e.g., PCT publication WO2014/153270; Kochenderfer, J. N. et al., J. Immunother. 32 (7), 689-702 (2009); Kochenderfer, J. N., et al., Blood, 116 (20), 4099-4102 (2010); PCT publication WO2014/031687; Bejcek, Cancer Research, 55, 2346-2351, 1995; or U.S. Pat. No. 7,446,190 (each of which is herein incorporated by reference in their entirety).

In one embodiment, the antigen binding domain against mesothelin is or may be derived from an antigen binding domain, e.g., CDRs, scFv, or VH and VL, of an antibody, antigen-binding fragment or CAR described in, e.g., PCT publication WO2015/090230 (In one embodiment the CAR is a CAR described in WO2015/090230, the contents of which are incorporated herein in their entirety). In some embodiments, the antigen binding domain against mesothelin is or is derived from an antigen binding portion, e.g., CDRs, scFv, or VH and VL, of an antibody, antigen-binding fragment, or CAR described in, e.g., PCT publication WO1997/025068, WO1999/028471, WO2005/014652, WO2006/099141, WO2009/045957, WO2009/068204, WO2013/142034, WO2013/040557, or WO2013/063419 (each of which is herein incorporated by reference in their entirety).

In one embodiment, an antigen binding domain against CD123 is or is derived from an antigen binding portion, e.g., CDRs, scFv or VH and VL, of an antibody, antigen-binding fragment or CAR described in, e.g., PCT publication WO2014/130635 (incorporated herein by reference in its entirety). In one embodiment, an antigen binding domain against CD123 is or is derived from an antigen binding portion, e.g., CDRs, scFv or VH and VL, of an antibody, antigen-binding fragment or CAR described in, e.g., PCT publication WO2016/028896 (incorporated herein by reference in its entirety); in some embodiments, the CAR is a CAR described in WO2016/028896. In one embodiment, an antigen binding domain against CD123 is or is derived from an antigen binding portion, e.g., CDRs, scFv, or VL and VH, of an antibody, antigen-binding fragment, or CAR described in, e.g., PCT publication WO1997/024373, WO2008/127735 (e.g., a CD123 binding domain of 26292, 32701, 37716 or 32703), WO2014/138805 (e.g., a CD123 binding domain of CSL362), WO2014/138819, WO2013/173820, WO2014/144622, WO2001/66139, WO2010/126066 (e.g., the CD123 binding domain of any of Old4, Old5, Old17, Old19, New102, or Old6), WO2014/144622, or US2009/0252742 (each of which is incorporated herein by reference in its entirety).

In one embodiment, an antigen binding domain against CD22 is an antigen binding portion, e.g., CDRs, of an antibody described in, e.g., Haso et al., Blood, 121(7): 1165-1174 (2013); Wayne et al., Clin Cancer Res 16(6): 1894-1903 (2010); Kato et al., Leuk Res 37(1):83-88 (2013); Creative BioMart (creativebiomart.net): MOM-18047-S(P).

In one embodiment, an antigen binding domain against CS-1 is an antigen binding portion, e.g., CDRs, of Elotuzumab (BMS), see e.g., Tai et al., 2008, Blood 112(4):1329-37; Tai et al., 2007, Blood. 110(5):1656-63.

In one embodiment, an antigen binding domain against CLL-1 is an antigen binding portion, e.g., CDRs or VH and VL, of an antibody, antigen-binding fragment or CAR described in, e.g., PCT publication WO2016/014535, the contents of which are incorporated herein in their entirety. In one embodiment, an antigen binding domain against CLL-1 is an antigen binding portion, e.g., CDRs, of an antibody available from R&D, ebiosciences, Abcam, for example, PE-CLL1-hu Cat #353604 (BioLegend); and PE-CLL1 (CLEC12A) Cat #562566 (BD).

In one embodiment, an antigen binding domain against CD33 is an antigen binding portion, e.g., CDRs, of an antibody described in, e.g., Bross et al., Clin Cancer Res 7(6):1490-1496 (2001) (Gemtuzumab Ozogamicin, hP67.6), Caron et al., Cancer Res 52(24):6761-6767 (1992) (Lintuzumab, HuM195), Lapusan et al., Invest New Drugs 30(3):1121-1131 (2012) (AVE9633), Aigner et al., Leukemia 27(5): 1107-1115 (2013) (AMG330, CD33 BiTE), Dutour et al., Adv hematol 2012: 683065 (2012), and Pizzitola et al., Leukemia doi:10.1038/Lue.2014.62 (2014). Exemplary CAR molecules that target CD33 are described herein, and are provided in WO2016/014576, e.g., in Table 2 of WO2016/014576 (incorporated by reference in its entirety).

In one embodiment, an antigen binding domain against GD2 is an antigen binding portion, e.g., CDRs, of an antibody described in, e.g., Mujoo et al., Cancer Res. 47(4):1098-1104 (1987); Cheung et al., Cancer Res 45(6):2642-2649 (1985), Cheung et al., J Clin Oncol 5(9):1430-1440 (1987), Cheung et al., J Clin Oncol 16(9):3053-3060 (1998), Handgretinger et al., Cancer Immunol Immunother 35(3):199-204 (1992). In some embodiments, an antigen binding domain against GD2 is an antigen binding portion of an antibody selected from mAb 14.18, 14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119.

In one embodiment, an antigen binding domain against BCMA is an antigen binding portion, e.g., CDRs, of an antibody described in, e.g., WO2012163805, WO200112812, and WO2003062401. In some embodiments, additional exemplary BCMA CAR constructs are generated using an antigen binding domain, e.g., CDRs, scFv, or VH and VL sequences from PCT Publication WO2012/0163805 (the contents of which are hereby incorporated by reference in its entirety). In some embodiments, additional exemplary BCMA CAR constructs are generated using an antigen binding domain, e.g., CDRs, scFv, or VH and VL sequences from PCT Publication WO2016/014565 (the contents of which are hereby incorporated by reference in its entirety). In some embodiments, additional exemplary BCMA CAR constructs are generated using an antigen binding domain, e.g., CDRs, scFv, or VH and VL sequences from PCT Publication WO2014/122144 (the contents of which are hereby incorporated by reference in its entirety). In some embodiments, additional exemplary BCMA CAR constructs are generated using the CAR molecules, and/or the BCMA binding domains (e.g., CDRs, scFv, or VH and VL sequences) from PCT Publication WO2016/014789 (the contents of which are hereby incorporated by reference in its entirety). In some embodiments, additional exemplary BCMA CAR constructs are generated using the CAR molecules, and/or the BCMA binding domains (e.g., CDRs, scFv, or VH and VL sequences) from PCT Publication WO2014/089335 (the contents of which are hereby incorporated by reference in its entirety). In some embodiments, additional exemplary BCMA CAR constructs are generated using the CAR molecules, and/or the BCMA binding domains (e.g., CDRs, scFv, or VH and VL sequences) from PCT Publication WO2014/140248 (the contents of which are hereby incorporated by reference in its entirety).

In one embodiment, an antigen binding domain against Tn antigen is an antigen binding portion, e.g., CDRs, of an antibody described in, e.g., US 2014/0178365, U.S. Pat. No. 8,440,798, Brooks et al., PNAS 107(22):10056-10061 (2010), and Stone et al., OncoImmunology 1(6):863-873 (2012).

In one embodiment, an antigen binding domain against PSMA is an antigen binding portion, e.g., CDRs, of an antibody described in, e.g., Parker et al., Protein Expr Purif 89(2):136-145 (2013), US 20110268656 (J591 ScFv); Frigerio et al, European J Cancer 49(9):2223-2232 (2013) (scFvD2B); WO 2006125481 (mAbs 3/A12, 3/E7 and 3/F11) and single chain antibody fragments (scFv A5 and D7).

In one embodiment, an antigen binding domain against ROR1 is an antigen binding portion, e.g., CDRs, of an antibody described in, e.g., Hudecek et al., Clin Cancer Res 19(12):3153-3164 (2013); WO 2011159847; and US20130101607.

In one embodiment, an antigen binding domain against FLT3 is an antigen binding portion, e.g., CDRs, of an antibody described in, e.g., WO2011076922, U.S. Pat. No. 5,777,084, EP0754230, US20090297529, and several commercial catalog antibodies (R&D, ebiosciences, Abcam).

In one embodiment, an antigen binding domain against TAG72 is an antigen binding portion, e.g., CDRs, of an antibody described in, e.g., Hombach et al., Gastroenterology 113(4):1163-1170 (1997); and Abcam ab691.

In one embodiment, an antigen binding domain against FAP is an antigen binding portion, e.g., CDRs, of an antibody described in, e.g., Ostermann et al., Clinical Cancer Research 14:4584-4592 (2008) (FAP5), US Pat. Publication No. 2009/0304718; sibrotuzumab (see e.g., Hofheinz et al., Oncology Research and Treatment 26(1), 2003); and Tran et al., J Exp Med 210(6):1125-1135 (2013).

In one embodiment, an antigen binding domain against CD38 is an antigen binding portion, e.g., CDRs, of daratumumab (see, e.g., Groen et al., Blood 116(21):1261-1262 (2010); MOR202 (see, e.g., U.S. Pat. No. 8,263,746); or antibodies described in U.S. Pat. No. 8,362,211.

In one embodiment, an antigen binding domain against CD44v6 is an antigen binding portion, e.g., CDRs, of an antibody described in, e.g., Casucci et al., Blood 122(20):3461-3472 (2013).

In one embodiment, an antigen binding domain against CEA is an antigen binding portion, e.g., CDRs, of an antibody described in, e.g., Chmielewski et al., Gastroenterology 143(4):1095-1107 (2012).

In one embodiment, an antigen binding domain against EPCAM is an antigen binding portion, e.g., CDRS, of an antibody selected from MT110, EpCAM-CD3 bispecific Ab (see, e.g., clinicaltrials.gov/ct2/show/NCT00635596); Edrecolomab; 3622W94; ING-1; and adecatumumab (MT201).

In one embodiment, an antigen binding domain against PRSS21 is an antigen binding portion, e.g., CDRs, of an antibody described in U.S. Pat. No. 8,080,650.

In one embodiment, an antigen binding domain against B7H3 is an antigen binding portion, e.g., CDRs, of an antibody MGA271 (Macrogenics).

In one embodiment, an antigen binding domain against KIT is an antigen binding portion, e.g., CDRs, of an antibody described in, e.g., U.S. Pat. No. 7,915,391, US20120288506, and several commercial catalog antibodies.

In one embodiment, an antigen binding domain against IL-13Ra2 is an antigen binding portion, e.g., CDRs, of an antibody described in, e.g., WO2008/146911, WO2004087758, several commercial catalog antibodies, and WO2004087758.

In one embodiment, an antigen binding domain against CD30 is an antigen binding portion, e.g., CDRs, of an antibody described in, e.g., U.S. Pat. No. 7,090,843 B1, and EP0805871.

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761; WO2005035577; and U.S. Pat. No. 6,437,098.

In one embodiment, an antigen binding domain against CD171 is an antigen binding portion, e.g., CDRs, of an antibody described in, e.g., Hong et al., J Immunother 37(2):93-104 (2014).

In one embodiment, an antigen binding domain against IL-11Ra is an antigen binding portion, e.g., CDRs, of an antibody available from Abcam (cat #ab55262) or Novus Biologicals (cat #EPR5446). In another embodiment, an antigen binding domain again IL-11Ra is a peptide, see, e.g., Huang et al., Cancer Res 72(1):271-281 (2012).

In one embodiment, an antigen binding domain against PSCA is an antigen binding portion, e.g., CDRs, of an antibody described in, e.g., Morgenroth et al., Prostate 67(10):1121-1131 (2007) (scFv 7F5); Nejatollahi et al., J of Oncology 2013 (2013), article ID 839831 (scFv C5-II); and US Pat Publication No. 20090311181.

In one embodiment, an antigen binding domain against VEGFR2 is an antigen binding portion, e.g., CDRs, of an antibody described in, e.g., Chinnasamy et al., J Clin Invest 120(11):3953-3968 (2010).

In one embodiment, an antigen binding domain against LewisY is an antigen binding portion, e.g., CDRs, of an antibody described in, e.g., Kelly et al., Cancer Biother Radiopharm 23(4):411-423 (2008) (hu3S193 Ab (scFvs)); Dolezal et al., Protein Engineering 16(1):47-56 (2003) (NC10 scFv).

In one embodiment, an antigen binding domain against CD24 is an antigen binding portion, e.g., CDRs, of an antibody described in, e.g., Maliar et al., Gastroenterology 143(5):1375-1384 (2012).

In one embodiment, an antigen binding domain against PDGFR-beta is an antigen binding portion, e.g., CDRs, of an antibody Abcam ab32570.

In one embodiment, an antigen binding domain against SSEA-4 is an antigen binding portion, e.g., CDRs, of antibody MC813 (Cell Signaling), or other commercially available antibodies.

In one embodiment, an antigen binding domain against CD20 is an antigen binding portion, e.g., CDRs, of the antibody Rituximab, Ofatumumab, Ocrelizumab, Veltuzumab, or GA101.

In one embodiment, an antigen binding domain against Folate receptor alpha is an antigen binding portion, e.g., CDRs, of the antibody IMGN853, or an antibody described in US20120009181; U.S. Pat. No. 4,851,332, LK26: U.S. Pat. No. 5,952,484.

In one embodiment, an antigen binding domain against ERBB2 (Her2/neu) is an antigen binding portion, e.g., CDRs, of the antibody trastuzumab, or pertuzumab.

In one embodiment, an antigen binding domain against MUC1 is an antigen binding portion, e.g., CDRs, of the antibody SAR566658.

In one embodiment, the antigen binding domain against EGFR is antigen binding portion, e.g., CDRs, of the antibody cetuximab, panitumumab, zalutumumab, nimotuzumab, or matuzumab. In one embodiment, the antigen binding domain against EGFRvIII is or may be derived from an antigen binding domain, e.g., CDRs, scFv, or VH and VL, of an antibody, antigen-binding fragment or CAR described in, e.g., PCT publication WO2014/130657 (In one embodiment the CAR is a CAR described in WO2014/130657, the contents of which are incorporated herein in their entirety).

In one embodiment, an antigen binding domain against NCAM is an antigen binding portion, e.g., CDRs, of the antibody clone 2-2B: MAB5324 (EMD Millipore)

In one embodiment, an antigen binding domain against Ephrin B2 is an antigen binding portion, e.g., CDRs, of an antibody described in, e.g., Abengozar et al., Blood 119(19):4565-4576 (2012).

In one embodiment, an antigen binding domain against IGF-I receptor is an antigen binding portion, e.g., CDRs, of an antibody described in, e.g., U.S. Pat. No. 8,344,112 B2; EP2322550 A1; WO 2006/138315, or PCT/US2006/022995.

In one embodiment, an antigen binding domain against CAIX is an antigen binding portion, e.g., CDRs, of the antibody clone 303123 (R&D Systems).

In one embodiment, an antigen binding domain against LMP2 is an antigen binding portion, e.g., CDRs, of an antibody described in, e.g., U.S. Pat. No. 7,410,640, or US20050129701.

In one embodiment, an antigen binding domain against gp100 is an antigen binding portion, e.g., CDRs, of the antibody HMB45, NKIbetaB, or an antibody described in WO2013165940, or US20130295007.

In one embodiment, an antigen binding domain against tyrosinase is an antigen binding portion, e.g., CDRs, of an antibody described in, e.g., U.S. Pat. No. 5,843,674; or US19950504048.

In one embodiment, an antigen binding domain against EphA2 is an antigen binding portion, e.g., CDRs, of an antibody described in, e.g., Yu et al., Mol Ther 22(1):102-111 (2014).

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761 A3; 20120276046; WO2005035577; or U.S. Pat. No. 6,437,098.

In one embodiment, an antigen binding domain against fucosyl GM1 is an antigen binding portion, e.g., CDRs, of an antibody described in, e.g., 0520100297138; or WO2007/067992.

In one embodiment, an antigen binding domain against sLe is an antigen binding portion, e.g., CDRs, of the antibody G193 (for lewis Y), see Scott A M et al, Cancer Res 60: 3254-61 (2000), also as described in Neeson et al, J Immunol May 2013 190 (Meeting Abstract Supplement) 177.10.

In one embodiment, an antigen binding domain against GM3 is an antigen binding portion, e.g., CDRs, of the antibody CA 2523449 (mAb 14F7).

In one embodiment, an antigen binding domain against HMWMAA is an antigen binding portion, e.g., CDRs, of an antibody described in, e.g., Kmiecik et al., Oncoimmunology 3(1):e27185 (2014) (PMID: 24575382) (mAb9.2.27); U.S. Pat. No. 6,528,481; WO2010033866; or US 20140004124.

In one embodiment, an antigen binding domain against o-acetyl-GD2 is an antigen binding portion, e.g., CDRs, of the antibody 8B6.

In one embodiment, an antigen binding domain against TEM1/CD248 is an antigen binding portion, e.g., CDRs, of an antibody described in, e.g., Marty et al., Cancer Lett 235(2):298-308 (2006); Zhao et al., J Immunol Methods 363(2):221-232 (2011).

In one embodiment, an antigen binding domain against CLDN6 is an antigen binding portion, e.g., CDRs, of the antibody IMAB027 (Ganymed Pharmaceuticals), see e.g., clinicaltrial.gov/show/NCT02054351.

In one embodiment, an antigen binding domain against TSHR is an antigen binding portion, e.g., CDRs, of an antibody described in, e.g., U.S. Pat. Nos. 8,603,466; 8,501,415; or U.S. Pat. No. 8,309,693.

In one embodiment, an antigen binding domain against GPRC5D is an antigen binding portion, e.g., CDRs, of the antibody FAB6300A (R&D Systems); or LS-A4180 (Lifespan Biosciences).

In one embodiment, an antigen binding domain against CD97 is an antigen binding portion, e.g., CDRs, of an antibody described in, e.g., U.S. Pat. No. 6,846,911; de Groot et al., J Immunol 183(6):4127-4134 (2009); or an antibody from R&D: MAB3734.

In one embodiment, an antigen binding domain against ALK is an antigen binding portion, e.g., CDRs, of an antibody described in, e.g., Mino-Kenudson et al., Clin Cancer Res 16(5):1561-1571 (2010).

In one embodiment, an antigen binding domain against polysialic acid is an antigen binding portion, e.g., CDRs, of an antibody described in, e.g., Nagae et al., J Biol Chem 288(47):33784-33796 (2013).

In one embodiment, an antigen binding domain against PLAC1 is an antigen binding portion, e.g., CDRs, of an antibody described in, e.g., Ghods et al., Biotechnol Appl Biochem 2013 doi:10.1002/bab.1177.

In one embodiment, an antigen binding domain against GloboH is an antigen binding portion of the antibody VK9; or an antibody described in, e.g., Kudryashov V et al, Glycoconj J. 15(3):243-9 (1998), Lou et al., Proc Natl Acad Sci USA 111(7):2482-2487 (2014); MBr1: Bremer E-G et al. J Biol Chem 259:14773-14777 (1984).

In one embodiment, an antigen binding domain against NY-BR-1 is an antigen binding portion, e.g., CDRs of an antibody described in, e.g., Jager et al., Appl Immunohistochem Mol Morphol 15(1):77-83 (2007).

In one embodiment, an antigen binding domain against WT-1 is an antigen binding portion, e.g., CDRs, of an antibody described in, e.g., Dao et al., Sci Transl Med 5(176):176ra33 (2013); or WO2012/135854.

In one embodiment, an antigen binding domain against MAGE-A1 is an antigen binding portion, e.g., CDRs, of an antibody described in, e.g., Willemsen et al., J Immunol 174(12):7853-7858 (2005) (TCR-like scFv).

In one embodiment, an antigen binding domain against sperm protein 17 is an antigen binding portion, e.g., CDRs, of an antibody described in, e.g., Song et al., Target Oncol 2013 Aug. 14 (PMID: 23943313); Song et al., Med Oncol 29(4):2923-2931 (2012).

In one embodiment, an antigen binding domain against Tie 2 is an antigen binding portion, e.g., CDRs, of the antibody AB33 (Cell Signaling Technology).

In one embodiment, an antigen binding domain against MAD-CT-2 is an antigen binding portion, e.g., CDRs, of an antibody described in, e.g., PMID: 2450952; U.S. Pat. No. 7,635,753.

In one embodiment, an antigen binding domain against Fos-related antigen 1 is an antigen binding portion, e.g., CDRs, of the antibody 12F9 (Novus Biologicals).

In one embodiment, an antigen binding domain against MelanA/MART1 is an antigen binding portion, e.g., CDRs, of an antibody described in, EP2514766 A2; or U.S. Pat. No. 7,749,719.

In one embodiment, an antigen binding domain against sarcoma translocation breakpoints is an antigen binding portion, e.g., CDRs, of an antibody described in, e.g., Luo et al, EMBO Mol. Med. 4(6):453-461 (2012).

In one embodiment, an antigen binding domain against TRP-2 is an antigen binding portion, e.g., CDRs, of an antibody described in, e.g., Wang et al, J Exp Med. 184(6):2207-16 (1996).

In one embodiment, an antigen binding domain against CYP1B1 is an antigen binding portion, e.g., CDRs, of an antibody described in, e.g., Maecker et al, Blood 102 (9): 3287-3294 (2003).

In one embodiment, an antigen binding domain against RAGE-1 is an antigen binding portion, e.g., CDRs, of the antibody MAB5328 (EMD Millipore).

In one embodiment, an antigen binding domain against human telomerase reverse transcriptase is an antigen binding portion, e.g., CDRs, of the antibody cat no: LS-B95-100 (Lifespan Biosciences)

In one embodiment, an antigen binding domain against intestinal carboxyl esterase is an antigen binding portion, e.g., CDRs, of the antibody 4F12: cat no: LS-B6190-50 (Lifespan Biosciences).

In one embodiment, an antigen binding domain against mut hsp70-2 is an antigen binding portion, e.g., CDRs, of the antibody Lifespan Biosciences: monoclonal: cat no: LS-C133261-100 (Lifespan Biosciences).

In one embodiment, an antigen binding domain against CD79a is an antigen binding portion, e.g., CDRs, of the antibody Anti-CD79a antibody [HM47/A9] (ab3121), available from Abcam; antibody CD79A Antibody #3351 available from Cell Signalling Technology; or antibody HPA017748-Anti-CD79A antibody produced in rabbit, available from Sigma Aldrich.

In one embodiment, an antigen binding domain against CD79b is an antigen binding portion, e.g., CDRs, of the antibody polatuzumab vedotin, anti-CD79b described in Dornan et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56^{th} ASH Annual Meeting and Exposition, San Francisco, Calif. Dec. 6-9, 2014.

In one embodiment, an antigen binding domain against CD72 is an antigen binding portion, e.g., CDRs, of the antibody J3-109 described in Myers, and Uckun, “An anti-CD72 immunotoxin against therapy-refractory B-lineage acute lymphoblastic leukemia.” Leuk Lymphoma. 1995 June; 18(1-2):119-22, or anti-CD72 (10D6.8.1, mIgG1) described in Polson et al., “Antibody-Drug Conjugates for the Treatment of Non-Hodgkins Lymphoma: Target and Linker-Drug Selection” Cancer Res Mar. 15, 2009 69; 2358.

In one embodiment, an antigen binding domain against LAIR1 is an antigen binding portion, e.g., CDRs, of the antibody ANT-301 LAIR1 antibody, available from ProSpec; or anti-human CD305 (LAIR1) Antibody, available from BioLegend.

In one embodiment, an antigen binding domain against FCAR is an antigen binding portion, e.g., CDRs, of the antibody CD89/FCARAntibody (Catalog #10414-H08H), available from Sino Biological Inc.

In one embodiment, an antigen binding domain against LILRA2 is an antigen binding portion, e.g., CDRs, of the antibody LILRA2 monoclonal antibody (M17), clone 3C7, available from Abnova, or Mouse Anti-LILRA2 antibody, Monoclonal (2D7), available from Lifespan Biosciences.

In one embodiment, an antigen binding domain against CD300LF is an antigen binding portion, e.g., CDRs, of the antibody Mouse Anti-CMRF35-like molecule 1 antibody, Monoclonal[UP-D2], available from BioLegend, or Rat Anti-CMRF35-like molecule 1 antibody, Monoclonal[234903], available from R&D Systems.

In one embodiment, an antigen binding domain against CLEC12A is an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1×CD3 BiTE Antibody” 53^{rd} ASH Annual Meeting and Exposition, Dec. 10-13, 2011, and MCLA-117 (Merus).

In one embodiment, an antigen binding domain against BST2 (also called CD317) is an antigen binding portion, e.g., CDRs, of the antibody Mouse Anti-CD317 antibody, Monoclonal[3H4], available from Antibodies-Online or Mouse Anti-CD317 antibody, Monoclonal[696739], available from R&D Systems.

In one embodiment, an antigen binding domain against EMR2 (also called CD312) is an antigen binding portion, e.g., CDRs, of the antibody Mouse Anti-CD312 antibody, Monoclonal[LS-B8033] available from Lifespan Biosciences, or Mouse Anti-CD312 antibody, Monoclonal[494025] available from R&D Systems.

In one embodiment, an antigen binding domain against LY75 is an antigen binding portion, e.g., CDRs, of the antibody Mouse Anti-Lymphocyte antigen 75 antibody, Monoclonal[HD30] available from EMD Millipore or Mouse Anti-Lymphocyte antigen 75 antibody, Monoclonal[A15797] available from Life Technologies.

In one embodiment, an antigen binding domain against GPC3 is an antigen binding portion, e.g., CDRs, of the antibody hGC33 described in Nakano K, Ishiguro T, Konishi H, et al. Generation of a humanized anti-glypican 3 antibody by CDR grafting and stability optimization. Anticancer Drugs. 2010 November; 21(10):907-916, or MDX-1414, HN3, or YP7, all three of which are described in Feng et al., “Glypican-3 antibodies: a new therapeutic target for liver cancer.” FEBS Lett. 2014 Jan. 21; 588(2):377-82.

In one embodiment, an antigen binding domain against FCRL5 is an antigen binding portion, e.g., CDRs, of the anti-FcRL5 antibody described in Elkins et al., “FcRL5 as a target of antibody-drug conjugates for the treatment of multiple myeloma” Mol Cancer Ther. 2012 October; 11(10):2222-32.

In one embodiment, an antigen binding domain against IGLL1 is an antigen binding portion, e.g., CDRs, of the antibody Mouse Anti-Immunoglobulin lambda-like polypeptide 1 antibody, Monoclonal[AT1G4] available from Lifespan Biosciences, Mouse Anti-Immunoglobulin lambda-like polypeptide 1 antibody, Monoclonal[HSL11] available from BioLegend.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed above.

In another aspect, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In one aspect, the antigen binding domain is humanized.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a VH4_4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3_1.25 germline sequence. In one embodiment, the framework region can comprise, one, two, three, four or five modifications, e.g., substitutions, e.g., from the amino acid at the corresponding murine sequence.

In some aspects, the portion of a CAR composition of the invention that comprises an antibody fragment is humanized with retention of high affinity for the target antigen and other favorable biological properties. According to one aspect of the invention, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., in the present invention, the ability to bind human a cancer associated antigen as described herein. In some embodiments, a humanized antibody or antibody fragment may have improved affinity and/or specificity of binding to human a cancer associated antigen as described herein.

In one aspect, the antigen binding domain of the invention is characterized by particular functional features or properties of an antibody or antibody fragment. For example, in one aspect, the portion of a CAR composition of the invention that comprises an antigen binding domain specifically binds a tumor antigen as described herein.

In one aspect, the anti-cancer associated antigen as described herein binding domain is a fragment, e.g., a single chain variable fragment (scFv). In one aspect, the anti-cancer associated antigen as described herein binding domain is a Fv, a Fab, a (Fab)2, or a bi-functional (e.g. bi-specific) hybrid antibody (e.g., Lanzavecchia et al., Eur. J. Immunol. 17, 105 (1987)). In one aspect, the antibodies and fragments thereof of the invention binds a cancer associated antigen as described herein protein with wild-type or enhanced affinity.

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO:22). In one embodiment, the linker can be (Gly₄Ser)₄ (SEQ ID NO:29) or (Gly₄Ser)₃ (SEQ ID NO:30). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracelluar, however, a fragment of such antigen (peptide) is presented on the surface of the cancer cells by MHC.

In one embodiment, an antigen binding domain against EGFRvIII is an antigen binding portion, e.g., CDRs, of a CAR, antibody or antigen-binding fragment thereof described in, e.g., PCT publication WO2014/130657 or US2014/0322275A1. In one embodiment, the CAR molecule comprises an EGFRvIII CAR, or an antigen binding domain according to Table 2 or SEQ ID NO:11 of WO 2014/130657, incorporated herein by reference, or a sequence substantially identical thereto (e.g., at least 85%, 90%, 95% or more identical thereto). The amino acid and nucleotide sequences encoding the EGFRvIII CAR molecules and antigen binding domains (e.g., including one, two, three VH CDRs; and one, two, three VL CDRs according to Kabat or Chothia), are specified in WO 2014/130657.

In one embodiment, an antigen binding domain against mesothelin is an antigen binding portion, e.g., CDRs, of an antibody, antigen-binding fragment or CAR described in, e.g., PCT publication WO2015/090230. In one embodiment, an antigen binding domain against mesothelin is an antigen binding portion, e.g., CDRs, of an antibody, antigen-binding fragment, or CAR described in, e.g., PCT publication WO1997/025068, WO1999/028471, WO2005/014652, WO2006/099141, WO2009/045957, WO2009/068204, WO2013/142034, WO2013/040557, or WO2013/063419.

In an embodiment, the CAR molecule comprises a mesothelin CAR described herein, e.g., a mesothelin CAR described in WO 2015/090230, incorporated herein by reference. In some embodiments, the mesothelin CAR comprises an amino acid, or has a nucleotide sequence shown in WO 2015/090230 incorporated herein by reference, or a sequence substantially identical to any of the aforesaid sequences (e.g., at least 85%, 90%, 95% or more identical to any of the aforesaid mesothelin CAR sequences). In one embodiment, the CAR molecule comprises a mesothelin CAR, or an antigen binding domain according to Tables 2-3 of WO 2015/090230, incorporated herein by reference, or a sequence substantially identical thereto (e.g., at least 85%, 90%, 95% or more identical thereto). The amino acid and nucleotide sequences encoding the mesothelin CAR molecules and antigen binding domains (e.g., including one, two, three VH CDRs; and one, two, three VL CDRs according to Kabat or Chothia), are specified in WO 2015/090230.

In one embodiment, an antigen binding domain against CD123 is an antigen binding portion, e.g., CDRs, of an antibody, antigen-binding fragment or CAR described in, e.g., PCT publication WO2016/028896. In one embodiment, an antigen binding domain against CD123 is an antigen binding portion, e.g., CDRs, of an antibody, antigen-binding fragment or CAR described in, e.g., PCT publication WO2014/130635. In one embodiment, an antigen binding domain against CD123 is an antigen binding portion, e.g., CDRs, of an antibody, antigen-binding fragment, or CAR described in, e.g., PCT publication WO2014/138805, WO2014/138819, WO2013/173820, WO2014/144622, WO2001/66139, WO2010/126066, WO2014/144622, or US2009/0252742.

In one embodiment, an antigen binding domain against CD123 is an antigen binding portion, e.g., CDRs, of an antibody, antigen-binding fragment or CAR described in, e.g., US2014/0322212A1 or US2016/0068601A1, both incorporated herein by reference. In some embodiments, the CD123 CAR comprises an amino acid, or has a nucleotide sequence shown in US2014/0322212A1 or US2016/0068601A1, both incorporated herein by reference, or a sequence substantially identical to any of the aforesaid sequences (e.g., at least 85%, 90%, 95% or more identical to any of the aforesaid CD123 CAR sequences). In one embodiment, the CAR molecule comprises a CD123 CAR (e.g., any of the CAR1-CAR8), or an antigen binding domain according to Tables 1-2 of WO 2014/130635, incorporated herein by reference, or a sequence substantially identical thereto (e.g., at least 85%, 90%, 95% or more identical to any of the aforesaid CD123 CAR sequences). The amino acid and nucleotide sequences encoding the CD123 CAR molecules and antigen binding domains (e.g., including one, two, three VH CDRs; and one, two, three VL CDRs according to Kabat or Chothia), are specified in WO 2014/130635.

In other embodiments, the CAR molecule comprises a CD123 CAR comprises a CAR molecule (e.g., any of the CAR123-1 to CAR123-4 and hzCAR123-1 to hzCAR123-32), or an antigen binding domain according to Tables 2, 6, and 9 of WO2016/028896, incorporated herein by reference, or a sequence substantially identical thereto (e.g., at least 85%, 90%, 95% or more identical to any of the aforesaid CD123 CAR sequences). The amino acid and nucleotide sequences encoding the CD123 CAR molecules and antigen binding domains (e.g., including one, two, three VH CDRs; and one, two, three VL CDRs according to Kabat or Chothia), are specified in WO2016/028896.

In one embodiment, an antigen binding domain against CD22 is an antigen binding portion, e.g., CDRs, of an antibody described in, e.g., Haso et al., Blood, 121(7): 1165-1174 (2013); Wayne et al., Clin Cancer Res 16(6): 1894-1903 (2010); Kato et al., Leuk Res 37(1):83-88 (2013); Creative BioMart (creativebiomart.net): MOM-18047-S(P).

In one embodiment, an antigen binding domain against CS-1 is an antigen binding portion, e.g., CDRs, of Elotuzumab (BMS), see e.g., Tai et al., 2008, Blood 112(4):1329-37; Tai et al., 2007, Blood. 110(5):1656-63.

In one embodiment, an antigen binding domain against CLL-1 is an antigen binding portion, e.g., CDRs, of an antibody available from R&D, ebiosciences, Abcam, for example, PE-CLL1-hu Cat #353604 (BioLegend); and PE-CLL1 (CLEC12A) Cat #562566 (BD).

In other embodiments, the CLL1 CAR includes a CAR molecule, or an antigen binding domain according to Table 2 of WO2016/014535, incorporated herein by reference. The amino acid and nucleotide sequences encoding the CLL-1 CAR molecules and antigen binding domains (e.g., including one, two, three VH CDRs; and one, two, three VL CDRs according to Kabat or Chothia), are specified in WO2016/014535.

In one embodiment, an antigen binding domain against CD33 is an antigen binding portion, e.g., CDRs, of an antibody described in, e.g., Bross et al., Clin Cancer Res 7(6):1490-1496 (2001) (Gemtuzumab Ozogamicin, hP67.6), Caron et al., Cancer Res 52(24):6761-6767 (1992) (Lintuzumab, HuM195), Lapusan et al., Invest New Drugs 30(3):1121-1131 (2012) (AVE9633), Aigner et al., Leukemia 27(5): 1107-1115 (2013) (AMG330, CD33 BiTE), Dutour et al., Adv hematol 2012: 683065 (2012), and Pizzitola et al., Leukemia doi:10.1038/Lue.2014.62 (2014).

In one embodiment, an antigen binding domain against CD33 is an antigen binding portion, e.g., CDRs, of an antibody described in, US2016/0096892A1, incorporated herein by reference. In some embodiments, the CD33 CAR comprises an amino acid, or has a nucleotide sequence shown in US2016/0096892A1, incorporated herein by reference, or a sequence substantially identical to any of the aforesaid sequences (e.g., at least 85%, 90%, 95% or more identical to any of the aforesaid CD33 CAR sequences). In other embodiments, the CD33 CAR CAR or antigen binding domain thereof can include a CAR molecule (e.g., any of CAR33-1 to CAR-33-9), or an antigen binding domain according to Table 2 or 9 of WO2016/014576, incorporated herein by reference, or a sequence substantially identical to any of the aforesaid sequences (e.g., at least 85%, 90%, 95% or more identical to any of the aforesaid CD33 CAR sequences). The amino acid and nucleotide sequences encoding the CD33 CAR molecules and antigen binding domains (e.g., including one, two, three VH CDRs; and one, two, three VL CDRs according to Kabat or Chothia), are specified in WO2016/014576.

In one embodiment, an antigen binding domain against GD2 is an antigen binding portion, e.g., CDRs, of an antibody described in, e.g., Mujoo et al., Cancer Res. 47(4):1098-1104 (1987); Cheung et al., Cancer Res 45(6):2642-2649 (1985), Cheung et al., J Clin Oncol 5(9):1430-1440 (1987), Cheung et al., J Clin Oncol 16(9):3053-3060 (1998), Handgretinger et al., Cancer Immunol Immunother 35(3):199-204 (1992). In some embodiments, an antigen binding domain against GD2 is an antigen binding portion of an antibody selected from mAb 14.18, 14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119.

In one embodiment, an antigen binding domain against BCMA is an antigen binding portion, e.g., CDRs, of an antibody, antigen-binding fragment or CAR described in, e.g., PCT publication WO2016/014565, e.g., the antigen binding portion of CAR BCMA-10 as described in WO2016/014565. In one embodiment, an antigen binding domain against BCMA is an antigen binding portion, e.g., CDRs, of an antibody, antigen-binding fragment or CAR described in, e.g., PCT publication WO2016/014789. In one embodiment, an antigen binding domain against BCMA is an antigen binding portion, e.g., CDRs, of an antibody described in, e.g., WO2012/163805, WO2001/12812, and WO2003/062401.

In other embodiment, the CAR molecule comprises a BCMA CAR molecule, or an antigen binding domain against BCMA described herein, e.g., a BCMA CAR described in US-2016-0046724-A1 or WO2016/014565. In some embodiments, the BCMA CAR comprises an amino acid, or has a nucleotide sequence of a CAR molecule, or an antigen binding domain according to US-2016-0046724-A1, or Table 1 or 16, SEQ ID NO: 271 or SEQ ID NO: 273 of WO2016/014565, incorporated herein by reference, or a sequence substantially identical to any of the aforesaid sequences (e.g., at least 85%, 90%, 95% or more identical to any of the aforesaid BCMA CAR sequences). The amino acid and nucleotide sequences encoding the BCMA CAR molecules and antigen binding domains (e.g., including one, two, three VH CDRs; and one, two, three VL CDRs according to Kabat or Chothia), are specified in WO2016/014565.

In one embodiment, an antigen binding domain against GFR ALPHA-4 CAR antigen is an antigen binding portion, e.g., CDRs, of an antibody described in, e.g., WO2016/025880, incorporated herein by reference. In one embodiment, the CAR molecule comprises an a GFR ALPHA-4 CAR, e.g., a CAR molecule, or an antigen binding domain according to Table 2 of WO2016/025880, incorporated herein by reference, or a sequence substantially identical to any of the aforesaid sequences (e.g., at least 85%, 90%, 95% or more identical to any of the aforesaid GFR ALPHA-4 sequences). The amino acid and nucleotide sequences encoding the GFR ALPHA-4 CAR molecules and antigen binding domains (e.g., including one, two, three VH CDRs; and one, two, three VL CDRs according to Kabat or Chothia), are specified in WO2016/025880.

In one embodiment, an antigen binding domain against Tn antigen is an antigen binding portion, e.g., CDRs, of an antibody described in, e.g., U.S. Pat. No. 8,440,798; Brooks et al., PNAS 107(22):10056-10061 (2010), and Stone et al., OncoImmunology 1(6):863-873 (2012).

In one embodiment, an antigen binding domain against PSMA is an antigen binding portion, e.g., CDRs, of an antibody described in, e.g., Parker et al., Protein Expr Purif 89(2):136-145 (2013), US 20110268656 (J591 ScFv); Frigerio et al, European J Cancer 49(9):2223-2232 (2013) (scFvD2B); WO 2006125481 (mAbs 3/A12, 3/E7 and 3/F11) and single chain antibody fragments (scFv A5 and D7).

In one embodiment, an antigen binding domain against ROR1 is an antigen binding portion, e.g., CDRs, of an antibody described in, e.g., Hudecek et al., Clin Cancer Res 19(12):3153-3164 (2013); WO 2011159847; and US20130101607.

In one embodiment, an antigen binding domain against FLT3 is an antigen binding portion, e.g., CDRs, of an antibody described in, e.g., WO2011076922, U.S. Pat. No. 5,777,084, EP0754230, US20090297529, and several commercial catalog antibodies (R&D, ebiosciences, Abcam).

In one embodiment, an antigen binding domain against TAG72 is an antigen binding portion, e.g., CDRs, of an antibody described in, e.g., Hombach et al., Gastroenterology 113(4):1163-1170 (1997); and Abcam ab691.

In one embodiment, an antigen binding domain against FAP is an antigen binding portion, e.g., CDRs, of an antibody described in, e.g., Ostermann et al., Clinical Cancer Research 14:4584-4592 (2008) (FAP5), US Pat. Publication No. 2009/0304718; sibrotuzumab (see e.g., Hofheinz et al., Oncology Research and Treatment 26(1), 2003); and Tran et al., J Exp Med 210(6):1125-1135 (2013).

In one embodiment, an antigen binding domain against CD38 is an antigen binding portion, e.g., CDRs, of daratumumab (see, e.g., Groen et al., Blood 116(21):1261-1262 (2010); MOR202 (see, e.g., U.S. Pat. No. 8,263,746); or antibodies described in U.S. Pat. No. 8,362,211.

In one embodiment, an antigen binding domain against CD44v6 is an antigen binding portion, e.g., CDRs, of an antibody described in, e.g., Casucci et al., Blood 122(20):3461-3472 (2013).

In one embodiment, an antigen binding domain against CEA is an antigen binding portion, e.g., CDRs, of an antibody described in, e.g., Chmielewski et al., Gastroenterology 143(4):1095-1107 (2012).

In one embodiment, an antigen binding domain against EPCAM is an antigen binding portion, e.g., CDRS, of an antibody selected from MT110, EpCAM-CD3 bispecific Ab (see, e.g., clinicaltrials.gov/ct2/show/NCT00635596); Edrecolomab; 3622W94; ING-1; and adecatumumab (MT201).

In one embodiment, an antigen binding domain against PRSS21 is an antigen binding portion, e.g., CDRs, of an antibody described in U.S. Pat. No. 8,080,650.

In one embodiment, an antigen binding domain against B7H3 is an antigen binding portion, e.g., CDRs, of an antibody MGA271 (Macrogenics).

In one embodiment, an antigen binding domain against KIT is an antigen binding portion, e.g., CDRs, of an antibody described in, e.g., U.S. Pat. No. 7,915,391, US20120288506, and several commercial catalog antibodies.

In one embodiment, an antigen binding domain against IL-13Ra2 is an antigen binding portion, e.g., CDRs, of an antibody described in, e.g., WO2008/146911, WO2004087758, several commercial catalog antibodies, and WO2004087758.

In one embodiment, an antigen binding domain against CD30 is an antigen binding portion, e.g., CDRs, of an antibody described in, e.g., U.S. Pat. No. 7,090,843 B1, and EP0805871.

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761; WO2005035577; and U.S. Pat. No. 6,437,098.

In one embodiment, an antigen binding domain against CD171 is an antigen binding portion, e.g., CDRs, of an antibody described in, e.g., Hong et al., J Immunother 37(2):93-104 (2014).

In one embodiment, an antigen binding domain against IL-11Ra is an antigen binding portion, e.g., CDRs, of an antibody available from Abcam (cat #ab55262) or Novus Biologicals (cat #EPR5446). In another embodiment, an antigen binding domain again IL-11Ra is a peptide, see, e.g., Huang et al., Cancer Res 72(1):271-281 (2012).

In one embodiment, an antigen binding domain against PSCA is an antigen binding portion, e.g., CDRs, of an antibody described in, e.g., Morgenroth et al., Prostate 67(10):1121-1131 (2007) (scFv 7F5); Nejatollahi et al., J of Oncology 2013 (2013), article ID 839831 (scFv C5-II); and US Pat Publication No. 20090311181.

In one embodiment, an antigen binding domain against VEGFR2 is an antigen binding portion, e.g., CDRs, of an antibody described in, e.g., Chinnasamy et al., J Clin Invest 120(11):3953-3968 (2010).

In one embodiment, an antigen binding domain against LewisY is an antigen binding portion, e.g., CDRs, of an antibody described in, e.g., Kelly et al., Cancer Biother Radiopharm 23(4):411-423 (2008) (hu3S193 Ab (scFvs)); Dolezal et al., Protein Engineering 16(1):47-56 (2003) (NC10 scFv).

In one embodiment, an antigen binding domain against CD24 is an antigen binding portion, e.g., CDRs, of an antibody described in, e.g., Maliar et al., Gastroenterology 143(5):1375-1384 (2012).

In one embodiment, an antigen binding domain against PDGFR-beta is an antigen binding portion, e.g., CDRs, of an antibody Abcam ab32570.

In one embodiment, an antigen binding domain against SSEA-4 is an antigen binding portion, e.g., CDRs, of antibody MC813 (Cell Signaling), or other commercially available antibodies.

In one embodiment, an antigen binding domain against CD20 is an antigen binding portion, e.g., CDRs, of the antibody Rituximab, Ofatumumab, Ocrelizumab, Veltuzumab, or GA101.

In one embodiment, an antigen binding domain against Folate receptor alpha is an antigen binding portion, e.g., CDRs, of the antibody IMGN853, or an antibody described in US20120009181; U.S. Pat. No. 4,851,332, LK26: U.S. Pat. No. 5,952,484.

In one embodiment, an antigen binding domain against ERBB2 (Her2/neu) is an antigen binding portion, e.g., CDRs, of the antibody trastuzumab, or pertuzumab.

In one embodiment, an antigen binding domain against MUC1 is an antigen binding portion, e.g., CDRs, of the antibody SAR566658.

In one embodiment, the antigen binding domain against EGFR is antigen binding portion, e.g., CDRs, of the antibody cetuximab, panitumumab, zalutumumab, nimotuzumab, or matuzumab.

In one embodiment, an antigen binding domain against NCAM is an antigen binding portion, e.g., CDRs, of the antibody clone 2-2B: MAB5324 (EMD Millipore).

In one embodiment, an antigen binding domain against Ephrin B2 is an antigen binding portion, e.g., CDRs, of an antibody described in, e.g., Abengozar et al., Blood 119(19):4565-4576 (2012).

In one embodiment, an antigen binding domain against IGF-I receptor is an antigen binding portion, e.g., CDRs, of an antibody described in, e.g., U.S. Pat. No. 8,344,112 B2; EP2322550 A1; WO 2006/138315, or PCT/US2006/022995.

In one embodiment, an antigen binding domain against CAIX is an antigen binding portion, e.g., CDRs, of the antibody clone 303123 (R&D Systems).

In one embodiment, an antigen binding domain against LMP2 is an antigen binding portion, e.g., CDRs, of an antibody described in, e.g., U.S. Pat. No. 7,410,640, or US20050129701.

In one embodiment, an antigen binding domain against gp100 is an antigen binding portion, e.g., CDRs, of the antibody HMB45, NKIbetaB, or an antibody described in WO2013165940, or US20130295007

In one embodiment, an antigen binding domain against tyrosinase is an antigen binding portion, e.g., CDRs, of an antibody described in, e.g., U.S. Pat. No. 5,843,674; or US19950504048.

In one embodiment, an antigen binding domain against EphA2 is an antigen binding portion, e.g., CDRs, of an antibody described in, e.g., Yu et al., Mol Ther 22(1):102-111 (2014).

In one embodiment, an antigen binding domain against GD3 is an antigen binding portion, e.g., CDRs, of an antibody described in, e.g., U.S. Pat. Nos. 7,253,263; 8,207,308; US 20120276046; EP1013761 A3; 20120276046; WO2005035577; or U.S. Pat. No. 6,437,098.

In one embodiment, an antigen binding domain against fucosyl GM1 is an antigen binding portion, e.g., CDRs, of an antibody described in, e.g., 0520100297138; or WO2007/067992.

In one embodiment, an antigen binding domain against sLe is an antigen binding portion, e.g., CDRs, of the antibody G193 (for lewis Y), see Scott A M et al, Cancer Res 60: 3254-61 (2000), also as described in Neeson et al, J Immunol May 2013 190 (Meeting Abstract Supplement) 177.10.

In one embodiment, an antigen binding domain against GM3 is an antigen binding portion, e.g., CDRs, of the antibody CA 2523449 (mAb 14F7).

In one embodiment, an antigen binding domain against HMWMAA is an antigen binding portion, e.g., CDRs, of an antibody described in, e.g., Kmiecik et al., Oncoimmunology 3(1):e27185 (2014) (PMID: 24575382) (mAb9.2.27); U.S. Pat. No. 6,528,481; WO2010033866; or US 20140004124.

In one embodiment, an antigen binding domain against o-acetyl-GD2 is an antigen binding portion, e.g., CDRs, of the antibody 8B6.

In one embodiment, an antigen binding domain against TEM1/CD248 is an antigen binding portion, e.g., CDRs, of an antibody described in, e.g., Marty et al., Cancer Lett 235(2):298-308 (2006); Zhao et al., J Immunol Methods 363(2):221-232 (2011).

In one embodiment, an antigen binding domain against CLDN6 is an antigen binding portion, e.g., CDRs, of the antibody IMAB027 (Ganymed Pharmaceuticals), see e.g., clinicaltrial.gov/show/NCT02054351.

In one embodiment, an antigen binding domain against TSHR is an antigen binding portion, e.g., CDRs, of an antibody described in, e.g., U.S. Pat. Nos. 8,603,466; 8,501,415; or U.S. Pat. No. 8,309,693.

In one embodiment, an antigen binding domain against GPRC5D is an antigen binding portion, e.g., CDRs, of the antibody FAB6300A (R&D Systems); or LS-A4180 (Lifespan Biosciences).

In one embodiment, an antigen binding domain against CD97 is an antigen binding portion, e.g., CDRs, of an antibody described in, e.g., U.S. Pat. No. 6,846,911; de Groot et al., J Immunol 183(6):4127-4134 (2009); or an antibody from R&D: MAB3734.

In one embodiment, an antigen binding domain against ALK is an antigen binding portion, e.g., CDRs, of an antibody described in, e.g., Mino-Kenudson et al., Clin Cancer Res 16(5):1561-1571 (2010).

In one embodiment, an antigen binding domain against polysialic acid is an antigen binding portion, e.g., CDRs, of an antibody described in, e.g., Nagae et al., J Biol Chem 288(47):33784-33796 (2013).

In one embodiment, an antigen binding domain against PLAC1 is an antigen binding portion, e.g., CDRs, of an antibody described in, e.g., Ghods et al., Biotechnol Appl Biochem 2013 doi:10.1002/bab.1177.

In one embodiment, an antigen binding domain against GloboH is an antigen binding portion of the antibody VK9; or an antibody described in, e.g., Kudryashov V et al, Glycoconj J. 15(3):243-9 (1998), Lou et al., Proc Natl Acad Sci USA 111(7):2482-2487 (2014); MBr1: Bremer E-G et al. J Biol Chem 259:14773-14777 (1984).

In one embodiment, an antigen binding domain against NY-BR-1 is an antigen binding portion, e.g., CDRs of an antibody described in, e.g., Jager et al., Appl Immunohistochem Mol Morphol 15(1):77-83 (2007).

In one embodiment, an antigen binding domain against WT-1 is an antigen binding portion, e.g., CDRs, of an antibody described in, e.g., Dao et al., Sci Transl Med 5(176):176ra33 (2013); or WO2012/135854.

In one embodiment, an antigen binding domain against MAGE-A1 is an antigen binding portion, e.g., CDRs, of an antibody described in, e.g., Willemsen et al., J Immunol 174(12):7853-7858 (2005) (TCR-like scFv).

In one embodiment, an antigen binding domain against sperm protein 17 is an antigen binding portion, e.g., CDRs, of an antibody described in, e.g., Song et al., Target Oncol 2013 Aug. 14 (PMID: 23943313); Song et al., Med Oncol 29(4):2923-2931 (2012).

In one embodiment, an antigen binding domain against Tie 2 is an antigen binding portion, e.g., CDRs, of the antibody AB33 (Cell Signaling Technology).

In one embodiment, an antigen binding domain against MAD-CT-2 is an antigen binding portion, e.g., CDRs, of an antibody described in, e.g., PMID: 2450952; U.S. Pat. No. 7,635,753.

In one embodiment, an antigen binding domain against Fos-related antigen 1 is an antigen binding portion, e.g., CDRs, of the antibody 12F9 (Novus Biologicals).

In one embodiment, an antigen binding domain against MelanA/MART1 is an antigen binding portion, e.g., CDRs, of an antibody described in, EP2514766 A2; or U.S. Pat. No. 7,749,719.

In one embodiment, an antigen binding domain against sarcoma translocation breakpoints is an antigen binding portion, e.g., CDRs, of an antibody described in, e.g., Luo et al, EMBO Mol. Med. 4(6):453-461 (2012).

In one embodiment, an antigen binding domain against TRP-2 is an antigen binding portion, e.g., CDRs, of an antibody described in, e.g., Wang et al, J Exp Med. 184(6):2207-16 (1996).

In one embodiment, an antigen binding domain against CYP1B1 is an antigen binding portion, e.g., CDRs, of an antibody described in, e.g., Maecker et al, Blood 102 (9): 3287-3294 (2003).

In one embodiment, an antigen binding domain against RAGE-1 is an antigen binding portion, e.g., CDRs, of the antibody MAB5328 (EMD Millipore).

In one embodiment, an antigen binding domain against human telomerase reverse transcriptase is an antigen binding portion, e.g., CDRs, of the antibody cat no: LS-B95-100 (Lifespan Biosciences)

In one embodiment, an antigen binding domain against intestinal carboxyl esterase is an antigen binding portion, e.g., CDRs, of the antibody 4F12: cat no: LS-B6190-50 (Lifespan Biosciences).

In one embodiment, an antigen binding domain against mut hsp70-2 is an antigen binding portion, e.g., CDRs, of the antibody Lifespan Biosciences: monoclonal: cat no: LS-C133261-100 (Lifespan Biosciences).

In one embodiment, an antigen binding domain against CD79a is an antigen binding portion, e.g., CDRs, of the antibody Anti-CD79a antibody [HM47/A9] (ab3121), available from Abcam; antibody CD79A Antibody #3351 available from Cell Signalling Technology; or antibody HPA017748-Anti-CD79A antibody produced in rabbit, available from Sigma Aldrich.

In one embodiment, an antigen binding domain against CD79b is an antigen binding portion, e.g., CDRs, of the antibody polatuzumab vedotin, anti-CD79b described in Dornan et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56^{th} ASH Annual Meeting and Exposition, San Francisco, Calif. Dec. 6-9, 2014.

In one embodiment, an antigen binding domain against CD72 is an antigen binding portion, e.g., CDRs, of the antibody J3-109 described in Myers, and Uckun, “An anti-CD72 immunotoxin against therapy-refractory B-lineage acute lymphoblastic leukemia.” Leuk Lymphoma. 1995 June; 18(1-2):119-22, or anti-CD72 (10D6.8.1, mIgG1) described in Polson et al., “Antibody-Drug Conjugates for the Treatment of Non-Hodgkins Lymphoma: Target and Linker-Drug Selection” Cancer Res Mar. 15, 2009 69; 2358.

In one embodiment, an antigen binding domain against LAIR1 is an antigen binding portion, e.g., CDRs, of the antibody ANT-301 LAIR1 antibody, available from ProSpec; or anti-human CD305 (LAIR1) Antibody, available from BioLegend.

In one embodiment, an antigen binding domain against FCAR is an antigen binding portion, e.g., CDRs, of the antibody CD89/FCARAntibody (Catalog #10414-H08H), available from Sino Biological Inc.

In one embodiment, an antigen binding domain against LILRA2 is an antigen binding portion, e.g., CDRs, of the antibody LILRA2 monoclonal antibody (M17), clone 3C7, available from Abnova, or Mouse Anti-LILRA2 antibody, Monoclonal (2D7), available from Lifespan Biosciences.

In one embodiment, an antigen binding domain against CD300LF is an antigen binding portion, e.g., CDRs, of the antibody Mouse Anti-CMRF35-like molecule 1 antibody, Monoclonal[UP-D2], available from BioLegend, or Rat Anti-CMRF35-like molecule 1 antibody, Monoclonal[234903], available from R&D Systems.

In one embodiment, an antigen binding domain against CLEC12A is an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1×CD3 BiTE Antibody” 53^{rd} ASH Annual Meeting and Exposition, Dec. 10-13, 2011, and MCLA-117 (Merus).

In one embodiment, an antigen binding domain against BST2 (also called CD317) is an antigen binding portion, e.g., CDRs, of the antibody Mouse Anti-CD317 antibody, Monoclonal[3H4], available from Antibodies-Online or Mouse Anti-CD317 antibody, Monoclonal[696739], available from R&D Systems.

In one embodiment, an antigen binding domain against EMR2 (also called CD312) is an antigen binding portion, e.g., CDRs, of the antibody Mouse Anti-CD312 antibody, Monoclonal[LS-B8033] available from Lifespan Biosciences, or Mouse Anti-CD312 antibody, Monoclonal[494025] available from R&D Systems.

In one embodiment, an antigen binding domain against LY75 is an antigen binding portion, e.g., CDRs, of the antibody Mouse Anti-Lymphocyte antigen 75 antibody, Monoclonal[HD30] available from EMD Millipore or Mouse Anti-Lymphocyte antigen 75 antibody, Monoclonal[A15797] available from Life Technologies.

In one embodiment, an antigen binding domain against GPC3 is an antigen binding portion, e.g., CDRs, of the antibody hGC33 described in Nakano K, Ishiguro T, Konishi H, et al. Generation of a humanized anti-glypican 3 antibody by CDR grafting and stability optimization. Anticancer Drugs. 2010 November; 21(10):907-916, or MDX-1414, HN3, or YP7, all three of which are described in Feng et al., “Glypican-3 antibodies: a new therapeutic target for liver cancer.” FEBS Lett. 2014 Jan. 21; 588(2):377-82.

In one embodiment, an antigen binding domain against FCRL5 is an antigen binding portion, e.g., CDRs, of the anti-FcRL5 antibody described in Elkins et al., “FcRL5 as a target of antibody-drug conjugates for the treatment of multiple myeloma” Mol Cancer Ther. 2012 October; 11(10):2222-32.

In one embodiment, an antigen binding domain against IGLL1 is an antigen binding portion, e.g., CDRs, of the antibody Mouse Anti-Immunoglobulin lambda-like polypeptide 1 antibody, Monoclonal[AT1G4] available from Lifespan Biosciences, Mouse Anti-Immunoglobulin lambda-like polypeptide 1 antibody, Monoclonal[HSL11] available from BioLegend.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed above.

In another aspect, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In one aspect, the antigen binding domain is humanized.

### CD19 CAR Constructs

Murine CD19 CAR constructs are described in PCT publication WO 2012/079000, incorporated herein by reference, and the amino acid sequence of the murine CD19 CAR and scFv constructs are shown in Table 2 below.

TABLE 2
Murine CD19 CAR Constructs

| | | |
|---|---|---|
| CTL019 | SEQ ID | MALPVTALLLPLALLLHAARPdiqmtqttsslsaslgdrvtiscrasqdiskylnw |
| Full - aa | NO: 81 | yqqkpdgtvklliyhtsrlhsgvpsrfsgsgsgtdysltisnleqediatyfcqqgntlpytfgggtk |
| | | leitggggsggggsggggsevklqesgpglvapsqslsvtctvsgvslpdygvswirqpprkgl |
| | | ewlgviwgsettyynsalksrltiikdnsksqvflkmnslqtddtaiyycakhyyyggsyamd |
| | | ywgqgtsvtvsstttpaprpptpaptiasqplslrpeacrpaaggavhtrgldfacdiyiwaplagt |
| | | cgvlllslvitlyckrgrkkllyifkqpfmrpvqttqeedgcscrfpeeeeggcelrvkfsrsadap |
| | | aykqgqnqlynelnlgrreeydvldkrrgrdpemggkprrknpqeglynelqkdkmaeayse |
| | | igmkgerrrgkghdglyqglstatkdtydalhmqalppr |
| CTL019 | SEQ ID | Diqmtqttsslsaslgdrvtiscrasqdiskylnwyqqkpdgtvklliyhtsrlhsgvpsrfsgsgs |
| scFv | NO: 52 | gtdysltisnleqediatyfcqqgntlpytfgggtkleitggggsggggsggggsevklqesgpgl |
| domain | | vapsqslsvtctvsgvslpdygvswirqpprkglewlgviwgsettyynsalksrltiikdnsksq |
| | | vflkmnslqtddtaiyycakhyyyggsyamdywgqgtsvtvss |
| mCAR1 | SEQ ID | QVQLLESGAELVRPGSSVKISCKASGYAFSSYWMNWVKQRPG |
| scFv | NO: 84 | QGLEWIGQIYPGDGDTNYNGKFKGQATLTADKSSSTAYMQLS |
| | | GLTSEDSAVYSCARKTISSVVDFYFDYWGQGTTVTGGGSGGG |
| | | SGGGSGGGSELVLTQSPKFMSTSVGDRVSVTCKASQNVGTNV |
| | | AWYQQKPGQSPKPLIYSATYRNSGVPDRFTGSGSGTDFTLTIT |
| | | NVQSKDLADYFCQYNRYPYTSFFFTKLEIKRRS |
| mCAR1 | SEQ ID | QVQLLESGAELVRPGSSVKISCKASGYAFSSYWMNWVKQRPG |
| Full - aa | NO: 85 | QGLEWIGQIYPGDGDTNYNGKFKGQATLTADKSSSTAYMQLS |
| | | GLTSEDSAVYSCARKTISSVVDFYFDYWGQGTTVTGGGSGGG |
| | | SGGGSGGGSELVLTQSPKFMSTSVGDRVSVTCKASQNVGTNV |
| | | AWYQQKPGQSPKPLIYSATYRNSGVPDRFTGSGSGTDFTLTIT |
| | | NVQSKDLADYFCQYNRYPYTSFFFTKLEIKRRSKIEVMYPPPYL |
| | | DNEKSNGTIIHVKGKHLCPSPLFPGPSKPFWVLVVVGGVLACY |
| | | SLLVTVAFIIFWVRSKRSRLLHSDYMNMTPRRPGPTRKHYQPY |
| | | APPRDFAAYRSRVKFSRSADAPAYQQGQNQLYNELNLGRREE |
| | | YDVLDKRRGRDPEMGGKPRRKNPQEGLYNELQKDKMAEAYS |
| | | EIGMKGERRRGKGHDGLYQGLSTATKDTYDALHMQALPPR |
| mCAR2 | SEQ ID | DIQMTQTTSSLSASLGDRVTISCRASQDISKYLNWYQQKPDGT |
| scFv | NO: 86 | VKLLIYHTSRLHSGVPSRFSGSGSGTDYSLTISNLEQEDIATYFC |
| | | QQGNTLPYTFGGGTKLEITGSTSGSGKPGSGEGSTKGEVKLQE |
| | | SGPGLVAPSQSLSVTCTVSGVSLPDYGVSWIRQPPRKGLEWLG |
| | | VIWGSETTYYNSALKSRLTIIKDNSKSQVFLKMNSLQTDDTAIY |
| | | YCAKHYYYGGSYAMDWGQGTSVTVSSE |
| mCAR2 | SEQ ID | DIQMTQTTSSLSASLGDRVTISCRASQDISKYLNWYQQKPDGT |
| CAR - aa | NO: 87 | VKLLIYHTSRLHSGVPSRFSGSGSGTDYSLTISNLEQEDIATYFC |
| | | QQGNTLPYTFGGGTKLEITGSTSGSGKPGSGEGSTKGEVKLQE |
| | | SGPGLVAPSQSLSVTCTVSGVSLPDYGVSWIRQPPRKGLEWLG |
| | | VIWGSETTYYNSALKSRLTIIKDNSKSQVFLKMNSLQTDDTAIY |
| | | YCAKHYYYGGSYAMDYWGQGTSVTVSSESKYGPPCPPCPMF |
| | | WVLVVVGGVLACYSLLVTVAFIIFWVKRGRKKLLYIFKQPFM |
| | | RPVQTTQEEDGCSCRFEEEEGGCELRVKFSRSADAPAYQQGQ |
| | | NQLYNELNLGRREEYDVLDKRRGRDPEMGGKPRRKNPQEGL |
| | | YNELQKDKMAEAYSEIGMKGERRRGKGHDGLYQGLSTATKD |
| | | TYDALHMQALPPRL |
| mCAR2 | SEQ ID | DIQMTQTT SSLSASLGDR VTISCRASQD ISKYLNWYQQ |
| Full - aa | NO: 88 | KPDGTVKLLI YHTSRLHSGV PSRFSGSGSG TDYSLTISNL |
| | | EQEDIATYFC QQGNTLPYTF GGGTKLEITG STSGSGKPGS |
| | | GEGSTKGEVK LQESGPGLVA PSQSLSVTCT VSGVSLPDYG |
| | | VSWIRQPPRK GLEWLGVIWG SETTYYNSAL KSRLTIIKDN |
| | | SKSQVFLKMN SLQTDDTAIY YCAKHYYYGG |
| | | SYAMDYWGQG TSVTVSSESK YGPPCPPCPM FWVLVVVGGV |
| | | LACYSLLVTV |
| | | AFIIFWVKRG RKKLLYIFKQ PFMRPVQTTQ EEDGCSCRFE |
| | | EEEGGCELRV KFSRSADAPA YQQGQNQLYN ELNLGRREEY |
| | | DVLDKRRGRD PEMGGKPRRK NPQEGLYNEL QKDKMAEAYS |
| | | EIGMKGERRR GKGHDGLYQG LSTATKDTYD ALHMQALPPR |
| | | LEGGGEGRGS LLTCGDVEEN PGPRMLLLVT SLLLCELPHP |
| | | AFLLIPRKVC NGIGIGEFKD SLSINATNIK HFKNCTSISG |
| | | DLHILPVAFR GDSFTHTPPL DPQELDILKT VKEITGFLLI |
| | | QAWPENRTDL HAFENLEIIR |
| | | GRTKQHGQFS LAVVSLNITS LGLRSLKEIS DGDVIISGNK |
| | | NLCYANTINW KKLFGTSGQK TKIISNRGEN SCKATGQVCH |
| | | ALCSPEGCWG PEPRDCVSCR NVSRGRECVD KCNLLEGEPR |
| | | EFVENSECIQ CHPECLPQAM NITCTGRGPD NCIQCAHYID |
| | | GPHCVKTCPA GVMGENNTLV WKYADAGHVC |
| | | HLCHPNCTYG CTGPGLEGCP TNGPKIPSIA TGMVGALLLL |
| | | LVVALGIGLF M |
| mCAR3 | SEQ ID | DIQMTQTTSSLSASLGDRVTISCRASQDISKYLNWYQQKPDGT |
| scFv | NO: 89 | VKLLIYHTSRLHSGVPSRFSGSGSGTDYSLTISNLEQEDIATYFC |
| | | QQGNTLPYTFGGGTKLEITGSTSGSGKPGSGEGSTKGEVKLQE |
| | | SGPGLVAPSQSLSVTCTVSGVSLPDYGVSWIRQPPRKGLEWLG |
| | | VIWGSETTYYNSALKSRLTIIKDNSKSQVFLKMNSLQTDDTAIY |
| | | YCAKHYYYGGSYAMDYWGQGTSVTVSS |
| mCAR3 | SEQ ID | DIQMTQTTSSLSASLGDRVTISCRASQDISKYLNWYQQKPDGT |
| Full - aa | NO: 90 | VKLLIYHTSRLHSGVPSRFSGSGSGTDYSLTISNLEQEDIATYFC |
| | | QQGNTLPYTFGGGTKLEITGSTSGSGKPGSGEGSTKGEVKLQE |
| | | SGPGLVAPSQSLSVTCTVSGVSLPDYGVSWIRQPPRKGLEWLG |
| | | VIWGSETTYYNSALKSRLTIIKDNSKSQVFLKMNSLQTDDTAIY |
| | | YCAKHYYYGGSYAMDYWGQGTSVTVSSAAAIEVMYPPPYLD |
| | | NEKSNGTIIHVKGKHLCPSPLFPGPSKPFWVLVVVGGVLACYS |
| | | LLVTVAFIIFWVRSKRSRLLHSDYMNMTPRRPGPTRKHYQPYA |
| | | PPRDFAAYRSRVKFSRSADAPAYQQGQNQLYNELNLGRREEY |
| | | DVLDKRRGRDPEMGGKPRRKNPQEGLYNELQKDKMAEAYSE |
| | | IGMKGERRRGKGHDGLYQGLSTATKDTYDALHMQALPPR |

CD19 CAR constructs containing humanized anti-CD19 scFv domains are described in PCT publication WO 2014/153270, incorporated herein by reference.

In an embodiment, the antigen binding domain comprises an anti-CD19 antibody, or fragment thereof, e.g., an scFv. For example, the antigen binding domain comprises a variable heavy chain and a variable light chain listed in Table 12. The linker sequence joining the variable heavy and variable light chains can be, e.g., any of the linker sequences described herein, or alternatively, can be GSTSGSGKPGSGEGSTKG (SEQ ID NO:45).

TABLE 12
Anti-CD 19 antibody binding domains

| | | |
|---|---|---|
| CD19 | huscFv1 | EIVMTQSPATLSLSPGERATLSCRASQDISKYLNWYQQKPGQAPRL |
| | | LIYHTSRLHSGIPARFSGSGSGTDYTLTISSLQPEDFAVYFCQQGNT |
| | | LPYTFGQGTKLEIKGGGGSGGGGSGGGGSQVQLQESGPGLVKPSE |
| | | TLSLTCTVSGVSLPDYGVSWIRQPPGKGLEWIGVIWGSETTYYSSS |
| | | LKSRVTISKDNSKNQVSLKLSSVTAADTAVYYCAKHYYYGGSYA |
| | | MDYWGQGTLVTVSS (SEQ ID NO: 24) |
| CD19 | huscFv2 | Eivmtqspatlslspgeratlscrasqdiskylnwyqqkpgqaprlliyhtsrlhsgiparfsgsgsgtd |
| | | ytltisslqpedfavyfcqqgntlpytfgqgtkleikggggsggggsggggsqvqlqesgpglvkpset |
| | | lsltctvsgvslpdygvswirqppgkglewigviwgsettyyqsslksrvtiskdnsknqvslklssvta |
| | | adtavyycakhyyyggsyamdywgqgtlvtvss (SEQ ID NO: 25) |
| CD19 | huscFv3 | Qvqlqesgpglvkpsetlsltctvsgvslpdygvswirqppgkglewigviwgsettyyssslksrvti |
| | | skdnsknqvslklssvtaadtavyycakhyyyggsyamdywgqgtlvtvssggggsggggsggg |
| | | gseivmtqspatlslspgeratlscrasqdiskylnwyqqkpgqaprlliyhtsrlhsgiparfsgsgsgt |
| | | dytltisslqpedfavyfcqqgntlpytfgqgtkleik (SEQ ID NO: 26) |
| CD19 | huscFv4 | Qvqlqesgpglvkpsetlsltctvsgvslpdygvswirqppgkglewigviwgsettyyqsslksrvti |
| | | skdnsknqvslklssvtaadtavyycakhyyyggsyamdywgqgtlvtvssggggsggggsggg |
| | | gseivmtqspatlslspgeratlscrasqdiskylnwyqqkpgqaprlliyhtsrlhsgiparfsgsgsgt |
| | | dytltisslqpedfavyfcqqgntlpytfgqgtkleik (SEQ ID NO: 27) |
| CD19 | huscFv5 | Eivmtqspatlslspgeratlscrasqdiskylnwyqqkpgqaprlliyhtsrlhsgiparfsgsgsgtd |
| | | ytltisslqpedfavyfcqqgntlpytfgqgtkleikggggsggggsggggsggggsqvqlqesgpgl |
| | | vkpsetlsltctvsgvslpdygvswirqppgkglewigviwgsettyyssslksrvtiskdnsknqvsl |
| | | klssvtaadtavyycakhyyyggsyamdywgqgtlvtvss (SEQ ID NO: 39) |
| CD19 | huscFv6 | Eivmtqspatlslspgeratlscrasqdiskylnwyqqkpgqaprlliyhtsrlhsgiparfsgsgsgtd |
| | | ytltisslqpedfavyfcqqgntlpytfgqgtkleikggggsggggsggggsggggsqvqlqesgpgl |
| | | vkpsetlsltctvsgvslpdygvswirqppgkglewigviwgsettyyqsslksrvtiskdnsknqvsl |
| | | klssvtaadtavyycakhyyyggsyamdywgqgtlvtvss (SEQ ID NO: 43) |
| CD19 | huscFv7 | Qvqlqesgpglvkpsetlsltctvsgvslpdvgvswirqppgkglewigviwgsettyyssslksrvti |
| | | skdnsknqvslklssvtaadtavyycakhyyyggsyamdywgqgtlvtvssggggsggggsggg |
| | | gsggggseivmtqspatlslspgeratlscrasqdiskylnwyqqkpgqaprlliyhtsrlhsgiparfs |
| | | gsgsgtdytltisslqpedfavyfcqqgntlpytfgqgtkleik (SEQ ID NO: 46) |
| CD19 | huscFv8 | Qvqlqesgpglvkpsetlsltctvsgvslpdygvswirqppgkglewigviwgsettyyqsslksrvti |
| | | skdnsknqvslklssvtaadtavyycakhyyyggsyamdywgqgtlvtvssggggsggggsggg |
| | | gsggggseivmtqspatlslspgeratlscrasqdiskylnwyqqkpgqaprlliyhtsrlhsgiparfs |
| | | gsgsgtdytltisslqpedfavyfcqqgntlpytfgqgtkleik (SEQ ID NO: 47) |
| CD19 | huscFv9 | Eivmtqspatlslspgeratlscrasqdiskylnwyqqkpgqaprlliyhtsrlhsgiparfsgsgsgtd |
| | | ytltisslqpedfavyfcqqgntlpytfgqgtkleikggggsggggsggggsggggsqvqlqesgpgl |
| | | vkpsetlsltctvsgvslpdygvswirqppgkglewigviwgsettyynsslksrvtiskdnsknqvsl |
| | | klssvtaadtavyycakhyyyggsyamdywgqgtlvtvss (SEQ ID NO: 48) |
| CD19 | Hu | Qvqlqesgpglvkpsetlsltctvsgvslpdygvswirqppgkglewigviwgsettyynsslksrvti |
| | scFv10 | skdnsknqvslklssvtaadtavyycakhyyyggsyamdywgqgtlvtvssggggsggggsggg |
| | | gsggggseivmtqspatlslspgeratlscrasqdiskylnwyqqkpgqaprlliyhtsrlhsgiparfs |
| | | gsgsgtdytltisslqpedfavyfcqqgntlpytfgqgtkleik (SEQ ID NO: 49) |
| CD19 | Hu | Eivmtqspatlslspgeratlscrasqdiskylnwyqqkpgqaprlliyhtsrlhsgiparfsgsgsgtd |
| | scFv11 | ytltisslqpedfavyfcqqgntlpytfgqgtkleikggggsggggsggggsqvqlqesgpglvkpset |
| | | lsltctvsgvslpdygvswirqppgkglewigviwgsettyynsslksrvtiskdnsknqvslklssvta |
| | | adtavyycakhyyyggsyamdywgqgtlvtvss (SEQ ID NO: 50) |
| CD19 | Hu | Qvqlqesgpglvkpsetlsltctvsgvslpdygvswirqppgkglewigviwgsettyynsslksrvti |
| | scFv12 | skdnsknqvslklssvtaadtavyycakhyyyggsyamdywgqgtlvtvssggggsggggsggg |
| | | gseivmtqspatlslspgeratlscrasqdiskylnwyqqkpgqaprlliyhtsrlhsgiparfsgsgsgt |
| | | dytltisslqpedfavyfcqqgntlpytfgqgtkleik (SEQ ID NO: 51) |
| CD19 | muCTL | Diqmtqttsslsaslgdrvtiscrasqdiskylnwyqqkpdgtvklliyhtsrlhsgvpsrfsgsgsgtd |
| | 019 | ysltisnleqediatyfcqqgntlpytfgggtkleitggggsggggsggggsevklqesgpglvapsqsl |
| | | svtctvsgyslpdygvswirqpprkglewlgviwgsettyynsalksrltiikdnsksqvflkmnslqt |
| | | ddtaiyycakhyyyggsyamdywgqgtsvtvss (SEQ ID NO: 52) |
| CD19 | SSJ25-C1 | QVQLLESGAELVRPGSSVKISCKASGYAFSSYWMNWVKQRPGQG |
| | VH | LEWIGQIYPGDGDTNYNGKFKGQATLTADKSSSTAYMQLSGLTSE |
| | sequence | DSAVYSCARKTISSVVDFYFDYWGQGTTVT (SEQ ID NO: 53) |
| CD19 | SSJ25-C1 | ELVLTQSPKFMSTSVGDRVSVTCKASQNVGTNVAWYQQKPGQSP |
| | VL | KPLIYSATYRNSGVPDRFTGSGSGTDFTLTITNVQSKDLADYFYFC |
| | sequence | QYNRYPYTSGGGTKLEIKRRS (SEQ ID NO: 54) |

Any known CD19 CAR, e.g., the CD19 antigen binding domain of any known CD19 CAR, in the art can be used in accordance with the present disclosure. For example, LG-740; CD19 CAR described in the U.S. Pat. Nos. 8,399,645; 7,446,190; Xu et al., LEUK LYMPHOMA. 2013 54(2):255-260 (2012); Cruz et al., BLOOD 122(17):2965-2973 (2013); Brentjens et al., BLOOD, 118(18):4817-4828 (2011); Kochenderfer et al., BLOOD 116(20):4099-102 (2010); Kochenderfer et al., BLOOD 122 (25):4129-39 (2013); and 16th Annu Meet Am Soc Gen Cell Ther (ASGCT) (May 15-18, Salt Lake City) 2013, Abst 10.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed or described above.

### Bispecific CARS

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

In certain embodiments, the antibody molecule is a multi-specific (e.g., a bispecific or a trispecific) antibody molecule. Protocols for generating bispecific or heterodimeric antibody molecules, and various configurations for bispecific antibody molecules, are described in, e.g., paragraphs 455-458 of WO2015/142675, filed Mar. 13, 2015, which is incorporated by reference in its entirety.

In one aspect, the bispecific antibody molecule is characterized by a first immunoglobulin variable domain sequence, e.g., a scFv, which has binding specificity for CD19, e.g., comprises a scFv as described herein, or comprises the light chain CDRs and/or heavy chain CDRs from a scFv described herein, and a second immunoglobulin variable domain sequence that has binding specificity for a second epitope on a different antigen.

### Chimeric TCR

In one aspect, the antibodies and antibody fragments of the present invention (e.g., CD19 antibodies and fragments) can be grafted to one or more constant domain of a T cell receptor (“TCR”) chain, for example, a TCR alpha or TCR beta chain, to create a chimeric TCR. Without being bound by theory, it is believed that chimeric TCRs will signal through the TCR complex upon antigen binding. For example, an scFv as disclosed herein, can be grafted to the constant domain, e.g., at least a portion of the extracellular constant domain, the transmembrane domain and the cytoplasmic domain, of a TCR chain, for example, the TCR alpha chain and/or the TCR beta chain. As another example, an antibody fragment, for example a VL domain as described herein, can be grafted to the constant domain of a TCR alpha chain, and an antibody fragment, for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of an antibody or antibody fragment may be grafted into a TCR alpha and/or beta chain to create a chimeric TCR. For example, the LCDRs disclosed herein may be grafted into the variable domain of a TCR alpha chain and the HCDRs disclosed herein may be grafted to the variable domain of a TCR beta chain, or vice versa. Such chimeric TCRs may be produced, e.g., by methods known in the art (For example, Willemsen R A et al, Gene Therapy 2000; 7: 1369-1377; Zhang T et al, Cancer Gene Ther 2004; 11: 487-496; Aggen et al, Gene Ther. 2012 April; 19(4):365-74).

### Non Antibody Scaffolds

In embodiments, the antigen binding domain comprises a non-antibody scaffold, e.g., a fibronectin, ankyrin, domain antibody, lipocalin, small modular immuno-pharmaceutical, maxybody, Protein A, or affilin. The non-antibody scaffold has the ability to bind to target antigen on a cell. In embodiments, the antigen binding domain is a polypeptide or fragment thereof of a naturally occurring protein expressed on a cell. In some embodiments, the antigen binding domain comprises a non-antibody scaffold. A wide variety of non-antibody scaffolds can be employed so long as the resulting polypeptide includes at least one binding region which specifically binds to the target antigen on a target cell.

Non-antibody scaffolds include: fibronectin (Novartis, MA), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (Scil Proteins GmbH, Halle, Germany).

In an embodiment the antigen binding domain comprises the extracellular domain, or a counter-ligand binding fragment thereof, of molecule that binds a counterligand on the surface of a target cell.

### Transmembrane Domain

In embodiments, a CAR described herein comprises a transmembrane domain that is fused to an extracellular sequence, e.g., an extracellular recognition element, which can comprise an antigen binding domain. In an embodiment, the transmembrane domain is one that naturally is associated with one of the domains in the CAR. In an embodiment, the transmembrane domain is one that is not naturally associated with one of the domains in the CAR.

A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region).

In embodiments, the transmembrane domain is one which minimizes interactions with other elements, e.g., other transmembrane domains. In some instances, the transmembrane domain minimizes binding of such domains to the transmembrane domains of the same or different surface membrane proteins, e.g., to minimize interactions with other members of the receptor complex. Suitable examples can be derived by selection or modification of amino acid substitution of a known transmembrane domain. In an embodiment, the transmembrane domain is capable of promoting homodimerization with another CAR on the cell surface.

The transmembrane domain may comprise a naturally occurring, or a non-naturally occurring synthetic sequence. Where naturally occurring, the transmembrane domain may be derived from any membrane-bound or transmembrane protein.

Transmembrane regions suitable for use in molecules described herein may be derived from any one or more of e.g., the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11 a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11 a, LFA-1, ITGAM, CD11b, ITGAX, CD11 c, ITGB1, CD29, ITGB2, CD18, LFA-1, ITGB7, TNFR2, DNAM1 (CD226), SLAMF4 (CD244, 2B4), CD84, CD96 (Tactile), CEACAM (e.g., CEACAM-1, CEACAM-3 and/or CEACAM-5), CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, or CD19. In an embodiment the transmembrane domain is derived from CD8. In an embodiment the transmembrane domain is derived from CD28. In one aspect, the transmembrane domain is a transmembrane domain from the sequence provided as SEQ ID NO: 12 or SEQ ID NO: 42.

In an embodiment, a sequence, e.g., a hinge or spacer sequence, can be disposed between a transmembrane domain and another sequence or domain to which it is fused. In embodiments, a variety of human hinges (aka “spacers”) can be employed as well, e.g., including but not limited to the human Ig (immunoglobulin) hinge. Optionally, a short oligo- or polypeptide linker, between 2 and 10 amino acids in length may form the linkage between the transmembrane domain and another domain, e.g., an intracellular signaling domain or costimulatory domain, of a CAR. A glycine-serine doublet provides a particularly suitable linker. In one aspect, the hinge or spacer is the amino acid sequence provided as SEQ ID NO: 4, SEQ ID NO: 6, or SEQ ID NO: 8. In one aspect, the hinge or spacer comprises a KIR2DS2 hinge.

In an embodiment, the transmembrane domain may be a non-naturally occurring sequence, in which case can comprise predominantly hydrophobic residues such as leucine and valine. In an embodiment, a triplet of phenylalanine, tryptophan and valine will be found at each end of a transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of GGGGSGGGGS (SEQ ID NO:10). In some embodiments, the linker is encoded by a nucleotide sequence of

(SEQ ID NO: 11)
GGTGGCGGAGGTTCTGGAGGTGGAGGTTCC.

### Cytoplasmic Domain

The cytoplasmic domain or region of the CAR includes an intracellular signaling domain. An intracellular signaling domain is generally responsible for activation of at least one of the normal effector functions of the immune cell in which the CAR has been introduced.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

### Primary Signaling Domain

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d. In one embodiment, a CAR of the invention comprises an intracellular signaling domain, e.g., a primary signaling domain of CD3-zeta, e.g., a CD3-zeta sequence described herein.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs. Further examples of molecules containing a primary intracellular signaling domain that are of particular use in the invention include those of DAP10, DAP12, and CD32.

A primary intracellular signaling domain comprises a functional fragment, or analog, of a primary stimulatory molecule (e.g., CD3 zeta—GenBank Acc. No. BAG36664.1). The primary intracellular signaling domain can comprise the entire intracellular region or a fragment of the intracellular region which is sufficient for generation of an intracellular signal when an antigen binding domain to which it is fused binds cognate antigen. In embodiments the primary intracellular signaling domain has at least 70, 75, 80, 85, 90, 95, 98, or 99% sequence identity with the entire intracellular region, or a fragment of the intracellular region which is sufficient for generation of an intracellular signal, of a naturally occurring primary stimulatory molecule, e.g., a human (GenBank Acc No. BAG36664.1), or other mammalian, e.g., a nonhuman species, e.g., rodent, monkey, ape or murine intracellular primary stimulatory molecule. In embodiments the primary intracellular signaling domain has at least 70, 75, 80, 85, 90, 95, 98, or 99% sequence identity with SEQ ID NO: 18 or SEQ ID NO: 20.

In embodiments, the primary intracellular signaling domain, has at least 70, 75, 80, 85, 90, 95, 96, 97, 98, or 99% identity with, or differs by no more than 30, 25, 20, 15, 10, 5, 4, 3, 2, or 1 amino acid residues from the corresponding residues of the entire intracellular region, or a fragment of the intracellular region which is sufficient for generation of an intracellular signal, of a naturally occurring human primary stimulatory molecule, e.g., a naturally occurring human primary stimulatory molecule disclosed herein.

### Costimulatory Signaling Domain

The intracellular signalling domain of the CAR can comprise the CD3-zeta signalling domain by itself or it can be combined with any other desired intracellular signalling domain(s) useful in the context of a CAR of the invention. For example, the intracellular signalling domain of the CAR can comprise a CD3 zeta chain portion and a costimulatory signaling domain. The costimulatory signaling domain refers to a portion of the CAR comprising the intracellular domain of a costimulatory molecule. In one embodiment, the intracellular domain is designed to comprise the signaling domain of CD3-zeta and the signaling domain of CD28. In one aspect, the intracellular domain is designed to comprise the signaling domain of CD3-zeta and the signaling domain of ICOS.

A costimulatory molecule is a cell surface molecule other than an antigen receptor or its ligands that is required for an efficient response of lymphocytes to an antigen. Examples of such molecules include MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, and the like. For example, CD27 costimulation has been demonstrated to enhance expansion, effector function, and survival of human CAR-expressing cell (e.g., T cell, NK cell) cells in vitro and augments human T cell persistence and antitumor activity in vivo (Song et al. BLOOD. 2012; 119(3):696-706). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11 c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKG2D and NKG2C.

The intracellular signaling sequences within the cytoplasmic portion of the CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

A costimulatory domain comprises a functional fragment, or analog, of a costimulatory molecule (e.g., ICOS, CD28, or 4-1BB). It can comprise the entire intracellular region or a fragment of the intracellular region which is sufficient for generation of an intracellular signal, e.g., when an antigen binding domain to which it is fused binds cognate antigen. In embodiments the costimulatory domain has at least 70%, 75%, 80%, 85%, 90%, 95%, 98%, or 99% sequence identity with the entire intracellular region, or a fragment of the intracellular region which is sufficient for generation of an intracellular signal, of a naturally occurring costimulatory molecule as described herein, e.g., a human, or other mammalian, e.g., a nonhuman species, e.g., rodent, monkey, ape or murine intracellular costimulatory molecule. In embodiments the costimulatory domain has at least 70%, 75%, 80%, 85%, 90%, 95%, 98%, or 99% sequence identity with SEQ ID NO: 14, SEQ ID NO: 16, SEQ ID NO: 40, or SEQ ID NO: 44.

In embodiments the costimulatory signaling domain, has at least 70, 75, 80, 85, 90, 95, 96, 97, 98, or 99% identity with, or differs by no more than 30, 25, 20, 15, 10, 5, 4, 3, 2, or 1 amino acid residues from the corresponding residues of the entire intracellular region, or a fragment of the intracellular region which is sufficient for generation of an intracellular signal, of, a naturally occurring human costimulatory molecule, e.g., a naturally occurring human costimulatory molecule disclosed herein.

Any of the CARs described herein can include one or more of the components listed in Table 1.

### Combination of CARs

In one aspect, the CAR-expressing cell described herein can further comprise a second CAR, e.g., a second CAR that includes a different antigen binding domain, e.g., to the same target or a different target (e.g., a target other than a cancer associated antigen described herein or a different cancer associated antigen described herein, e.g., CD19, CD33, CLL-1, CD34, FLT3, or folate receptor beta). In one embodiment, the second CAR includes an antigen binding domain to a target expressed the same cancer cell type as the cancer associated antigen. In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, ICOS, CD27 or OX-40, onto the first CAR, and the primary signaling domain, e.g., CD3 zeta, on the second CAR can limit the CAR activity to cells where both targets are expressed. In one embodiment, the CAR expressing cell comprises a first cancer associated antigen CAR that includes an antigen binding domain that binds a target antigen described herein, a transmembrane domain and a costimulatory domain and a second CAR that targets a different target antigen (e.g., an antigen expressed on that same cancer cell type as the first target antigen) and includes an antigen binding domain, a transmembrane domain and a primary signaling domain. In another embodiment, the CAR expressing cell comprises a first CAR that includes an antigen binding domain that binds a target antigen described herein, a transmembrane domain and a primary signaling domain and a second CAR that targets an antigen other than the first target antigen (e.g., an antigen expressed on the same cancer cell type as the first target antigen) and includes an antigen binding domain to the antigen, a transmembrane domain and a costimulatory signaling domain.

In one embodiment, the CAR-expressing cell comprises a CAR described herein (e.g., a CD19 CAR) and an inhibitory CAR. In one embodiment, the inhibitory CAR comprises an antigen binding domain that binds an antigen found on normal cells but not cancer cells, e.g., normal cells that also express CLL. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta).

In one embodiment, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, e.g., as a fragment, e.g., an scFv, that does not form an association with the antigen binding domain of the second CAR, e.g., the antigen binding domain of the second CAR is a VHH.

In some embodiments, when present on the surface of a cell, binding of the antigen binding domain of the first CAR to its cognate antigen is not substantially reduced by the presence of the second CAR. In some embodiments, binding of the antigen binding domain of the first CAR to its cognate antigen in the presence of the second CAR is 85%, 90%, 95%, 96%, 97%, 98% or 99% of binding of the antigen binding domain of the first CAR to its cognate antigen in the absence of the second CAR.

In some embodiments, when present on the surface of a cell, the antigen binding domains of the first CAR said second CAR, associate with one another less than if both were scFv antigen binding domains. In some embodiments, the antigen binding domains of the first CAR and the second CAR, associate with one another 85%, 90%, 95%, 96%, 97%, 98% or 99% less than if both were scFv antigen binding domains.

### CAR-Expressing Cells

The CARs described herein are expressed on cells, e.g., immune effector cells, e.g., T cells. For example, a nucleic acid construct of a CAR described herein is transduced to a T cell. In embodiments, the cells expressing the CARs described herein are an in vitro transcribed RNA CAR T cell.

Sources of Cells, e.g., T cells

Prior to expansion and genetic modification or other modification, a source of cells, e.g., immune effector cells, e.g., T cells or NK cells, can be obtained from a subject. Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, the cells obtained as described in this section are subjected to an assay described herein, e.g., one or more biomarkers are assayed.

In certain aspects of the present disclosure, immune effector cells, e.g., T cells or NK cells, can be obtained from a unit of blood collected from a subject using any number of techniques known to the skilled artisan, such as Ficoll™ separation. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi: 10.1038/cti 0.2014.31.

In one aspect, T cells are isolated from peripheral blood lymphocytes by lysing the red blood cells and depleting the monocytes, for example, by centrifugation through a PERCOLL™ gradient or by counterflow centrifugal elutriation.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. In embodiments, the population of T regulatory depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, e.g., IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depletion reagent from Miltenyi™. In one embodiment, the ratio of cells to CD25 depletion reagent is 1e7 cells to 20 uL, or 1e7 cells to 15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of subject relapse. In an embodiment, a patient is pre-treated with one or more therapies that reduce T_{REG} cells prior to collection of cells for CAR-expressing cell (e.g., T cell, NK cell) product manufacturing, thereby reducing the risk of patient relapse to CAR-expressing cell (e.g., T cell, NK cell) treatment (e.g., CTL019 treatment). Methods of depleting T_{REG} cells are known in the art. Methods of decreasing T_{REG} cells include, but are not limited to, cyclophosphamide, anti-GITR antibody, CD25-depletion, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In an embodiment, a patient is pre-treated with cyclophosphamide prior to collection of cells for CAR-expressing cell (e.g., T cell, NK cell) product manufacturing, thereby reducing the risk of patient relapse to CAR-expressing cell treatment (e.g., CTL019 treatment). In an embodiment, a patient is pre-treated with an anti-GITR antibody prior to collection of cells for CAR-expressing cell (e.g., T cell, NK cell) product manufacturing, thereby reducing the risk of patient relapse to CAR-expressing cell treatment (e.g., CTL019 treatment).

In an embodiment, the CAR-expressing cell (e.g., T cell, NK cell) manufacturing process is modified to deplete T_{REG} cells prior to manufacturing of the CAR-expressing cell (e.g., T cell, NK cell) product (e.g., a CTL019 product). In an embodiment, CD25-depletion is used to deplete T_{REG} cells prior to manufacturing of the CAR-expressing cell (e.g., T cell, NK cell) product (e.g., a CTL019 product).

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include B7-H1, B&-1, CD160, P1H, 2B4, PD1, TIM3, CEACAM (e.g., CEACAM-1, CEACAM-3 and/or CEACAM-5), LAG3, TIGIT, CTLA-4, BTLA and LAIR1. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

Methods described herein can include a positive selection step. For example, T cells can isolated by incubation with anti-CD³/anti-CD28 (e.g., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours, e.g., 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In one embodiment, a T cell population can be selected that expresses one or more of IFN-7, TNFα, IL-17A, IL-2, IL-3, IL-4, IL-5, IL-6, IL-9, IL-21, CCL20, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712. In an embodiment, the T cell population expresses cytokine CCL20, IL-17a, IL-6, and combinations thereof.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 10 billion cells/ml, 9 billion/ml, 8 billion/ml, 7 billion/ml, 6 billion/ml, or 5 billion/ml is used. In one aspect, a concentration of 1 billion cells/ml is used. In yet one aspect, a concentration of cells from 75, 80, 85, 90, 95, or 100 million cells/ml is used. In further aspects, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, the immune effector cells expressing a CAR molecule, e.g., a CAR molecule described herein, are obtained from a subject that has received a low, immune enhancing dose of an mTOR inhibitor. In an embodiment, the population of immune effector cells, e.g., T cells, to be engineered to express a CAR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

In one embodiment, a T cell population is diacylglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

### Allogeneic CAR

In embodiments described herein, the immune effector cell can be an allogeneic immune effector cell, e.g., T cell. For example, the cell can be an allogeneic T cell, e.g., an allogeneic T cell lacking expression of a functional T cell receptor (TCR) and/or human leukocyte antigen (HLA), e.g., HLA class I and/or HLA class II.

A T cell lacking a functional TCR can be, e.g., engineered such that it does not express any functional TCR on its surface, engineered such that it does not express one or more subunits that comprise a functional TCR (e.g., engineered such that it does not express (or exhibits reduced expression) of TCR alpha, TCR beta, TCR gamma, TCR delta, TCR epsilon, and/or TCR zeta) or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class 1 and/or HLA class II, is downregulated. In some embodiments, downregulation of HLA may be accomplished by reducing or eliminating expression of beta-2 microglobulin (B2M).

In some embodiments, the T cell can lack a functional TCR and a functional HLA, e.g., HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not expresses or expresses at low levels an inhibitory molecule, e.g. by any method described herein. For example, the cell can be a cell that does not express or expresses at low levels an inhibitory molecule, e.g., that can decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta). Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, WIC class I, WIC class II, GAL9, adenosine, and TGF beta), in a cell, e.g., T cell.

Expression systems for siRNA and shRNAs, and exemplary shRNAs, are described, e.g., in paragraphs 649 and 650 of International Application WO2015/142675, filed Mar. 13, 2015, which is incorporated by reference in its entirety.

### CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, WIC class II, GAL9, adenosine, and TGF beta), in a cell, e.g., T cell.

The CRISPR/Cas system, and uses thereof, are described, e.g., in paragraphs 651-658 of International Application WO2015/142675, filed Mar. 13, 2015, which is incorporated by reference in its entirety.

TALEN to Inhibit TCR and/or HLA

“TALEN” or “TALEN to HLA and/or TCR” or “TALEN to inhibit HLA and/or TCR” refers to a transcription activator-like effector nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, WIC class II, GAL9, adenosine, and TGF beta), in a cell, e.g., T cell.

TALENs, and uses thereof, are described, e.g., in paragraphs 659-665 of International Application WO2015/142675, filed Mar. 13, 2015, which is incorporated by reference in its entirety.

Zinc Finger Nuclease to Inhibit HLA and/or TCR

“ZFN” or “Zinc Finger Nuclease” or “ZFN to HLA and/or TCR” or “ZFN to inhibit HLA and/or TCR” refer to a zinc finger nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF beta), in a cell, e.g., T cell.

ZFNs, and uses thereof, are described, e.g., in paragraphs 666-671 of International Application WO2015/142675, filed Mar. 13, 2015, which is incorporated by reference in its entirety.

### Telomerase Expression

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in an embodiment, an immune effector cell, e.g., a T cell, ectopically expresses a telomerase subunit, e.g., the catalytic subunit of telomerase, e.g., TERT, e.g., hTERT. In some aspects, this disclosure provides a method of producing a CAR-expressing cell, comprising contacting a cell with a nucleic acid encoding a telomerase subunit, e.g., the catalytic subunit of telomerase, e.g., TERT, e.g., hTERT. The cell may be contacted with the nucleic acid before, simultaneous with, or after being contacted with a construct encoding a CAR.

In one aspect, the disclosure features a method of making a population of immune effector cells (e.g., T cells, NK cells). In an embodiment, the method comprises: providing a population of immune effector cells (e.g., T cells or NK cells), contacting the population of immune effector cells with a nucleic acid encoding a CAR; and contacting the population of immune effector cells with a nucleic acid encoding a telomerase subunit, e.g., hTERT, under conditions that allow for CAR and telomerase expression.

In an embodiment, the nucleic acid encoding the telomerase subunit is DNA. In an embodiment, the nucleic acid encoding the telomerase subunit comprises a promoter capable of driving expression of the telomerase subunit. hTERT amino acid and nucleotide sequences as well as methods of using hTERT in methods of making a population of immune effector cells expressing a CAR are described in International Application WO 2016/057705 filed on 7 Oct. 2015, the entire contents of which are hereby incorporated by reference.

### Activation and Expansion of Immune Effector Cells (e.g., T Cells)

Immune effector cells such as T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. In some embodiments, immune effector cells are subjected to an assay as described herein (e.g., one or more biomarkers are assayed) before, during, or after activation, or before, during, or after expansion.

Generally, a population of immune effector cells may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain aspects, the primary stimulatory signal and the costimulatory signal for the T cell may be provided by different protocols. For example, the agents providing each signal may be in solution or coupled to a surface.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect, more anti-CD28 antibody is bound to the particles than anti-CD3 antibody, i.e., the ratio of CD3:CD28 is less than one. In certain aspects, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one preferred aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used. Additional ratios of particles to cells and methods of activating T cells are disclosed in International Application WO 2016/057705 filed on 7 Oct. 2015, the entire contents of which are hereby incorporated by reference.

In further aspects, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one aspect the cells (for example, 10⁴ to 10⁹ T cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain aspects, it may be desirable to significantly decrease the volume in which particles and cells are mixed together (i.e., increase the concentration of cells), to ensure maximum contact of cells and particles. For example, in one aspect, a concentration of about 10 billion cells/ml, 9 billion/ml, 8 billion/ml, 7 billion/ml, 6 billion/ml, 5 billion/ml, or 2 billion cells/ml is used. In one aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain aspects. For example, using high concentration of cells allows more efficient selection of CD8+ T cells that normally have weaker CD28 expression.

In one embodiment, cells transduced with a nucleic acid encoding a CAR, e.g., a CAR described herein, are expanded, e.g., by a method described herein. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. In one embodiment, the cells are expanded for a period of 8 days or less, e.g., 7, 6 or 5 days. In one embodiment, the cells, e.g., a CD19 CAR cell described herein, are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In one embodiment, the cells, e.g., a CD19 CAR cell described herein, expanded for 5 days show at least a one, two, three or four fold increase in cells doublings upon antigen stimulation as compared to the same cells expanded in culture for 9 days under the same culture conditions. In one embodiment, the cells, e.g., the cells expressing a CD19 CAR described herein, are expanded in culture for 5 days, and the resulting cells exhibit higher proinflammatory cytokine production, e.g., IFN-γ and/or GM-CSF levels, as compared to the same cells expanded in culture for 9 days under the same culture conditions. In one embodiment, the cells, e.g., a CD19 CAR cell described herein, expanded for 5 days show at least a one, two, three, four, five, tenfold or more increase in pg/ml of proinflammatory cytokine production, e.g., IFN-γ and/or GM-CSF levels, as compared to the same cells expanded in culture for 9 days under the same culture conditions.

Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In some embodiments, the cells are cultured in the presence of IL-2, a interleukin-15 (IL-15) polypeptide, a interleukin-15 receptor alpha (IL-15Ra) polypeptide, IL-7 or any combination thereof. In some embodiments, the cells are cultured in the presence of a combination of both a IL-15 polypeptide and a IL-15Ra polypeptide (e.g., hetIL-15). In some embodiments, the cells are cultured in the presence of hetIL-15 and IL-2 or IL-7, or both IL-2 and IL7. In some embodiments, the cells are cultured in the presence of IL-2. In some embodiments, the cells are cultured in the presence of IL-7 and/or IL-15 polypeptide

In one embodiment, the cells are expanded in an appropriate media (e.g., media described herein) that includes one or more interleukin that result in at least a 200-fold (e.g., 200-fold, 250-fold, 300-fold, 350-fold) increase in cells over a 14 day expansion period, e.g., as measured by a method described herein such as flow cytometry. In one embodiment, the cells are expanded in the presence IL-15 and/or IL-7 (e.g., IL-15 and IL-7).

In embodiments, methods described herein, e.g., CAR-expressing cell manufacturing methods, comprise removing T regulatory cells, e.g., CD25+ T cells, from a cell population, e.g., using an anti-CD25 antibody, or fragment thereof, or a CD25-binding ligand, IL-2. Methods of removing T regulatory cells, e.g., CD25+ T cells, from a cell population are described herein. In embodiments, the methods, e.g., manufacturing methods, further comprise contacting a cell population (e.g., a cell population in which T regulatory cells, such as CD25+ T cells, have been depleted; or a cell population that has previously contacted an anti-CD25 antibody, fragment thereof, or CD25-binding ligand) with IL-15 and/or IL-7. For example, the cell population (e.g., that has previously contacted an anti-CD25 antibody, fragment thereof, or CD25-binding ligand) is expanded in the presence of IL-15 and/or IL-7.

In some embodiments a CAR-expressing cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15, during the manufacturing of the CAR-expressing cell, e.g., ex vivo. In embodiments, a CAR-expressing cell described herein is contacted with a composition comprising a IL-15 polypeptide during the manufacturing of the CAR-expressing cell, e.g., ex vivo. In embodiments, a CAR-expressing cell described herein is contacted with a composition comprising a combination of both a IL-15 polypeptide and a IL-15 Ra polypeptide during the manufacturing of the CAR-expressing cell, e.g., ex vivo. In embodiments, a CAR-expressing cell described herein is contacted with a composition comprising hetIL-15 during the manufacturing of the CAR-expressing cell, e.g., ex vivo.

In one embodiment the CAR-expressing cell described herein is contacted with a composition comprising hetIL-15 during ex vivo expansion. In an embodiment, the CAR-expressing cell described herein is contacted with a composition comprising an IL-15 polypeptide during ex vivo expansion. In an embodiment, the CAR-expressing cell described herein is contacted with a composition comprising both an IL-15 polypeptide and an IL-15Ra polypeptide during ex vivo expansion. In one embodiment the contacting results in the survival and proliferation of a lymphocyte subpopulation, e.g., CD8+ T cells.

In one embodiment, the cells are cultured (e.g., expanded, simulated, and/or transduced) in media comprising serum. The serum may be, e.g., human AB serum (hAB). In some embodiments, the hAB serum is present at about 2%, about 5%, about 2-3%, about 3-4%, about 4-5%, or about 2-5%. As shown in Example 15 herein, 2% and 5% serum are each suitable levels that allow for many fold expansion of T cells. Furthermore, as shown in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31, medium containing 2% human AB serum is suitable for ex vivo expansion of T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

In some embodiments, cells transduced with a nucleic acid encoding a CAR, e.g., a CAR described herein, can be selected for administration based upon, e.g., protein expression levels of one or more of CCL20, GM-CSF, IFNγ, IL-10, IL-13, IL-17a, IL-2, IL-21, IL-4, IL-5, IL-6, IL-9, TNFα and/or combinations thereof. In some embodiments, cells transduced with a nucleic acid encoding a CAR, e.g., a CAR described herein, can be selected for administration based upon, e.g., protein expression levels of CCL20, IL-17a, IL-6 and combinations thereof.

Once a CAR described herein is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a CAR are described in further detail below.

Western blot analysis of CAR expression in primary T cells can be used to detect the presence of monomers and dimers, e.g., as described in paragraph 695 of International Application WO2015/142675, filed Mar. 13, 2015, which is herein incorporated by reference in its entirety.

In vitro expansion of CAR′ T cells following antigen stimulation can be measured by flow cytometry. For example, a mixture of CD4⁺ and CD8⁺ T cells are stimulated with αCD3/αCD28 aAPCs followed by transduction with lentiviral vectors expressing GFP under the control of the promoters to be analyzed. Exemplary promoters include the CMV IE gene, EF-1α, ubiquitin C, or phosphoglycerokinase (PGK) promoters. GFP fluorescence is evaluated on day 6 of culture in the CD4⁺ and/or CD8⁺ T cell subsets by flow cytometry. See, e.g., Milone ET AL., MOLECULAR THERAPY 17(8): 1453-1464 (2009). Alternatively, a mixture of CD4⁺ and CD8⁺ T cells are stimulated with αCD3/αCD28 coated magnetic beads on day 0, and transduced with CAR on day 1 using a bicistronic lentiviral vector expressing CAR along with eGFP using a 2A ribosomal skipping sequence. Cultures are re-stimulated with either a cancer associate antigen as described herein ⁺K562 cells (K562—a cancer associate antigen as described herein), wild-type K562 cells (K562 wild type) or K562 cells expressing hCD32 and 4-1BBL in the presence of antiCD3 and anti-CD28 antibody (K562-BBL-3/28) following washing. Exogenous IL-2 is added to the cultures every other day at 100 IU/ml. GFP⁺ T cells are enumerated by flow cytometry using bead-based counting. See, e.g., Milone et al., MOLECULAR THERAPY 17(8): 1453-1464 (2009).

Sustained CAR⁺ T cell expansion in the absence of re-stimulation can also be measured. See, e.g., Milone et al., MOLECULAR THERAPY 17(8): 1453-1464 (2009). Briefly, mean T cell volume (fl) is measured on day 8 of culture using a Coulter Multisizer III particle counter, a Nexcelom Cellometer Vision or Millipore Scepter, following stimulation with αCD3/αCD28 coated magnetic beads on day 0, and transduction with the indicated CAR on day 1.

Animal models can also be used to measure a CAR-expressing cell (e.g., T cell, NK cell) activity, e.g., as described in paragraph 698 of International Application WO2015/142675, filed Mar. 13, 2015, which is herein incorporated by reference in its entirety.

Dose dependent CAR treatment response can be evaluated, e.g., as described in paragraph 699 of International Application WO2015/142675, filed Mar. 13, 2015, which is herein incorporated by reference in its entirety.

Assessment of cell proliferation and cytokine production has been previously described, e.g., as described in paragraph 700 of International Application WO2015/142675, filed Mar. 13, 2015, which is herein incorporated by reference in its entirety.

In another embodiment, potency of a cell (e.g., T cell, NK cell) population (e.g. a CAR-expressing cell) product, e.g., a CD19 CAR-expressing cell (e.g., T cell, NK cell) cell product, e.g., CTL019 cells) is assessed using a Luminex® panel of cytokines to determine cytokine expression levels. Cell (e.g., T cell, NK cell) populations (e.g, a manufactured CAR-expressing cell) cell product, e.g., a CD19 CAR-expressing cell product, e.g., CTL019 cells) are activated in vitro by CD19-expressing K562 (K562-19) cells, which mimic CD19-expressing B cells in CLL. Following cell (e.g., T cell, NK cell) activation, cytokine expression profiles are measured in the co-cultured cell media and potency of activated cells (e.g., a CAR-expressing cell product, e.g., a CD19 CAR-expressing cell product, e.g., CTL019 cells) is correlated with expression of different cytokines including, but not limited to CCL-20/MIP-3a, GM-CSF, IFNγ, IL-10, IL-13, IL-17a, IL-2, IL-21, IL-4, IL-5, IL-6, IL-9, TNFα and/or combinations thereof.

In an embodiment, cytokine expression levels are informative with regards to the potency of a cell (e.g., T cell, NK cell) population (e.g., to kill tumor cells). In an embodiment, cytokine expression levels described herein are used to improve a cell (e.g., T cell, NK cell) population (e.g., a CAR-expressing cell product, e.g., a CD 19 CAR-expressing cell product, e.g., CTL019 cells) prior to infusion in patients. In an embodiment, cytokine expression levels described herein provide an endpoint during optimization of the manufacturing process.

Cytotoxicity can be assessed by a standard 51Cr-release assay, e.g., as described in paragraph 701 of International Application WO2015/142675, filed Mar. 13, 2015, which is herein incorporated by reference in its entirety.

Imaging technologies can be used to evaluate specific trafficking and proliferation of CARs in tumor-bearing animal models, e.g., as described in paragraph 702 of International Application WO2015/142675, filed Mar. 13, 2015, which is herein incorporated by reference in its entirety.

Other assays, including those described in the Example section herein as well as those that are known in the art can also be used to evaluate the CARs described herein.

Alternatively, or in combination to the methods disclosed herein, methods and compositions for one or more of: detection and/or quantification of CAR-expressing cells (e.g., in vitro or in vivo (e.g., clinical monitoring)); immune cell expansion and/or activation; and/or CAR-specific selection, that involve the use of a CAR ligand, are disclosed. In one exemplary embodiment, the CAR ligand is an antibody that binds to the CAR molecule, e.g., binds to the extracellular antigen binding domain of CAR (e.g., an antibody that binds to the antigen binding domain, e.g., an anti-idiotypic antibody; or an antibody that binds to a constant region of the extracellular binding domain). In other embodiments, the CAR ligand is a CAR antigen molecule (e.g., a CAR antigen molecule as described herein).

In one aspect, a method for detecting and/or quantifying CAR-expressing cells is disclosed. For example, the CAR ligand can be used to detect and/or quantify CAR-expressing cells in vitro or in vivo (e.g., clinical monitoring of CAR-expressing cells in a patient, or dosing a patient). The method includes:

providing the CAR ligand (optionally, a labelled CAR ligand, e.g., a CAR ligand that includes a tag, a bead, a radioactive or fluorescent label);

acquiring the CAR-expressing cell (e.g., acquiring a sample containing CAR-expressing cells, such as a manufacturing sample or a clinical sample);

contacting the CAR-expressing cell with the CAR ligand under conditions where binding occurs, thereby detecting the level (e.g., amount) of the CAR-expressing cells present. Binding of the CAR-expressing cell with the CAR ligand can be detected using standard techniques such as FACS, ELISA and the like.

In another aspect, a method of expanding and/or activating cells (e.g., immune effector cells) is disclosed. The method includes:

providing a CAR-expressing cell (e.g., a first CAR-expressing cell or a transiently expressing CAR cell);

contacting said CAR-expressing cell with a CAR ligand, e.g., a CAR ligand as described herein), under conditions where immune cell expansion and/or proliferation occurs, thereby producing the activated and/or expanded cell population.

In certain embodiments, the CAR ligand is present on (e.g., is immobilized or attached to a substrate, e.g., a non-naturally occurring substrate). In some embodiments, the substrate is a non-cellular substrate. The non-cellular substrate can be a solid support chosen from, e.g., a plate (e.g., a microtiter plate), a membrane (e.g., a nitrocellulose membrane), a matrix, a chip or a bead. In embodiments, the CAR ligand is present in the substrate (e.g., on the substrate surface). The CAR ligand can be immobilized, attached, or associated covalently or non-covalently (e.g., cross-linked) to the substrate. In one embodiment, the CAR ligand is attached (e.g., covalently attached) to a bead. In the aforesaid embodiments, the immune cell population can be expanded in vitro or ex vivo. The method can further include culturing the population of immune cells in the presence of the ligand of the CAR molecule, e.g., using any of the methods described herein.

In other embodiments, the method of expanding and/or activating the cells further comprises addition of a second stimulatory molecule, e.g., CD28. For example, the CAR ligand and the second stimulatory molecule can be immobilized to a substrate, e.g., one or more beads, thereby providing increased cell expansion and/or activation.

In yet another aspect, a method for selecting or enriching for a CAR expressing cell is provided. The method includes contacting the CAR expressing cell with a CAR ligand as described herein; and selecting the cell on the basis of binding of the CAR ligand.

In yet other embodiments, a method for depleting, reducing and/or killing a CAR expressing cell is provided. The method includes contacting the CAR expressing cell with a CAR ligand as described herein; and targeting the cell on the basis of binding of the CAR ligand, thereby reducing the number, and/or killing, the CAR-expressing cell. In one embodiment, the CAR ligand is coupled to a toxic agent (e.g., a toxin or a cell ablative drug). In another embodiment, the anti-idiotypic antibody can cause effector cell activity, e.g., ADCC or ADC activities.

Exemplary anti-CAR antibodies that can be used in the methods disclosed herein are described, e.g., in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference.

In some aspects and embodiments, the compositions and methods herein are optimized for a specific subset of T cells, e.g., as described in US Serial No. PCT/US2015/043219 filed Jul. 31, 2015, the contents of which are incorporated herein by reference in their entirety. In some embodiments, the optimized subsets of T cells display an enhanced persistence compared to a control T cell, e.g., a T cell of a different type (e.g., CD8⁺ or CD4⁺) expressing the same construct.

In some embodiments, a CD4⁺ T cell comprises a CAR described herein, which CAR comprises an intracellular signaling domain suitable for (e.g., optimized for, e.g., leading to enhanced persistence in) a CD4⁺ T cell, e.g., an ICOS domain. In some embodiments, a CD8⁺ T cell comprises a CAR described herein, which CAR comprises an intracellular signaling domain suitable for (e.g., optimized for, e.g., leading to enhanced persistence of) a CD8⁺ T cell, e.g., a 4-1BB domain, a CD28 domain, or another costimulatory domain other than an ICOS domain. In some embodiments, the CAR described herein comprises an antigen binding domain described herein, e.g., a CAR comprising an antigen binding domain.

In an aspect, described herein is a method of treating a subject, e.g., a subject having cancer. The method includes administering to said subject, an effective amount of:

1) a CD4⁺ T cell comprising a CAR (the CAR^{CD4+})

comprising:

an antigen binding domain, e.g., an antigen binding domain described herein;

a transmembrane domain; and

an intracellular signaling domain, e.g., a first costimulatory domain, e.g., an ICOS domain; and

2) a CD8⁺ T cell comprising a CAR (the CAR^{CD8+}) comprising:

an antigen binding domain, e.g., an antigen binding domain described herein;

a transmembrane domain; and

an intracellular signaling domain, e.g., a second costimulatory domain, e.g., a 4-1BB domain, a CD28 domain, or another costimulatory domain other than an ICOS domain;

wherein the CAR^{CD4+} and the CAR^{CD8+} differ from one another.

Optionally, the method further includes administering:

3) a second CD8+ T cell comprising a CAR (the second CAR^{CD8+}) comprising:

an antigen binding domain, e.g., an antigen binding domain described herein;

a transmembrane domain; and

an intracellular signaling domain, wherein the second CAR^{CD8+} comprises an intracellular signaling domain, e.g., a costimulatory signaling domain, not present on the CAR^{CD8+}, and, optionally, does not comprise an ICOS signaling domain.

### RNA Transfection

Disclosed herein are methods for producing an in vitro transcribed RNA CAR. RNA CAR and methods of using the same are described, e.g., in paragraphs 553-570 of in International Application WO2015/142675, filed Mar. 13, 2015, which is herein incorporated by reference in its entirety.

In one embodiment, the in vitro transcribed RNA CAR can be introduced to a cell as a form of transient transfection. The RNA may have a 3′ UTR, a 5′ UTR, or both. The 5′ UTR may contain a Kozak sequence. The RNA may comprise an IRES. The RNA may comprise a 5′ cap. The RNA may comprise a polyA sequence. RNA can be produced using a DNA template that comprises a promoter, e.g., a T7, T7, or SP6 promoter. RNA can be introduced into target cells using any of a number of different methods, for instance, commercially available methods which include, but are not limited to, electroporation, the Gene Pulser II, Multiporator, cationic liposome mediated transfection using lipofection, polymer encapsulation, peptide mediated transfection, or biolistic particle delivery systems such as “gene guns”.

### Non-Viral Delivery Methods

In some aspects, non-viral methods can be used to deliver a nucleic acid encoding a CAR described herein into a cell or tissue or a subject. Suitable non-viral delivery methods include transposons (e.g., Sleeping Beauty, piggyBac, and pT2-based transposons). Exemplary non-viral delivery methods and methods of using the same are described, e.g., in paragraphs 571-579 of International Application WO2015/142675, filed Mar. 13, 2015, which is herein incorporated by reference in its entirety.

### Methods of Manufacture/Production

In one aspect, methods of manufacturing a CAR-expressing cell according to the invention are disclosed herein (e.g., in “Source of Cells” and “Activation and Expansion of Cells”).

In an embodiment, a method of manufacturing a CAR-expressing cell is provided. The method comprises:
providing a preparation of a CAR-expressing cell (e.g., a plurality of CAR-expressing immune effector cells, such as a T cells, or an NK cells) (e.g., a CD19 CAR-expressing cell as described herein, such as, e.g., CTL019);
acquiring a value for the level of one or more of CD27, CD45RO, CCR7, HLA-DR, CD127 and CD95 to obtain a gene expression pattern for the sample;
(optionally) comparing the obtained gene expression pattern to that of a historical record of gene expression;

determining a difference between the obtained and historical gene expression; and
recording the determined difference in a quality control record.

In an embodiment, provided methods comprise steps of providing a CAR-expressing cell (e.g., T cell, NK cell) preparation (e.g., a CD19 CAR-expressing cell (e.g., T cell, NK cell) as described herein, such as, e.g., CTL019);
determining the levels of expression of one or more of CD27, CD45RO, CCR7, HLA-DR, CD127 and CD95 to obtain a gene expression pattern (e.g., a gene signature) for the sample;
correlating the gene signature with patient response to a CAR-expressing cell (e.g., T cell, NK cell) therapy (e.g. a CD19 CAR-expressing cell (e.g., T cell, NK cell) as described herein, such as, e.g., CTL019);
and optimizing the CAR-expressing cell (e.g., T cell, NK cell) preparation based on the correlation of the gene signature and patient response prior to infusion into patients.

In an embodiment, provided methods comprise a step of providing a blood sample, e.g., a T cell sample, from a subject having cancer.

In an embodiment, provided methods further comprise a step of comparing the obtained gene expression pattern difference with that of a reference sample.

In an embodiment, a reference sample is a CAR-expressing cell (e.g., T cell, NK cell) preparation (e.g., a CD19 CAR-expressing cell as described herein, such as, e.g., CTL019) from a different batch of cells producing the therapeutic CAR-expressing cell preparation.

In an embodiment, a reference sample is a healthy donor sample with a manufactured CAR-expressing cell (e.g., T cell, NK cell) product (e.g., a CD19 CAR-expressing cell as described herein, such as, e.g., CTL019). In an embodiment, a reference sample is a healthy donor sample with a manufactured CD19 CAR-expressing cell product, such as, e.g., CTL019 product.

In an embodiment, provided methods further comprise a step of recording the result of the comparing in a quality control record for the therapeutic CAR-expressing cell (e.g., T cell, NK cell) preparation.

In an embodiment, the determined difference is compared with a historical record of the reference sample.

In an embodiment, the CAR-expressing cell (e.g., T cell, NK cell) preparation is a CD19 CAR-expressing cell (e.g., CTL019) preparation.

In an embodiment, the CAR-expressing cell (e.g., T cell, NK cell) preparation comprises a CD19 CAR-expressing cell (e.g., CTL019) preparation.

In an embodiment, the CAR-expressing cell (e.g., T cell, NK cell) preparation consists of a CD19 CAR-expressing cell (e.g., CTL019) preparation.

In an aspect, a method is provided, comprising:

providing a blood sample, e.g., a T cell sample, from a subject having cancer;

determining the levels of expression of one or more of CD27, CD45RO, CCR7, HLA-DR, CD127 and CD95 to obtain a gene expression pattern for the sample;

comparing the obtained gene expression pattern to that of a reference value, e.g., a historical record of gene expression;

determining a difference between the obtained and the reference value; and

recording the determined difference in a quality control record.

The method can comprise a step of comparing the obtained gene expression pattern difference with that of a reference sample.

In some embodiments, the methods disclosed herein further include administering a T cell depleting agent after treatment with the cell (e.g., an immune effector cell as described herein), thereby reducing (e.g., depleting) the CAR-expressing cells (e.g., the CD19CAR-expressing cells). Such T cell depleting agents can be used to effectively deplete CAR-expressing cells (e.g., CD19CAR-expressing cells) to mitigate toxicity. In some embodiments, the CAR-expressing cells were manufactured according to a method herein, e.g., assayed (e.g., before or after transfection or transduction) according to a method herein.

In some embodiments, the T cell depleting agent is administered one, two, three, four, or five weeks after administration of the cell, e.g., the population of immune effector cells, described herein.

In one embodiment, the T cell depleting agent is an agent that depletes CAR-expressing cells, e.g., by inducing antibody dependent cell-mediated cytotoxicity (ADCC) and/or complement-induced cell death. For example, CAR-expressing cells described herein may also express an antigen (e.g., a target antigen) that is recognized by molecules capable of inducing cell death, e.g., ADCC or complement-induced cell death. For example, CAR expressing cells described herein may also express a target protein (e.g., a receptor) capable of being targeted by an antibody or antibody fragment. Examples of such target proteins include, but are not limited to, EpCAM, VEGFR, integrins (e.g., integrins ανβ3, α4, αI3/4β3, α4β7, α5β1, ανβ3, αν), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/IgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

In some embodiments, the CAR expressing cell co-expresses the CAR and the target protein, e.g., naturally expresses the target protein or is engineered to express the target protein. For example, the cell, e.g., the population of immune effector cells, can include a nucleic acid (e.g., vector) comprising the CAR nucleic acid (e.g., a CAR nucleic acid as described herein) and a nucleic acid encoding the target protein.

In one embodiment, the T cell depleting agent is a CD52 inhibitor, e.g., an anti-CD52 antibody molecule, e.g., alemtuzumab.

In other embodiments, the cell, e.g., the population of immune effector cells, expresses a CAR molecule as described herein (e.g., CD19CAR) and the target protein recognized by the T cell depleting agent. In one embodiment, the target protein is CD20. In embodiments where the target protein is CD20, the T cell depleting agent is an anti-CD20 antibody, e.g., rituximab.

In further embodiments of any of the aforesaid methods, the methods further include transplanting a cell, e.g., a hematopoietic stem cell, or a bone marrow, into the mammal.

In another aspect, the invention features a method of conditioning a mammal prior to cell transplantation. The method includes administering to the mammal an effective amount of the cell comprising a CAR nucleic acid or polypeptide, e.g., a CD19 CAR nucleic acid or polypeptide. In some embodiments, the cell transplantation is a stem cell transplantation, e.g., a hematopoietic stem cell transplantation, or a bone marrow transplantation. In other embodiments, conditioning a subject prior to cell transplantation includes reducing the number of target-expressing cells in a subject, e.g., CD19-expressing normal cells or CD19-expressing cancer cells.

### Nucleic Acid Constructs Encoding a CAR

Nucleic acid molecules encoding one or more CAR constructs can be introduced into an immune effector cell (e.g., a T cell) as described herein. In one aspect, the nucleic acid molecule is provided as a messenger RNA transcript. In one aspect, the nucleic acid molecule is provided as a DNA construct.

In some embodiments, a nucleic acid described herein is introduced into a cell that has been assayed by a method described herein, e.g., one or more biomarkers has been assayed. In some embodiments, a cell comprising a nucleic acid described herein is assayed by a method described herein, e.g., one or more biomarkers has been assayed.

The nucleic acid molecules described herein can be a DNA molecule, an RNA molecule, or a combination thereof. In one embodiment, the nucleic acid molecule is an mRNA encoding a CAR polypeptide as described herein. In other embodiments, the nucleic acid molecule is a vector that includes any of the aforesaid nucleic acid molecules.

Nucleic acid molecules can encode, e.g., a CAR molecule described herein, and can comprise, e.g., a nucleic acid sequence described herein, e.g., in Table 1, Table 12 or Table 13.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Also described are vectors in which a nucleic acid of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (w), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the invention is an adenoviral vector (A5/35). In another embodiment, the expression of nucleic acids encoding CARs can be accomplished using of transposons such as sleeping beauty, CRISPR, CAS9, and zinc finger nucleases. See below June et al. 2009 NATURE REVIEWS IMMUNOLOGY 9.10: 704-716, is incorporated herein by reference.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1α, ubiquitin C, or phosphoglycerokinase (PGK) promoters.

An example of a promoter that is capable of expressing a CAR transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from transgenes cloned into a lentiviral vector. See, e.g., Milone et al., MOL. THER. 17(8): 1453-1464 (2009). In one aspect, the EF1a promoter comprises the sequence provided as SEQ ID NO:11.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired.

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. Reporter genes are described, e.g., in paragraph 599 of International Application WO2015/142675, filed Mar. 13, 2015, which is herein incorporated by reference in its entirety.

In embodiments, the vector may comprise two or more nucleic acid sequences encoding a CAR, e.g., a CAR described herein, e.g., a CD19 CAR, and a second CAR, e.g., an inhibitory CAR or a CAR that specifically binds to an antigen other than CD19. In such embodiments, the two or more nucleic acid sequences encoding the CAR are encoded by a single nucleic molecule in the same frame and as a single polypeptide chain. In this aspect, the two or more CARs, can, e.g., be separated by one or more peptide cleavage sites. (e.g., an auto-cleavage site or a substrate for an intracellular protease). Examples of peptide cleavage sites include T2A, P2A, E2A, or F2A sites.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means, e.g., those described in paragraphs 601-603 of International Application WO2015/142675, filed Mar. 13, 2015, which is herein incorporated by reference in its entirety.

In the case where a non-viral delivery system is utilized, an exemplary delivery vehicle is a liposome. The use of lipid formulations is contemplated for the introduction of the nucleic acids into a host cell (in vitro, ex vivo or in vivo), and is described, e.g., in paragraphs 604-605 of International Application WO2015/142675, filed Mar. 13, 2015, which is herein incorporated by reference in its entirety.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

### Therapeutic Methods

In one aspect, the disclosure provides methods for treating a disease associated with expression of a tumor antigen described herein. In some embodiments, immune effector cells are assayed by a method described herein, e.g., one or more biomarkers is assayed, and the cells are administered to a subject as part of a treatment described herein. For example, the immune effector cells can be administered as part of a combination therapy described herein.

In one aspect, the present disclosure provides methods of treating cancer (e.g., a hematological cancer such as ALL and CLL) by providing to the subject in need thereof immune effector cells (e.g., T cells, NK cells) that are engineered to express a CAR. In one embodiment, the cancer to be treated is a B cell malignancy. In one embodiment, the cancer to be treated is ALL (acute lymphoblastic leukemia), CLL (chronic lymphocytic leukemia), DLBCL (diffuse large B-cell lymphoma), MCL (Mantle cell lymphoma, or MINI (multiple myeloma).

In one aspect, the disclosure provides methods of treating cancer (e.g., a hematological cancer such as ALL and CLL) by providing to the subject in need thereof immune effector cells (e.g., T cells, NK cells) that are engineered to express a CD19 CAR, wherein the cancer cells express CD19. In one embodiment, the cancer to be treated is a B cell malignancy. In one embodiment, the cancer to be treated is ALL (acute lymphoblastic leukemia), CLL (chronic lymphocytic leukemia), DLBCL (diffuse large B-cell lymphoma), MCL (Mantle cell lymphoma), Hodgkin lymphoma, or MM (multiple myeloma).

In one aspect, the present invention provides methods of treating cancer (e.g., a hematological cancer such as ALL and CLL) by providing to the subject in need thereof immune effector cells (e.g., T cells, NK cells) that are engineered to express a CD22 CAR, wherein the cancer cells express CD22. In one embodiment, the cancer to be treated is a B cell malignancy. In one embodiment, the cancer to be treated is ALL (acute lymphoblastic leukemia), CLL (chronic lymphocytic leukemia), DLBCL (diffuse large B-cell lymphoma), MCL (Mantle cell lymphoma), Hodgkin lymphoma, or MM (multiple myeloma).

In one aspect, the present invention provides methods of treating cancer (e.g., a hematological cancer such as ALL and CLL) by providing to the subject in need thereof immune effector cells (e.g., T cells, NK cells) that are engineered to express a CD20 CAR, wherein the cancer cells express CD20. In one embodiment, the cancer to be treated is a B cell malignancy. In one embodiment, the cancer to be treated is ALL (acute lymphoblastic leukemia), CLL (chronic lymphocytic leukemia), DLBCL (diffuse large B-cell lymphoma), MCL (Mantle cell lymphoma), Hodgkin lymphoma, or MM (multiple myeloma).

In one aspect, the present invention provides methods of treating cancer (e.g., a hematological cancer such as ALL and CLL) by providing to the subject in need thereof immune effector cells (e.g., T cells, NK cells) that are engineered to express a ROR1 CAR, wherein the cancer cells express ROR1. In one embodiment, the cancer to be treated is a B cell malignancy. In one embodiment, the cancer to be treated is ALL (acute lymphoblastic leukemia), CLL (chronic lymphocytic leukemia), DLBCL (diffuse large B-cell lymphoma), MCL (Mantle cell lymphoma), Hodgkin lymphoma, or MM (multiple myeloma).

The disclosure includes a type of cellular therapy where immune effector cells (e.g., T cells, NK cells) are genetically modified (e.g., via transduction of a lentiviral vector) to express a CAR and the CAR-expressing cell is infused to a recipient in need thereof. The infused cell is able to kill tumor cells in the recipient. Unlike antibody therapies, CAR-modified immune effector cells (e.g., T cells, NK cells) are able to replicate in vivo resulting in long-term persistence that can lead to sustained tumor control. CAR-expressing cells (e.g., T cells or NK cells) generated using lentiviral vectors will have stable CAR expression. In various aspects, the immune effector cells (e.g., T cells, NK cells) administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell to the patient.

The invention also includes a type of cellular therapy where immune effector cells (e.g., T cells, NK cells) are modified, e.g., by in vitro transcribed RNA, to transiently express a CAR and the CAR-expressing cell is infused to a recipient in need thereof. CAR-expressing cells (e.g., T cells, NK cells) generated through transduction of CAR RNA (e.g., by transfection or electroporation) transiently express RNA CARs for 4, 5, 6, 7, 8, 9, 10, 11, 12, 13, 14, 15 days after transduction. The infused cell is able to kill tumor cells in the recipient. Thus, in various aspects, the immune effector cells (e.g., T cells, NK cells) administered to the patient, is present for less than one month, e.g., three weeks, two weeks, one week, after administration of the T cell to the patient.

In one aspect, the present disclosure provides methods of treating cancer (e.g., a hematological cancer such as ALL and CLL) by providing to the subject in need thereof immune effector cells (e.g., T cells, NK cells) that are engineered to express a CAR, e.g., a CAR described herein.

In one embodiment, the present disclosure provides methods of treating cancer (e.g., a hematological cancer such as ALL and CLL) by providing to the subject in need thereof immune effector cells (e.g., T cells, NK cells) that are engineered to express a CAR that specifically targets or binds to a tumor antigen (or cancer associated antigen) described herein, wherein the subject has been identified as a responder or partial responder. In other embodiments, the methods provide treating a cancer (e.g., a hematological cancer such as ALL and CLL) as a partial responder or non-responder by providing to the subject a cancer therapy other than a CAR therapy, e.g., providing the subject a treatment that is the standard of care for that particular type of cancer. In yet another embodiment, the method of treatment includes altering the manufacturing of a CAR-expressing cell to enrich for naïve T cells, e.g., as described herein, for a subject identified as a partial responder or non-responder prior to administering a CAR-expressing cell, e.g., a CAR-expressing cell described herein.

In one embodiment, the immune effector cells (e.g., T cells, NK cells) are engineered to express CD19 CAR, for treating a subject having cancer (e.g., a hematological cancer such as ALL and CLL), wherein the cancer cells express CD19. In one embodiment, the cancer to be treated is ALL or CLL. The CD19 CAR molecules to be expressed in an immune effector cell can comprise any anti-CD19 antigen binding domain in the art (e.g., those provided in Table 12) in combination with any of the CAR domains described herein to generate a full CAR construct. For example, the full CAR construct is a CAR listed in Table 13. Table 13 provides the exemplary full CD19 CAR constructs generated using the various CAR domains (e.g., transmembrane and intracellular signaling domains) listed in Table 12, and the anti-CD19 antigen binding domains listed in Table 12. Amino acid sequences are designated (aa) and nucleic acid sequences are designated (nt).

TABLE 13
CD19 CAR Constructs

| | |
|---|---|
| Name | Sequence |

CAR 1

| | |
|---|---|
| 104875 | atggccctccctgtcaccgccctgctgcttccgctggctcttctgctccacgccgctcggcccgaaattgtgatgacc |
| CAR 1 - | cagtcacccgccactcttagcctttcacccggtgagcgcgcaaccctgtcttgcagagcctcccaagacatctcaaa |
| Full - nt | ataccttaattggtatcaacagaagcccggacaggctcctcgccttctgatctaccacaccagccggctccattctgg |
| | aatccctgccaggttcagcggtagcggatctgggaccgactacaccctcactatcagctcactgcagccagaggac |
| | ttcgctgtctatttctgtcagcaagggaacaccctgccctacacctttggacagggcaccaagctcgagattaaaggt |
| | ggaggtggcagcggaggaggtgggtccggcggtggaggaagccaggtccaactccaagaaagcggaccgggt |
| | cttgtgaagccatcagaaactctttcactgacttgtactgtgagcggagtgtctctccccgattacggggtgtcttggat |
| | cagacagccaccggggaagggtctggaatggattggagtgatttggggctctgagactacttactactcttcatccct |
| | caagtcacgcgtcaccatctcaaaggacaactctaagaatcaggtgtcactgaaactgtcatctgtgaccgcagccg |
| | acaccgccgtgtactattgcgctaagcattactattatggcgggagctacgcaatggattactggggacagggtactc |
| | tggtcaccgtgtccagcaccactaccccagcaccgaggccacccaccccggctcctaccatcgcctcccagcctct |
| | gtccctgcgtccggaggcatgtagacccgcagctggtggggccgtgcatacccggggtcttgacttcgcctgcgat |
| | atctacatttgggcccctctggctggtacttgcggggtcctgctgctttcactcgtgatcactctttactgtaagcgcggt |
| | cggaagaagctgctgtacatctttaagcaacccttcatgaggcctgtgcagactactcaagaggaggacggctgttc |
| | atgccggttcccagaggaggaggaaggcggctgcgaactgcgcgtgaaattcagccgcagcgcagatgctccag |
| | cctacaagcaggggcagaaccagctctacaacgaactcaatcttggtcggagagaggagtacgacgtgctggaca |
| | agcggagaggacgggacccagaaatgggcgggaagccgcgcagaaagaatccccaagagggcctgtacaacg |
| | agctccaaaaggataagatggcagaagcctatagcgagattggtatgaaaggggaacgcagaagaggcaaaggc |
| | cacgacggactgtaccagggactcagcaccgccaccaaggacacctatgacgctcttcacatgcaggccctgccg |
| | cctcgg (SEQ ID NO: 55) |
| 104875 | MALPVTALLLPLALLLHAARPeivmtqspatlslspgeratlscrasqdiskylnwyqqkpgqa |
| CAR 1 - | prlliyhtsrlhsgiparfsgsgsgtdytltisslqpedfavyfcqqgntlpytfgqgtkleikggggsggggsggg |
| Full - aa | gsqvqlqesgpglvkpsetlsltctvsgvslpdygvswirqppgkglewigviwgsettyyssslksrvtiskdn |
| | sknqvslklssvtaadtavyycakhyyyggsyamdywgqgtlvtvsstttpaprpptpaptiasqplslrpeacr |
| | paaggavhtrgldfacdiyiwaplagtcgvlllslvitlyckrgrkkllyifkqpfmrpvqttqeedgcscrfpeeee |
| | ggcelrvkfsrsadapaykqgqnqlynelnlgrreeydvldkrrgrdpemggkprrknpqeglynelqkdkm |
| | aeayseigmkgerrrgkghdglyqglstatkdtydalhmqalppr (SEQ ID NO: 56) |

CAR 2

| | |
|---|---|
| 104876 | atggccctccctgtcaccgccctgctgcttccgctggctcttctgctccacgccgctcggcccgaaattgtgatgacc |
| CAR 2 - | cagtcacccgccactcttagcctttcacccggtgagcgcgcaaccctgtcttgcagagcctcccaagacatctcaaa |
| Full - nt | ataccttaattggtatcaacagaagcccggacaggctcctcgccttctgatctaccacaccagccggctccattctgg |
| | aatccctgccaggttcagcggtagcggatctgggaccgactacaccctcactatcagctcactgcagccagaggac |
| | ttcgctgtctatttctgtcagcaagggaacaccctgccctacacctttggacagggcaccaagctcgagattaaaggt |
| | ggaggtggcagcggaggaggtgggtccggcggtggaggaagccaggtccaactccaagaaagcggaccgggt |
| | cttgtgaagccatcagaaactctttcactgacttgtactgtgagcggagtgtctctccccgattacggggtgtcttggat |
| | cagacagccaccggggaagggtctggaatggattggagtgatttggggctctgagactacttactaccaatcatccc |
| | tcaagtcacgcgtcaccatctcaaaggacaactctaagaatcaggtgtcactgaaactgtcatctgtgaccgcagcc |
| | gacaccgccgtgtactattgcgctaagcattactattatggcgggagctacgcaatggattactggggacagggtact |
| | ctggtcaccgtgtccagcaccactaccccagcaccgaggccacccaccccggctcctaccatcgcctcccagcctc |
| | tgtccctgcgtccggaggcatgtagacccgcagctggtggggccgtgcatacccggggtcttgacttcgcctgcga |
| | tatctacatttgggcccctctggctggtacttgcggggtcctgctgctttcactcgtgatcactctttactgtaagcgcgg |
| | tcggaagaagctgctgtacatctttaagcaacccttcatgaggcctgtgcagactactcaagaggaggacggctgttc |
| | atgccggttcccagaggaggaggaaggcggctgcgaactgcgcgtgaaattcagccgcagcgcagatgctccag |
| | cctacaagcaggggcagaaccagctctacaacgaactcaatcttggtcggagagaggagtacgacgtgctggaca |
| | agcggagaggacgggacccagaaatgggcgggaagccgcgcagaaagaatccccaagagggcctgtacaacg |
| | agctccaaaaggataagatggcagaagcctatagcgagattggtatgaaaggggaacgcagaagaggcaaaggc |
| | cacgacggactgtaccagggactcagcaccgccaccaaggacacctatgacgctcttcacatgcaggccctgccg |
| | cctcgg (SEQ ID NO: 57) |
| 104876 | MALPVTALLLPLALLLHAARPeivmtqspatlslspgeratlscrasqdiskylnwyqqkpgqa |
| CAR 2 - | prlliyhtsrlhsgiparfsgsgsgtdytltisslqpedfavyfcqqgntlpytfgqgtkleikggggsggggsggg |
| Full - aa | gsqvqlqesgpglvkpsetlsltctvsgvslpdygvswirqppgkglewigviwgsettyyqsslksrvtiskdn |
| | sknqvslklssvtaadtavyycakhyyggsyamdywgqgtlvtvsstttpaprpptpaptiasqplslrpeacr |
| | paaggavhtrgldfacdiyiwaplagtcgvlllslvitlyckrgrkkllyifkqpfmrpvqttqeedgcscrfpeeee |
| | ggcelrvkfsrsadapaykqgqnqlynelnlgrreeydvldkrrgrdpemggkprrknpqeglynelqkdkm |
| | aeayseigmkgerrrgkghdglyqglstatkdtydalhmqalppr (SEQ ID NO: 58) |

CAR 3

| | |
|---|---|
| 104877 | atggctctgcccgtgaccgcactcctcctgccactggctctgctgcttcacgccgctcgcccacaagtccagcttcaa |
| CAR 3 - | gaatcagggcctggtctggtgaagccatctgagactctgtccctcacttgcaccgtgagcggagtgtccctcccaga |
| Full - nt | ctacggagtgagctggattagacagcctcccggaaagggactggagtggatcggagtgatttggggtagcgaaac |
| | cacttactattcatcttccctgaagtcacgggtcaccatttcaaaggataactcaaagaatcaagtgagcctcaagctct |
| | catcagtcaccgccgctgacaccgccgtgtattactgtgccaagcattactactatggagggtcctacgccatggact |
| | actggggccagggaactctggtcactgtgtcatctggtggaggaggtagcggaggaggcgggagcggtggaggt |
| | ggctccgaaatcgtgatgacccagagccctgcaaccctgtccctttctcccggggaacgggctaccctttcttgtcgg |
| | gcatcacaagatatctcaaaatacctcaattggtatcaacagaagccgggacaggcccctaggcttcttatctaccac |
| | acctctcgcctgcatagcgggattcccgcacgctttagcgggtctggaagcgggaccgactacactctgaccatctc |
| | atctctccagcccgaggacttcgccgtctacttctgccagcagggtaacaccctgccgtacaccttcggccagggca |
| | ccaagcttgagatcaaaaccactactcccgctccaaggccacccacccctgccccgaccatcgcctctcagccgctt |
| | tccctgcgtccggaggcatgtagacccgcagctggtggggccgtgcatacccggggtcttgacttcgcctgcgatat |
| | ctacatttgggcccctctggctggtacttgcggggtcctgctgctttcactcgtgatcactctttactgtaagcgcggtc |
| | ggaagaagctgctgtacatctttaagcaacccttcatgaggcctgtgcagactactcaagaggaggacggctgttcat |
| | gccggttcccagaggaggaggaaggcggctgcgaactgcgcgtgaaattcagccgcagcgcagatgctccagc |
| | ctacaagcaggggcagaaccagctctacaacgaactcaatcttggtcggagagaggagtacgacgtgctggacaa |
| | gcggagaggacgggacccagaaatgggcgggaagccgcgcagaaagaatccccaagagggcctgtacaacga |
| | gctccaaaaggataagatggcagaagcctatagcgagattggtatgaaaggggaacgcagaagaggcaaaggcc |
| | acgacggactgtaccagggactcagcaccgccaccaaggacacctatgacgctcttcacatgcaggccctgccgc |
| | ctcgg (SEQ ID NO: 59) |
| 104877 | MALPVTALLLPLALLLHAARPqvqlqesgpglvkpsetlsltctvsgvslpdygvswirqppgk |
| CAR 3 - | glewigviwgsettyyssslksrvtiskdnsknqvslklssvtaadtavyycakhyyggsyamdywgqgtl |
| Full - aa | vtvssggggsggggsggggseivmtqspatlslspgeratlscrasqdiskylnwyqqkpgqaprlliyhtsrl |
| | hsgiparfsgsgsgtdytltisslqpedfavyfcqqgntlpytfgqgtkleiktttpaprpptpaptiasqplslrpea |
| | crpaaggavhtrgldfacdiyiwaplagtcgvlllslvitlyckrgrkkllyifkqpfmrpvqttqeedgcscrfpee |
| | eeggcelrvkfsrsadapaykqgqnqlynelnlgrreeydvldkrrgrdpemggkprrknpqeglynelqkdk |
| | maeayseigmkgerrrgkghdglyqglstatkdtydalhmqalppr (SEQ ID NO: 60) |

CAR 4

| | |
|---|---|
| 104878 | atggctctgcccgtgaccgcactcctcctgccactggctctgctgcttcacgccgctcgcccacaagtccagcttcaa |
| CAR 4 - | gaatcagggcctggtctggtgaagccatctgagactctgtccctcacttgcaccgtgagcggagtgtccctcccaga |
| Full - nt | ctacggagtgagctggattagacagcctcccggaaagggactggagtggatcggagtgatttggggtagcgaaac |
| | cacttactatcaatcttccctgaagtcacgggtcaccatttcaaaggataactcaaagaatcaagtgagcctcaagctc |
| | tcatcagtcaccgccgctgacaccgccgtgtattactgtgccaagcattactactatggagggtcctacgccatggac |
| | tactggggccagggaactctggtcactgtgtcatctggtggaggaggtagcggaggaggcgggagcggtggagg |
| | tggctccgaaatcgtgatgacccagagccctgcaaccctgtccctttctcccggggaacgggctaccctttcttgtcg |
| | ggcatcacaagatatctcaaaatacctcaattggtatcaacagaagccgggacaggcccctaggcttcttatctacca |
| | cacctctcgcctgcatagcgggattcccgcacgctttagcgggtctggaagcgggaccgactacactctgaccatct |
| | catctctccagcccgaggacttcgccgtctacttctgccagcagggtaacaccctgccgtacaccttcggccagggc |
| | accaagcttgagatcaaaaccactactcccgctccaaggccacccacccctgccccgaccatcgcctctcagccgc |
| | tttccctgcgtccggaggcatgtagacccgcagctggtggggccgtgcatacccggggtcttgacttcgcctgcgat |
| | atctacatttgggcccctctggctggtacttgcggggtcctgctgctttcactcgtgatcactctttactgtaagcgcggt |
| | cggaagaagctgctgtacatctttaagcaacccttcatgaggcctgtgcagactactcaagaggaggacggctgttc |
| | atgccggttcccagaggaggaggaaggcggctgcgaactgcgcgtgaaattcagccgcagcgcagatgctccag |
| | cctacaagcaggggcagaaccagctctacaacgaactcaatcttggtcggagagaggagtacgacgtgctggaca |
| | agcggagaggacgggacccagaaatgggcgggaagccgcgcagaaagaatccccaagagggcctgtacaacg |
| | agctccaaaaggataagatggcagaagcctatagcgagattggtatgaaaggggaacgcagaagaggcaaaggc |
| | cacgacggactgtaccagggactcagcaccgccaccaaggacacctatgacgctcttcacatgcaggccctgccg |
| | cctcgg (SEQ ID NO: 61) |
| 104878 | MALPVTALLLPLALLLHAARPqvqlqesgpglvkpsetlsltctvsgvslpdygvswirqppgk |
| CAR 4 - | glewigviwgsettyyqsslksrvtiskdnsknqvslklssvtaadtavyycakhyyyggsyamdywgqgtl |
| Full - aa | vtvssggggsggggsggggseivmtqspatlslspgeratlscrasqdiskylnwyqqkpgqaprlliyhtsrl |
| | hsgiparfsgsgsgtdytltisslqpedfavyfcqqgntlpytfgqgtkleiktttpaprpptpaptiasqplslrpea |
| | crpaaggavhtrgldfacdiyiwaplagtcgvlllslvitlyckrgrkkllyifkqpfmrpvqttqeedgcscrfpee |
| | eeggcelrvkfsrsadapaykqgqnqlynelnlgrreeydvldkrrgrdpemggkprrknpqeglynelqkdk |
| | maeayseigmkgerrrgkghdglyqglstatkdtydalhmqalppr (SEQ ID NO: 62) |

CAR 5

| | |
|---|---|
| CAR5 | eivmtqspatlslspgeratlscrasqdiskylnwyqqkpgqaprlliyhtsrlhsgiparfsgsgsgtdytltisslq |
| scFy | pedfavyfcqqgntlpytfgqgtkleikggggsggggsggggsggggsqvqlqesgpglvkpsetlsltctvsgv |
| domain | slpdygvswirqppgkglewigviwgsettyyssslksrvtiskdnsknqvslklssvtaadtavyycakhyyy |
| | ggsyamdywgqgtlvtvss (SEQ ID NO: 63) |
| 104879 | atggccctccctgtcaccgccctgctgcttccgctggctcttctgctccacgccgctcggcccgaaattgtgatgacc |
| CAR 5 - | cagtcacccgccactcttagcctttcacccggtgagcgcgcaaccctgtcttgcagagcctcccaagacatctcaaa |
| Full - nt | ataccttaattggtatcaacagaagcccggacaggctcctcgccttctgatctaccacaccagccggctccattctgg |
| | aatccctgccaggttcagcggtagcggatctgggaccgactacaccctcactatcagctcactgcagccagaggac |
| | ttcgctgtctatttctgtcagcaagggaacaccctgccctacacctttggacagggcaccaagctcgagattaaaggt |
| | ggaggtggcagcggaggaggtgggtccggcggtggaggaagcggcggaggcgggagccaggtccaactcca |
| | agaaagcggaccgggtcttgtgaagccatcagaaactctttcactgacttgtactgtgagcggagtgtctctccccga |
| | ttacggggtgtcttggatcagacagccaccggggaagggtctggaatggattggagtgatttggggctctgagacta |
| | cttactactcttcatccctcaagtcacgcgtcaccatctcaaaggacaactctaagaatcaggtgtcactgaaactgtca |
| | tctgtgaccgcagccgacaccgccgtgtactattgcgctaagcattactattatggcgggagctacgcaatggattac |
| | tggggacagggtactctggtcaccgtgtccagcaccactaccccagcaccgaggccacccaccccggctcctacc |
| | atcgcctcccagcctctgtccctgcgtccggaggcatgtagacccgcagctggtggggccgtgcatacccggggtc |
| | ttgacttcgcctgcgatatctacatttgggcccctctggctggtacttgcggggtcctgctgctttcactcgtgatcactc |
| | tttactgtaagcgcggtcggaagaagctgctgtacatctttaagcaacccttcatgaggcctgtgcagactactcaaga |
| | ggaggacggctgttcatgccggttcccagaggaggaggaaggcggctgcgaactgcgcgtgaaattcagccgca |
| | gcgcagatgctccagcctacaagcaggggcagaaccagctctacaacgaactcaatcttggtcggagagaggagt |
| | acgacgtgctggacaagcggagaggacgggacccagaaatgggcgggaagccgcgcagaaagaatccccaag |
| | agggcctgtacaacgagctccaaaaggataagatggcagaagcctatagcgagattggtatgaaaggggaacgca |
| | gaagaggcaaaggccacgacggactgtaccagggactcagcaccgccaccaaggacacctatgacgctcttcac |
| | atgcaggccctgccgcctcgg (SEQ ID NO: 64) |
| 104879 | MALPVTALLLPLALLLHAARPeivmtqspatlslspgeratlscrasqdiskylnwyqqkpgqa |
| CAR 5 - | prlliyhtsrlhsgiparfsgsgsgtdytltisslqpedfavyfcqqgntlpytfgqgtkleikggggsggggsggg |
| Full - aa | gsggggsqvqlqesgpglvkpsetlsltctvsgvslpdygvswirqppgkglewigviwgsettyyssslksrvt |
| | iskdnsknqvslklssvtaadtavyycakhyyyggsyamdywgqgtlvtvsstttpaprpptpaptiasqplslr |
| | peacrpaaggavhtrgldfacdiyiwaplagtcgvlllslvitlyckrgrkkllyifkqpfmrpvqttqeedgcscrf |
| | peeeeggcelrvkfsrsadapaykqgqnqlynelnlgrreeydvldkrrgrdpemggkprrknpqeglynelq |
| | kdkmaeayseigmkgerrrgkghdglyqglstatkdtydalhmqalppr (SEQ ID NO: 65) |

CAR6

| | |
|---|---|
| 104880 | atggccctccctgtcaccgccctgctgcttccgctggctcttctgctccacgccgctcggcccgaaattgtgatgacc |
| CAR6 - | cagtcacccgccactcttagcctttcacccggtgagcgcgcaaccctgtcttgcagagcctcccaagacatctcaaa |
| Full - nt | ataccttaattggtatcaacagaagcccggacaggctcctcgccttctgatctaccacaccagccggctccattctgg |
| | aatccctgccaggttcagcggtagcggatctgggaccgactacaccctcactatcagctcactgcagccagaggac |
| | ttcgctgtctatttctgtcagcaagggaacaccctgccctacacctttggacagggcaccaagctcgagattaaaggt |
| | ggaggtggcagcggaggaggtgggtccggcggtggaggaagcggaggcggagggagccaggtccaactcca |
| | agaaagcggaccgggtcttgtgaagccatcagaaactctttcactgacttgtactgtgagcggagtgtctctccccga |
| | ttacggggtgtcttggatcagacagccaccggggaagggtctggaatggattggagtgatttggggctctgagacta |
| | cttactaccaatcatccctcaagtcacgcgtcaccatctcaaaggacaactctaagaatcaggtgtcactgaaactgtc |
| | atctgtgaccgcagccgacaccgccgtgtactattgcgctaagcattactattatggcgggagctacgcaatggatta |
| | ctggggacagggtactctggtcaccgtgtccagcaccactaccccagcaccgaggccacccaccccggctcctac |
| | catcgcctcccagcctctgtccctgcgtccggaggcatgtagacccgcagctggtggggccgtgcatacccggggt |
| | cttgacttcgcctgcgatatctacatttgggcccctctggctggtacttgcggggtcctgctgctttcactcgtgatcact |
| | ctttactgtaagcgcggtcggaagaagctgctgtacatctttaagcaacccttcatgaggcctgtgcagactactcaag |
| | aggaggacggctgttcatgccggttcccagaggaggaggaaggcggctgcgaactgcgcgtgaaattcagccgc |
| | agcgcagatgctccagcctacaagcaggggcagaaccagctctacaacgaactcaatcttggtcggagagagga |
| | gtacgacgtgctggacaagcggagaggacgggacccagaaatgggcgggaagccgcgcagaaagaatcccca |
| | agagggcctgtacaacgagctccaaaaggataagatggcagaagcctatagcgagattggtatgaaaggggaac |
| | gcagaagaggcaaaggccacgacggactgtaccagggactcagcaccgccaccaaggacacctatgacgctctt |
| | cacatgcaggccctgccgcctcgg (SEQ ID NO: 66) |
| 104880 | MALPVTALLLPLALLLHAARPeivmtqspatlslspgeratlscrasqdiskylnwyqqkpgqa |
| CAR6 - | prlliyhtsrlhsgiparfsgsgsgtdytltisslqpedfavyfcqqgntlpytfgqgtkleikggggsggggsggg |
| Full - aa | gsggggsqvqlqesgpglvkpsetlsltctvsgvslpdygvswirqppgkglewigviwgsettyyqsslksrv |
| | tiskdnsknqvslklssvtaadtavyycakhyyyggsyamdywgqgtlvtvsstttpaprpptpaptiasqplsl |
| | rpeacrpaaggavhtrgldfacdiyiwaplagtcgvlllslvitlyckrgrkkllyifkqpfmrpvqttqeedgcscr |
| | fpeeeeggcelrvkfsrsadapaykqgqnqlynelnlgrreeydvldkrrgrdpemggkprrknpqeglynelq |
| | kdkmaeayseigmkgerrrgkghdglyqglstatkdtydalhmqalppr (SEQ ID NO: 67) |

CAR 7

| | |
|---|---|
| 104881 | atggctctgcccgtgaccgcactcctcctgccactggctctgctgcttcacgccgctcgcccacaagtccagcttcaa |
| CAR 7 | gaatcagggcctggtctggtgaagccatctgagactctgtccctcacttgcaccgtgagcggagtgtccctcccaga |
| Full - nt | ctacggagtgagctggattagacagcctcccggaaagggactggagtggatcggagtgatttggggtagcgaaac |
| | cacttactattcatcttccctgaagtcacgggtcaccatttcaaaggataactcaaagaatcaagtgagcctcaagctct |
| | catcagtcaccgccgctgacaccgccgtgtattactgtgccaagcattactactatggagggtcctacgccatggact |
| | actggggccagggaactctggtcactgtgtcatctggtggaggaggtagcggaggaggcgggagcggtggaggt |
| | ggctccggaggtggcggaagcgaaatcgtgatgacccagagccctgcaaccctgtccctttctcccggggaacgg |
| | gctaccattcttgtcgggcatcacaagatatctcaaaatacctcaattggtatcaacagaagccgggacaggcccct |
| | aggcttcttatctaccacacctctcgcctgcatagcgggattcccgcacgctttagcgggtctggaagcgggaccga |
| | ctacactctgaccatctcatctctccagcccgaggacttcgccgtctacttctgccagcagggtaacaccctgccgta |
| | caccttcggccagggcaccaagcttgagatcaaaaccactactcccgctccaaggccacccacccctgccccgac |
| | catcgcctctcagccgctttccctgcgtccggaggcatgtagacccgcagctggtggggccgtgcatacccggggt |
| | cttgacttcgcctgcgatatctacatttgggcccctctggctggtacttgcggggtcctgctgctttcactcgtgatcact |
| | ctttactgtaagcgcggtcggaagaagctgctgtacatctttaagcaacccttcatgaggcctgtgcagactactcaag |
| | aggaggacggctgttcatgccggttcccagaggaggaggaaggcggctgcgaactgcgcgtgaaattcagccgc |
| | agcgcagatgctccagcctacaagcaggggcagaaccagctctacaacgaactcaatcttggtcggagagagga |
| | gtacgacgtgctggacaagcggagaggacgggacccagaaatgggcgggaagccgcgcagaaagaatcccca |
| | agagggcctgtacaacgagctccaaaaggataagatggcagaagcctatagcgagattggtatgaaaggggaac |
| | gcagaagaggcaaaggccacgacggactgtaccagggactcagcaccgccaccaaggacacctatgacgctctt |
| | cacatgcaggccctgccgcctcgg (SEQ ID NO: 68) |
| 104881 | MALPVTALLLPLALLLHAARPqvqlqesgpglvkpsetlsltctvsgvslpdygvswirqppgk |
| CAR 7 | glewigviwgsettyyssslksrvtiskdnsknqvslklssvtaadtavyycakhyyyggsyamdywgqgtl |
| Full-aa | vtvssggggsggggsggggsggggseivmtqspatlslspgeratlscrasqdiskylnwyqqkpgqaprlliy |
| | htsrlhsgiparfsgsgsgtdytltisslqpedfavyfcqqgntlpytfgqgtkleiktttpaprpptpaptiasqpls |
| | lrpeacrpaaggavhtrgldfacdiyiwaplagtcgvlllslvitlyckrgrkkllyifkqpfmrpvqttqeedgcsc |
| | rfpeeeeggcelrvkfsrsadapaykqgqnqlynelnlgrreeydvldkrrgrdpemggkprrknpqeglynel |
| | qkdkmaeayseigmkgerrrgkghdglyqglstatkdtydalhmqalppr (SEQ ID NO: 69) |

CAR 8

| | |
|---|---|
| 104882 | atggctctgcccgtgaccgcactcctcctgccactggctctgctgcttcacgccgctcgcccacaagtccagcttcaa |
| CAR 8 - | gaatcagggcctggtctggtgaagccatctgagactctgtccctcacttgcaccgtgagcggagtgtccctcccaga |
| Full - nt | ctacggagtgagctggattagacagcctcccggaaagggactggagtggatcggagtgatttggggtagcgaaac |
| | cacttactatcaatcttccctgaagtcacgggtcaccatttcaaaggataactcaaagaatcaagtgagcctcaagctc |
| | tcatcagtcaccgccgctgacaccgccgtgtattactgtgccaagcattactactatggagggtcctacgccatggac |
| | tactggggccagggaactctggtcactgtgtcatctggtggaggaggtagcggaggaggcgggagcggtggagg |
| | tggctccggaggcggtgggtcagaaatcgtgatgacccagagccctgcaaccctgtccctttctcccggggaacgg |
| | gctaccctttcttgtcgggcatcacaagatatctcaaaatacctcaattggtatcaacagaagccgggacaggcccct |
| | aggcttcttatctaccacacctctcgcctgcatagcgggattcccgcacgctttagcgggtctggaagcgggaccga |
| | ctacactctgaccatctcatctctccagcccgaggacttcgccgtctacttctgccagcagggtaacaccctgccgta |
| | caccttcggccagggcaccaagcttgagatcaaaaccactactcccgctccaaggccacccacccctgccccgac |
| | catcgcctctcagccgctttccctgcgtccggaggcatgtagacccgcagctggtggggccgtgcatacccggggt |
| | cttgacttcgcctgcgatatctacatttgggcccctctggctggtacttgcggggtcctgctgctttcactcgtgatcact |
| | ctttactgtaagcgcggtcggaagaagctgctgtacatctttaagcaacccttcatgaggcctgtgcagactactcaag |
| | aggaggacggctgttcatgccggttcccagaggaggaggaaggcggctgcgaactgcgcgtgaaattcagccgc |
| | agcgcagatgctccagcctacaagcaggggcagaaccagctctacaacgaactcaatcttggtcggagagagga |
| | gtacgacgtgctggacaagcggagaggacgggacccagaaatgggcgggaagccgcgcagaaagaatcccca |
| | agagggcctgtacaacgagctccaaaaggataagatggcagaagcctatagcgagattggtatgaaaggggaac |
| | gcagaagaggcaaaggccacgacggactgtaccagggactcagcaccgccaccaaggacacctatgacgctctt |
| | cacatgcaggccctgccgcctcgg (SEQ ID NO: 70) |
| 104882 | MALPVTALLLPLALLLHAARPqvqlqesgpglvkpsetlsltctvsgvslpdygvswirqppgk |
| CAR 8 - | glewigviwgsettyyqsslksrvtiskdnsknqvslklssvtaadtavyycakhyyggsyamdywgqgtl |
| Full - aa | vtvssggggsggggsggggsggggseivmtqspatlslspgeratlscrasqdiskylnwyqqkpgqaprlliy |
| | htsrlhsgiparfsgsgsgtdytltisslqpedfavyfcqqgntlpytfgqgtkleiktttpaprpptpaptiasqpls |
| | lrpeacrpaaggavhtrgldfacdiyiwaplagtcgvlllslvitlyckrgrkkllyifkqpfmrpvqttqeedgcsc |
| | rfpeeeeggcelrvkfsrsadapaykqgqnqlynelnlgrreeydvldkrrgrdpemggkprrknpqeglynel |
| | qkdkmaeayseigmkgerrrgkghdglyqglstatkdtydalhmqalppr (SEQ ID NO: 71) |

CAR 9

| | |
|---|---|
| 105974 | atggccctccctgtcaccgccctgctgcttccgctggctcttctgctccacgccgctcggcccgaaattgtgatgacc |
| CAR 9 - | cagtcacccgccactcttagcctttcacccggtgagcgcgcaaccctgtcttgcagagcctcccaagacatctcaaa |
| Full - nt | ataccttaattggtatcaacagaagcccggacaggctcctcgccttctgatctaccacaccagccggctccattctgg |
| | aatccctgccaggttcagcggtagcggatctgggaccgactacaccctcactatcagctcactgcagccagaggac |
| | ttcgctgtctatttctgtcagcaagggaacaccctgccctacacctttggacagggcaccaagctcgagattaaaggt |
| | ggaggtggcagcggaggaggtgggtccggcggtggaggaagcggaggcggtgggagccaggtccaactcca |
| | agaaagcggaccgggtcttgtgaagccatcagaaactctttcactgacttgtactgtgagcggagtgtctctccccga |
| | ttacggggtgtcttggatcagacagccaccggggaagggtctggaatggattggagtgatttggggctctgagacta |
| | cttactacaactcatccctcaagtcacgcgtcaccatctcaaaggacaactctaagaatcaggtgtcactgaaactgtc |
| | atctgtgaccgcagccgacaccgccgtgtactattgcgctaagcattactattatggcgggagctacgcaatggatta |
| | ctggggacagggtactctggtcaccgtgtccagcaccactaccccagcaccgaggccacccaccccggctcctac |
| | catcgcctcccagcctctgtccctgcgtccggaggcatgtagacccgcagctggtggggccgtgcatacccggggt |
| | cttgacttcgcctgcgatatctacatttgggcccctctggctggtacttgcggggtcctgctgctttcactcgtgatcact |
| | ctttactgtaagcgcggtcggaagaagctgctgtacatctttaagcaacccttcatgaggcctgtgcagactactcaag |
| | aggaggacggctgttcatgccggttcccagaggaggaggaaggcggctgcgaactgcgcgtgaaattcagccgc |
| | agcgcagatgctccagcctacaagcaggggcagaaccagctctacaacgaactcaatcttggtcggagagagga |
| | gtacgacgtgctggacaagcggagaggacgggacccagaaatgggcgggaagccgcgcagaaagaatcccca |
| | agagggcctgtacaacgagctccaaaaggataagatggcagaagcctatagcgagattggtatgaaaggggaac |
| | gcagaagaggcaaaggccacgacggactgtaccagggactcagcaccgccaccaaggacacctatgacgctctt |
| | cacatgcaggccctgccgcctcgg (SEQ ID NO: 72) |
| 105974 | MALPVTALLLPLALLLHAARPeivmtqspatlslspgeratlscrasqdiskylnwyqqkpgqa |
| CAR 9 - | prlliyhtsrlhsgiparfsgsgsgtdytltisslqpedfavyfcqqgntlpytfgqgtkleikggggsggggsggg |
| Full - aa | gsggggsqvqlqesgpglvkpsetlsltctvsgvslpdygvswirqppgkglewigviwgsettyynsslksrv |
| | tiskdnsknqvslklssvtaadtavyycakhyyyggsvamdywgqgtlvtvsstttpaprpptpaptiasqplsl |
| | rpeacrpaaggavhtrgldfacdiyiwaplagtcgvlllslvitlyckrgrkkllyifkqpfmrpvqttqeedgcscr |
| | fpeeeeggcelrvkfsrsadapaykqgqnqlynelnlgrreeydvldkrrgrdpemggkprrknpqeglynelq |
| | kdkmaeayseigmkgerrrgkghdglyqglstatkdtydalhmqalppr (SEQ ID NO: 73) |

CAR10

| | |
|---|---|
| 105975 | atggccctccctgtcaccgccctgctgcttccgctggctcttctgctccacgccgctcggcccgaaattgtgatgacc |
| CAR 10 | cagtcacccgccactcttagcctttcacccggtgagcgcgcaaccctgtcttgcagagcctcccaagacatctcaaa |
| Full - nt | ataccttaattggtatcaacagaagcccggacaggctcctcgccttctgatctaccacaccagccggctccattctgg |
| | aatccctgccaggttcagcggtagcggatctgggaccgactacaccctcactatcagctcactgcagccagaggac |
| | ttcgctgtctatttctgtcagcaagggaacaccctgccctacacctttggacagggcaccaagctcgagattaaaggt |
| | ggaggtggcagcggaggaggtgggtccggcggtggaggaagcggaggcggtgggagccaggtccaactcca |
| | agaaagcggaccgggtcttgtgaagccatcagaaactctttcactgacttgtactgtgagcggagtgtctctccccga |
| | ttacggggtgtcttggatcagacagccaccggggaagggtctggaatggattggagtgatttggggctctgagacta |
| | cttactacaactcatccctcaagtcacgcgtcaccatctcaaaggacaactctaagaatcaggtgtcactgaaactgtc |
| | atctgtgaccgcagccgacaccgccgtgtactattgcgctaagcattactattatggcgggagctacgcaatggatta |
| | ctggggacagggtactctggtcaccgtgtccagcaccactaccccagcaccgaggccacccaccccggctcctac |
| | catcgcctcccagcctctgtccctgcgtccggaggcatgtagacccgcagctggtggggccgtgcatacccggggt |
| | cttgacttcgcctgcgatatctacatttgggcccctctggctggtacttgcggggtcctgctgctttcactcgtgatcact |
| | ctttactgtaagcgcggtcggaagaagctgctgtacatctttaagcaacccttcatgaggcctgtgcagactactcaag |
| | aggaggacggctgttcatgccggttcccagaggaggaggaaggcggctgcgaactgcgcgtgaaattcagccgc |
| | agcgcagatgctccagcctacaagcaggggcagaaccagctctacaacgaactcaatcttggtcggagagagga |
| | gtacgacgtgctggacaagcggagaggacgggacccagaaatgggcgggaagccgcgcagaaagaatcccca |
| | agagggcctgtacaacgagctccaaaaggataagatggcagaagcctatagcgagattggtatgaaaggggaac |
| | gcagaagaggcaaaggccacgacggactgtaccagggactcagcaccgccaccaaggacacctatgacgctctt |
| | cacatgcaggccctgccgcctcgg (SEQ ID NO: 74) |
| 105975 | MALPVTALLLPLALLLHAARPEIVMTQSPATLSLSPGERATLSCRASQDIS |
| CAR 10 | KYLNWYQQKPGQAPRLLIYHTSRLHSGIPARFSGSGSGTDYTLTISSLQP |
| Full - aa | EDFAVYFCQQGNTLPYTFGQGTKLEIKGGGGSGGGGSGGGGSGGGGSQ |
| | VQLQESGPGLVKPSETLSLTCTVSGVSLPDYGVSWIRQPPGKGLEWIGVI |
| | WGSETTYYNSSLKSRVTISKDNSKNQVSLKLSSVTAADTAVYYCAKHY |
| | YYGGSYAMDYWGQGTLVTVSSTTTPAPRPPTPAPTIASQPLSLRPEACRP |
| | AAGGAVHTRGLDFACDIYIWAPLAGTCGVLLLSLVITLYCKRGRKKLLYI |
| | FKQPFMRPVQTTQEEDGCSCRFPEEEEGGCELRVKFSRSADAPAYKQGQ |
| | NQLYNELNLGRREEYDVLDKRRGRDPEMGGKPRRKNPQEGLYNELQKD |
| | KMAEAYSEIGMKGERRRGKGHDGLYQGLSTATKDTYDALHMQALPPR |
| | (SEQ ID NO: 75) |

CAR11

| | |
|---|---|
| 105976 | atggctctgcccgtgaccgcactcctcctgccactggctctgctgcttcacgccgctcgcccacaagtccagcttcaa |
| CAR 11 | gaatcagggcctggtctggtgaagccatctgagactctgtccctcacttgcaccgtgagcggagtgtccctcccaga |
| Full - nt | ctacggagtgagctggattagacagcctcccggaaagggactggagtggatcggagtgatttggggtagcgaaac |
| | cacttactataactcttccctgaagtcacgggtcaccatttcaaaggataactcaaagaatcaagtgagcctcaagctc |
| | tcatcagtcaccgccgctgacaccgccgtgtattactgtgccaagcattactactatggagggtcctacgccatggac |
| | tactggggccagggaactctggtcactgtgtcatctggtggaggaggtagcggaggaggcgggagcggtggagg |
| | tggctccggaggtggcggaagcgaaatcgtgatgacccagagccctgcaaccctgtccctttctcccggggaacg |
| | ggctaccctttcttgtcgggcatcacaagatatctcaaaatacctcaattggtatcaacagaagccgggacaggcccc |
| | taggcttcttatctaccacacctctcgcctgcatagcgggattcccgcacgctttagcgggtctggaagcgggaccga |
| | ctacactctgaccatctcatctctccagcccgaggacttcgccgtctacttctgccagcagggtaacaccctgccgta |
| | caccttcggccagggcaccaagcttgagatcaaaaccactactcccgctccaaggccacccacccctgccccgac |
| | catcgcctctcagccgctttccctgcgtccggaggcatgtagacccgcagctggtggggccgtgcatacccggggt |
| | cttgacttcgcctgcgatatctacatttgggcccctctggctggtacttgcggggtcctgctgctttcactcgtgatcact |
| | ctttactgtaagcgcggtcggaagaagctgctgtacatctttaagcaacccttcatgaggcctgtgcagactactcaag |
| | aggaggacggctgttcatgccggttcccagaggaggaggaaggcggctgcgaactgcgcgtgaaattcagccgc |
| | agcgcagatgctccagcctacaagcaggggcagaaccagctctacaacgaactcaatcttggtcggagagagga |
| | gtacgacgtgctggacaagcggagaggacgggacccagaaatgggcgggaagccgcgcagaaagaatcccca |
| | agagggcctgtacaacgagctccaaaaggataagatggcagaagcctatagcgagattggtatgaaaggggaac |
| | gcagaagaggcaaaggccacgacggactgtaccagggactcagcaccgccaccaaggacacctatgacgctctt |
| | cacatgcaggccctgccgcctcgg (SEQ ID NO: 76) |
| 105976 | MALPVTALLLPLALLLHAARPQVQLQESGPGLVKPSETLSLTCTVSGVSL |
| CAR 11 | PDYGVSWIRQPPGKGLEWIGVIWGSETTYYNSSLKSRVTISKDNSKNQV |
| Full - aa | SLKLSSVTAADTAVYYCAKHYYYGGSYAMDYWGQGTLVTVSSGGGGS |
| | GGGGSGGGGSGGGGSEIVMTQSPATLSLSPGERATLSCRASQDISKYLN |
| | WYQQKPGQAPRLLIYHTSRLHSGIPARFSGSGSGTDYTLTISSLQPEDFAV |
| | YFCQQGNTLPYTFGQGTKLEIKTTTPAPRPPTPAPTIASQPLSLRPEACRP |
| | AAGGAVHTRGLDFACDIYIWAPLAGTCGVLLLSLVITLYCKRGRKKLLYI |
| | FKQPFMRPVQTTQEEDGCSCRFPEEEEGGCELRVKFSRSADAPAYKQGQ |
| | NQLYNELNLGRREEYDVLDKRRGRDPEMGGKPRRKNPQEGLYNELQKD |
| | KMAEAYSEIGMKGERRRGKGHDGLYQGLSTATKDTYDALHMQALPPR |
| | (SEQ ID NO: 77) |

CAR12

| | |
|---|---|
| 105977 | atggccctccctgtcaccgccctgctgcttccgctggctcttctgctccacgccgctcggcccgaaattgtgatgacc |
| CAR 12 - | cagtcacccgccactcttagcctttcacccggtgagcgcgcaaccctgtcttgcagagcctcccaagacatctcaaa |
| Full - nt | ataccttaattggtatcaacagaagcccggacaggctcctcgccttctgatctaccacaccagccggctccattctgg |
| | aatccctgccaggttcagcggtagcggatctgggaccgactacaccctcactatcagctcactgcagccagaggac |
| | ttcgctgtctatttctgtcagcaagggaacaccctgccctacacctttggacagggcaccaagctcgagattaaaggt |
| | ggaggtggcagcggaggaggtgggtccggcggtggaggaagccaggtccaactccaagaaagcggaccgggt |
| | cttgtgaagccatcagaaactctttcactgacttgtactgtgagcggagtgtctctccccgattacggggtgtcttggat |
| | cagacagccaccggggaagggtctggaatggattggagtgatttggggctctgagactacttactacaactcatccc |
| | tcaagtcacgcgtcaccatctcaaaggacaactctaagaatcaggtgtcactgaaactgtcatctgtgaccgcagcc |
| | gacaccgccgtgtactattgcgctaagcattactattatggcgggagctacgcaatggattactggggacagggtact |
| | ctggtcaccgtgtccagcaccactaccccagcaccgaggccacccaccccggctcctaccatcgcctcccagcctc |
| | tgtccctgcgtccggaggcatgtagacccgcagctggtggggccgtgcatacccggggtcttgacttcgcctgcga |
| | tatctacatttgggcccctctggctggtacttgcggggtcctgctgctttcactcgtgatcactctttactgtaagcgcgg |
| | tcggaagaagctgctgtacatctttaagcaacccttcatgaggcctgtgcagactactcaagaggaggacggctgttc |
| | atgccggttcccagaggaggaggaaggcggctgcgaactgcgcgtgaaattcagccgcagcgcagatgctccag |
| | cctacaagcaggggcagaaccagctctacaacgaactcaatcttggtcggagagaggagtacgacgtgctggaca |
| | agcggagaggacgggacccagaaatgggcgggaagccgcgcagaaagaatccccaagagggcctgtacaacg |
| | agctccaaaaggataagatggcagaagcctatagcgagattggtatgaaaggggaacgcagaagaggcaaaggc |
| | cacgacggactgtaccagggactcagcaccgccaccaaggacacctatgacgctcttcacatgcaggccctgccg |
| | cctcgg (SEQ ID NO: 78) |
| 105977 | MALPVTALLLPLALLLHAARPEIVMTQSPATLSLSPGERATLSCRASQDIS |
| CAR 12 - | KYLNWYQQKPGQAPRLLIYHTSRLHSGIPARFSGSGSGTDYTLTISSLQP |
| Full - aa | EDFAVYFCQQGNTLPYTFGQGTKLEIKGGGGSGGGGSGGGGSQVQLQE |
| | SGPGLVKPSETLSLTCTVSGVSLPDYGVSWIRQPPGKGLEWIGVIWGSET |
| | TYYNSSLKSRVTISKDNSKNQVSLKLSSVTAADTAVYYCAKHYYYGGS |
| | YAMDYWGQGTLVTVSSTTTPAPRPPTPAPTIASQPLSLRPEACRPAAGGA |
| | VHTRGLDFACDIYIWAPLAGTCGVLLLSLVITLYCKRGRKKLLYIFKQPF |
| | MRPVQTTQEEDGCSCRFPEEEEGGCELRVKFSRSADAPAYKQGQNQLYN |
| | ELNLGRREEYDVLDKRRGRDPEMGGKPRRKNPQEGLYNELQKDKMAEA |
| | YSEIGMKGERRRGKGHDGLYQGLSTATKDTYDALHMQALPPR (SEQ ID |
| | NO: 79) |

CTL019

| | |
|---|---|
| CTL019 | atggccttaccagtgaccgccttgctcctgccgctggccttgctgctccacgccgccaggccggacatccagatgac |
| Full - nt | acagactacatcctccctgtctgcctctctgggagacagagtcaccatcagttgcagggcaagtcaggacattagtaa |
| | atatttaaattggtatcagcagaaaccagatggaactgttaaactcctgatctaccatacatcaagattacactcaggag |
| | tcccatcaaggttcagtggcagtgggtctggaacagattattctctcaccattagcaacctggagcaagaagatattgc |
| | cacttacttttgccaacagggtaatacgcttccgtacacgttcggaggggggaccaagctggagatcacaggtggc |
| | ggtggctcgggcggtggtgggtcgggtggcggcggatctgaggtgaaactgcaggagtcaggacctggcctggt |
| | ggcgccctcacagagcctgtccgtcacatgcactgtctcaggggtctcattacccgactatggtgtaagctggattcg |
| | ccagcctccacgaaagggtctggagtggctgggagtaatatggggtagtgaaaccacatactataattcagctctca |
| | aatccagactgaccatcatcaaggacaactccaagagccaagttttcttaaaaatgaacagtctgcaaactgatgaca |
| | cagccatttactactgtgccaaacattattactacggtggtagctatgctatggactactggggccaaggaacctcagt |
| | caccgtctcctcaaccacgacgccagcgccgcgaccaccaacaccggcgcccaccatcgcgtcgcagcccctgt |
| | ccctgcgcccagaggcgtgccggccagcggcggggggcgcagtgcacacgagggggctggacttcgcctgtg |
| | atatctacatctgggcgcccttggccgggacttgtggggtccttctcctgtcactggttatcaccctttactgcaaacgg |
| | ggcagaaagaaactcctgtatatattcaaacaaccatttatgagaccagtacaaactactcaagaggaagatggctgt |
| | agctgccgatttccagaagaagaagaaggaggatgtgaactgagagtgaagttcagcaggagcgcagacgcccc |
| | cgcgtacaagcagggccagaaccagctctataacgagctcaatctaggacgaagagaggagtacgatgttttggac |
| | aagagacgtggccgggaccctgagatggggggaaagccgagaaggaagaaccctcaggaaggcctgtacaatg |
| | aactgcagaaagataagatggcggaggcctacagtgagattgggatgaaaggcgagcgccggaggggcaaggg |
| | gcacgatggcctttaccagggtctcagtacagccaccaaggacacctacgacgcccttcacatgcaggccctgccc |
| | cctcgc (SEQ ID NO: 80) |
| CTL019 | MALPVTALLLPLALLLHAARPdiqmtqttsslsaslgdrvtiscrasqdiskylnwyqqkpdgtv |
| Full - aa | klliyhtsrlhsgvpsrfsgsgsgtdysltisnleqediatyfcqqgntlpytfgggtkleitggggsggggsggggs |
| | evklqesgpglvapsqslsvtctvsgvslpdygvswirqpprkglewlgviwgsettyynsalksrltiikdnsks |
| | qvflkmnslqtddtaiyycakhyyyggsyamdywgqgtsvtvsstttpaprpptpaptiasqplslrpeacrpaa |
| | ggavhtrgldfacdiyiwaplagtcgvlllslvitlyckrgrkkllyifkqpfmrpvqttqeedgcscrfpeeeegg |
| | celrvkfsrsadapaykqgqnqlynelnlgrreeydvldkrrgrdpemggkprrknpqeglynelqkdkmaea |
| | yseigmkgerrrgkghdglyqglstatkdtydalhmqalppr (SEQ ID NO: 81) |

CD19 Associated Diseases and/or Disorders

In one aspect, the disclosure provides methods for treating cancer, e.g., a cancer associated with CD19 expression, with a CAR-expressing cell (e.g., T cell, NK cell) therapy. Exemplary cancers include, but are not limited to e.g., one or more acute leukemias including but not limited to, e.g., B-cell acute lymphocytic leukemia (“B-ALL”), T-cell acute lymphocytic leukemia (“T-ALL”), acute lymphocytic leukemia (ALL); one or more chronic leukemias including but not limited to, e.g., chronic myelogenous leukemia (CML), chronic lymphocytic leukemia (CLL). Additional cancers or hematologic conditions associated with expression of CD19 include, but are not limited to, e.g., B cell promyelocytic leukemia, blastic plasmacytoid dendritic cell neoplasm, Burkitt lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further, a disease associated with CD19 expression include, but not limited to, e.g., atypical and/or non-classical cancers, malignancies, precancerous conditions or proliferative diseases associated with expression of CD19.

In one embodiment, the disclosure provides methods for treating CLL.

In another embodiment, the disclosure provides methods for treating ALL.

In another embodiment, the disclosure provides methods for treating B-cell ALL.

In one aspect, the disclosure provides methods of treating a responder (e.g., a complete responder and partial responder) having cancer (e.g., a hematological cancer such as ALL and CLL) with a CAR-expressing cell (e.g., T cell, NK cell) (e.g., a CD19 CAR-expressing cell (e.g., T cell, NK cell) as described herein, such as, e.g., CTL019). In an embodiment, the disclosure provides methods of treating a responder (e.g., a complete responder and partial responder) with a CAR-expressing cell (e.g., T cell, NK cell) in combination with another therapeutic agent, e.g., another therapeutic agent described herein (e.g., another CAR, e.g., another CAR described herein, an inhibitory CAR, e.g., an inhibitory CAR described herein, a kinase inhibitor (e.g., a kinase inhibitor described herein, e.g., an mTOR inhibitor, a BTK inhibitor), a checkpoint inhibitor, e.g., a checkpoint inhibitor described herein, a standard of care therapy, etc.). The combination can be, e.g., with any agent described herein. In an embodiment, after a CAR-expressing cell (e.g., T cell, NK cell) treatment, e.g., an initial CAR-expressing cell (e.g., T cell, NK cell) treatment, a partial responder is tested by any one of the methods described herein, such as, e.g., a CD19 CAR-expressing cell (e.g., T cell, NK cell) gene set signature, and if status has not changed and/or is down-graded to, e.g., a non-responder, then the subject is administered an alternative therapy, e.g., a standard of care for the particular cancer.

In one aspect, the disclosure provides methods of treating a non-responder having cancer (e.g., a hematological cancer such as ALL and CLL) with a CAR-expressing cell (e.g., T cell, NK cell) (e.g., a CD19 CAR-expressing cell (e.g., T cell, NK cell) as described herein, such as, e.g., CTL019). In an embodiment, the disclosure provides methods of treating a non-responder with a CAR-expressing cell (e.g., T cell, NK cell) in combination with another therapeutic agent, e.g., another therapeutic agent described herein (e.g., another CAR, e.g., another CAR described herein, an inhibitory CAR, e.g., an inhibitory CAR described herein, a kinase inhibitor (e.g., a kinase inhibitor described herein, e.g., an mTOR inhibitor, a BTK inhibitor), a checkpoint inhibitor, e.g., a checkpoint inhibitor described herein, a standard of care therapy, etc.). The combination can be, e.g., with any agent described herein. In an embodiment, after a CAR-expressing cell (e.g., T cell, NK cell) treatment, e.g., an initial CAR-expressing cell (e.g., T cell, NK cell) treatment, a non-responder is tested by any one of the methods described herein, such as, e.g., a CD19 CAR-expressing cell (e.g., T cell, NK cell) gene set signature, and if status has changed and/or is up-graded to, e.g., a partial-responder, e.g., a complete responder, then the subject is administered an alternative therapy described herein.

In an embodiment, the disclosure provides methods of treating cancer (e.g., a hematological cancer such as ALL or CLL) comprising steps of: (1) identifying a partial responder subject and/or non-responder subject, (2) administering to the partial responder subject and/or non-responder subject an mTOR inhibitor described herein, such as, e.g., RAD001 and rapamycin, e.g., at a dose and/or dosing schedule described herein; and (3) administering a CAR (e.g., a CD19 CAR described herein, such as, e.g., CTL019), e.g., subsequent to the administration of the mTOR inhibitor, thus treating the cancer. In an embodiment, the method further includes administering the mTOR inhibitor and/or the CAR in combination with one or more checkpoint inhibitors described here, such as, e.g., a PD1 inhibitor.

In an embodiment, the disclosure provides methods of treating cancer (e.g., a hematological cancer such as ALL or CLL) comprising steps of: (1) identifying a partial responder subject (e.g., patient) and/or non-responder subject (e.g., patient), (2) enriching the T cell population of the partial responder subject and/or non-responder subject by selecting for a less exhausted and/or more naïve T cell population, (3) introducing (e.g., by transforming, transducing, infecting, electroporating, etc.) a CAR (e.g., a CD19 CAR described herein, such as, e.g., CTL019) into said enriched T cell population thus transforming the subject's T cell population; and (4) administering the CAR-expressing T cell population into the partial responder subject and/or non-responder subject, thus treating the cancer.

In an embodiment, the disclosure provides methods of treating cancer (e.g., a hematological cancer such as ALL or CLL) comprising steps of: (1) identifying a partial responder subject (e.g., patient) and/or non-responder subject (e.g., patient), (2) reevaluating a partial responder subject and/or non-responder subject (e.g., patient) at a later time period for naïve T cells and/or less exhausted phenotype, and (3), e.g., if the subject has an increase in naïve T cells and/or a less exhausted phenotype, administering a CAR-expressing cell (e.g., T cell, NK cell) therapy (e.g., a CD19 CAR-expressing cell (e.g., T cell, NK cell) as described herein, such as, e.g., CTL019), thus treating the cancer. In an embodiment, a later time period comprises at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 1 day, at least 2 days, at least 3 days, at least 4 days, at least 5 days, at least 6 days, at least 1 week, at least 2 weeks, at least 3 weeks, at least 1 month, at least 2 months, at least 3 months, at least 4 months, at least 5 months, at least 6 months, at least 7 months at least 8 months, at least 9 months, at least 10 months, at least 11 months, at least 1 year, at least 2 years, at least 3 years, at least 4 years, at least 5 years, at least 6 years, at least 7 years, at least 8 years, at least 9 years, at least 10 years, or 11 years or more.

In an embodiment, the disclosure provides methods of treating cancer (e.g., a hematological cancer such as ALL or CLL) comprising the steps of (1) identifying partial responders and/or non-responders; and (2) treating with an alternative therapy, e.g., a standard of care for the particular cancer (e.g., the standard of care for ALL or CLL). In an embodiment, a partial responder is treated only with the standard of care (e.g., the standard of care for a hematological cancer such as ALL or CLL) in the absence of treatment with a CAR. In an embodiment, a non-responder is treated only with the standard of care (e.g., the standard of care for a hematological cancer such as ALL or CLL) in the absence of treatment with a CAR.

In an embodiment, standard of care for CLL includes, but is not limited to exemplary therapies described herein, e.g., described in Table A, and combinations thereof.

TABLE A
Exemplary therapies for CLL

| | w/o del | | |
|---|---|---|---|
| | (11q) or | del | del |
| | del(17p) | (17p) | (11q) |
| First line ≥ 70 yrs with comorbidities | | | |
| Obinutuzumab + chlorambucil | X | X | X |
| Rituxan + chlorambucil | X | | X |
| Rituxan | X | | |
| Chlorambucil | X | | |
| Fludarabine ± Rituxan | X | X | |
| Cladribine | X | | |
| Bendamustine ± Rituxan | X | | X |
| PCR (pentostatin, cyclophosphamide, Rituxan) | X | | X |
| First Line < 70 years without significant | | | |
| comorbidities | | | |
| FCR (Fludarabine, cyclophosphamide, Rituxan) | X | X | X |
| FR (Fludarabine, Rituxan) | X | X | |
| PCR | X | | X |
| Bendamustine ± Rituxan | X | | X |
| Obinutuzumab + chlorambucil | X | X | X |
| Second line- Relapsed/Refractory ≥ 70 years | | | |
| Imbruvica | X | X | X |
| Reduced-dose FCR | X | | X |
| Reduced-dose PCRR | X | | X |
| Bendamustine ± Rituxan | X | | X |
| Ofatumumab | X | X | X |
| Alemutuzumab + Rituxan | X | X | X |
| High dose methylprednisone (HDMP) + | X | X | X |
| rituximab | | | |
| Lenalidomide + Rituxan | X | X | X |
| Dose dense rituximab | X | | X |
| Second line- Relapsed/Refractory < years | | | |
| without significant comorbidities | | | |
| Imbruvica | X | X | X |
| FCR (Fludarabine, cyclophosphaide, Rituxan) | X | | X |
| PCR | X | | X |
| Bendamustine ± Rituxan | X | | X |
| Fludarabine + alemtuzumab | X | | X |
| R-CHOP (Rituxan, cyclophosphamide, | X | X | X |
| dosorubicin, vincristine, prednisone) | | | |
| Ofatumumab | X | X | X |
| OFAR (oxaliplatin, Fludara, cytarabine, Rituxan) | X | X | X |
| HDMP + rituximab | X | X | X |
| Lenalidomide + Rituxan | X | X | X |

In an embodiment, standard of care for CLL includes (1) radiation therapy, (2) chemotherapy, (3) surgery (e.g., removal of the spleen), (4) targeted therapy, (5) stem cell transplantation, and combinations thereof. In an embodiment, the standard of care comprises external radiation therapy. In an embodiment, the standard of care comprises internal radiation therapy (e.g., a radioactive substance sealed in needles, wires or catheters, for example, that are placed directly into or near the cancer).

In an embodiment, standard of care for ALL includes, but is not limited to exemplary therapies described herein, e.g., described in Table B, and combinations thereof

TABLE B
Exemplary therapies for ALL

First Line
RCHOP (Rituxan, cyclophosphamide, doxorubicin, vincristine, prednisone)
Dose dense RCHOP 14 (category 3)
Dose adjusted EPOCH (etoposide, prednisone, vincristine, cyclophosphamide,
doxorubicin) + Rituxan
First Line Therapy for subjects with Poor left ventricular function or very frail
RCEPP (rituximab, cyclophosphamide, etoposide, prednisone, procarbazine)
RCEOP (rituximab, cyclophosphamide, etoposide, vincristine, prednisone)
RCNOP (rituximab, cyclophosphamide, mitoxantrone, vincristine, prednisone)
RCEOP (rituximab, cyclophosphamide, etoposide, vincristine, prednisone)
Dose adjusted EPOCH (etoposide, prednisone, vincristine, cyclophosphamide,
doxorubicin) + Rituxan
Second line- proceed to high dose therapy with autologous stem cell rescue
DHAP (dexamethasone, cisplatin, cytarabine) ± Rituxan
ESHAP (etoposide, methylprednisolone, cytarabine, cisplatin) ± Rituxan
GDP (gemcitabine, dexamethasone, cisplatin) ± Rituxan
GemOx (gemcitabine, oxaliplatin) ± Rituxan
ICE (ifosfamide, carboplatin, etoposide) + Rituxan
MINE (mesna, ifosfamide, mitoxantrone, etoposide) ± Rituxan
Second-line therapy (non-candidates for high-dose therapy)
CEPP (cyclophosphamide, etoposide, prednisone, procarbazine) ± Rituxan
CEOP (cyclophosphamide, etoposide, vincristine, prednisone) ± Rituxan
DA-EPOCH ± Rituxan
Revlimid ± Rituxan
Rituxan
GemOx ± Rituxan
GDP ± Rituxan
Bendamustine + Rituxan

In an embodiment, standard of care for ALL includes (1) chemotherapy, (2) radiation therapy, (3) stem cell transplantation, (4) biological therapy, (5) targeted therapy, and combinations thereof.

In an embodiment, the standard of care includes, but is not limited to, fludarabine with cyclophosphamide (FC); fludarabine with rituximab (FR); fludarabine, cyclophosphamide, and rituximab (FCR); cyclophosphamide, doxorubicin, vincristine and prednisone (CHOP); and combinations thereof. General chemotherapeutic agents considered for use include, but are not limited to anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), vinorelbine (Navelbine®), and combinations thereof.

In an embodiment, chemotherapy comprises an antimetabolite, including, but not limited to, folic acid antagonists (also referred to herein as antifolates), pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), cytarabine liposomal (also known as Liposomal Ara-C, DepoCyt™); decitabine (Dacogen®); hydroxyurea (Hydrea®, Droxia™ and Mylocel™); mercaptopurine (Puri-Nethol®), pralatrexate (Folotyn™) capecitabine (Xeloda®), nelarabine (Arranon®), azacitidine (Vidaza®) and gemcitabine (Gemzar®). Suitable antimetabolites include, e.g., 5-fluorouracil (Adrucil®, Efudex®, Fluoroplex®), floxuridine (FUDF®), capecitabine (Xeloda®), pemetrexed (Alimta®), raltitrexed (Tomudex®) and gemcitabine (Gemzar®), and combinations thereof. In an embodiment, the purine analogue is fludarabine.

In an embodiment, chemotherapy comprises an alkylating agent including, but not limited to nitrogen mustards, ethylenimine derivatives, alkyl sulfonates, nitrosoureas and triazenes, uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®) and combinations thereof. Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); Bendamustine HCl (Treanda®) and combinations thereof. In an embodiment, the alkylating agent is bendamustine. In an embodiment, the alkylating agent is cyclophosphamide.

In an embodiment, the chemotherapeutic agent is a kinase inhibitor, e.g., a tyrosine kinase inhibitor including, but not limited to, erlotinib hydrochloride (Tarceva®); linifanib (N-[4-(3-amino-1H-indazol-4-yl)phenyl]-N-[2-fluoro-5-methylphenyl)urea, also known as ABT 869, available from Genentech); sunitinib malate (Sutent®); bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, and described in U.S. Pat. No. 6,780,996); dasatinib (Sprycel®); pazopanib (Votrient®); sorafenib (Nexavar®); zactima (ZD6474); and imatinib or imatinib mesylate (Gilvec® and Gleevec®). In one embodiment, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In one embodiment, the kinase inhibitor is a CDK4 inhibitor selected from aloisine A; flavopiridol or HMR-1275, 2-(2-chlorophenyl)-5,7-dihydroxy-8-[(3 S,4R)-3-hydroxy-1-methyl-4-piperidinyl]-4-chromenone; crizotinib (PF-02341066; 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00); 1-methyl-54-[2-[5-(trifluoromethyl)-1H-imidazol-2-yl]-4-pyridinyl]oxy]-N44-(trifluoromethyl)phenyl]-1H-benzimidazol-2-amine (RAF265); indisulam (E7070); roscovitine (CYC202); palbociclib (PD0332991); dinaciclib (SCH727965); N-[5-[[(5-tert-butyloxazol-2-yl)methyl]thio]thiazol-2-yl]piperidine-4-carboxamide (BMS 387032); 4-[[9-chloro-7-(2,6-difluorophenyl)-5H-pyrimido[5,4-d][2]benzazepin-2-yl]amino]-benzoic acid (MLN8054); 543-(4,6-difluoro-1H-benzimidazol-2-yl)-1H-indazol-5-yl]-N-ethyl-4-methyl-3-pyridinemethanamine (AG-024322); 4-(2,6-dichlorobenzoylamino)-1H-pyrazole-3-carboxylic acid N-(piperidin-4-yl)amide (AT7519); 4-[2-methyl-1-(1-methylethyl)-1H-imidazol-5-yl]-N-[4-(methylsulfonyl)phenyl]-2-pyrimidinamine (AZD5438); and XL281 (BMS908662). In one embodiment, the kinase inhibitor is an MNK inhibitor selected from CGP052088; 4-amino-3-(p-fluorophenylamino)-pyrazolo [3,4-d] pyrimidine (CGP57380); cercosporamide; ETC-1780445-2; and 4-amino-5-(4-fluoroanilino)-pyrazolo [3,4-d] pyrimidine.

In an embodiment, targeted therapy includes, but is not limited to an anti-CD20 antibody or functional fragment thereof, such as, e.g., rituximab (Riuxan® and MabThera®); tositumomab (Bexxar®); and ofatumumab (Arzerra®), and combinations thereof. In one embodiment, the targeted therapy includes, but is not limited to, an anti-CD52 antibody or functional fragment thereof such as, e.g., alemtuzumab (Campath®).

In an embodiment, biologic therapy comprises immunotherapy. Exemplary anthracyclines include, without limitation, doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; desacetylravidomycin and combinations thereof.

In an embodiment, stem cell transplantation comprises an autogeneic stem cell transplant. In an embodiment, stem cell transplantation comprises an allogenic stem cell transplant. In an embodiment, stem cell transplantation comprises allogeneic bone marrow transplantation. In an embodiment, stem cell transplantation comprises a hematopoietic stem cell transplantation (HSCT). In an embodiment, hematopoietic stem cells are derived from various tissues including, but not limited to bone marrow, peripheral blood, umbilical cord blood, and combinations thereof.

In an embodiment, the provided methods comprise determining if the subject is identified as having a statistically significant difference in expression level of one or more of a CD27 biomarker, a CD45RO biomarker, a CCR7 biomarker, a CD27 biomakrer, a HLA-DR biomarker, a CD95 biomarker, or a CD127 biomarker, relative to a reference level, and administering to the subject a therapeutically effective dose of a CAR-expressing cell, e.g., a T cell or NK cell. In an embodiment, a CAR-expressing cell (e.g., T cell, NK cell) is a CD19 CAR-expressing cell (e.g., T cell, NK cell) described herein such as, e.g., CTL019.

In one aspect, the disclosure provides methods for treating a disease associated with CD19 expression. In one aspect, the invention provides methods for treating a disease wherein part of the tumor is negative for CD19 and part of the tumor is positive for CD19. For example, provided methods are useful for treating subjects that have undergone treatment for a disease associated with elevated expression of CD19, wherein the subject that has undergone treatment for elevated levels of CD19 exhibits a disease associated with elevated levels of CD19.

In one aspect, provided methods comprise a vector comprising CD19 CAR operably linked to promoter for expression in mammalian cells (e.g., T cells or NK cells). In one aspect, provided methods comprise a recombinant cell (e.g., T cell or NK cell) expressing a CD19 CAR for use in treating CD19-expressing tumors, wherein the recombinant T cell expressing the CD19 CAR is termed a CD19 CAR-expressing cell. In one aspect, a CD19 CAR-expressing cell (e.g., T cell, NK cell) administered according to provided methods is capable of contacting a tumor cell with at least one CD19 CAR expressed on its surface such that the CAR-expressing cell targets the tumor cell and growth of the tumor is inhibited.

In one aspect, the disclosure features to a method of inhibiting growth of a CD19-expressing tumor cell, comprising contacting the tumor cell with a CD19 CAR-expressing cell (e.g., T cell, NK cell) described herein such that the CAR-expressing cell is activated in response to the antigen and targets the cancer cell, wherein the growth of the tumor is inhibited.

In one aspect, the disclosure includes a type of cellular therapy where T cells are genetically modified to express a CAR and the CAR-expressing cell (e.g., T cell, NK cell) is infused to a recipient in need thereof. The infused cell is able to kill tumor cells in the recipient. Unlike antibody therapies, CAR-modified cells (e.g., T cells or NK cells) are able to replicate in vivo resulting in long-term persistence that can lead to sustained tumor control. In various aspects, the cells administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the cell to the patient.

The disclosure also includes a type of cellular therapy where cells (e.g., T cells, NK cells) are modified, e.g., by in vitro transcribed RNA, to transiently express a chimeric antigen receptor (CAR) and the CAR-expressing cell (e.g., T cell, NK cell) is infused to a recipient in need thereof. The infused cell is able to kill tumor cells in the recipient. Thus, in various aspects, the cells administered to the patient, are present for less than one month, e.g., three weeks, two weeks, one week, after administration of the cell (e.g., T cell, NK cell) to the patient.

Without wishing to be bound by any particular theory, the anti-tumor immunity response elicited by the CAR-modified cells (e.g., T cells, NK cells) may be an active or a passive immune response, or alternatively may be due to a direct vs indirect immune response. In one aspect, the CAR transduced T cells exhibit specific proinflammatory cytokine secretion and potent cytolytic activity in response to human cancer cells expressing the CD19, resist soluble CD19 inhibition, mediate bystander killing and mediate regression of an established human tumor. For example, antigen-less tumor cells within a heterogeneous field of CD19-expressing tumor may be susceptible to indirect destruction by CD19-redirected T cells that has previously reacted against adjacent antigen-positive cancer cells.

In one aspect, the fully-human CAR-modified cells (e.g., T cells, NK cells) described herein may be a type of vaccine for ex vivo immunization and/or in vivo therapy in a mammal. In one aspect, the mammal is a human.

With respect to ex vivo immunization, at least one of the following occurs in vitro prior to administering the cell into a subject: i) expansion of the cells, ii) introducing a nucleic acid encoding a CAR to the cells or iii) cryopreservation of the cells.

Ex vivo procedures are well known in the art and are discussed more fully below. Briefly, cells are isolated from a subject (e.g., a human) and genetically modified (i.e., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

### Hematologic Cancer

Hematological cancer conditions are types of cancer such as leukemia and malignant lymphoproliferative conditions that affect blood, bone marrow and the lymphatic system.

Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CIVIL) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

Lymphoma is a group of blood cell tumors that develop from lymphocytes. Exemplary lymphomas include non-Hodgkin lymphoma and Hodgkin lymphoma.

The present disclosure provides for compositions and methods for treating cancer. In one aspect, the cancer is a hematologic cancer including but is not limited to a leukemia or a lymphoma. In one aspect, the CAR-expressing cells (e.g., T cells, NK cells) of the invention may be used to treat cancers and malignancies such as, but not limited to, e.g., acute leukemias including but not limited to, e.g., B-cell acute lymphoid leukemia (“B-ALL”), T-cell acute lymphoid leukemia (“T-ALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell promyelocytic leukemia, blastic plasmacytoid dendritic cell neoplasm, Burkitt lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further a disease associated with CD19 expression includes, but not limited to, e.g., atypical and/or non-classical cancers, malignancies, precancerous conditions or proliferative diseases expressing CD19.

The present disclosure also provides methods for inhibiting the proliferation or reducing a CD19-expressing cell population, the methods comprising contacting a population of cells comprising a CD19-expressing cell with a CD19 CAR-expressing cell (e.g., T cell, NK cell) described herein that binds to the CD19-expressing cell. In a specific aspect, the disclosure provides methods for inhibiting the proliferation or reducing the population of cancer cells expressing CD19, the methods comprising contacting the CD19-expressing cancer cell population with a CD19 CAR-expressing cell (e.g., T cell, NK cell) described herein that binds to the CD19-expressing cell. In one aspect, the present disclosure provides methods for inhibiting the proliferation or reducing the population of cancer cells expressing CD19, the methods comprising contacting the CD19-expressing cancer cell population with a CD19 CAR-expressing cell (e.g., T cell, NK cell) described herein that binds to the CD19-expressing cell. In certain aspects, the anti-CD19 CAR-expressing cell (e.g., T cell, NK cell) reduces the quantity, number, amount or percentage of cells and/or cancer cells by at least 25%, at least 30%, at least 40%, at least 50%, at least 65%, at least 75%, at least 85%, at least 95%, or at least 99% in a subject with or animal model for myeloid leukemia or another cancer associated with CD19-expressing cells relative to a negative control. In one aspect, the subject is a human.

The present disclosure also provides methods for preventing, treating and/or managing a disease associated with CD19-expressing cells (e.g., a hematologic cancer or atypical cancer expressing CD19), the methods comprising administering to a subject in need a CAR-expressing cell (e.g., T cell, NK cell) described herein that binds to the CD19-expressing cell. In one aspect, the subject is a human. Non-limiting examples of disorders associated with CD19-expressing cells include autoimmune disorders (such as lupus), inflammatory disorders (such as allergies and asthma) and cancers (such as hematological cancers or atypical cancers expressing CD19).

The present disclosure also provides methods for preventing, treating and/or managing a disease associated with CD19-expressing cells, the methods comprising administering to a subject in need a CD19 CAR-expressing cell (e.g., T cell, NK cell) described herein that binds to the CD19-expressing cell. In one aspect, the subject is a human.

The present disclosure provides methods for preventing relapse of cancer associated with CD19-expressing cells (e.g., a hematological cancer such as ALL and CLL), the methods comprising administering to a subject in need thereof a CD19 CAR-expressing cell (e.g., T cell, NK cell) described herein that binds to the CD19-expressing cell. In one aspect, the methods comprise administering to the subject in need thereof an effective amount of a CD19 CAR-expressing cell (e.g., T cell, NK cell) described herein that binds to the CD19-expressing cell in combination with an effective amount of another therapy.

### CAR Mediated Response

In some embodiments, response to a CAR-expressing cell therapy (e.g., a CAR19-expressing cell therapy), comprises a cellular response, e.g., a response mediated by cells, e.g., T cells, e.g., cytotoxic T cells, e.g., as described herein. In some embodiments, a cellular response comprises: (i) secretion of molecules (e.g., cytokines, chemokines or ligands), or (ii) cytotoxic activity, e.g., cell killing activity.

In some embodiments, response to a CAR-expressing cell therapy (e.g., a CAR19-expressing cell therapy), comprising a cellular response is, e.g., controlled or maintained, by one or more immune effector cells, e.g., one or more immune effector cells expressing a CAR therapy described herein, e.g., a CAR19-expressing cell therapy. In some embodiments, the one or more immune effector cells expressing the CAR therapy comprises T cells (e.g., CD8+ or CD4+ T cells) or NK cells. In some embodiments, the one or more immune effector cells expressing the CAR therapy comprises T cells, e.g., CD8+ or CD4+ T cells, which are activated, e.g., HLA-DR expressing CD8+ or CD4+ T cells. In some embodiments, the one or more immune effector cells expressing the CAR therapy comprises a T cell, e.g., a CD8+ or CD4+ T cell, e.g., a memory T cell (e.g., a memory stem cell (T_{SCM}), a central memory T cell (T_{CM}), or an effector memory T cell (T_{EM})). In some embodiments, the one or more immune effector cells expressing the CAR therapy comprises a CD4+ or a CD8+ effector memory T cell (T_{EM}). In some embodiments, a T_{EM} comprises one or more (or all) of the following markers: CD45RA−/+, CD45ROhigh, CD62L−, CCR7−, CD95+, CD122+, CD27−, CD28−, CD57+, KLRG-1high, short/intermediate telomere length (or any combination of two, three, four, five, six, seven, eight, nine, or all of the aforesaid T_{EM} markers).

Phenotypic markers associated with T_{SCM}, T_{CM} and T_{EM} are disclosed in, e.g., Maus, M. et al. (2014) Annu. Rev. Immunol. 32:189-225 (see for example, FIG. 3), incorporated by reference herein. In some embodiments, a T_{SCM} comprises one or more (or all) of the following markers: CD45RA+, CD45RO−, CD62Lhigh, CCR7high, CD95+, CD122+, CD27high, CD28 high, CD57−, KLRG-1−, or long telomere length (or any combination of two, three, four, five, six, seven, eight, nine, or all of the aforesaid TSCM markers). In some embodiments, a T_{CM} comprises one or more (or all) of the following markers: CD45RA−, CD45ROhigh, CD62Lhigh, CCR7+, CD95+, CD122 high, CD27+, CD28 high, CD57−, KLRG-1−/+, or long/intermediate telomere length (or any combination of two, three, four, five, six, seven, eight, nine, or all of the aforesaid T_{CM} markers). In some embodiments, a T_{EM} comprises one or more (or all) of the following markers: CD45RA−/+, CD45ROhigh, CD62L−, CCR7−, CD95+, CD122+, CD27−, CD28−, CD57+, KLRG-1high, short/intermediate telomere length (or any combination of two, three, four, five, six, seven, eight, nine, or all of the aforesaid T_{EM} markers).

In some embodiments, the one or more immune effector cells expressing the CAR therapy which, e.g., control or maintain, a response to the CAR-expressing cell therapy comprises a clonal or oligoclonal population of cells. In some embodiments, a clonal population comprises a population of cells, e.g., an expanded population of cells, originating from one cell, e.g., one cell expressing a CAR therapy. In some embodiments, an oligoclonal population comprises a population of cells, e.g., an expanded population of cells, originating from more than one, e.g., at least 2, 3, 4, 5 or more cells, e.g., about 5-5000 cells, expressing a CAR therapy. In some embodiments, each cell expressing a CAR therapy comprises one integration site, e.g., virus integration site, CAR encoding virus integration site.

In some embodiments, response to a CAR-expressing cell therapy, e.g., a CAR19-expressing cell therapy, comprises one or more phases of response, e.g., an early phase and/or a late phase. In some embodiments, the early phase occurs, e.g., at least about 1-30 days (e.g., about 1-5, 5-10, 10-15, 15-20, 20-25, 25-30 days) or about 1-6 months (e.g., about 1, 2, 3, 4, 5, or 6 months) after administration of the CAR-expressing cell therapy. In some embodiments, the late phase occurs, e.g., at least about e.g., at least about 1-60 days (e.g., about 1-10, 10-20, 20-30, 30-40, 40-50, or 50-60 days) or about 1-12 months (e.g., about 1, 2, 3, 4, 5, 6, 7, 8, 9, 10, 11, or 12 months) after administration of the CAR-expressing cell therapy. In some embodiments, the early phase occurs before, or is preceded by the late phase. In some embodiments, there is an overlap between the early phase and the late phase, e.g., a time period in when both phases occur. In some embodiments, the early phase occurs at least about e.g., at least about 1-30 days (e.g., about 1-5, 5-10, 10-15, 15-20, 20-25, 25-30 days) or about 1-6 months (e.g., about 1, 2, 3, 4, 5, or 6 months) before the commencement, e.g., start, of the late phase.

In some embodiments, response to a CAR-expressing cell therapy, e.g., a CAR19-expressing cell therapy, comprises an early phase. In some embodiments, the early phase occurs, e.g., at least about 1-30 days (e.g., about 1-5, 5-10, 10-15, 15-20, 20-25, 25-30 days) or about 1-6 months (e.g., about 1, 2, 3, 4, 5, or 6 months) after administration of the CAR-expressing cell therapy. In some embodiments, response to the early phase is, e.g., controlled or maintained, by one or more immune effector cells, e.g., one or more immune effector cells expressing a CAR therapy, e.g., a CAR19-expressing cell therapy. In some embodiments, the one or more immune effector cells expressing the CAR therapy comprise T cells, e.g., CD8+ T cells, which are activated, e.g., HLA-DR expressing CD8+ T cells. In some embodiments, the CD8+ T cells expressing the CAR therapy express markers associated with a T_{EM} phenotype, e.g., one or more (or all) of the following markers: CD45RA−/+, CD45ROhigh, CD62L−, CCR7−, CD95+, CD122+, CD27−, CD28−, CD57+, KLRG-1high, short/intermediate telomere length (or any combination of two, three, four, five, six, seven, eight, nine, or all of the aforesaid T_{EM} markers). In some embodiments, the CD8+ T cells expressing the CAR therapy express markers associated with a T_{CM} phenotype, e.g., one or more (or all) of the following markers: CD45RA−, CD45ROhigh, CD62Lhigh, CCR7+, CD95+, CD122 high, CD27+, CD28 high, CD57−, KLRG-1−/+, or long/intermediate telomere length (or any combination of two, three, four, five, six, seven, eight, nine, or all of the aforesaid T_{CM} markers). In some embodiments, the CD8+ T cells expressing the CAR therapy which, e.g., control or maintain, response to a CAR-expressing cell therapy expand in the early phase of response.

In some embodiments, response to a CAR-expressing cell therapy, e.g., a CAR19-expressing cell therapy, comprises a late phase. In some embodiments, the late phase occurs, e.g., at least about e.g., at least about 1-60 days (e.g., about 1-10, 10-20, 20-30, 30-40, 40-50, or 50-60 days) or about 1-12 months (e.g., about 1, 2, 3, 4, 5, 6, 7, 8, 9, 10, 11, or 12 months) after administration of the CAR-expressing cell therapy. In some embodiments, response to the late phase is, e.g., controlled or maintained, by one or more immune effector cells, e.g., one or more immune effector cells expressing a CAR therapy, e.g., a CAR19-expressing cell therapy. In some embodiments, the one or more immune effector cells expressing the CAR therapy comprises T cells, e.g., CD4+ T cells. In some embodiments, the CD4+ T cells expressing the CAR therapy express markers associated with a T_{EM} phenotype, e.g., one or more (or all) of the following markers: CD45RA−/+, CD45ROhigh, CD62L−, CCR7−, CD95+, CD122+, CD27−, CD28−, CD57+, KLRG-1high, short/intermediate telomere length (or any combination of two, three, four, five, six, seven, eight, nine, or all of the aforesaid T_{EM} markers). In some embodiments, the CD4+ T cells expressing the CAR therapy express markers associated with a T_{CM} phenotype, e.g., one or more (or all) of the following markers: CD45RA−, CD45ROhigh, CD62Lhigh, CCR7+, CD95+, CD122 high, CD27+, CD28 high, CD57−, KLRG-1−/+, or long/intermediate telomere length (or any combination of two, three, four, five, six, seven, eight, nine, or all of the aforesaid T_{CM} markers). In some embodiments, the CD4+ T cells expressing the CAR therapy which, e.g., control or maintain, response to a CAR-expressing cell therapy expand in the late phase of response.

In some embodiments, a CAR-expressing cell therapy described herein, e.g., a CAR19-expressing cell therapy described herein, results in remission, e.g., long-term remission, e.g., remission lasting at least about 2-20 years, e.g., at least about 3-19, 4-18, 5-17, 6-16, 7-15, 8-14, 9-13, 10-12 years, or at least about 2-5, 5-10, 10-15, or 15-20 years, in the subject. In some embodiments, a subject in remission has a response, e.g., a complete response, partial response or stable disease, to the CAR-expressing cell therapy. In some embodiments the subject has a hematological cancer described herein, e.g., a relapsed and or refractory hematological cancer e.g., ALL, CLL, NHL, or DLBCL.

In some embodiments, a subject who has, has been identified as having, or is predicted to have a response (e.g., a complete response, partial response or stable disease), to a CAR-expressing therapy, e.g., a CAR19-expressing therapy, has an increase, e.g., in number or fold, of functional and/or activated T cells (e.g., as described herein), prior to administration of the CAR-expressing therapy. In some embodiments, the increase in functional and/or activated T cells, is compared to a subject who does not have, or has not been identified as having a response to the CAR-expressing therapy. In some embodiments, an activated T cell is as described herein. In some embodiments, a functional T cell has one or more characteristics described herein. In some embodiments, the increase in functional and/or activated T cells, comprises about a 1-50 fold increase, e.g., about 1-5, 5-10, 10-15, 15-20, 20-30, 30-40, or 40-50 fold, increase in functional and/or activated T cells. In some embodiments, the increase in functional and/or activated T cells, comprises about a 5% or more increase, e.g., about 10, 20, 30, 40, 50, 60, 70, 80, 90% or 99% increase, in the number of functional and/or activated T cells. In some embodiments, the increase in functional and/or activated T cells is measured in a sample from the subject, e.g., a blood or bone marrow sample. In some embodiments, the increase in functional and/or activated T cells is measured according to an assay described in Example 6. In some embodiments, the increase in functional and/or activated T cells results is predictive of a response to the CAR-expressing cell therapy.

### Combination Therapy

It will be appreciated that any cancer therapy as described above and herein, can be administered in combination with one or more additional therapies to treat and/or reduce the symptoms of cancer described herein. The pharmaceutical compositions can be administered concurrently with, prior to, or subsequent to, one or more other additional therapies or therapeutic agents. In an embodiment, a CAR-expressing cell described herein may be used in combination with other known agents and therapies. Administered “in combination”, as used herein, means that two (or more) different treatments are delivered to the subject during the course of the subject affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

The CAR therapy and/or other therapeutic agents, procedures or modalities can be administered during periods of active disorder, or during a period of remission or less active disease. The CAR therapy can be administered before the other treatment, concurrently with the treatment, post-treatment, or during remission of the disorder.

When administered in combination, the CAR therapy and the additional agent (e.g., second or third agent), or all, can be administered in an amount or dose that is higher, lower or the same than the amount or dosage of each agent used individually, e.g., as a monotherapy. In certain embodiments, the administered amount or dosage of the CAR therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the CAR therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In further aspects, a CAR-expressing cell described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation peptide vaccine, such as that described in Izumoto et al. 2008 J NEUROSURG 108:963-971.

In one embodiment, a CAR-expressing cell described herein can be used in combination with a chemotherapeutic agent. Exemplary chemotherapeutic agents include those described in paragraphs 873-874 of International Application WO2015/142675, filed Mar. 13, 2015, which is herein incorporated by reference in its entirety, and combinations thereof.

Exemplary alkylating agents include, without limitation, those described in paragraph 875 of International Application WO2015/142675, filed Mar. 13, 2015, which is herein incorporated by reference in its entirety, and combinations thereof.

Exemplary mTOR inhibitors include, without limitation, RAD001, temsirolimus; ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.0^{4,9}] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-(SEQ ID NO: 91), inner salt (SF1126, CAS 936487-67-1), XL765 and combinations thereof.

Exemplary immunomodulators include, without limitation, those described in paragraph 882 of International Application WO2015/142675, filed Mar. 13, 2015, which is herein incorporated by reference in its entirety, and combinations thereof.

Exemplary anthracyclines include, without limitation, those described in paragraph 883 of International Application WO2015/142675, filed Mar. 13, 2015, which is herein incorporated by reference in its entirety, and combinations thereof.

Exemplary vinca alkaloids include, without limitation, those described in paragraph 884 of International Application WO2015/142675, filed Mar. 13, 2015, which is herein incorporated by reference in its entirety, and combinations thereof.

Exemplary proteosome inhibitors include, without limitation, those described in paragraph 884 of International Application WO2015/142675, filed Mar. 13, 2015, which is herein incorporated by reference in its entirety, and combinations thereof.

In some embodiments, a CAR-expressing cell described herein is administered in combination with an oncolytic virus. In embodiments, oncolytic viruses are capable of selectively replicating in and triggering the death of or slowing the growth of a cancer cell. In some cases, oncolytic viruses have no effect or a minimal effect on non-cancer cells. An oncolytic virus includes but is not limited to an oncolytic adenovirus, oncolytic Herpes Simplex Viruses, oncolytic retrovirus, oncolytic parvovirus, oncolytic vaccinia virus, oncolytic Sinbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)).

In an embodiment, cells expressing a CAR described herein are administered to a subject in combination with a molecule that decreases the Treg cell population. Methods that decrease the number of (e.g., deplete) Treg cells are known in the art and include, e.g., CD25 depletion, cyclophosphamide administration, modulating GITR function. Without wishing to be bound by theory, it is believed that reducing the number of Treg cells in a subject prior to apheresis or prior to administration of a CAR-expressing cell described herein reduces the number of unwanted immune cells (e.g., Tregs) in the tumor microenvironment and reduces the subject's risk of relapse.

In one embodiment, a CAR expressing cell described herein are administered to a subject in combination with a molecule targeting GITR and/or modulating GITR functions, such as a GITR agonist and/or a GITR antibody that depletes regulatory T cells (Tregs). In embodiments, cells expressing a CAR described herein are administered to a subject in combination with cyclophosphamide. In one embodiment, the GITR binding molecules and/or molecules modulating GITR functions (e.g., GITR agonist and/or Treg depleting GITR antibodies) are administered prior to administration of the CAR-expressing cell. For example, in one embodiment, the GITR agonist can be administered prior to apheresis of the cells. In embodiments, cyclophosphamide is administered to the subject prior to administration (e.g., infusion or re-infusion) of the CAR-expressing cell or prior to apheresis of the cells. In embodiments, cyclophosphamide and an anti-GITR antibody are administered to the subject prior to administration (e.g., infusion or re-infusion) of the CAR-expressing cell or prior to apheresis of the cells. In one embodiment, the subject has cancer (e.g., a solid cancer or a hematological cancer such as ALL or CLL). In an embodiment, the subject has CLL. In embodiments, the subject has ALL. In embodiments, the subject has a solid cancer, e.g., a solid cancer described herein. Exemplary GITR agonists include, without limitation, GITR fusion proteins and anti-GITR antibodies (e.g., bivalent anti-GITR antibodies) such as, e.g., a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In an embodiment, a CAR expressing cell described herein, such as, e.g., a CD19 CAR-expressing cell (e.g., T cell, NK cell), e.g., CTL019, is administered to a subject, e.g., a subject identified as a partial responder or non-responder, in combination with a GITR agonist, e.g., a GITR agonist described herein. In an embodiment, the GITR agonist is administered prior to the CAR-expressing cell. For example, in an embodiment, the GITR agonist can be administered prior to apheresis of the cells. In an embodiment, the subject has cancer (e.g., a hematological cancer such as ALL and CLL). In an embodiment, the subject has ALL. In an embodiment, the subject has CLL.

In an embodiment, a CAR expressing cell described herein, such as, e.g., a CD19 CAR-expressing cell (e.g., T cell, NK cell), e.g., CTL019 is administered to a subject, e.g., a subject identified as a partial responder or non-responder, in combination with an mTOR inhibitor, e.g., an mTOR inhibitor described herein, e.g., a target of the rapamycin signaling pathway such as RAD001. In an embodiment, the mTOR inhibitor is administered prior to the CAR-expressing cell. For example, in an embodiment, the mTOR inhibitor can be administered prior to apheresis of the cells. In an embodiment, the subject has cancer (e.g., a hematological cancer such as ALL and CLL). In an embodiment, the subject has ALL. In an embodiment, the subject has CLL.

### Kinase Inhibitor

In one embodiment, a CAR-expressing cell described herein may be used in a treatment regimen in combination with a kinase inhibitor, e.g., a CDK4 inhibitor, a BTK inhibitor, an MNK inhibitor, an mTOR inhibitor, an ITK inhibitor, etc. In one embodiment, the subject is a complete responder, and the subject is administered a treatment regimen that includes administration of a CAR-expressing cell described herein in combination with a kinase inhibitor, e.g., a kinase inhibitor described herein, e.g., at a dose or dosing schedule described herein. In one embodiment, the subject is a partial responder or a non-responder, and the subject is administered a treatment regimen that includes administration of a CAR-expressing cell described herein in combination with a kinase inhibitor, e.g., a kinase inhibitor described herein, e.g., at a dose or dosing schedule described herein.

In an embodiment, the kinase inhibitor is a CDK4 inhibitor, e.g., a CDK4 inhibitor described herein, e.g., a CDK4/6 inhibitor, such as, e.g., 6-Acetyl-8-cyclopentyl-5-methyl-2-(5-piperazin-1-yl-pyridin-2-ylamino)-8H-pyrido[2,3-d]pyrimidin-7-one, hydrochloride (also referred to as palbociclib or PD0332991). In one embodiment, the kinase inhibitor is a BTK inhibitor, e.g., a BTK inhibitor described herein, such as, e.g., ibrutinib. In one embodiment, the kinase inhibitor is an mTOR inhibitor, e.g., an mTOR inhibitor described herein, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor described herein. In one embodiment, the kinase inhibitor is a MNK inhibitor, e.g., a MNK inhibitor described herein, such as, e.g., 4-amino-5-(4-fluoroanilino)-pyrazolo [3,4-d] pyrimidine. The MNK inhibitor can be, e.g., a MNK1a, MNK1b, MNK2a and/or MNK2b inhibitor.

In one embodiment, the kinase inhibitor is a CDK4 inhibitor selected from aloisine A; flavopiridol or HMR-1275, 2-(2-chlorophenyl)-5,7-dihydroxy-8-[(3S,4R)-3-hydroxy-1-methyl-4-piperidinyl]-4-chromenone; crizotinib (PF-02341066; 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00); 1-methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265); indisulam (E7070); roscovitine (CYC202); palbociclib (PD0332991); dinaciclib (SCH727965); N45-[[(5-tert-butyloxazol-2-yl)methyl]thio]thiazol-2-yl]piperidine-4-carboxamide (BMS 387032); 4-[[9-chloro-7-(2,6-difluorophenyl)-5H-pyrimido[5,4-d][2]benzazepin-2-yl]amino]-benzoic acid (MLN8054); 543-(4,6-difluoro-1H-benzimidazol-2-yl)-1H-indazol-5-yl]-N-ethyl-4-methyl-3-pyridinemethanamine (AG-024322); 4-(2,6-dichlorobenzoylamino)-1H-pyrazole-3-carboxylic acid N-(piperidin-4-yl)amide (AT7519); 4-[2-methyl-1-(1-methylethyl)-1H-imidazol-5-yl]-N44-(methylsulfonyl)phenyl]-2-pyrimidinamine (AZD5438); and XL281 (BMS908662).

In one embodiment, the kinase inhibitor is a CDK4 inhibitor, e.g., palbociclib (PD0332991), and the palbociclib is administered at a dose of about 50 mg, 60 mg, 70 mg, 75 mg, 80 mg, 90 mg, 100 mg, 105 mg, 110 mg, 115 mg, 120 mg, 125 mg, 130 mg, 135 mg (e.g., 75 mg, 100 mg or 125 mg) daily for a period of time, e.g., daily for 14-21 days of a 28 day cycle, or daily for 7-12 days of a 21 day cycle. In one embodiment, 1, 2, 3, 4, 5, 6, 7, 8, 9, 10, 11, 12 or more cycles of palbociclib are administered.

In one embodiment, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13.

In one embodiment, the kinase inhibitor is a BTK inhibitor, e.g., ibrutinib (PCI-32765), and the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle, or daily for 28 day cycle. In one embodiment, 1, 2, 3, 4, 5, 6, 7, 8, 9, 10, 11, 12 or more cycles of ibrutinib are administered.

In one embodiment, the kinase inhibitor is an mTOR inhibitor selected from temsirolimus; ridaforolimus (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.0^{4,9}] hexatriaconta-16,24,26,28-tetraen-12-yl]propyl]-2-methoxycyclohexyl dimethylphosphinate, also known as AP23573 and MK8669; everolimus (RAD001); rapamycin (AY22989); simapimod; (5-{2,4-bis[(3S)-3-methylmorpholin-4-yl]pyrido[2,3-d]pyrimidin-7-yl}-2-methoxyphenyl)methanol (AZD8055); 2-amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126); and XL765.

In one embodiment, the kinase inhibitor is an mTOR inhibitor, e.g., rapamycin, and the rapamycin is administered at a dose of about 3 mg, 4 mg, 5 mg, 6 mg, 7 mg, 8 mg, 9 mg, 10 mg (e.g., 6 mg) daily for a period of time, e.g., daily for 21 day cycle, or daily for 28 day cycle. In one embodiment, 1, 2, 3, 4, 5, 6, 7, 8, 9, 10, 11, 12 or more cycles of rapamycin are administered. In one embodiment, the kinase inhibitor is an mTOR inhibitor, e.g., everolimus and the everolimus is administered at a dose of about 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg (e.g., 10 mg) daily for a period of time, e.g., daily for 28 day cycle. In one embodiment, 1, 2, 3, 4, 5, 6, 7, 8, 9, 10, 11, 12 or more cycles of everolimus are administered.

In one embodiment, the kinase inhibitor is an MNK inhibitor selected from CGP052088; 4-amino-3-(p-fluorophenylamino)-pyrazolo [3,4-d] pyrimidine (CGP57380); cercosporamide; ETC-1780445-2; and 4-amino-5-(4-fluoroanilino)-pyrazolo [3,4-d] pyrimidine.

In some embodiments of the methods, uses, and compositions herein, the BTK inhibitor is a BTK inhibitor described in International Application WO/2015/079417, which is herein incorporated by reference in its entirety. For instance, in some embodiments, the BTK inhibitor is a compound of formula (I) or a pharmaceutically acceptable salt thereof;

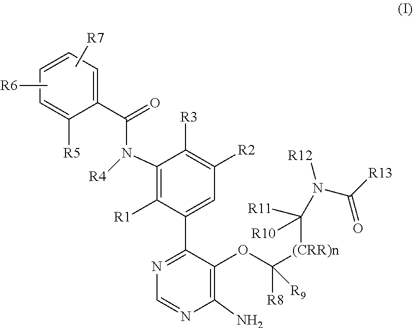

wherein,

R1 is hydrogen, C1-C6 alkyl optionally substituted by hydroxy;

R2 is hydrogen or halogen;

R3 is hydrogen or halogen;

R4 is hydrogen;

R5 is hydrogen or halogen;

or R4 and R5 are attached to each other and stand for a bond, —CH2-, —CH2-CH2-, —CH═CH—, —CH═CH—CH2-; —CH2-CH═CH—; or —CH2-CH2-CH2-;

R6 and R7 stand independently from each other for H, C1-C6 alkyl optionally substituted by hydroxyl, C3-C6 cycloalkyl optionally substituted by halogen or hydroxy, or halogen;

R8, R9, R, R′, R10 and R11 independently from each other stand for H, or C1-C6 alkyl optionally substituted by C1-C6 alkoxy; or any two of R8, R9, R, R′, R10 and R11 together with the carbon atom to which they are bound may form a 3-6 membered saturated carbocyclic ring;

R12 is hydrogen or C1-C6 alkyl optionally substituted by halogen or C1-C6 alkoxy; or R12 and any one of R8, R9, R, R′, R10 or R11 together with the atoms to which they are bound may form a 4, 5, 6 or 7 membered azacyclic ring, which ring may optionally be substituted by halogen, cyano, hydroxyl, C1-C6 alkyl or C1-C6 alkoxy;

n is 0 or 1; and

R13 is C2-C6 alkenyl optionally substituted by C1-C6 alkyl, C1-C6 alkoxy or N,N-di-C1-C6 alkyl amino; C2-C6 alkynyl optionally substituted by C1-C6 alkyl or C1-C6 alkoxy; or C2-C6 alkylenyl oxide optionally substituted by C1-C6 alkyl.

Low, Immune Enhancing, Dose of an mTOR Inhibitor

Methods described herein can use a low, immune enhancing, dose of an mTOR inhibitor e.g., an allosteric mTOR inhibitor, including rapalogs such as RAD001. Administration of a low, immune enhancing, dose of an mTOR inhibitor (e.g., a dose that is insufficient to completely suppress the immune system, but sufficient to improve immune function) can optimize the performance of immune effector cells, e.g., T cells or CAR-expressing cells, in the subject. Methods for measuring mTOR inhibition, dosages, treatment regimens, and suitable pharmaceutical compositions are described in U.S. Patent Application No. 2015/0140036, filed Nov. 13, 2014, hereby incorporated by reference.

In an embodiment, administration of a low, immune enhancing, dose of an mTOR inhibitor can result in one or more of the following:
i) a decrease in the number of PD-1 positive immune effector cells;
ii) an increase in the number of PD-1 negative immune effector cells;
iii) an increase in the ratio of PD-1 negative immune effector cells/PD-1 positive immune effector cells;
iv) an increase in the number of naive T cells;
v) an increase in the expression of one or more of the following markers: CD62L^{high}, CD127^{high}, CD27⁺, and BCL2, e.g., on memory T cells, e.g., memory T cell precursors;
vi) a decrease in the expression of KLRG1, e.g., on memory T cells, e.g., memory T cell precursors; or
vii) an increase in the number of memory T cell precursors, e.g., cells with any one or combination of the following characteristics: increased CD62L^{high}, increased CD127^{high}, increased CD27⁺, decreased KLRG1, and increased BCL2;
   and wherein any of the foregoing, e.g., i), ii), iii), iv), v), vi), or vii), occurs e.g., at least transiently, e.g., as compared to a non-treated subject.

In another embodiment, administration of a low, immune enhancing, dose of an mTOR inhibitor results in increased or prolonged proliferation of CAR-expressing cells, e.g., in culture or in a subject, e.g., as compared to non-treated CAR-expressing cells or a non-treated subject. In embodiments, increased proliferation is associated with in an increase in the number of CAR-expressing cells. In another embodiment, administration of a low, immune enhancing, dose of an mTOR inhibitor results in increased killing of cancer cells by CAR-expressing cells, e.g., in culture or in a subject, e.g., as compared to non-treated CAR-expressing cells or a non-treated subject. In embodiments, increased killing of cancer cells is associated with in a decrease in tumor volume.

In one embodiment, the cells expressing a CAR molecule, e.g., a CAR molecule described herein, are administered in combination with a low, immune enhancing dose of an mTOR inhibitor, e.g., an allosteric mTOR inhibitor, e.g., RAD001, or a catalytic mTOR inhibitor. For example, administration of the low, immune enhancing, dose of the mTOR inhibitor can be initiated prior to administration of a CAR-expressing cell described herein; completed prior to administration of a CAR-expressing cell described herein; initiated at the same time as administration of a CAR-expressing cell described herein; overlapping with administration of a CAR-expressing cell described herein; or continuing after administration of a CAR-expressing cell described herein.

Alternatively or in addition, administration of a low, immune enhancing, dose of an mTOR inhibitor can optimize immune effector cells to be engineered to express a CAR molecule described herein. In such embodiments, administration of a low, immune enhancing, dose of an mTOR inhibitor, e.g., an allosteric inhibitor, e.g., RAD001, or a catalytic inhibitor, is initiated or completed prior to harvest of immune effector cells, e.g., T cells or NK cells, to be engineered to express a CAR molecule described herein, from a subject.

In another embodiment, immune effector cells, e.g., T cells or NK cells, to be engineered to express a CAR molecule described herein, e.g., after harvest from a subject, or CAR-expressing immune effector cells, e.g., T cells or NK cells, e.g., prior to administration to a subject, can be cultured in the presence of a low, immune enhancing, dose of an mTOR inhibitor.

In an embodiment, administering to the subject a low, immune enhancing, dose of an mTOR inhibitor comprises administering, e.g., once per week, e.g., in an immediate release dosage form, 0.1 to 20, 0.5 to 10, 2.5 to 7.5, 3 to 6, or about 5, mgs of RAD001, or a bioequivalent dose thereof. In an embodiment, administering to the subject a low, immune enhancing, dose of an mTOR inhibitor comprises administering, e.g., once per week, e.g., in a sustained release dosage form, 0.3 to 60, 1.5 to 30, 7.5 to 22.5, 9 to 18, or about 15 mgs of RAD001, or a bioequivalent dose thereof.

In an embodiment, a dose of an mTOR inhibitor is associated with, or provides, mTOR inhibition of at least 5 but no more than 90%, at least 10 but no more than 90%, at least 15, but no more than 90%, at least 20 but no more than 90%, at least 30 but no more than 90%, at least 40 but no more than 90%, at least 50 but no more than 90%, at least 60 but no more than 90%, at least 70 but no more than 90%, at least 5 but no more than 80%, at least 10 but no more than 80%, at least 15, but no more than 80%, at least 20 but no more than 80%, at least 30 but no more than 80%, at least 40 but no more than 80%, at least 50 but no more than 80%, at least 60 but no more than 80%, at least 5 but no more than 70%, at least 10 but no more than 70%, at least 15, but no more than 70%, at least 20 but no more than 70%, at least 30 but no more than 70%, at least 40 but no more than 70%, at least 50 but no more than 70%, at least 5 but no more than 60%, at least 10 but no more than 60%, at least 15, but no more than 60%, at least 20 but no more than 60%, at least 30 but no more than 60%, at least 40 but no more than 60%, at least 5 but no more than 50%, at least 10 but no more than 50%, at least 15, but no more than 50%, at least 20 but no more than 50%, at least 30 but no more than 50%, at least 40 but no more than 50%, at least 5 but no more than 40%, at least 10 but no more than 40%, at least 15, but no more than 40%, at least 20 but no more than 40%, at least 30 but no more than 40%, at least 35 but no more than 40%, at least 5 but no more than 30%, at least 10 but no more than 30%, at least 15, but no more than 30%, at least 20 but no more than 30%, or at least 25 but no more than 30%.

The extent of mTOR inhibition can be conveyed as, or corresponds to, the extent of P70 S6 kinase inhibition, e.g., the extent of mTOR inhibition can be determined by the level of decrease in P70 S6 kinase activity, e.g., by the decrease in phosphorylation of a P70 S6 kinase substrate. The level of mTOR inhibition can be evaluated by various methods, such as measuring P70 S6 kinase activity by the Boulay assay, as described in U.S. Patent Application No. 2015/01240036, hereby incorporated by reference, or as described in U.S. Pat. No. 7,727,950, hereby incorporated by reference; measuring the level of phosphorylated S6 by western blot; or evaluating a change in the ratio of PD1 negative immune effector cells to PD1 positive immune effector cells.

As used herein, the term “mTOR inhibitor” refers to a compound or ligand, or a pharmaceutically acceptable salt thereof, which inhibits the mTOR kinase in a cell. In an embodiment, an mTOR inhibitor is an allosteric inhibitor. Allosteric mTOR inhibitors include the neutral tricyclic compound rapamycin (sirolimus), rapamycin-related compounds, that is compounds having structural and functional similarity to rapamycin including, e.g., rapamycin derivatives, rapamycin analogs (also referred to as rapalogs) and other macrolide compounds that inhibit mTOR activity. In an embodiment, an mTOR inhibitor is a catalytic inhibitor.

Rapamycin is a known macrolide antibiotic produced by Streptomyces hygroscopicus having the structure shown in Formula A.

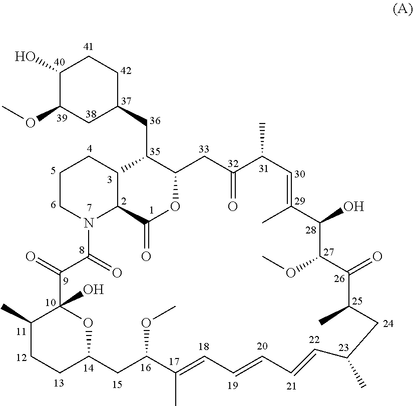 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

See, e.g., McAlpine, J. B., et al., J. Antibiotics (1991) 44: 688; Schreiber, S. L., et al., J. Am. Chem. Soc. (1991) 113: 7433; U.S. Pat. No. 3,929,992. There are various numbering schemes proposed for rapamycin. To avoid confusion, when specific rapamycin analogs are named herein, the names are given with reference to rapamycin using the numbering scheme of formula A.

Rapamycin analogs useful in the invention are, for example, O-substituted analogs in which the hydroxyl group on the cyclohexyl ring of rapamycin is replaced by OR₁ in which R₁ is hydroxyalkyl, hydroxyalkoxyalkyl, acylaminoalkyl, or aminoalkyl; e.g. RAD001, also known as, everolimus as described in U.S. Pat. No. 5,665,772 and WO94/09010 the contents of which are incorporated by reference. Other suitable rapamycin analogs include those substituted at the 26- or 28-position. The rapamycin analog may be an epimer of an analog mentioned above, particularly an epimer of an analog substituted in position 40, 28 or 26, and may optionally be further hydrogenated, e.g. as described in U.S. Pat. No. 6,015,815, WO95/14023 and WO99/15530 the contents of which are incorporated by reference, e.g. ABT578 also known as zotarolimus or a rapamycin analog described in U.S. Pat. No. 7,091,213, WO98/02441 and WO01/14387 the contents of which are incorporated by reference, e.g. AP23573 also known as ridaforolimus.

Examples of rapamycin analogs suitable for use in the present invention from U.S. Pat. No. 5,665,772 include, but are not limited to, 40-O-benzyl-rapamycin, 40-O-(4′-hydroxymethyl)benzyl-rapamycin, 40-O-[4′-(1,2-dihydroxyethyl)]benzyl-rapamycin, 40-O-allyl-rapamycin, 40-O-[3′-(2,2-dimethyl-1,3-dioxolan-4(S)-yl)-prop-2′-en-1′-yl]-rapamycin, (2′E,4'S)-40-O-(4′,5′-dihydroxypent-2′-en-1′-yl)-rapamycin, 40-O-(2-hydroxy)ethoxycarbonylmethyl-rapamycin, 40-O-(2-hydroxy)ethyl-rapamycin, 40-O-(3-hydroxy)propyl-rapamycin, 40-O-(6-hydroxy)hexyl-rapamycin, 40-O-[2-(2-hydroxy)ethoxy]ethyl-rapamycin, 40-O-[(3S)-2,2-dimethyldioxolan-3-yl]methyl-rapamycin, 40-O-[(2S)-2,3-dihydroxyprop-1-yl]-rapamycin, 40-O-(2-acetoxy)ethyl-rapamycin, 40-042-nicotinoyloxy)ethyl-rapamycin, 40-O-[2-(N-morpholino)acetoxy]ethyl-rapamycin, 40-O-(2-N-imidazolylacetoxy)ethyl-rapamycin, 40-O-[2-(N-methyl-N′-piperazinyl)acetoxy]ethyl-rapamycin, 39-O-desmethyl-39,40-O,O-ethylene-rapamycin, (26R)-26-dihydro-40-O-(2-hydroxy)ethyl-rapamycin, 40-O-(2-aminoethyl)-rapamycin, 40-O-(2-acetaminoethyl)-rapamycin, 40-O-(2-nicotinamidoethyl)-rapamycin, 40-O-(2-(N-methyl-imidazo-2′-ylcarbethoxamido)ethyl)-rapamycin, 40-O-(2-ethoxycarbonylaminoethyl)-rapamycin, 40-O-(2-tolylsulfonamidoethyl)-rapamycin and 40-O-[2-(4′,5′-dicarboethoxy-1′,2′,3′-triazol-1′-yl)-ethyl]-rapamycin.

Other rapamycin analogs useful in the present invention are analogs where the hydroxyl group on the cyclohexyl ring of rapamycin and/or the hydroxy group at the 28 position is replaced with an hydroxyester group are known, for example, rapamycin analogs found in U.S. RE44,768, e.g. temsirolimus.

Other rapamycin analogs useful in the preset invention include those wherein the methoxy group at the 16 position is replaced with another substituent, preferably (optionally hydroxy-substituted) alkynyloxy, benzyl, orthomethoxybenzyl or chlorobenzyl and/or wherein the mexthoxy group at the 39 position is deleted together with the 39 carbon so that the cyclohexyl ring of rapamycin becomes a cyclopentyl ring lacking the 39 position methyoxy group; e.g. as described in WO95/16691 and WO96/41807 the contents of which are incorporated by reference. The analogs can be further modified such that the hydroxy at the 40-position of rapamycin is alkylated and/or the 32-carbonyl is reduced.

Rapamycin analogs from WO95/16691 include, but are not limited to, 16-demthoxy-16-(pent-2-ynyl)oxy-rapamycin, 16-demthoxy-16-(but-2-ynyl)oxy-rapamycin, 16-demthoxy-16-(propargyl)oxy-rapamycin, 16-demethoxy-16-(4-hydroxy-but-2-ynyl)oxy-rapamycin, 16-demthoxy-16-benzyloxy-40-O-(2-hydroxyethyl)-rapamycin, 16-demthoxy-16-benzyloxy-rapamycin, 16-demethoxy-16-ortho-methoxybenzyl-rapamycin, 16-demethoxy-40-O-(2-methoxyethyl)-16-pent-2-ynyl)oxy-rapamycin, 39-demethoxy-40-desoxy-39-formyl-42-nor-rapamycin, 39-demethoxy-40-desoxy-39-hydroxymethyl-42-nor-rapamycin, 39-demethoxy-40-desoxy-39-carboxy-42-nor-rapamycin, 39-demethoxy-40-desoxy-39-(4-methyl-piperazin-1-yl)carbonyl-42-nor-rapamycin, 39-demethoxy-40-desoxy-39-(morpholin-4-yl)carbonyl-42-nor-rapamycin, 39-demethoxy-40-desoxy-39-[N-methyl, N-(2-pyridin-2-yl-ethyl)]carbamoyl-42-nor-rapamycin and 39-demethoxy-40-desoxy-39-(p-toluenesulfonylhydrazonomethyl)-42-nor-rapamycin.

Rapamycin analogs from WO96/41807 include, but are not limited to, 32-deoxo-rapamycin, 16-O-pent-2-ynyl-32-deoxo-rapamycin, 16-O-pent-2-ynyl-32-deoxo-40-O-(2-hydroxy-ethyl)-rapamycin, 16-O-pent-2-ynyl-32-(S)-dihydro-40-O-(2-hydroxyethyl)-rapamycin, 32(S)-dihydro-40-O-(2-methoxy)ethyl-rapamycin and 32(S)-dihydro-40-O-(2-hydroxyethyl)-rapamycin.

Another suitable rapamycin analog is umirolimus as described in US2005/0101624 the contents of which are incorporated by reference.

RAD001, otherwise known as everolimus (Afinitor®), has the chemical name (1R,9S,12S,15R,16E,18R,19R,21R,23 S,24E,26E,28E,30S,32S,35R)-1,18-dihydroxy-12-{(1R)-2-[(1 S,3R,4R)-4-(2-hydroxyethoxy)-3-methoxycyclohexyl]-1-methylethyl}-19,30-dimethoxy-15,17,21,23,29,35-hexamethyl-11,36-dioxa-4-aza-tricyclo[30.3.1.04,9]hexatriaconta-16,24,26,28-tetraene-2,3,10,14,20-pentaone

Further examples of allosteric mTOR inhibitors include sirolimus (rapamycin, AY-22989), 40-[3-hydroxy-2-(hydroxymethyl)-2-methylpropanoate]-rapamycin (also called temsirolimus or CCI-779) and ridaforolimus (AP-23573/MK-8669). Other examples of allosteric mTor inhibitors include zotarolimus (ABT578) and umirolimus.

Alternatively or additionally, catalytic, ATP-competitive mTOR inhibitors have been found to target the mTOR kinase domain directly and target both mTORC1 and mTORC2. These are also more effective inhibitors of mTORC1 than such allosteric mTOR inhibitors as rapamycin, because they modulate rapamycin-resistant mTORC1 outputs such as 4EBP1-T37/46 phosphorylation and cap-dependent translation.

Catalytic inhibitors include: BEZ235 or 2-methyl-244-(3-methyl-2-oxo-8-quinolin-3-yl-2,3-dihydro-imidazo[4,5-c]quinolin-1-yl)-phenyl]-propionitrile, or the monotosylate salt form. the synthesis of BEZ235 is described in WO2006/122806; CCG168 (otherwise known as AZD-8055, Chresta, C. M., et al., Cancer Res, 2010, 70(1), 288-298) which has the chemical name {5-[2,4-bis-((S)-3-methyl-morpholin-4-yl)-pyrido[2,3d]pyrimidin-7-yl]-2-methoxy-phenyl}-methanol; 3-[2,4-bis[(3 S)-3-methylmorpholin-4-yl]pyrido[2,3-d]pyrimidin-7-yl]-N-methylbenzamide (WO09104019); 3-(2-aminobenzo[d]oxazol-5-yl)-1-isopropyl-1H-pyrazolo[3,4-d]pyrimidin-4-amine (WO10051043 and WO2013023184); A N-(3-(N-(3-((3,5-dimethoxyphenyl)amino)quinoxaline-2-yl)sulfamoyl)phenyl)-3-methoxy-4-methylbenzamide (WO07044729 and WO12006552); PKI-587 (Venkatesan, A. M., J. Med. Chem., 2010, 53, 2636-2645) which has the chemical name 1-[4-[4-(dimethylamino)piperidine-1-carbonyl]phenyl]-3-[4-(4,6-dimorpholino-1,3,5-triazin-2-yl)phenyl]urea; GSK-2126458 (ACS Med. Chem. Lett., 2010, 1, 39-43) which has the chemical name 2,4-difluoro-N-{2-methoxy-5-[4-(4-pyridazinyl)-6-quinolinyl]-3-pyridinyl}benzenesulfonamide; 5-(9-isopropyl-8-methyl-2-morpholino-9H-purin-6-yl)pyrimidin-2-amine (WO10114484); (E)-N-(8-(6-amino-5-(trifluoromethyl)pyridin-3-yl)-1-(6-(2-cyanopropan-2-yl)pyridin-3-yl)-3-methyl-1H-imidazo[4,5-c]quinolin-2(3H)-ylidene)cyanamide (WO12007926).

Further examples of catalytic mTOR inhibitors include 8-(6-methoxy-pyridin-3-yl)-3-methyl-1-(4-piperazin-1-yl-3-trifluoromethyl-phenyl)-1,3-dihydro-imidazo[4,5-c]quinolin-2-one (WO2006/122806) and Ku-0063794 (Garcia-Martinez J M, et al., Biochem J., 2009, 421(1), 29-42. Ku-0063794 is a specific inhibitor of the mammalian target of rapamycin (mTOR).) WYE-354 is another example of a catalytic mTor inhibitor (Yu K, et al. (2009). Biochemical, Cellular, and In vivo Activity of Novel ATP-Competitive and Selective Inhibitors of the Mammalian Target of Rapamycin. Cancer Res. 69(15): 6232-6240).

mTOR inhibitors useful according to the present invention also include prodrugs, derivatives, pharmaceutically acceptable salts, or analogs thereof of any of the foregoing.

mTOR inhibitors, such as RAD001, may be formulated for delivery based on well-established methods in the art based on the particular dosages described herein. In particular, U.S. Pat. No. 6,004,973 (incorporated herein by reference) provides examples of formulations useable with the mTOR inhibitors described herein.

### Inhibitory Molecule Inhibitors/Checkpoint Inhibitors

In one embodiment, the subject can be administered an agent which enhances the activity of a CAR-expressing cell. For example, in one embodiment, the agent can be an agent which inhibits a checkpoint molecule. Checkpoint molecules, e.g., Programmed Death 1 (PD1), can, in some embodiments, decrease the ability of a CAR-expressing cell to mount an immune effector response. Examples of inhibitory molecules, e.g., checkpoint molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta). In embodiments, the CAR-expressing cell described herein comprises a switch costimulatory receptor, e.g., as described in WO 2013/019615, which is incorporated herein by reference in its entirety.

The methods described herein can include administration of a CAR-expressing cell in combination with a checkpoint inhibitor. In one embodiment, the subject is a complete responder. In another embodiment, the subject is a partial responder or non-responder, and, e.g., in some embodiments, the checkpoint inhibitor is administered prior to the CAR-expressing cell, e.g., two weeks, 12 days, 10 days, 8 days, one week, 6 days, 5 days, 4 days, 3 days, 2 days or 1 day before administration of the CAR-expressing cell. In some embodiments, the checkpoint inhibitor is administered concurrently with the CAR-expressing cell.

Inhibition of a checkpoint molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., a siRNA or shRNA, a clustered regularly interspaced short palindromic repeats (CRISPR), a transcription-activator like effector nuclease (TALEN), or a zinc finger endonuclease (ZFN), e.g., as described herein, can be used to inhibit expression of a checkpoint molecule in the CAR-expressing cell. In an embodiment, the inhibitor is a shRNA. In an embodiment, the checkpoint molecule is inhibited within a CAR-expressing cell. In these embodiments, a dsRNA molecule that inhibits expression of the checkpoint molecule is linked to the nucleic acid that encodes a component, e.g., all of the components, of the CAR.

In one embodiment, the inhibitor of an inhibitory signal can be, e.g., an antibody or antibody fragment that binds to a checkpoint molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206). In an embodiment, the agent is an antibody or antibody fragment that binds to TIM3. In an embodiment, the agent is an antibody or antibody fragment that binds to LAG3. In an embodiment, the agent is an antibody or antibody fragment that binds to CEACAM.

PD1 is an inhibitory member of the CD28 family of receptors that also includes CD28, CTLA-4, ICOS, and BTLA. PD1 is expressed on activated B cells, T cells and myeloid cells (Agata et al. 1996 INT. IMMUNoL 8:765-75). Two ligands for PD1, PD-L1 and PD-L2 have been shown to downregulate T cell activation upon binding to PD1 (Freeman et a. 2000 J Exp Med 192:1027-34; Latchman et al. 2001 NAT IMMUNOL 2:261-8; Carter et al. 2002 EUR J IMMUNOL 32:634-43). PD-L1 is abundant in human cancers (Dong et al. 2003 J MOL MED 81:281-7; Blank et al. 2005 CANCER IMMUNOL. IMMUNOTHER. 54:307-314; Konishi et al. 2004 CLIN CANCER RES 10:5094). Immune suppression can be reversed by inhibiting the local interaction of PD1 with PD-L1.

Antibodies, antibody fragments, and other inhibitors of PD1, PD-L1 and PD-L2 are available in the art and may be used combination with a CAR described herein, e.g., a CD19 CAR described herein. For example, nivolumab (also referred to as BMS-936558 or MDX1106; Bristol-Myers Squibb) is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and WO2006/121168. Pidilizumab (CT-011; Cure Tech) is a humanized IgG1k monoclonal antibody that binds to PD1 Pidilizumab and other humanized anti-PD1 monoclonal antibodies are disclosed in WO2009/101611. Lambrolizumab (also referred to as MK03475; Merck) is a humanized IgG4 monoclonal antibody that binds to PD1. Lambrolizumab and other humanized anti-PD1 antibodies are disclosed in U.S. Pat. No. 8,354,509 and WO2009/114335. MDPL3280A (Genentech/Roche) is a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906. Other anti-PD-L1 binding agents include YW243.55.570 (heavy and light chain variable regions are shown in SEQ ID NOs 20 and 21 in WO2010/077634) and MDX-1 105 (also referred to as BMS-936559, and, e.g., anti-PD-L1 binding agents disclosed in WO2007/005874). AMP-224 (B7-DCIg; Amplimmune; e.g., disclosed in WO2010/027827 and WO2011/066342), is a PD-L2 Fc fusion soluble receptor that blocks the interaction between PD1 and B7-H1. Other anti-PD1 antibodies include AMP 514 (Amplimmune), among others, e.g., anti-PD1 antibodies disclosed in U.S. Pat. No. 8,609,089, US 2010028330, and/or US 20120114649.

In one embodiment, the anti-PD-1 antibody or fragment thereof is an anti-PD-1 antibody molecule as described in US 2015/0210769, entitled “Antibody Molecules to PD-1 and Uses Thereof,” incorporated by reference in its entirety. In one embodiment, the anti-PD-1 antibody molecule includes at least one, two, three, four, five or six CDRs (or collectively all of the CDRs) from a heavy and light chain variable region from an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or encoded by the nucleotide sequence in Table 1, or a sequence substantially identical (e.g., at least 80%, 85%, 90%, 92%, 95%, 97%, 98%, 99% or higher identical) to any of the aforesaid sequences; or closely related CDRs, e.g., CDRs which are identical or which have at least one amino acid alteration, but not more than two, three or four alterations (e.g., substitutions, deletions, or insertions, e.g., conservative substitutions).

In yet another embodiment, the anti-PD-1 antibody molecule comprises at least one, two, three or four variable regions from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum 10, BAP049-hum 11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or encoded by the nucleotide sequence in Table 1; or a sequence substantially identical (e.g., at least 80%, 85%, 90%, 92%, 95%, 97%, 98%, 99% or higher identical) to any of the aforesaid sequences.

TIM3 (T cell immunoglobulin-3) also negatively regulates T cell function, particularly in IFN-g-secreting CD4+T helper 1 and CD8+T cytotoxic 1 cells, and plays a critical role in T cell exhaustion. Inhibition of the interaction between TIM3 and its ligands, e.g., galectin-9 (Gal9), phosphotidylserine (PS), and HMGB1, can increase immune response. Antibodies, antibody fragments, and other inhibitors of TIM3 and its ligands are available in the art and may be used combination with a CD19 CAR described herein. For example, antibodies, antibody fragments, small molecules, or peptide inhibitors that target TIM3 binds to the IgV domain of TIM3 to inhibit interaction with its ligands. Antibodies and peptides that inhibit TIM3 are disclosed in WO2013/006490 and US20100247521. Other anti-TIM3 antibodies include humanized versions of RMT3-23 (disclosed in Ngiow et al., 2011, Cancer Res, 71:3540-3551), and clone 8B.2C12 (disclosed in Monney et al., 2002, Nature, 415:536-541). Bi-specific antibodies that inhibit TIM3 and PD-1 are disclosed in US20130156774.

In one embodiment, the anti-TIM3 antibody or fragment thereof is an anti-TIM3 antibody molecule as described in US 2015/0218274, entitled “Antibody Molecules to TIM3 and Uses Thereof,” incorporated by reference in its entirety. In one embodiment, the anti-TIM3 antibody molecule includes at least one, two, three, four, five or six CDRs (or collectively all of the CDRs) from a heavy and light chain variable region from an antibody chosen from any of ABMTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences, or closely related CDRs, e.g., CDRs which are identical or which have at least one amino acid alteration, but not more than two, three or four alterations (e.g., substitutions, deletions, or insertions, e.g., conservative substitutions).

In yet another embodiment, the anti-TIM3 antibody molecule comprises at least one, two, three or four variable regions from an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1˜4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences.

In other embodiments, the agent which enhances the activity of a CAR-expressing cell is a CEACAM inhibitor (e.g., CEACAM-1, CEACAM-3, and/or CEACAM-5 inhibitor). In one embodiment, the inhibitor of CEACAM is an anti-CEACAM antibody molecule. Exemplary anti-CEACAM-1 antibodies are described in WO 2010/125571, WO 2013/082366 WO 2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10:1371/journal.pone.0021146), or crossreacts with CEACAM-1 and CEACAM-5 as described in, e.g., WO 2013/054331 and US 2014/0271618.

Without wishing to be bound by theory, carcinoembryonic antigen cell adhesion molecules (CEACAM), such as CEACAM-1 and CEACAM-5, are believed to mediate, at least in part, inhibition of an anti-tumor immune response (see e.g., Markel et al. J Immunol. 2002 Mar. 15; 168(6):2803-10; Markel et al. J Immunol. 2006 Nov. 1; 177(9):6062-71; Markel et al. Immunology. 2009 February; 126(2):186-200; Markel et al. Cancer Immunol Immunother. 2010 February; 59(2):215-30; Ortenberg et al. Mol Cancer Ther. 2012 June; 11(6):1300-10; Stern et al. J Immunol. 2005 Jun. 1; 174(11):6692-701; Zheng et al. PLoS One. 2010 Sep. 2; 5(9). pii: e12529). For example, CEACAM-1 has been described as a heterophilic ligand for TIM-3 and as playing a role in TIM-3-mediated T cell tolerance and exhaustion (see e.g., WO 2014/022332; Huang, et al. (2014) Nature doi:10.1038/nature13848). In embodiments, co-blockade of CEACAM-1 and TIM-3 has been shown to enhance an anti-tumor immune response in xenograft colorectal cancer models (see e.g., WO 2014/022332; Huang, et al. (2014), supra). In other embodiments, co-blockade of CEACAM-1 and PD-1 reduce T cell tolerance as described, e.g., in WO 2014/059251. Thus, CEACAM inhibitors can be used with the other immunomodulators described herein (e.g., anti-PD-1 and/or anti-TIM-3 inhibitors) to enhance an immune response against a cancer, e.g., a melanoma, a lung cancer (e.g., NSCLC), a bladder cancer, a colon cancer an ovarian cancer, and other cancers as described herein.

LAG3 (lymphocyte activation gene-3 or CD223) is a cell surface molecule expressed on activated T cells and B cells that has been shown to play a role in CD8+ T cell exhaustion. Antibodies, antibody fragments, and other inhibitors of LAG3 and its ligands are available in the art and may be used combination with a CD19 CAR described herein. For example, BMS-986016 (Bristol-Myers Squib) is a monoclonal antibody that targets LAG3. IMP701 (Immutep) is an antagonist LAG3 antibody and IMP731 (Immutep and GlaxoSmithKline) is a depleting LAG3 antibody. Other LAG3 inhibitors include IMP321 (Immutep), which is a recombinant fusion protein of a soluble portion of LAG3 and Ig that binds to MHC class II molecules and activates antigen presenting cells (APC). Other antibodies are disclosed, e.g., in WO2010/019570.

In some embodiments, the agent which enhances the activity of a CAR-expressing cell can be, e.g., a fusion protein comprising a first domain and a second domain, wherein the first domain is a checkpoint molecule, or fragment thereof, and the second domain is a polypeptide that is associated with a positive signal, e.g., a polypeptide comprising an intracellular signaling domain as described herein (also referred to herein as an inhibitory CAR or iCAR). In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the CAR. In another embodiment, the fusion protein is expressed by a cell, e.g., a T cell that does not express a CAR, e.g., a CD19 CAR.

In one embodiment, the extracellular domain (ECD) of a checkpoint molecule, e.g., a checkpoint molecule described herein such as, e.g., Programmed Death 1 (PD1), can be fused to a transmembrane domain and intracellular signaling domain described herein, e.g., an intracellular signaling domain comprising a costimulatory signaling domain such as, e.g., 41BB OX40, Cd28, CD27, and/or a primary signaling domain, e.g., of CD3 zeta. In one embodiment, the inhibitory CAR, e.g., e.g., PD1 CAR, can be used in combination with another CAR, e.g., CD19CAR (e.g., a CD19RCAR). In one embodiment, the PD1 RCAR (or PD1 CAR) improves the persistence of the T cell. Examples of inhibitory molecules include PD1, PD-L1, PD-L2, CTLA4, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (e.g., TGF beta). In one embodiment, the inhibitory molecule CAR comprises a first polypeptide, e.g., of an inhibitory molecule such as PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MEW class II, GAL9, adenosine, and TGF (e.g., TGF beta), or a fragment of any of these (e.g., at least a portion of an extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein).

In one embodiment, the inhibitory molecule CAR comprises the extracellular domain (ECD) of PD1 fused to a transmembrane domain and intracellular signaling domains such as 41BB and CD3 zeta (also referred to herein as a PD1 CAR). In one embodiment, the PD1 CAR improves the persistence of the cell CAR-expressing cell. In one embodiment, the PD1 CAR comprises the extracellular domain of PD1 indicated in SEQ ID NO: 44. In one embodiment, the PD1 CAR comprises, the amino acid sequence of SEQ ID NO:40.

In one embodiment, the PD1 CAR comprises the amino acid sequence provided as SEQ ID NO: 41.

In one embodiment, the PD1 CAR, e.g., the PD1 CAR described herein, is encoded by a nucleic acid sequence provided as SEQ ID NO: 42, or at least the comprises the nucleic acid sequence encoding the extracellular domain of PD1 (provided as SEQ ID NO: 101).

In embodiments, the inhibitory extracellular domain, has at least 70, 75, 80, 85, 90, 95, 96, 97, 98, or 99% identity with, or differs by no more than 30, 25, 20, 15, 10, 5, 4, 3, 2, or 1 amino acid residues from the corresponding residues of a naturally occurring human inhibitory molecule, e.g., a naturally occurring human primary stimulatory molecule disclosed herein.

In an embodiment, a nucleic acid molecule that encodes a dsRNA molecule that inhibits expression of the molecule that modulates or regulates, e.g., inhibits, T-cell function is operably linked to a promoter, e.g., a H1- or a U6-derived promoter such that the dsRNA molecule that inhibits expression of the molecule that modulates or regulates, e.g., inhibits, T-cell function is expressed, e.g., is expressed within a CAR-expressing cell. See e.g., Tiscornia G., “Development of Lentiviral Vectors Expressing siRNA,” Chapter 3, in Gene Transfer: Delivery and Expression of DNA and RNA (eds. Friedmann and Rossi). Cold Spring Harbor Laboratory Press, Cold Spring Harbor, N.Y., USA, 2007; Brummelkamp T R, et al. (2002) Science 296: 550-553; Miyagishi M, et al. (2002) Nat. Biotechnol. 19: 497-500. In an embodiment the nucleic acid molecule that encodes a dsRNA molecule that inhibits expression of the molecule that modulates or regulates, e.g., inhibits, T-cell function is present on the same vector, e.g., a lentiviral vector, that comprises a nucleic acid molecule that encodes a component, e.g., all of the components, of the CAR. In such an embodiment, the nucleic acid molecule that encodes a dsRNA molecule that inhibits expression of the molecule that modulates or regulates, e.g., inhibits, T-cell function is located on the vector, e.g., the lentiviral vector, 5′- or 3′- to the nucleic acid that encodes a component, e.g., all of the components, of the CAR. The nucleic acid molecule that encodes a dsRNA molecule that inhibits expression of the molecule that modulates or regulates, e.g., inhibits, T-cell function can be transcribed in the same or different direction as the nucleic acid that encodes a component, e.g., all of the components, of the CAR.

In an embodiment the nucleic acid molecule that encodes a dsRNA molecule that inhibits expression of the molecule that modulates or regulates, e.g., inhibits, T-cell function is present on a vector other than the vector that comprises a nucleic acid molecule that encodes a component, e.g., all of the components, of the CAR. In an embodiment, the nucleic acid molecule that encodes a dsRNA molecule that inhibits expression of the molecule that modulates or regulates, e.g., inhibits, T-cell function it transiently expressed within a CAR-expressing cell. In an embodiment, the nucleic acid molecule that encodes a dsRNA molecule that inhibits expression of the molecule that modulates or regulates, e.g., inhibits, T-cell function is stably integrated into the genome of a CAR-expressing cell. In an embodiment, the molecule that modulates or regulates, e.g., inhibits, T-cell function is PD-1.

In embodiments, the agent that enhances the activity of a CAR-expressing cell, e.g., inhibitor of an inhibitory molecule, is administered in combination with an allogeneic CAR, e.g., an allogeneic CAR described herein (e.g., described in the Allogeneic CAR section herein).

### Natural Killer Cell Receptor (NKR) CARs

In an embodiment, the CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), e.g., KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), e.g., NKp30, NKp44, NKp46; signaling lymphocyte activation molecule (SLAM) family of immune cell receptors, e.g., CD48, CD229, 2B4, CD84, NTB-A, CRACC, BLAME, and CD2F-10; Fc receptor (FcR), e.g., CD16, and CD64; and Ly49 receptors, e.g., LY49A, LY49C. The NKR-CAR molecules described herein may interact with an adaptor molecule or intracellular signaling domain, e.g., DAP12. Exemplary configurations and sequences of CAR molecules comprising NKR components are described in International Publication No. WO2014/145252, the contents of which are hereby incorporated by reference.

### Split CAR

In some embodiments, the CAR-expressing cell uses a split CAR. The split CAR approach is described in more detail in publications WO2014/055442 and WO2014/055657, incorporated herein by reference. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

### Strategies for Regulating Chimeric Antigen Receptors

There are many ways CAR activities can be regulated. In some embodiments, a regulatable CAR (RCAR) where the CAR activity can be controlled is desirable to optimize the safety and efficacy of a CAR therapy. There are many ways CAR activities can be regulated. For example, inducible apoptosis using, e.g., a caspase fused to a dimerization domain (see, e.g., Di Stasa et al., N Engl. J. Med. 2011 Nov. 3; 365(18):1673-1683), can be used as a safety switch in the CAR therapy of the instant invention. In one embodiment, the cells (e.g., T cells or NK cells) expressing a CAR of the present invention further comprise an inducible apoptosis switch, wherein a human caspase (e.g., caspase 9) or a modified version is fused to a modification of the human FKB protein that allows conditional dimerization. In the presence of a small molecule, such as a rapalog (e.g., AP 1903, AP20187), the inducible caspase (e.g., caspase 9) is activated and leads to the rapid apoptosis and death of the cells (e.g., T cells or NK cells) expressing a CAR of the present invention. Examples of a caspase-based inducible apoptosis switch (or one or more aspects of such a switch) have been described in, e.g., US2004040047; US20110286980; US20140255360; WO1997031899; WO2014151960; WO2014164348; WO2014197638; WO2014197638; all of which are incorporated by reference herein.

In another example, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, e.g., Song et al. Cancer Gene Ther. 2008; 15(10):667-75; Clinical Trial Id. No. NCT02107963; and Di Stasi et al. N. Engl. J. Med. 2011; 365:1673-83.

Alternative strategies for regulating the CAR therapy of the instant invention include utilizing small molecules or antibodies that deactivate or turn off CAR activity, e.g., by deleting CAR-expressing cells, e.g., by inducing antibody dependent cell-mediated cytotoxicity (ADCC). For example, CAR-expressing cells described herein may also express an antigen that is recognized by molecules capable of inducing cell death, e.g., ADCC or complement-induced cell death. For example, CAR expressing cells described herein may also express a receptor capable of being targeted by an antibody or antibody fragment. Examples of such receptors include EpCAM, VEGFR, integrins (e.g., integrins ανβ3, α4, αI3/4β3, α4β7, α5β1, ανβ3, αν), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/IgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

For example, a CAR-expressing cell described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the CAR-expressing cells (see, e.g., WO2011/056894, and Jonnalagadda et al., Gene Ther. 2013; 20(8)853-860). Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., Blood. 2014; 124(8)1277-1287). Other methods for depleting CAR-expressing cells described herein include administration of CAMPATH, a monoclonal anti-CD52 antibody that selectively binds and targets mature lymphocytes, e.g., CAR-expressing cells, for destruction, e.g., by inducing ADCC. In other embodiments, the CAR-expressing cell can be selectively targeted using a CAR ligand, e.g., an anti-idiotypic antibody. In some embodiments, the anti-idiotypic antibody can cause effector cell activity, e.g., ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

In other embodiments, a CAR-expressing cell described herein may also express a target protein recognized by the T cell depleting agent. In one embodiment, the target protein is CD20 and the T cell depleting agent is an anti-CD20 antibody, e.g., rituximab. In such embodiment, the T cell depleting agent is administered once it is desirable to reduce or eliminate the CAR-expressing cell, e.g., to mitigate the CAR induced toxicity. In other embodiments, the T cell depleting agent is an anti-CD52 antibody, e.g., alemtuzumab, as described in the Examples herein.

In other embodiments, an RCAR comprises a set of polypeptides, typically two in the simplest embodiments, in which the components of a standard CAR described herein, e.g., an antigen binding domain and an intracellular signalling domain, are partitioned on separate polypeptides or members. In some embodiments, the set of polypeptides include a dimerization switch that, upon the presence of a dimerization molecule, can couple the polypeptides to one another, e.g., can couple an antigen binding domain to an intracellular signalling domain. In one embodiment, a CAR of the present invention utilizes a dimerization switch as those described in, e.g., WO2014127261, which is incorporated by reference herein. Additional description and exemplary configurations of such regulatable CARs are provided herein and in, e.g., paragraphs 527-551 of International Publication No. WO 2015/090229 filed Mar. 13, 2015, which is incorporated by reference in its entirety. In some embodiments, an RCAR involves a switch domain, e.g., a FKBP switch domain, as set out SEQ ID NO: 92, or comprise a fragment of FKBP having the ability to bind with FRB, e.g., as set out in SEQ ID NO: 93. In some embodiments, the RCAR involves a switch domain comprising a FRB sequence, e.g., as set out in SEQ ID NO: 94, or a mutant FRB sequence, e.g., as set out in any of SEQ ID Nos. 95-100.

Co-Expression of CAR with a Chemokine Receptor

In embodiments, the CAR-expressing cell described herein further comprises a chemokine receptor molecule. Transgenic expression of chemokine receptors CCR2b or CXCR2 in T cells enhances trafficking to CCL2- or CXCL1-secreting solid tumors including melanoma and neuroblastoma (Craddock et al., J Immunother. 2010 October; 33(8):780-8 and Kershaw et al., Hum Gene Ther. 2002 Nov. 1; 13(16):1971-80). Thus, without wishing to be bound by theory, it is believed that chemokine receptors expressed in CAR-expressing cells that recognize chemokines secreted by tumors, e.g., solid tumors, can improve homing of the CAR-expressing cell to the tumor, facilitate the infiltration of the CAR-expressing cell to the tumor, and enhances antitumor efficacy of the CAR-expressing cell. The chemokine receptor molecule can comprise a naturally occurring or recombinant chemokine receptor or a chemokine-binding fragment thereof. A chemokine receptor molecule suitable for expression in a CAR-expressing cell described herein include a CXC chemokine receptor (e.g., CXCR1, CXCR2, CXCR3, CXCR4, CXCR5, CXCR6, or CXCR7), a CC chemokine receptor (e.g., CCR1, CCR2, CCR3, CCR4, CCR5, CCR6, CCR7, CCR8, CCR9, CCR10, or CCR11), a CX3C chemokine receptor (e.g., CX3CR1), a XC chemokine receptor (e.g., XCR1), or a chemokine-binding fragment thereof. In one embodiment, the chemokine receptor molecule to be expressed with a CAR described herein is selected based on the chemokine(s) secreted by the tumor. In one embodiment, the CAR-expressing cell described herein further comprises, e.g., expresses, a CCR2b receptor or a CXCR2 receptor. In an embodiment, the CAR described herein and the chemokine receptor molecule are on the same vector or are on two different vectors. In embodiments where the CAR described herein and the chemokine receptor molecule are on the same vector, the CAR and the chemokine receptor molecule are each under control of two different promoters or are under the control of the same promoter.

### Split CAR

In some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

### Pharmaceutical Compositions and Treatments

Pharmaceutical compositions may comprise a CAR-expressing cell, e.g., a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions can be, e.g., formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., a contaminant described in paragraph 1009 of International Application WO2015/142675, filed Mar. 13, 2015, which is herein incorporated by reference in its entirety.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the immune effector cells (e.g., T cells, NK cells) described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., NEW ENG. J. OF MED. 319:1676, 1988).

In some embodiments, a dose of CAR cells (e.g., CD19 CAR cells) comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises about 1.1×10⁶-1.8×10⁷ cells/kg. In some embodiments, a dose of CAR cells (e.g., CD19 CAR cells) comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 CAR cells) comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 CAR cells) comprises up to about 1×10⁷, 2×10⁷, 5×10⁷, 1×10⁸, 2×10⁸, 5×10⁸, 1×10⁹, 2×10⁹, or 5×10⁹ cells.

In certain aspects, it may be desired to administer activated immune effector cells (e.g., T cells, NK cells) to a subject and then subsequently redraw blood (or have an apheresis performed), activate immune effector cells (e.g., T cells, NK cells) therefrom according to the present disclosure, and reinfuse the patient with these activated and expanded immune effector cells (e.g., T cells, NK cells). This process can be carried out multiple times every few weeks. In certain aspects, immune effector cells (e.g., T cells, NK cells) can be activated from blood draws of from 10 cc to 400 cc. In certain aspects, immune effector cells (e.g., T cells, NK cells) are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the T cell compositions described herein are administered to a patient by intradermal or subcutaneous injection. In one aspect, the T cell compositions described herein are administered by i.v. injection. The compositions of immune effector cells (e.g., T cells, NK cells) may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more CAR constructs described herein may be introduced, thereby creating a CAR T cell of the present disclosure. Subjects in need thereof may subsequently undergo standard treatment with high dose chemotherapy followed by peripheral blood stem cell transplantation. In certain aspects, following or concurrent with the transplant, subjects receive an infusion of the expanded CAR T cells described herein. In an additional aspect, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. A suitable daily dose is 1 to 10 mg per day although in some instances larger doses of up to 40 mg per day may be used (described in U.S. Pat. No. 6,120,766).

In one embodiment, the CAR is introduced into immune effector cells (e.g., T cells, NK cells), e.g., using in vitro transcription, and the subject (e.g., human) receives an initial administration of CAR immune effector cells (e.g., T cells, NK cells) of the invention, and one or more subsequent administrations of the CAR immune effector cells (e.g., T cells, NK cells) of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR immune effector cells (e.g., T cells, NK cells) described herein are administered to the subject (e.g., human) per week, e.g., 2, 3, or 4 administrations of the CAR immune effector cells (e.g., T cells, NK cells) of the invention are administered per week. In one embodiment, the subject (e.g., human subject) receives more than one administration of the CAR immune effector cells (e.g., T cells, NK cells) per week (e.g., 2, 3 or 4 administrations per week) (also referred to herein as a cycle), followed by a week of no CAR immune effector cells (e.g., T cells, NK cells) administrations, and then one or more additional administration of the CAR immune effector cells (e.g., T cells, NK cells) (e.g., more than one administration of the CAR immune effector cells (e.g., T cells, NK cells) per week) is administered to the subject. In another embodiment, the subject (e.g., human subject) receives more than one cycle of CAR immune effector cells (e.g., T cells, NK cells), and the time between each cycle is less than 10, 9, 8, 7, 6, 5, 4, or 3 days. In one embodiment, the CAR immune effector cells (e.g., T cells, NK cells) are administered every other day for 3 administrations per week. In one embodiment, the CAR immune effector cells (e.g., T cells, NK cells) described herein are administered for at least two, three, four, five, six, seven, eight or more weeks.

In one aspect, CAR-expressing cells (e.g., T cells, NK cells) as described herein such as, e.g., CD19 CAR-expressing cells, e.g., CTL019 are generated using lentiviral viral vectors, such as lentivirus. CAR-expressing cells generated that way can have stable CAR expression.

In one aspect, CAR-expressing cells (e.g., T cells, NK cells) transiently express CAR vectors for 4, 5, 6, 7, 8, 9, 10, 11, 12, 13, 14, 15 days after transduction. Transient expression of CARs can be effected by RNA CAR vector delivery. In one aspect, the CAR RNA is transduced into the cell by electroporation.

A potential issue that can arise in patients being treated using transiently expressing CAR cells, e.g., T cells (particularly with murine scFv bearing CAR-expressing cells (e.g., T cells, NK cells)) is anaphylaxis after multiple treatments. Without being bound by this theory, it is believed that such an anaphylactic response might be caused by a patient developing humoral anti-CAR response, i.e., anti-CAR antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-CAR antibody response during the course of transient CAR therapy (such as those generated by RNA transductions), CAR-expressing cell (e.g., T cell, NK cell) infusion breaks should not last more than ten to fourteen days.

In some embodiments of any of the aforesaid methods, the method further includes administering one or more doses of a cell (e.g., an immune cell containing a CAR nucleic acid or CAR polypeptide as described herein), to a mammal (e.g., a mammal having a cancer, e.g., a mammal that is or is identified as being a responder, complete responder, partial responder, non-responder, relapser, or non-relapser according to the methods herein). In some embodiments, the one or more doses of CAR cells (e.g., CD19 CAR cells) comprises at least about 1×10⁶, 5×10⁶, 1×10⁷, 2×10⁷, 5×10⁷, 1×10⁸, 2×10⁸, 5×10⁸, 1×10⁹, 2×10⁹, or 5×10⁹ cells.

In one embodiment, up to 10, 9, 8, 7, 6, 5, 4, 3, or 2 doses of cells are administered. In other embodiments, one, two, three, four, five or 6 doses of the cells are administered to the mammal, e.g., in a treatment interval of one, two, three, four or more weeks. In one embodiment, up to 6 doses are administered in two weeks. The doses may the same or different. In one embodiment, a lower dose is administered initially, followed by one or more higher doses. In one exemplary embodiment, the lower dose is about 1×10⁵ to 1×10⁹ cells/kg, or 1×10⁶ to 1×10⁸ cells/kg; and the higher dose is about 2×10⁵ to 2×10⁹ cells/kg or 2×10⁶ to 2×10⁸ cells/kg, followed by 3-6 doses of about 4×10⁵ to 4×10⁹ cells/kg, or 4×10⁶ to 4×10⁸ cells/kg.

In some embodiments, the CAR-expressing cell therapy, e.g., CAR19 expressing cell therapy, comprises a plurality of cells. In some embodiments, the CAR-expressing cell therapy, e.g., CAR19 expressing cell therapy, is administered in a single infusion or a split-dose infusion. In some embodiments, the CAR-expressing cell therapy, e.g., CAR19 expressing cell therapy, is administered in a single infusion. In some embodiments, the CAR19-expressing cell therapy is administered at a dosage of about 0.1×10⁸, 0.2×10⁸, 0.3×10⁸, 0.4×10⁸, 0.5×10⁸, 0.6×10⁸, 0.7×10⁸, 0.8×10⁸, 0.9×10⁸, 1×10⁸, 2×10⁸, 3×10⁸, 4×10⁸, 5×10⁸, 6×10⁸, 7×10⁸, 8×10⁸, 9×10⁸ cells, e.g., about 5×10⁸ cells, e.g., about 5×10⁸ cells in a single infusion. In some embodiments, the CAR19-expressing cell therapy is administered at a dosage of about 0.1×10⁸, 0.2×10⁸, 0.3×10⁸, 0.4×10⁸, 0.5×10⁸, 0.6×10⁸, 0.7×10⁸, 0.8×10⁸, 0.9×10⁸, 1×10⁸, 1.5×10⁸, 2×10⁸, 2.5×10⁸, 3×10⁸, 3.5×10⁸, 4×10⁸, 5×10⁸, 6×10⁸, 7×10⁸, 8×10⁸, 9×10⁸ or 1×10⁹×10⁸ (e.g., CD19 CAR-expressing cells), in a single infusion. In some embodiments, the CAR19-expressing cell therapy is administered at a dosage of about e.g., 0.6-6×10⁸ or 1-5×10⁸, e.g., about 0.8-5.8, 1-5.6, 1.2-5.4, 1.4-5.2, 1.6-5, 1.8-4.8, 2-4.6, 2.2-4.5, 2.4-4.3, 2.6-4.1, 2.8-3.9, 3-3.7, 3.1-3.6×10⁸ cells in a single infusion. In some embodiments, the CAR19-expressing cell therapy is administered at a dosage of about 0.6-6×10⁸ or 1-5×10⁸ cells in a single infusion.

In some embodiments (e.g., when treating DLBCL, e.g., relapsed or refractory DLBCL), the CAR19-expressing cell therapy is administered at a dosage of about 0.6-6×10⁸ cells, e.g., about 0.8-5.8, 1-5.6, 1.2-5.4, 1.4-5.2, 1.6-5, 1.8-4.8, 2-4.6, 2.2-4.5, 2.4-4.3, 2.6-4.1, 2.8-3.9, 3-3.7, 3.1-3.6, 0.6-1, 1-1.5, 1.5-2, 2-2.5, 2.5-3, 3-3.5, 3.5-4, 4-4.5, 4.5-5, 5-5.5, or 5.5-6×10⁸ cells, in a single infusion. In some embodiments, the CAR19-expressing cell therapy is not administrated to a subject with primary central nervous system lymphoma.

In some embodiments (e.g., when treating ALL, e.g., B-cell ALL (B-ALL), e.g., pediatricy or young adult B-ALL), the CAR19-expressing cell therapy is administered at a dosage of about 0.2×10⁶ to 2.5×10⁸ cells in a single infusion. In some embodiments (e.g., when treating ALL, e.g., B-cell ALL (B-ALL), e.g., pediatricy or young adult B-ALL), the CAR19-expressing cell therapy is administered at a dosage of about 0.2-5.0×10⁶ (e.g., about 0.2-0.5, 0.5-1.0, 1.0-1.5, 1.5-2.0, 2.0-2.5, 2.5-3, 3-3.5., 3.5-4, 4-4.5, 4.5-5×10⁶ cells) when the subject weighs ≤50 kg. In some embodiments (e.g., when treating ALL, e.g., B-cell ALL (B-ALL), e.g., pediatricy or young adult B-ALL), the CAR19-expressing cell therapy is administered at a dosage of about 0.1-2.5×10⁸ (e.g., about 0.1-0.5, 0.5-1, 1-1.5, 1.5-2, 2-2.5×10⁸) when the subject weighs >50 kg.

In some embodiments (e.g., when treating FL, e.g., relapsed or refractory FL), the CAR19-expressing cell therapy is administered at a dosage of about 0.6-6×10⁸ cells, e.g., about 0.8-5.8, 1-5.6, 1.2-5.4, 1.4-5.2, 1.6-5, 1.8-4.8, 2-4.6, 2.2-4.5, 2.4-4.3, 2.6-4.1, 2.8-3.9, 3-3.7, 3.1-3.6, 0.6-1, 1-1.5, 1.5-2, 2-2.5, 2.5-3, 3-3.5, 3.5-4, 4-4.5, 4.5-5, 5-5.5, or 5.5-6×10⁸ cells, in a single infusion. In some embodiments, the CAR19-expressing cell therapy is administered at a dosage of 0.6-6×10⁸ CAR19 expressing cells in a single infusion.

### Lymphodepletion

In embodiments, lymphodepletion is performed on a subject, e.g., prior to administering one or more cells that express a CAR described herein. In embodiments, the lymphodepletion regimen comprises administering one or more of melphalan, cytoxan, bendamustine, cyclophosphamide, and fludarabine. In some embodiments, the lymphodepletion regimen is also referred to as a lymphodepleting chemotherapy or a lymphodepleting therapy.

In embodiments, the lymphodepletion regimen comprises administering cyclophosphamide. In embodiments, cyclophosphamide is administered daily, e.g., for 2 or 3 days, at a dosage of about 200-700 mg/m² (e.g., 250-650, 300-600, 350-550, 400-500, 200-300, 400-600, or 450-550 mg/m², e.g., about 250 mg/m² or 500 mg/m²), e.g., intravenously. In some embodiments, cyclophosphamide is administered at a dosage of about 250 mg/m² per day, for 3 days. In some embodiments, cyclophosphamide is administered at a dosage of about 500 mg/m² per day, for 2 days.

In embodiments, the lymphodepletion regimen comprises administering fludarabine. In embodiments, fludarabine is administered daily, e.g., for 3 or 4 days, at a dosage of about 10-50 mg/m² (e.g., 20-30, 25-40 or 25-35 mg/m², e.g., about 25 mg/m² or 30 mg/m²), e.g., intravenously. In some embodiments, fludarabine is administered at a dosage of about 30 mg/m² per day, for 4 days. In some embodiments, fludarabine is administered at a dosage of about 25 mg/m² per day, for 3 days.

In embodiments, the lymphodepletion regimen comprises administering cyclophosphamide and fludarabine. In some embodiments, the lymphodepletion comprises administering 500 mg/m² cyclophosphamide daily for 2 days and 30 mg/m² fludarabine daily for 3 days. In some embodiments, the lymphodepletion regimen comprises administering 250 mg/m² cyclophosphamide daily for 3 days, e.g., 3 doses, and 25 mg/m² fludarabine daily for 3 days, e.g., 3 doses. In some embodiments, the lymphodepletion regimen is initiated with the administration of the first dose of fludarabine. In some embodiments, cyclophosphamide and fludarabine are administered on the same day. In some embodiments, cyclophosphamide and fludarabine are not administered on the same day. In some embodiments, the daily dosages are administered on consecutive days. In embodiments, the subject is administered CAR-expressing cells about 1-14 days, e.g., 2-13, 3-12, 4-11, 5-10, 2-11, or 2-6 days, after completion of the lymphodepletion regimen. In some embodiments, the lymphodepletion regimen is administered to the subject about 1 week, e.g., about 10, 9, 8, 7, 6, 5, 4, 3, or 2 days, prior to administration of CAR-expressing cells. In some embodiments, the subject has a cancer, e.g., a hematological cancer as described herein. In some embodiments, the hematological cancer is a relapsed or refractory cancer. In some embodiments, the hematological cancer is a leukemia or a lymphoma, e.g., a relapsed or refractory leukemia or lymphoma. In some embodiments, the subject is a pediatric subject or a young adult. In some embodiments, the subject is an adult. In some embodiments, the lymphoma is a DLBCL, e.g., a relapsed or refractory DLBCL (e.g., r/r DLBCL), e.g., a CD19+r/r DLBCL. In some embodiments, the subject is an adult and the lymphoma is an r/r DLBCL. In some embodiments, the lymphoma is a follicular lymphoma (FL), e.g., relapsed or refractory FL. In some embodiments, the subject is an adult, e.g., at least 18 years of age, and the lymphoma is a relapsed or refractory FL.

In some embodiments, when the subject has FL, e.g., relapsed or refractory FL, the lymphodepletion regimen comprises administering 250 mg/m² cyclophosphamide daily for 3 days, e.g., 3 doses, and 25 mg/m² fludarabine daily for 3 days, e.g., 3 doses, starting with the first dose of fludarabine.

In some embodiments, when the subject has DLBCL, e.g., relapsed or refractory DLBCL, the lymphodepletion regimen comprises administering 250 mg/m² cyclophosphamide daily for 3 days, e.g., 3 doses, and 25 mg/m² fludarabine daily for 3 days, e.g., 3 doses, starting with the first dose of fludarabine.

In embodiments, the lymphodepletion regimen comprises administering bendamustine. In some embodiments, bendamustine is administered daily, e.g., for 2 days, at a dosage of about 75-125 mg/m2 (e.g., 75-100 or 100-125 mg/m², e.g., about 90 mg/m²), e.g., intravenously. In some embodiments, bendamustine is administered at a dosage of 90 mg/m² daily, e.g., for 2 days. In some embodiments, the subject has a cancer, e.g., a hematological cancer as described herein. In some embodiments, the hematological cancer is a relapsed or refractory cancer. In some embodiments, the hematological cancer is a leukemia or a lymphoma. In some embodiments, the lymphoma is a DLBCL, e.g., a relapsed/refractory DLBCL (e.g., r/r DLBCL), e.g., a CD 19+r/r DLBCL. In some embodiments, the subject is an adult and the lymphoma is an r/r DLBCL. In some embodiments, the lymphoma is a follicular lymphoma (FL), e.g., relapsed or refractory FL. In some embodiments, the subject is an adult, e.g., at least 18 years of age, and the lymphoma is a relapsed or refractory FL. In embodiments, the subject is administered CAR-expressing cells about 1-14 days, e.g., 2-13, 3-12, 4-11, 5-10, 2-11, or 2-6 days, after completion of the lymphodepletion regimen. In some embodiments, the lymphodepletion regimen is administered to the subject about 1 week, e.g., about 10, 9, 8, 7, 6, 5, 4, 3, or 2 days, prior to administration of CAR-expressing cells.

In embodiments, the subject is administered a first lymphodepletion regimen and/or a second lymphodepletion regimen. In embodiments, the first lymphodepletion regimen is administered before the second lymphodepletion regimen. In embodiments, the second lymphodepletion regimen is administered before the first lymphodepletion regimen. In embodiments, the first lymphodepletion regimen comprises cyclophosphamide and fludarabine, e.g., 250 mg/m² cyclophosphamide daily for 3 days, and 25 mg/m² fludarabine daily for 3 days. In embodiments, the second lymphodepletion regimen comprises bendamustine, e.g., 90 mg/m² daily, e.g., for 2 days. In embodiments, the second lymphodepletion regimen is administered as an alternate lymphodepletion regimen. In some embodiments, the second lymphodepletion regimen, e.g., comprising bendamustine, is administered as an alternate lymphodepletion regimen, e.g., if a subject has experienced adverse effects, e.g., hemorrhagic cystitis (e.g., Grade 4 hemorrhagic cystitis), to a lymphodepletion regimen comprising cyclophosphamide, or if a subject shows or has shown resistance to a cyclophoshamide-containing regimen. In some embodiments, the subject has a cancer, e.g., a hematological cancer as described herein. In some embodiments, the hematological cancer is a relapsed or refractory cancer. In some embodiments, the hematological cancer is a leukemia or a lymphoma, e.g., a relapsed or refractory leukemia or lymphoma. In some embodiments, the subject is a pediatric subject or a young adult. In some embodiments, the subject is an adult. In some embodiments, the lymphoma is a DLBCL, e.g., a relapsed or refractory DLBCL (e.g., r/r DLBCL), e.g., a CD19+r/r DLBCL. In some embodiments, the subject is an adult and the lymphoma is an r/r DLBCL. In some embodiments, the lymphoma is a follicular lymphoma (FL), e.g., relapsed or refractory FL. In some embodiments, the subject is an adult, e.g., at least 18 years of age, and the lymphoma is a FL, e.g., relapsed or refractory FL.

In embodiments, the lymphodepletion comprises administering bendamustine (e.g., at about 90 mg/m², e.g., daily ×2), cyclophosphamide and fludarabine (e.g., at about 200 mg/m² cyclophosphamide and about 20 mg/m² fludarabine, e.g., daily ×3), XRT and cyclophosphamide (e.g., at about 400 cGy XRT and about 1 g/m² cyclophosphamide), cyclophosphamide (e.g., about 1 g/m² or 1.2 g/m² cyclophosphamide, e.g., over 4 days), carboplatin and gemcitabine, or modified EPOCH.

In some embodiments, a subject is not administered a lymphodepletion regimen, e.g., lymphodepleting chemotherapy, if the patient has a white blood cell count (WBC) of less than about 5-0.5×10⁹/L, e.g., about 4-0.4, 3-0.3, 2-0.2 or or 1.5-0.5×10⁹/L, e.g., about 1×10⁹/L. In some embodiments, the WBC count is obtained, e.g., within 1 week, e.g., 6, 5, 4, 3, 2, or 1 days, prior to CAR cell administration, e.g., infusion. In some embodiments, a subject is not administered a lymphodepletion regimen, e.g., as described herein, if the subject has cytopenia, e.g., WBC of <100 cells/μl, or absolute lymphocyte count (ALC) of <200/μl. In some embodiments, the subject has a cancer, e.g., a hematological cancer as described herein. In some embodiments, the hematological cancer is a relapsed or refractory cancer. In some embodiments, the hematological cancer is a leukemia or a lymphoma, e.g., a relapsed or refractory leukemia or lymphoma. In some embodiments, the subject is a pediatric subject or a young adult. In some embodiments, the subject is an adult. In some embodiments, the lymphoma is a DLBCL, e.g., a relapsed or refractory DLBCL (e.g., r/r DLBCL), e.g., a CD19+r/r DLBCL. In some embodiments, the subject is an adult and the lymphoma is an r/r DLBCL. In some embodiments, the lymphoma is a follicular lymphoma (FL), e.g., relapsed or refractory FL. In some embodiments, the subject is an adult, e.g., at least 18 years of age, and the lymphoma is a relapsed or refractory FL.

In embodiments, a lymphodepleting chemotherapy is administered to the subject prior to, concurrently with, or after administration (e.g., infusion) of CAR cells, e.g., cells described herein. In an example, the lymphodepleting chemotherapy is administered to the subject prior to administration of CAR cells. For example, the lymphodepleting chemotherapy ends 1-4 days (e.g., 1, 2, 3, or 4 days) prior to CAR cell infusion. In embodiments, multiple doses of CAR cells are administered, e.g., as described herein. For example, a single dose comprises about 5×10⁸ CAR cells. In embodiments, a lymphodepleting chemotherapy is administered to the subject prior to, concurrently with, or after administration (e.g., infusion) of a CAR-expressing cell described herein.

In one embodiment, the one or more doses of the cells are administered after one or more lymphodepleting therapies, e.g., a lymphodepleting chemotherapy. In one embodiment, the lymphodepleting therapy includes a chemotherapy (e.g., cyclophosphamide).

In one embodiment, the one or more doses is followed by a cell transplant, e.g., an allogeneic or autologous hematopoietic stem cell transplant. For example, the allogeneic hematopoietic stem cell transplant occurs between about 20 to about 35 days, e.g., between about 23 and 33 days.

### Biopolymer Delivery Methods

In some embodiments, one or more CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic. Exemplary biopolymers are described, e.g., in paragraphs 1004-1006 of International Application WO2015/142675, filed Mar. 13, 2015, which is herein incorporated by reference in its entirety.

### EXEMPLIFICATION

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### Example 1: Biomarkers for Predicting Response in CLL

In this example, apharesis samples from 31 patients with advanced, heavily pre-treated and high-risk CLL who received at least one dose of CART19 cells were evaluated for biomarkers that can, e.g., predict response to therapy. Of the 31 patients, 7 were complete responders (CR) and 24 were non-responders (NR).

The samples were subjected to flow cytometry based characterization, including assessment of immune checkpoint inhibitors and T-cell differentiation and analysis of expression of markers as detailed below. In this study, the analysis was focused on CD8+ T cell populations. The objective of this study was to identify correlates of response with phenotype. It was observed, e.g., that efficacy of CART therapy was, e.g., not related to patient age, prior therapy, preipipheral tumor burden, or p53 status.

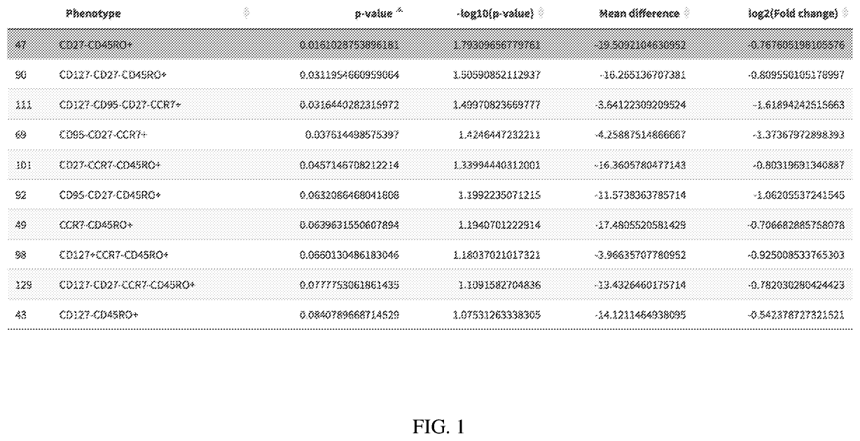

The analysis revealed phenotypes that were present at a statistically significant manner in patients with CR vs NR (FIG. 1). Among the phenotypic markers analyzed were, CD27, CD45RO, CCR7, CD127, HLA-DR and CD95. For example, in some embodiments, NRs showed a higher percentage of CD27−CD45RO+ cells as compared to CRs, with a p-value of 0.016 (FIG. 1). In some embodiments, NRs showed a higher percentage of CD27−CD45RO+CD127− cells as compared to CRs, with a p-value of 0.031. In some embodiments, NRs showed a higher percentage of CD27−CCR7+CD95−CD127− cells as compared to CRs, with a p-value of 0.031. In some embodiments, NRs showed a higher percentage of CD27−CCR7+CD95− cells as compared to CRs, with a p-value of 0.038. In some embodiments, NRs showed a higher percentage of CD27−CD45RO+ CCR7− as compared to CRs, with a p-value of 0.046.

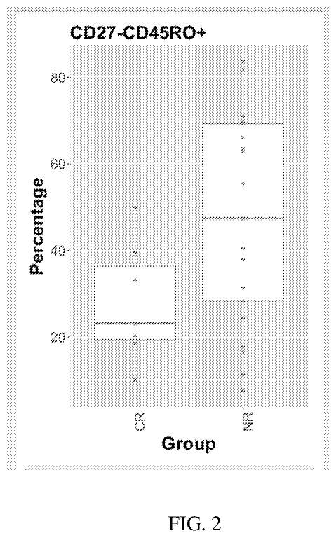

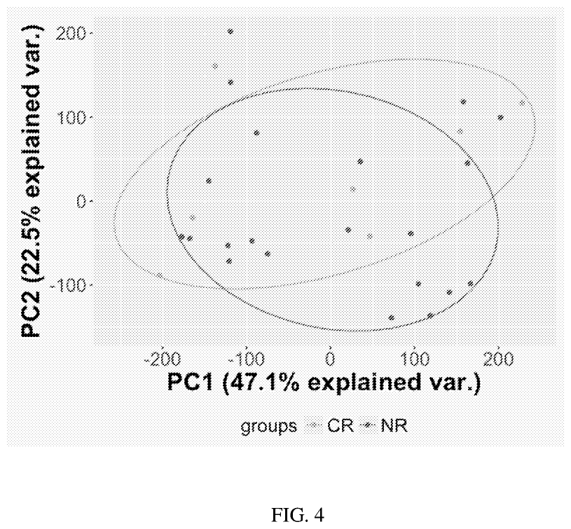

As stated above and shown in FIG. 2, CR patient samples demonstrated a lower percentage of cells expressing CD27−CD45RO+ markers compared to NR patient samples. In contrast, CR patient samples demonstrated a higher percentage of stem cell like T cells having the CD27+CD45RO− markers as compared to NR patient samples (FIG. 3). Principal component analysis demonstrated that CR and NR patient samples can be separated, e.g., distinguished, based on expression of biomarkers, e.g., biomarkers described herein (FIG. 4). In some embodiments, NRs showed a higher percentage of CCR7+ HLA-DR− cells as compared to CRs.

In summary, this example demonstrates the use of biomarkers to, e.g., predict response to therapy, e.g., CART therapy, e.g., CTL019 therapy.

### Example 2: Severe Neurotoxicity is Common and May be Predictable after Chimeric Antigen Receptor Modified T-Cell Therapy Against CD19 (CART-19) Therapy in a Pediatric and Young Adult Cohort

### Introduction

Chimeric antigen receptor (CAR)-modified T-cell therapy against CD19 (CART-19) is a promising new strategy for treating B-cell malignancies in adults and children (see Refs 1-4). There are currently multiple CART-19 products in clinical trials or approved for clinical use, each with a distinctive signaling domain. One such CART-19 produce is CTL019, manufactured at University of Pennsylvania Clinical Cell and Vaccine Production Facility and subsequently developed into Kymriah/Tisagenlecleucel by Novartis which was utilized in this study. Kymriah (tisagenlecleucel) is currently FDA approved for the treatment of patients up to age 25 years with refractory B-cell precursor acute lymphoblastic leukemia (ALL). Efficacy of this therapy is dependent upon T-cell activation and expansion following infusion, often resulting in a significant systemic pro-inflammatory cytokine release syndrome (CRS). Neurotoxicity, such as severe encephalopathy, seizures, ataxia, and other focal deficits, have been reported following CART-19 therapy, however the incidence and neuropathogenesis of these symptoms in children and young adults is not known (see References 5-6).

Some investigators have hypothesized that neurotoxicity following CART-19 infusion is a direct result of CRS (see References 5-7) One group recently reported that 53/133 (40%) of adults who received CART-19 experienced a neurologic adverse event (AE) after infusion ranging from a mild symptom (e.g. transient delirium, headache) to death. 48/53 (91%) of adults with any neurologic AE also had CRS; among the 5/53 adults without CRS, neurologic AEs were mild. Risk factors for any neurologic AE included younger age (age 18-40), a high burden of tumor, CD19+ cells in bone marrow, a high CAR-T cell dose, and pre-existing neurologic comorbidity. Early onset of CRS after CART-19 was specifically associated with higher risk of subsequently developing a severe neurologic AE. Those adults with severe neurologic AEs demonstrated evidence of endothelial activation and systemic capillary leak, which the authors postulated may represent an increase in blood-brain barrier permeability, thus allowing passive transfer of inflammatory mediators of CRS into the CNS. However, neurologic AEs were not prevented by aggressive CRS therapy with dexamethasone and/or IL-6-receptor antagonism (tocilizumab), suggesting that while related, there may be partially independent mechanisms for neurotoxicity and CRS. There are no comparable data in pediatrics (See References 8-9).

A better understanding of the pathogenesis of neurotoxicity in CART-19 therapy across the age span is critically important in order to later identify age-specific potential neurotoxicity mitigation strategies in this highly-effective cancer treatment, and to ensure that neurotoxicity is not ultimately a limiting factor of effective CAR T-cell therapies in general (see References 5, 10 and 11). In a pediatric and young adult population, this study thus aimed to (1) characterize the incidence of neurotoxicity following CART19; (2) define the relationship between neurotoxicity and CRS; and (3) identify predictive biomarkers for the development of neurotoxicity following CART-19.

### Methods

### Study Design and Setting.

A retrospective chart review was performed using data from 51 subjects who received CART-19 infusion at The Childrens Hospital of Philadelphia (CHOP) between Jan. 1, 2010 and Dec. 1, 2015 as a part of a safety/feasibility clinical trial (NCT01626495). Details on the phase I/IIa clinical trial design and CART-19 product are described elsewhere (see Reference 9). The cohort included males and females of any race or ethnicity between the ages of 1-24 years old, who had at least two months of clinical follow up post-infusion, and serum cytokine concentrations measured within the first two months post-infusion. Subjects with ongoing, active CNS oncologic disease (defined by blasts in cerebrospinal fluid), or with encephalopathy attributed to other medical causes post CART-19 infusion, such as sepsis and electrolyte derangements, were excluded.

### Data Collection

Original data collection for NCT01626495 was approved by The CHOP institutional review board on May 18, 2016. Written informed consent was obtained from all subjects or their legal guardians. Data were originally obtained through comprehensive medical evaluations and collection of blood samples. All data for the present sub-study were obtained through the NCT01626495 investigators and additional review of subjects' electronic medical records (EMR) at CHOP. Patient data from their EMR, including oncologic treatment history prior to CART-19 infusion, neurologic and developmental history, hospital course following infusion, pre-infusion and post-infusion neuroimaging and post-infusion EEG records (when available) were recorded onto a standardized data collection form. All data were decoded and maintained in secure databases.

### Study Definitions

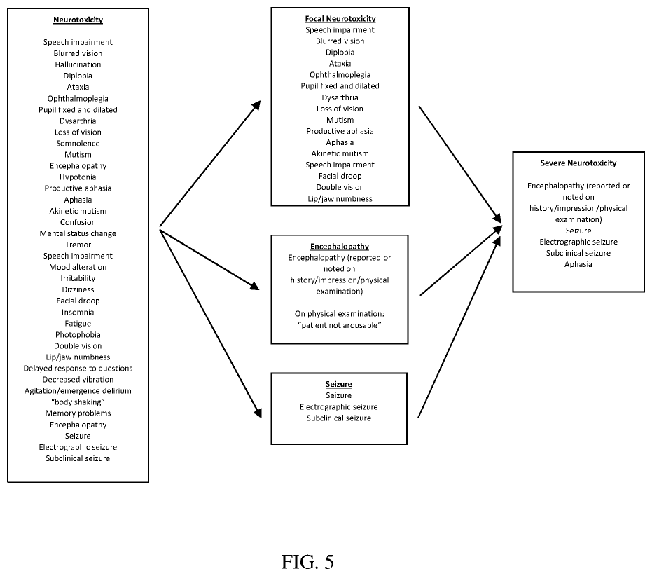

Neurotoxicity was defined as, e.g., any new encephalopathy, focal deficit, seizure, or other clearly defined neurologic symptom, documented in the medical record in, e.g., the first 60 days following CART-19 infusion. Headache, hallucinations, and isolated delirium were excluded from this definition as these can often be seen secondarily in systemic illness and may not represent a primary CNS pathology. Subcategories of neurotoxicity used in analysis are defined in FIG. 5.

Cytokine release syndrome was defined and graded as previously described (see Reference 9) on an interval scale of 0-5, with 0 representing no reaction, and 5 representing death. Neurologic comorbidities, including a history of seizure, need for anticonvulsant medication prior to CART-19 infusion, prior neurologic deficit (defined by a history of a resolved or persistent focal neurologic deficit not due to an ongoing CNS oncologic process) were recorded.

### Laboratory Assessments.

Techniques for serum cytokine, chemokine and soluble cytokine receptor quantification in the NCT01626495 study are described in Reference 9. Briefly, serum was collected approximately on days 1, 4, 5, 9, 10, 14, 17, 21, 28 post-CART-19 infusion in every subject, and processed in the Translational and Correlative Studies Laboratory at the University of Pennsylvania. Cytokine markers were additionally measures on the serum samples from 10 healthy volunteers. Peripheral blood and bone marrow sample were collected and delivered to the laboratory within 2 hours of the sample draw. Serum was isolated by centrifugation and places in 130 uL aliquots. Samples were stored in −80° C. freezer. Serum cytokine and soluble cytokine receptor levels were quantified using a Luminex bead array and a FlexMAP 3D system (Luminex, Austin, Tex.). Data acquisition and analysis was performed with xPONENT software (Luminex).

### Data Analysis and Statistical Methods:

Data were analyzed using Stata version 14.0 (StataCorp, College Station, Tex., 2015), and R version 3.4.0 (R Core Team, Vienna, Austria 2017). Non parametric analysis methods were used given small sample sizes to improve accuracy of statistical estimates and to reduce sensitivity of analyses to statistical outliers. Baseline characteristics of subjects were summarized overall and by neurotoxicity outcome. Continuous variables were described using median and intraquartile range (IQR), and intergroup differences were evaluated using the Wilcoxan rank-sum tests. Categorical variables were described using counts and percents, and intergroup differences were compared using the Fisher exact test. Outcome associations with continuous variables were tested using the Wilcoxon rank-sum test, and with binary variables using the Fisher exact test. The associations between CRS grade and neurotoxicity outcomes were evaluated using the Cochran Armitage test for trend. Unless otherwise stated, statistical tests were two-sided and done at a 0.05 level.

Serial cytokine measurements were summarized as the peak measurement over the first three days post-infusion (3-day peak), as well as the peak measurement over the first 35 days post-infusion (35-day peak) in order to capture early and overall trends of these biomarkers during the period when CART-19 recipients experience neurotoxicity. Values less than the lower limit of detection were recorded as half the lower limit.

In univariate analysis, the association between 3-day or 35-day peak values and the occurrence of either any neurotoxicity or severe neurotoxicity was tested using the Wilcoxon rank-sum test. For each hypothesized association (e.g. three-day peak cytokine level and severe neurotoxicity) a Holm's adjustment for multiple comparisons was included to account for the 43 different cytokines measured. Results were organized into those cytokines elevated in both CRS and neurotoxicity, and those associated with either CRS or neurotoxicity alone.

Models to predict the incidence of neurotoxicity over the first 35-days post-infusion were then created using multivariate analyses. Two modeling approaches were considered. First, a regularized regression elastic net procedure, which accounts for the high dimension of the predictors, was applied to fit a logistic model for the development of neurotoxicity (yes/no) from the candidate predictors: the three-day peak value for each of the 43 cytokines measured and the baseline clinical characteristics (age, sex, history of seizure, and prior neurologic deficit). Two subjects were excluded in this portion of the analysis due to inadequate cytokine measurements. Two secondary outcomes (severe neurotoxicity, encephalopathy) were then similarly assessed. Forward selection logistic regression modeling using Akaike information criteria (AIC) was then performed among the selected predictors to examine a further reduced model. In a second approach, prediction models for the same outcomes and candidate predictors were fit using a classification tree method. Tree models were fit with the tree package in the R statistical software, applying the default deviance split method.

### Standard Protocol Approvals, Registrations, and Patient Consents.

The Children's Hospital of Philadelphia institutional review board approved this sub-study of NCT01626495 data, with a waiver of informed consent (May 18, 2016).

### Results

Cohort demographics and key clinical characteristic of the overall cohort, and subgroups including those with and without severe neurotoxicity are summarized in Table 14. Among the 51 participants examined, 49% percent were male, with a median age of 11.5 years (range 4-22). The most common underlying malignancy was B-cell ALL with no CNS involvement (30/51, 59%), followed by B-cell ALL with known CNS involvement (16/51, 31%). Overall, as expected, all patients had previously refractory leukemia despite multiple modalities of treatment, with a median of 2 relapses prior to CART-19 infusion, prior stem cell or bone marrow transplantation in 34/51 (67%), and a history of brain radiation in 13/48 (27%). A history of neurologic comorbidities, including provoked seizures (16%), need for short-course or standing anti-epileptic drugs (6%), and static or resolved neurologic deficits (20%; including cranial neuropathies, motor deficits, cerebellar abnormalities, and cognitive impairment), were also common. The most common prior neurologic deficits noted were cranial neuropathies (70%). No one in this cohort died in the 2 month follow-up time period following CART-19 infusion.

TABLE 14
Demographics and Clinical Characteristics of the Study Population

| | | | By All Neurotoxicity | | | | | By Severe Neurotoxicity* | | | | |
|---|---|---|---|---|---|---|---|---|---|---|---|---|
| | | | No | | Yes | | | No | | Yes | | |
| | All* | | (n = 28) | | (n = 23) | | p- | (n = 30) | | (n = 21) | | p- |
| Characteristic | (n = 51) | | n(%) | | n(%) | | value^{#} | n (%) | | n (%) | | value^{#} |
| Male sex | 25 | (49.0) | 15 | (53.6) | 10 | (43.5) | 0.58 | 16 | (53.3) | 9 | (42.9) | 0.57 |
| Age | 11.5 | (8, 15) | 11.5 | (8.75, 15) | 9 | (4, 15) | 0.95 | 12 | (9, 15) | 9 | (8, 15) | 0.66 |

Oncologic History:
Malignancy:

| | | | | | | | | | | | | |
|---|---|---|---|---|---|---|---|---|---|---|---|---|
| B-cell ALL with no known CNS | 30 | (58.8) | 17 | (60.7) | 13 | (56.5) | 0.80 | 17 | (56.7) | 13 | (61.9) | 0.83 |
| B-cell ALL with known CNS | 16 | (31.4) | 9 | (32.1) | 7 | (30.4) | | 10 | (33.3) | 6 | (28.6) | |
| Primary refractory B-cell ALL | 1 | (2.0) | 0 | (0.0) | 1 | (4.3) | | 0 | (0.0) | 1 | (4.8) | |
| T-cell ALL | 3 | (5.9) | 1 | (3.6) | 2 | (8.7) | | 2 | (6.7) | 1 | (4.8) | |
| other | 1 | (2.0) | 1 | (3.6) | 0 | (0.0) | | 1 | (3.3) | 0 | (0.0) | |
| Number of relapses | 2 | (2, 3) | 2.5 | (2, 3) | 2 | (2, 3) | 0.80 | 2.5 | (2, 3) | 2 | (2, 3) | 0.17 |
| History of transplant | 34 | (66.7) | 21 | (75.0) | 13 | (56.5) | 0.23 | 22 | (73.3) | 12 | (57.1) | 0.25 |
| History of blinatumamab | 4 | (7.8) | 0 | (0.0) | 4 | (17.4) | 0.11 | 1 | (3.3) | 3 | (14.3) | 0.33 |
| History of brain radiation | 13 | (25.5) | 6 | (21.4) | 7 | (30.4) | 0.53 | 7 | (23.3) | 6 | (28.6) | 0.75 |

Neurologic Comorbidities:

| | | | | | | | | | | | | |
|---|---|---|---|---|---|---|---|---|---|---|---|---|
| History of seizures | 8 | (15.7) | 4 | (14.3) | 4 | (17.4) | 1 | 4 | (13.3) | 4 | (19.0) | 0.70 |
| Prior neurologic deficit | 10 | (19.6) | 3 | (10.7) | 7 | (30.4) | 0.15 | 2 | (6.7) | 8 | (38.1) | 0.01 |
| History of AED use | 3 | (5.9) | 1 | (3.6) | 2 | (8.7) | 0.58 | 1 | (3.3) | 2 | (9.5) | 0.56 |

*Categorical variables are described using n (%). Continuous variables are described using median (IQR).
^{#}p-values comparing those with and without severe neurotoxicity were calculated using Fisher's exact tests for categorical variables and Wilcoxon rank-sum tests for continuous variables

Neurotoxicity following CART-19 infusion was common in this pediatric population, occurring in 23/51 (45%) of subjects, 21/23 of these cases (41% of the total cohort) were severe (Table 2). The most common type of neurotoxicity was encephalopathy, seen in 19/51 (37%) subjects, followed by focal deficits (14/51, 27%), and seizures (4/51, 8%). Types of focal deficits observed included aphasia (6/51, 12%), and less commonly, vision change (n=5/51, 9.8%), facial droop (n=2/51, 3.9%), and others. Of note, there were no known cases of posterior reversible encephalopathy syndrome (PRES), or of cerebral edema; the latter are severe sequelae reported in CART-19 treated adults. However, neuroimaging was not routinely obtained unless clinically indicated, so some cases may have been missed. There was no significant difference in demographics, oncologic history, or existing neurologic comorbidities prior to CART-19 infusion between subjects that developed any neurotoxicity and those that did not (Table 14). In some embodiments, a history of prior neurologic deficit was associated, e.g., with severe neurotoxicity, specifically (p=0.01) (Table 14).

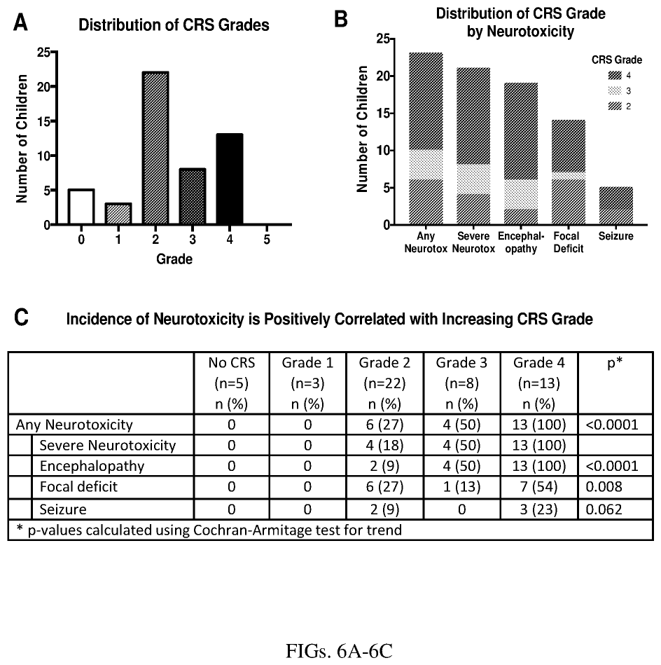

CRS was common, as previously reported (see Reference 9) occurring in 47/51 (92%) subjects at a median of 3 days after CART-19, infusion, ranging in grade from 1-4 (FIGS. 6A-6C). This is a consistent incidence and timing as previously reported in adult cohorts (See References 1-7, 9, 12-14). Neurotoxicity onset occurred, e.g., at a median of 6 days post-infusion lagging behind CRS by, e.g., a median of 3 additional days. Most neurotoxicity was brief and self-limited. In some cases encephalopathy persisted the longest at a median of 4 days (IQR 3-9.5 days).

A positive association was observed between, e.g., incidence of neurotoxicity and increasing grade of CRS (p<0.0001) (FIG. 6A-6C). An IL-6 receptor antagonist (tocilizumab) was administered a median of 5 days post infusion in 10% of subjects with grade 2 or 3 CRS, and to all subjects with grade 4 CRS to treat CRS symptoms. Neurotoxicity was seen in 67% of subjects who received tocilizumab versus 20% of those who did not receive tocilizumab (p=0.061). This analysis did not control for the probable confounding by indication or severity of CRS.

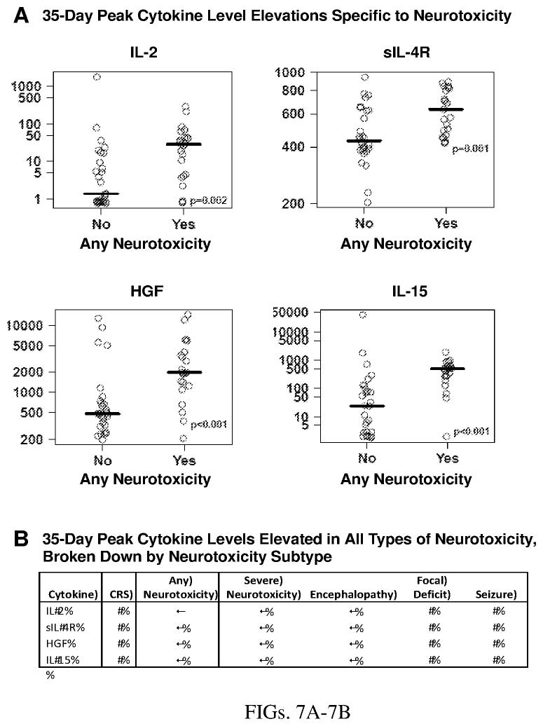

Three-day peak cytokine levels were, e.g., not significantly different between children with and without neurotoxicity. The exception to this was, e.g., soluble tumor necrosis factor receptor-1 (sTNFR-1), which was significantly higher in those who developed encephalopathy compared with those who did not. In some embodiments, the 35-day peak cytokine levels were significantly higher in subjects who developed neurotoxicity compared with those that did not. While many of these cytokines were elevated in both neurotoxicity and CRS, interleukin 2 (IL-2), soluble interleukin 4 receptor (sIL-4R), hepatocyte growth factor (HGF), and interleukin 15 (IL-15) were elevated, e.g., in neurotoxicity alone (FIGS. 7A-7B).

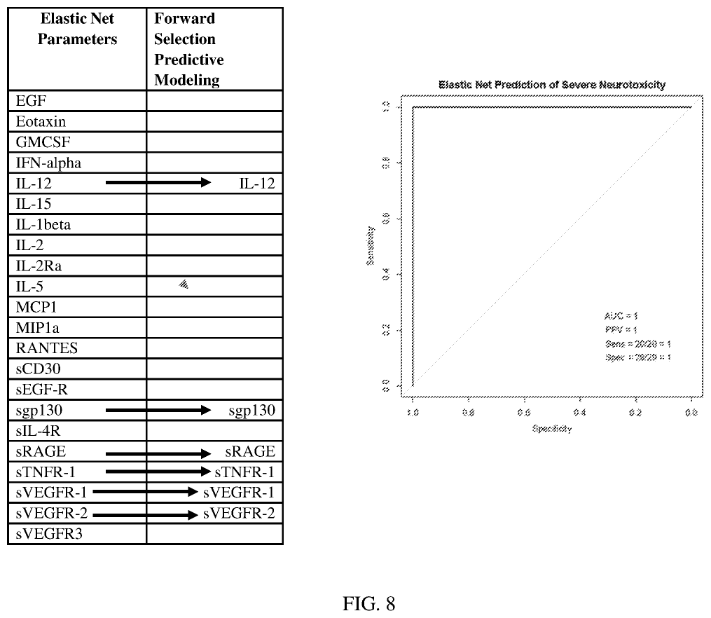

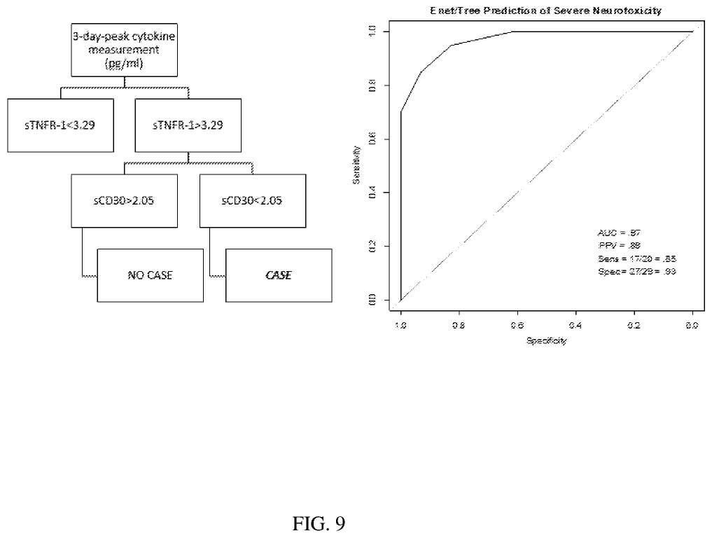

Next, predictive models were used to identify potential biomarkers for neurotoxicity, e.g., severe neurotoxicity. Starting with baseline clinical characteristics and three-day peak cytokine levels of the 43 cytokines measured (in order to identify potential biomarkers for risk of neurotoxicity prior to onset of clinical signs), the elastic net procedure identified 22 significant predictive cytokines (FIG. 8). When these 22 cytokines were incorporated into the forward selection regression prediction model, 6 cytokines (interleukin-12 (IL-12), soluble glycoprotein 130 (sgp130), soluble receptor for advanced glycation end-products (sRAGE), soluble tumor necrosis factor 1 (sTNFR-1), and soluble vascular endothelial growth factors 1 and 2 (sVEGFR-1 and sVEGFR-2) were confirmed as predictive. Using this model, a prediction of those who developed severe neurotoxicity with a positive predictive value of 1 and a sensitivity and specificity of 100% was made. The same model fitting procedure was then applied to a randomly permuted outcome. The area under the curve (AUC) for the resulting prediction model was 0.66, representing inflation over the expected 0.50 for an uncorrelated outcome, suggesting an over-fit model. Using the alternate regression tree-based predictive strategy, three-day peak levels of sTNFR-1, sCD30 had a positive predictive value of 0.89, a sensitivity of 85% and a specificity of 93% (FIG. 9). Given that this was also based upon a limited sample size, like the elastic net procedure, it is also prone to overfitting.

### Discussion

This Example discloses the first study to examine neurotoxicity following CART-19 therapy in a pediatric and young adult population. It has been demonstrated in this Example that neurotoxicity incidence in the month following CART-19 is, e.g., common in children and young adults (45%) and is positively correlated with CRS grade. However, there are specific differences in 35-day peak cytokine patterns among children with CRS alone and those with neurotoxicity, suggesting, e.g., at least a partially independent pathophysiology.

Here, a higher incidence of neurotoxicity (45%) is reported compared to prior studies in adults following CART-19 infusion (e.g., see reference 5), despite the exclusion of more minor reported neurologic adverse events such as headache and delirium. Additionally, specific clinical presentations of neurotoxicity differed in some ways in these children and young adults compared to prior adult studies. For example, aphasia was noted in only 6 (12%) subjects in this cohort, which is lower than the incidence of previously described language disturbance in adults (34%), and there were no known cases of cerebral edema in this cohort. There are several possible explanations for these differences in incidence and phenotypes. First, there may be, e.g., differing underlying ontogenetic vulnerabilities to development of neurotoxicity of CAR T-cell infusions in the developing pediatric brain compared to that of the adult. Alternatively, this pediatric and young adult cohort may have, e.g., had differing neurologic co-morbidities prior to CART-19 infusion compared to adults, which may have impacted development of neurotoxicity. Further study of neurotoxicity following CAR T-cell infusions should examine age-specific epidemiology and pathogenesis of neurotoxicity.

CART-19 is an effective new therapy for B-cell malignancies in children and adults. When these genetically engineered autologous T-cells encounter CD19+ cells in vivo, a signaling domain built into the CAR promotes rapid CAR T-cell proliferation, cytokine production, and lysis of CD19+ target cells, very effectively and killing tumor cells. Efficacy of CART-19 therapy is thus, e.g., dependent on a robust T-cell activation and expansion. However, the same T-cell activation and expansion results in, e.g., a significant systemic pro-inflammatory CRS, which can be, e.g., life-threatening. Prior studies examining the mechanism of CRS have identified specific therapeutic targets, such as IL-6 receptor antagonism.

Some investigators have postulated that neurotoxicity following CART-19 is directly attributable to CRS. One possible mechanism for this hypothesis is based on the observation that individuals with severe neurotoxicity have evidence of endothelial activation and increased blood-brain barrier (BBB) permeability compared to those without (see reference 5). This permeable BBB may then permit passive transfer of inflammatory cytokines into the CNS instigating a local inflammatory response and tissue dysfunction. In some embodiments, our data may support this concept, with, e.g., specifically elevated endothelial factors (VEGF, VEGFR) in both neurotoxicity and CRS, and one elevated endothelial factor (HGF) in neurotoxicity alone. In addition, multiple other endothelial activation factors, e.g., sRAGE, VEGFR-1 and VEGFR-2 were predictive of neurotoxicity in our elastic net model. However, this mechanism does not explain why the 35-day peak cytokine profiles differ between children with CRS alone and those with neurotoxicity. Moreover, clinically, not everyone with CRS develops neurotoxicity, nor does everyone with neurotoxicity have CRS. Finally, neurotoxicity is not clearly mitigated by CRS treatment strategies. Whether the latter observation is because CRS treatment is initiated after neuropathogenesis has already begun (median one day between tocilizumab administration and neurotoxicity onset in this cohort), treatment does not effectively cross the blood brain barrier and permeate the CNS, or if neurotoxicity is simply a separate mechanism, remains unknown.

There are other potential mechanisms of neurotoxicity following CART-19 infusion, which may be related to, but not a direct result of, CRS. One possibility suggested by the data disclosed herein is, e.g., a role of natural killer (NK) cell-mediated inflammation, given selectively elevated IL-2 and IL-15 in those with neurotoxicity. IL-2 and IL-15 are structurally related cytokines which for NK cells, T cells, and B cells. Addition of either cytokine to human peripheral blood monocytes results in selective expansion of NK cells and T cells expressing various NK receptors (CD16, CD161, CD158a, CD158b, KIR3DL1, and CD94), but not conventional B or T cell (see reference 15). While the role of NK cells in the periphery is well described, there are few data on the role of NK cells in the CNS in health or disease. In one model of murine experimental autoimmune encephalomyelitis, NK cells in the CNS produced large amounts of CCL2, a chemoattractant for microglia (see references 16-18). In human in vitro models, activated NK cells appear to lyse resting microglia but spare activated microglia (see references 17, 19 and 20) In some embodiments, if higher levels of, e.g., IL-2 and IL-15 are, e.g., activating NK cells in some CART-19 recipients, NK cells may then, e.g., shift CNS immune homeostasis in favor of activated microglial subtypes, and thus, e.g., make affected individuals more susceptible to neurologic injury (See also references 21-24). The reason for the observed elevation in, e.g., IL-2 and IL-15 in some individuals remains unknown. This hypothesis could be further explored using, e.g., PET imaging with radio-ligands binding to activated microglial.

Another possibility is that some children and young adults may have, e.g., a selective vulnerability to neurotoxicity following an older, static injury. For example, we found that a history prior neurologic deficit was associated with developing severe neurotoxicity post-CART-19 infusion. However, clinical history was not a significant factor in later predictive models. These analyses may be discrepant because they were underpowered. Neurologic history was not always fully delineated prior to CART-19 infusion, and baseline screening neuroimaging was not routinely obtained. Thus, the data may be underestimating the prevalence of pre-existing neurologic injury, and subsequent impact on post-infusion neurotoxicity. Future prospective studies of all CAR T-cell therapy should, e.g., include a standardized, comprehensive baseline neurologic history, examination, and screening neuroimaging evaluation to better understand clinical risk factors for neurotoxicity.

Identifying who is at risk for neurotoxicity before it occurs will be critical to the ongoing clinical care of these children. The elastic net predictive model disclosed herein, identified a group of six three-day peak cytokines that collectively had, e.g., a positive predictive value of one for later development of severe neurotoxicity. The alternate tree-based predictive strategy identified a two-cytokine three-day peak (high sTNFR-1 and low sCD30) model that had a positive predictive value of 0.89. Notably, these two cytokines were also identified in the elastic net procedure, suggesting they may be valid predictive markers. sCD30 is a member of the TNF receptor superfamily and a marker of Th2 polarization (See reference 26). Low levels of sCD30 and high levels of the classic, pro-inflammatory sTNFR-1 may therefore represent a shift towards a more inflammatory Th1 immune response that may be neurotoxic (see references, 16, 27-30). A previously published tree-based prediction model that included some patients from this cohort demonstrated different predictive markers for CRS (IL-10, overall disease burden), supporting, e.g., the concept of a unique pathophysiology between CRS and neurotoxicity (See reference 9). Validation and replication of these findings in a future prospective cohort will be important. In some embodiments, if elevations in sTNFR-1 and lower sCD30 are, e.g., causative of neurotoxicity, e.g., the TNF pathway may be targeted with existing FDA-approved immunomodulatory therapy.

The study disclosed in this Example has certain limitations. First, the sample size was a small sample of CART-19 treated pediatric and young adult patients at the institution. Therefore, analyses may be underpowered, and truly significant associations may, e.g., therefore be underestimated. Non-parametric analyses were thus used to analyze the data. Second, information regarding clinical history, neurotoxicity incidence, and management was retrospective, limited to the existing documentation of the care teams, rather than a prospective standardized assessment. Thus, some historical features may have been missed or misclassified, and some outcome measures may have been misclassified if not documented clearly in medical records. While this could not be corrected for, stringent definitions were applied to neurotoxicity outcomes, e.g., excluding more minor symptoms that may have been included in prior studies. Thus, for outcomes, it is more likely that the analysis disclosed herein has missed some cases, rather than misclassifying, which should bias the data towards the null hypothesis. Third, cytokine data was limited to set time points, so true three-day and 35-day peak values may have been missed by this sampling. However, any bias generated by this method should be non-differential across subjects. In addition, in order to capture true cytokine peaks, daily measurements would be required and such frequent phlebotomy would be logistically prohibitive, particularly for outpatient subjects. Fourth, serum cytokine measurements may not reflect cytokines measurements in the CSF or brain parenchyma, which may more accurately reflect neuropathophysiology following CART-19 infusion. Future studies that rigorously examine CSF cytokine profiles would be informative. Fifth, statistical creation of predictive models for neurotoxicity may be over-fit as described above. However, two different strategies were used to control for multiple comparisons and multiple predictive models, which both identified sTNFR-1 as a potential biomarker thus strengthening the trend of the findings. Finally, neurotoxicity was grouped in broad categories for analysis given the small sample size. However, it is possible that different types of neurotoxicity have distinct neuropathophysiologies. Therefore, findings may have been inadvertently obscured in specific subgroups of neurotoxicity. Future larger studies will be needed to fully examine this potential limitation. Of note, given that each CAR product has a distinctive signaling domain, it is also possible that, e.g., the neurotoxicity noted may be distinctive for each product.

In summary, this Example demonstrated a high incidence of neurotoxicity in the first month following CART-19 infusion in a cohort of 51 pediatric and young adult patients. While this incidence is, e.g., positively associated with higher grade of CRS, differing cytokine profiles between children with neurotoxicity and those with isolated CRS suggest, e.g., an independent pathophysiology in neurotoxicity that may not, e.g., be targeted by CRS therapy alone. Peak levels of sTNFR-1 and sCD30 in the first three days post-infusion may identify children for neurotoxicity, but whether this is causative or simply associated is unknown. Further studies will determine whether targeted therapy against the TNF pathway can, e.g., reduce the risk of neurotoxicity, analogous to the clinical benefit of anti-IL6 therapy for CRS. These data thus provide the foundation for future pediatric investigation into targeted therapy to treat or prevent neurotoxicity following CART-19 therapy, or more broadly, other CAR T-cell therapies.

### REFERENCES FOR EXAMPLE 2 DISCLOSED ABOVE

1. Porter D L, Levine B L, Kalos M, Bagg A, June C H. Chimeric antigen receptor-modified T cells in chronic lymphoid leukemia. N Engl J Med. 2011; 365(8):725-733. doi:10.1056/NEJMoa1103849.
2. Lee D W, Barrett D M, Mackall C, Orentas R, Grupp S A. The future is now: chimeric antigen receptors as new targeted therapies for childhood cancer. Clin Cancer Res. 2012; 18(10):2780-2790. doi:10.1158/1078-0432.CCR-11-1920.
3. Lee D W, Kochenderfer J N, Stetler-Stevenson M, et al. T cells expressing CD19 chimeric antigen receptors for acute lymphoblastic leukaemia in children and young adults: a phase 1 dose-escalation trial. Lancet. 2015; 385(9967):517-528. doi:10.1016/S0140-6736(14)61403-3.
4. Barrett D M, Grupp S A, June C H. Chimeric Antigen Receptor- and TCR-Modified T Cells Enter Main Street and Wall Street. J Immunol. 2015; 195(3):755-761. doi: 10.4049/jimmunol.1500751.
5. Gust J, Hay K A, Hanafi L-A, et al. Endothelial Activation and Blood-Brain Barrier Disruption in Neurotoxicity after Adoptive Immunotherapy with CD19 CAR-T Cells. Cancer Discov. October 2017. doi:10.1158/2159-8290.CD-17-0698.
6. Davila M L, Sauter C, Brentjens R. CD19-Targeted T Cells for Hematologic Malignancies: Clinical Experience to Date. Cancer J. 2015; 21(6):470-474. doi:10.1097/PPO.0000000000000153.
7. Maude S L, Barrett D, Teachey D T, Grupp S A. Managing cytokine release syndrome associated with novel T cell-engaging therapies. Cancer J. 2014; 20(2):119-122. doi:10.1097/PPO.0000000000000035.
8. Lee D W, Kochenderfer J N, Stetler-Stevenson M, et al. T cells expressing CD19 chimeric antigen receptors for acute lymphoblastic leukaemia in children and young adults: a phase 1 dose-escalation trial. Lancet (London, England). 2015; 385(9967):517-528. doi: 10.1016/S0140-6736(14)61403-3.
9. Teachey D T, Lacey S F, Shaw P A, et al. Identification of Predictive Biomarkers for
Cytokine Release Syndrome after Chimeric Antigen Receptor T-cell Therapy for Acute Lymphoblastic Leukemia Clinical Description of Patients. 2016; (June). doi:10.1158/2159-8290.CD-16-0040.
10. Sadelain M, Brentjens R, Riviere I. The basic principles of chimeric antigen receptor design. Cancer Discov. 2013; 3(4):388-398. doi:10.1158/2159-8290.CD-12-0548.
11. Sadelain M. From the guest editor: The rise of CAR therapy: the CD19 paradigm, and beyond. Introduction. Cancer J. 2014; 20(2):105-106. doi:10.1097/PPO.0000000000000042.
12. Teachey D T, Rheingold S R, Maude S L, et al. Cytokine release syndrome after blinatumomab treatment related to abnormal macrophage activation and ameliorated with cytokine-directed therapy. Blood. 2013; 121(26):5154-5157. doi:10.1182/blood-2013-02-485623.
13. Barrett D M, Teachey D T, Grupp S A. Toxicity management for patients receiving novel T-cell engaging therapies. Curr Opin Pediatr. 2014; 26(1):43-49. doi:10.1097/MOP.0000000000000043.
14. Grupp S a., Kalos M, Barrett D, et al. Chimeric Antigen Receptor-Modified T Cells for Acute Lymphoid Leukemia. N Engl J Med. 2013; 368(16):1509-1518. doi:10.1056/NEJMoa1215134.
15. Dunne J, Lynch S, O'Farrelly C, et al. Selective expansion and partial activation of human NK cells and NK receptor-positive T cells by IL-2 and IL-15. J Immunol. 2001; 167(6):3129-3138.
16. Shi C, Pamer E G. Monocyte recruitment during infection and inflammation. Nat Rev Immunol. 2011; 11(11):762-774. doi:10.1038/nri3070.
17. Shi F-D, Ljunggren H-G, La Cava A, Van Kaer L. Organ-specific features of natural killer cells. Nat Rev Immunol. 2011; 11(10):658-671. doi:10.1038/nri3065.
18. Hao J, Liu R, Piao W, et al. Central nervous system (CNS)-resident natural killer cells suppress Th17 responses and CNS autoimmune pathology. J Exp Med. 2010; 207(9):1907-1921. doi:10.1084/jem.20092749.
19. Lunemann A, Lunemann J D, Roberts S, et al. Human NK cells kill resting but not activated microglia via NKG2D- and NKp46-mediated recognition. J Immunol. 2008; 181(9):6170-6177.
20. Lunemann J D, Munz C. Do natural killer cells accelerate or prevent autoimmunity in multiple sclerosis? Brain. 2008; 131 (Pt 7):1681-1683. doi:10.1093/brain/awn132.
21. Durafourt B A, Moore C S, Zammit D A, et al. Comparison of Polarization Properties of Human Adult Microglia and Blood-Derived Macrophages. 2012; 727 (January):717-727. doi:10.1002/glia.22298.
22. Cherry J D, Olschowka J A, Banion M K O. Neuroinflammation and M2 microglia: the good, the bad, and the inflamed. 2014; 11(1):1-15. doi:10.1186/1742-2094-11-98.
23. London A, Cohen M, Schwartz M. Microglia and monocyte-derived macrophages: functionally distinct populations that act in concert in CNS plasticity and repair. 2013; 7 (April):1-10. doi:10.3389/fncel.2013.00034.
24. Orihuela R, Mcpherson C A, Harry G J. polarization and metabolic states. 2016:649-665. doi:10.1111/bph.13139.
25. Attwells S, Setiawan E, Wilson A A, et al. Inflammation in the Neurocircuitry of Obsessive-Compulsive Disorder. 2017; 74(8):833-840. doi:10.1001/jamapsychiatry.2017.1567.
26. Shinoda K, Sun X, Oyamada A, et al. CD30 ligand is a new therapeutic target for central nervous system autoimmunity. J Autoimmun. 2015; 57:14-23. doi:10.1016/j.jaut.2014.11.005.
27. Wang W-Y, Tan M-S, Yu J-T, Tan L. Role of pro-inflammatory cytokines released from microglia in Alzheimer's disease. Ann Transl Med. 2015; 3(10):136. doi: 10.3978/j.issn.2305-5839.2015.03.49.
28. Carlson N G, Wieggel W A, Chen J, Bacchi A, Rogers S W, Gahring L C. Inflammatory cytokines IL-1 alpha, IL-1 beta, IL-6, and TNF-alpha impart neuroprotection to an excitotoxin through distinct pathways. J Immunol. 1999; 163(7):3963-3968.
29. Downen M, Amaral T D, Hua L L, Zhao M L, Lee S C. Neuronal death in cytokine-activated primary human brain cell culture: role of tumor necrosis factor-alpha. Glia. 1999; 28(2):114-127.
30. Feuerstein G Z, Liu T, Barone F C. Cytokines, inflammation, and brain injury: role of tumor necrosis factor-alpha. Cerebrovasc Brain Metab Rev. 1994; 6(4):341-360.

### Example 3: Biomarker Analysis of Tisagenlecleucel Pre-Infusion Biopsies of Adult Patients with Relapsed or Refractory (R/R) Diffuse Large B-Cell Lymphoma (DLBCL)

This Example describes the correlation of tisagenlecleucel (CTL019) efficacy with CD19 and immune checkpoint protein expression in pre-infusion biopsies of DLBCL patients.

Methods: Exploratory analyses were conducted in archival formalin-fixed, paraffin-embedded tumor tissue samples collected months prior to infusion. 92 patients were evaluated for efficacy as of Sep. 6, 2017; 81 patients had baseline biopsy results included in this analysis. Of these samples, the majority were collected within 1 year preinfusion (8 from 1-3 months, 26 from 3-6 months, and 25 from 6 months to 1 year). Quantitative immunofluorescence and image analyses by AQUA Technology were used to evaluate relative levels of CD19, presence of total PD-L1+ cells, and frequency of T cells and non-cells positive for immune checkpoint molecules (PD1, LAG3, TIM3). PD1/PD-L1 interaction scores, defined as the proportion of PD1 positive cells co-localized with PD-L1 positive cells, were also derived. Results were correlated with response.

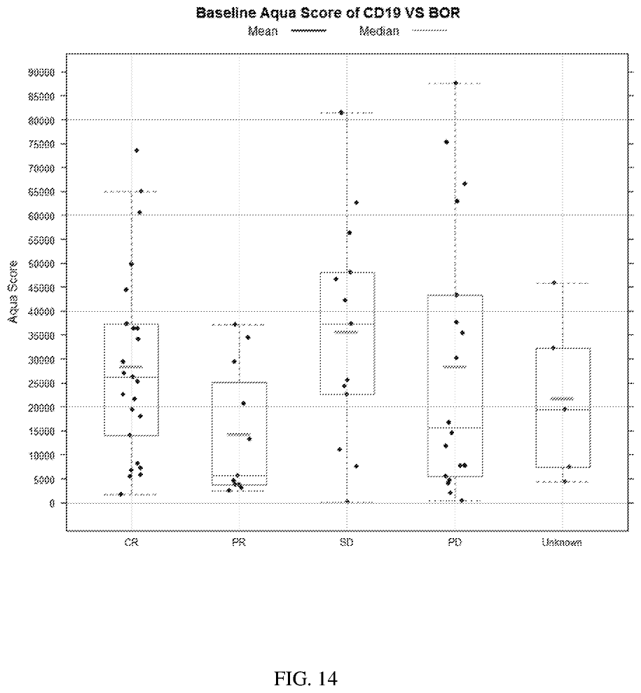

Results: Response to tisagenlecleucel was observed in both patients whose tumor samples showed unequivocal CD19-positive expression (best overall response rate (ORR) 49% [95% CI, 34-64]) and patients with low/negative CD19 expression (best ORR 52% [95% CI, 31-73]) (FIG. 14). No apparent differences were observed among the best overall response (BOR) groups (CR, PR, SD, PD, unknown) in median or mean levels of the percentage of PD-L1+, PD1+, LAG3+, or TIM3+ cells. Similarly, no apparent differences were observed between BOR groups in median and mean levels of: proportion of PD1+, LAG3+, and TIM3+ T cells, and PD1/PD-L1 interaction scores.

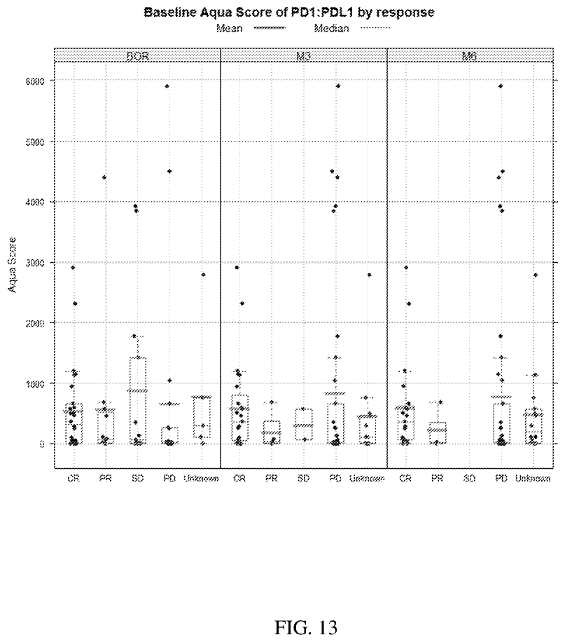

Despite no apparent differences, some of the markers described above showed, e.g., higher expression in a small subset of patients for whom tisagenlecleucel had reduced efficacy. The 5 patients with the highest PD1/PD-L1 interaction score did not respond to tisagenlecleucel or relapsed by month 3 (FIG. 13). Likewise, the 10 patients with highest proportion of LAG3+ T cells did not respond to tisagenlecleucel or relapsed by month 3-6 (by objective response criteria or according to clinical criteria) (data not shown). Furthermore, out of 8 pts with the highest percent (>30%) of PD1+ cells (in areas selected based on high PD-L1 expression), only 2 responded to tisagenlecleucel and 1 progressed at month 3. The majority of PD1+ cells in these 8 patients were not T cells, as the percentage of T cells was much lower than the percentage of PD1+ cells. (data not shown)

Conclusion: This Example describes exploratory biomarker data showing similar response rates across all CD19 expression levels. Furthermore, a small subset of patients with the highest levels of PD1/PD-L1 interaction score, PD1+ cells, and high proportion of LAG3+ T cells (among T cells present), did not respond to tisagenlecleucel or have early relapse. These observations require further investigation, but nonetheless raise the possibility that patients with a high PD1/PD-L1 interaction score, a high proportion of PD1+ cells, and/or a high proportion of LAG3+ cells present among T cells, may have, e.g., a reduced tisagenlecleucel efficacy.

### Example 4: A Phase II Clinical Trial to Determine the Efficacy and Safety of CTL019 in Adult Patients with Refractory or Relapsed Follicular Lymphoma (FL)

### Rationale

Despite recent progress, follicular lymphoma (FL) remains incurable. A recent analysis showed that FL patients who relapsed within 2 years of R-CHOP therapy (approximately 20% of all patients) had poor prognosis, with a 5-year OS of 50% versus 90% in those who relapsed later (Casulo et al., (2015) Early Relapse of Follicular Lymphoma After Rituximab Plus Cyclophosphamide, Doxorubicin, Vincristine, and Prednisone Defines Patients at High Risk for Death: An Analysis From the National LymphoCare Study. J Clin Oncol; 33: 2516-2522). These data challenge the indolent behavior of FL and emphasize the need for novel therapies.

CD19 represents an attractive therapeutic target because it is widely expressed on malignant B-cells, including FL. Tisagenlecleucel (CTL019) consists of autologous T cells that are genetically modified ex vivo via lentiviral transduction to express a chimeric antigen receptor (CAR) consisting of a CD19 antigen recognition domain attached to intracellular signaling domains that mediate T-cell activation. Data from patients with B-cell acute lymphoblastic leukemia (ALL), chronic lymphocytic leukemia (CLL) and DLBCL show an anti-tumor activity of tisagenlecleucel. In Study A2101J, 14 patients with refractory FL were infused with tisagenlecleucel. Ten (71%) patients achieved complete response (CR) and maintained it after 28.6 months of follow up.

### Study Design

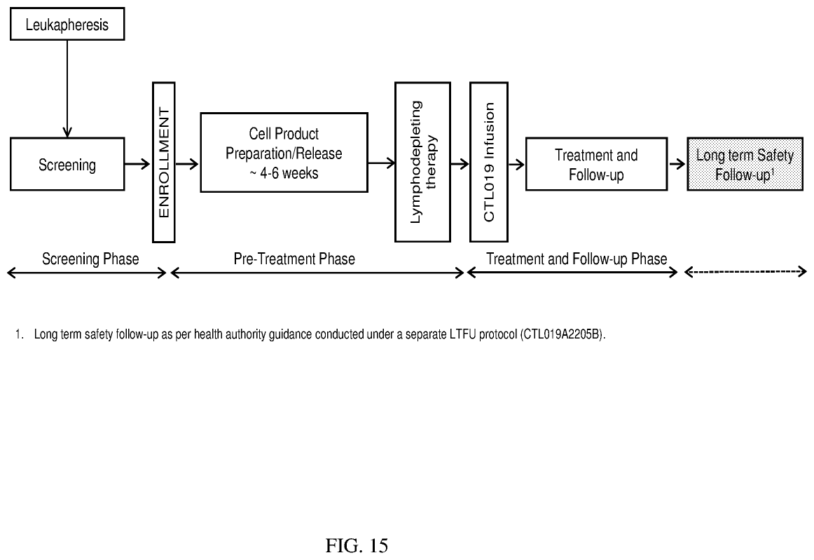

This single arm, multi-center, phase II study is designed to determine the efficacy and safety of tisagenlecleucel (CTL019) in adult patients with FL, e.g., relapsed or refractory FL, who failed at least 2 prior systemic therapies, including an anti-CD20 antibody (e.g., rituximab) and an alkylating agent. Patients who were treated with other FL-targeting agents (including PI3K inhibitors) and patients who relapsed after autologous HSCT will also be included. Treatment and Follow-up Phase will include infusion and safety and efficacy follow-up for 24 months. Efficacy will be evaluated using PET/CT/MRI based on Lugano classification response criteria. The study design is shown in FIG. 15.

### Inclusion Criteria

≥18 years of age
FL (Grade 1, 2, 3A) confirmed histologically by central pathology review before tisagenlecleucel infusion
FL meeting one of the following criteria:
   Refractory to a second line or later line of systemic therapy (including an anti-CD20 antibody and an alkylating agent) or relapsed within 6 months after completion of a second line or later line of systemic therapy
   Relapsed during anti-CD20 antibody maintenance (following at least two lines of therapies as above) or within 6 months after maintenance completion
   Relapsed after autologous HSCT
Radiographically measurable disease at screening defined as:
   At least one nodal lesion greater than 20 mm in the long axis, regardless of the length of the short axis AND/OR
   Extranodal lesions (outside lymph node or nodal mass, including liver and spleen) greater than 10 mm in long AND short axis
ECOG performance status that is either 0 or 1 at screening

### Study Treatment

The recommended dose in this trial is a single infusion of 0.6−6.0×10⁸ CAR-positive viable T cells, which is based on data from studies in CLL, ALL and NHL. The dose-response, dose-safety, and dose-cellular kinetics analyses were performed using the data obtained from relapsed or refractory DLBCL patients (data cut-off date: 8 Mar. 2017) in Phase II study (Study C2201) to assess the impact of dose on exposure, response, and selected safety endpoints in order to select safe and efficacious doses for use in the prescribing setting (commercial) and Study E2202. Across the dose range studied, dose and exposure were independent. Additionally, responses were observed across the full range of doses from 0.6 to 6.0×10⁸ CAR-positive viable T cells. The probability of any grade neurologic events and time to resolution of cytopenia were not impacted by dose. There was an increase in probability of any grade cytokine release syndrome (CRS) and grade 3 or 4 CRS, e.g., severe CRS with increasing dose; however, the probability of grade 3 or 4 CRS was comparable across the dose range of 5.0 to 6.0×10⁸ CAR-positive viable T cells. The model estimates from logistic regression analysis showed that the probability of grade 3 or 4, CRS for dose of 5.0×10⁸ cells and 6.0×10⁸ cells were comparable, i.e., 0.389 and 0.462, respectively. In addition, CRS is generally manageable with CRS management or therapies, e.g., as described herein.

### Lymphodepleting Chemotherapy

It is anticipated that many patients will have been receiving chemotherapy for relapsed or resistant disease. Prior to tisagenlecleucel infusion, each patient should receive lymphodepleting chemotherapy. This may be omitted in case of significant cytopenia (e.g. WBC <1,000 cells/μL, absolute lymphocyte count <200/μL) or any condition that, in the investigator's opinion, precludes lymphodepleting chemotherapy. When given, lymphodepleting chemotherapy should be started 1 week before tisagenlecleucel infusion so that the tisagenlecleucel cells will be given 2 to 6 days after completion of the lymphodepleting chemotherapy. The chemotherapy start date will vary based on the selected chemotherapy. The purpose of this chemotherapy is, e.g., to induce lymphopenia in order to facilitate engraftment and homeostatic expansion of tisagenlecleucel cells. For lymphodepleting chemotherapy, cyclophosphamide-based regimens are preferred agents as there is the most experience with the use of these agents in facilitating adoptive immunotherapy. The first option as lymphodepleting regimen is Fludarabine (25 mg/m² intravenously [i.v.] daily for 3 doses) and cyclophosphamide (250 mg/m² i.v. daily for 3 doses starting with the first dose of fludarabine).

Side effects of fludarabine can include nervous system events of seizure, agitation, blindness, coma and death. Instances of life-threatening and sometimes fatal autoimmune phenomena such as hemolytic anemia, autoimmune thrombocytopenia/thrombocytopenic purpura (ITP), Evans syndrome, and acquired hemophilia have been reported to occur after one or more cycles of treatment with fludarabine phosphate injection. It may also decrease bone marrow function.

Cyclophosphamide toxicities include cardiac dysfunction. Acute cardiac toxicity has been reported with doses as low as 2.4 g/m² to as high as 26 g/m², usually as a portion of an intensive antineoplastic multi-drug regimen or in conjunction with transplantation procedures. In a few instances with high doses of Cyclophosphamide, severe, and sometimes fatal, congestive heart failure has occurred after the first Cyclophosphamide dose. Severe marrow suppression is seen and occasional anaphylactic reactions have been reported. Hemorrhagic cystitis, pulmonary toxicity (pneumonitis, pulmonary fibrosis and pulmonary veno-occlusive disease leading to respiratory failure) and veno-occlusive liver disease may occur.

If there was previous grade IV hemorrhagic cystitis with cyclophosphamide, or the patient demonstrated resistance to a previous cyclophosphamide-containing regimen, then the following regimen should be used Bendamustine 90 mg/m² i.v. daily for 2 days.
Side effects of bendamustine include severely decreased bone marrow function, nausea, vomiting and diarrhea; jaundice may occur, including without other signs of hepatic dysfunction. Fatal and serious cases of liver injury have been reported. No other regimen is allowed for lymphodepletion.

Prior to CTL019 infusion, laboratory assessments, e.g., as described herein, will be performed. In some embodiments, the laboratory assessments will be performed prior to CLT019 infusion, e.g., on day 1. Exemplary laboratory assessments, test category and test methods are detailed in Table 9 below.

TABLE 9
Tests performed in laboratory assessments

| Test category | Test name/method |
|---|---|
| Cytokines | Serum cytokine panel, PD1, PDL1 (peripheral blood) |
| Immunogenicity | Presence of immunogenicity (cellular/humoral) pre/ |
| | post-tisagenlecleucel infusion |
| Tisagenlecleucel | Tisagenlecleucel PK by q-PCR or flow cytometry |
| cellular kinetics | (peripheral blood, bone marrow aspirate) |
| Tumor clonal | Deep sequencing (tumor biopsy, peripheral blood, |
| typing | circulating tumor DNA) |
| Persistence of | Tisagenlecleucel transgene by qPCR |
| CLT019 | |
| transgene | |
| sequences in | |
| relevant tissues | |
| Peripheral blood | Immunophenotyping and leukocyte gene expression |
| molecular | profiling |
| characterization | |
| Minimal Residual | Ig deep sequencing |
| Disease | |
| Additional | Flow cytometry on pre-leukapheresis peripheral |
| assessments | blood and leukapheresis product (ALC, absolute |
| | CD45⁺/CD3⁺, CD45⁺/CD3⁺/CD28⁻/CD27⁻, |
| | CD4⁺/CD25⁺, CD45⁺/CD14⁺). |

### Biomarker Assessment

Biomarker assessment can also be performed as described below. For example, for tumor biopsy samples, confirmation of follicular lymphoma on tissue biopsy by immunohistochemical markers and grading by morphology will be performed by a central laboratory. Quantification of CD19, PD-1 and PD-L1 expression on baseline tumor biopsies will be performed using quantitative immuno-fluorescence and/or other methods (e.g. immunohistochemistry and/or gene expression analysis). The same assessments may be conducted in on-study biopsies. Tumor samples collected in the course of the study may be used for additional exploratory biomarker analysis aimed at identification of potential biomarkers predictive of response to study treatment. Additional potential assessments could be conducted to evaluate: 1) identification and localization of immune cells subsets (e.g. T cells, Treg cells, macrophages), 2) expression and/or localization of additional immunohistochemical markers. Tumor samples may be also used for gene expression profiling (using e.g. nanostring, RNAseq) to correlate immune expression signatures or mutation profiles with response.

With regards to soluble immune markers, the serum levels of inflammatory cytokines and other soluble factors will be assessed pre and post tisagenlecleucel administration. These data will be used to retrospectively identify candidate serum markers potentially correlated with tisagenlecleucel efficacy, CRS severity and possible CNS toxicity.

For peripheral blood samples, the effect of tisagenlecleucel therapy will be measured in peripheral blood to assess on-target effect on levels of CD19 positive B cells. Peripheral blood leukocytes characterization will include immunophenotyping, T cell subset frequency, transcriptome analysis and SNP analysis. Comprehensive DNA sequencing is within scope of these analyses (in accordance with local regulations); at a minimum, targeted sequencing of genes relevant to the tisagenlecleucel mechanism of action will be conducted. In some embodiments, CTL019 immunophenotyping, e.g. in peripheral blood, will be performed by, e.g., deep sequencing, e.g., immunoglobulin (Ig) deep sequencing. Analysis of peripheral blood leukocyte characteristics will be performed to identify potential markers associated with tisagenlecleucel efficacy, expansion and safety. The correlation between characteristics of tisagenlecleucel cell product and leukapheresis product with PK parameters, clinical efficacy and safety endpoints will also be explored. Composition of T cell subsets and cell lineages in peripheral blood and leukapheresis product with progressive disease may also be assessed.

Included within the scope of this study is the assessment of Minimal Residual Disease and tumor evolution. To identify potential markers of tumor cell evolution and tisagenlecleucel efficacy by measuring Minimal Residual Disease (MRD), Ig deep sequencing will be performed on whole blood, circulating tumor DNA (ctDNA) isolated from plasma or tumor when available. Samples assessed will be aliquots obtained from peripheral blood and from plasma. Tumor samples will be collected at Screening, at month 3 and in relation to relapse, when tumor biopsy is performed.

### Study Objective and Endpoints

The primary objective of this study is to evaluate the efficacy of tisagenlecleucel therapy as measured, e.g., by complete response rate (CRR) determined by Independent Review Committee (IRC) in the efficacy analysis set (EAS) based on Lugano 2014 classification response criteria (Cheson et al., (2014) Recommendations for initial evaluation, staging, and response assessment of Hodgkin and non-Hodgkin lymphoma: the Lugano classification. J Clin Oncol; 32(27): 3059-68).

Other objectives, e.g., exploratory objectives, and endpoints are summarized in Table 10.

TABLE 10
Exploratory objectivs and endpoints

| Exploratory Objective | Endpoint |
|---|---|
| Characterize B cell levels over time and | B cell levels, cellular kinetics, and clinical response (PFS) |
| relationship with transgene persistence, | |
| clinical response | |
| Summarize rituximab PK and explore the | Rituximab concentrations (pre-/post- tisagenlecleucel |
| relationship between rituximab PK, B- | infusion) categorized by clinical response (ORR) |
| cells and clinical response | B-cell levels and tisagenlecleucel kinetics |
| Describe composition of T cell subsets | Correlation of levels of CD4-positive/tisagenlecleucel- |
| (immunophenotyping in peripheral blood), | positive/CD3-positive/CD8-positive T cells and other |
| summarized by clinical response and | leukocyte subsets with clinical endpoints |
| relation to cellular kinetics | |
| Describe the profile of blood soluble | Concentrations of soluble factors in blood and |
| immune factors (e.g. IL-6, gamma | correlation with CRS grade and clinical endpoints |
| interferon, sgp130, IL-13, and sIL-6 R) and | |
| their correlation with CRS grade | |
| Relationship between tisagenlecleucel dose | Dose CAR-positive viable T cells |
| and exposure, dose-response and exposure | Exposure (Cmax, AUC0-28 d, AUC0-84 d etc) |
| response | Efficacy (ORR, DOR) |
| | Safety (CRS, neurologic events, cytopenias) |
| To evaluate the impact of intrinsic and | Intrinsic factors (age, gender, ethnicity, etc) and Extrinsic |
| extrinsic factors on cellular kinetics | factors(prior SCT, number of lines of prior therapy, |
| | cytogenetics) |
| | Cellular kinetics (Cmax, Tmax, AUC0-28 d, AUC0-84 d, |
| | other parameters as appropriate) |
| To assess the impact of anti-cytokine | Cellular kinetic parameters in patients with and without |
| agents on cellular kinetics | anti-cytokine use |
| | Cellular kinetic profile by tocilizumab and/or steroids |
| Characterize tocilizumab PK and sIL6R | Tocilizumab PK (Cmax, Tmax, AUCs, and other PK |
| levels by CRS grade | parameters as appropriate) by CRS grade |
| Characterize the relationship between | Product attributes (viability, transduction efficiency, Total |
| select product attributes and in vivo | T cells, IFN gamma) |
| expansion/persistence and dose | Cellular kinetics (Cmax, AUC0-28 d, AUC0-84 d, Clast, Tlast) |
| | Dose (Total CAR positive viable T cell dose) |
| Describe the effect of tocilizumab and | Clinical CRS adverse events and laboratory measures of |
| other anti-cytokine therapies on cytokine | CRS (e.g. IL-6, C-reactive protein (CRP) and ferritin |
| levels in patients with CRS | concentrations) by anti-cytokine therapy |
| Evaluate efficacy in sub-populations (prior | Descriptive summary of clinical outcomes (CRR, ORR, |
| PI3K inhibitor treatment, IG to | DOR, PFS, OS) by sub-populations |
| tisagenlecleucel, overexpression in Bcl-2 | |
| and c-myc) | |
| Evaluate the proportion of patients who | Percentage of patients who undergo HSCT after |
| undergo hematopoietic SCT after | tisagenlecleucel therapy |
| tisagenlecleucel therapy | |
| Describe hospital resource utilization | Number of patients with hospitalized infusion, total number |
| | of hospitalizations, and length of stay |

### Example 5: Long-Term Remission of CLL Sustained by Anti-CD19 Chimeric Antigen Receptor T (CTL019) Cell Clones

It was recently demonstrated that remission in 41 CLL patients treated with the CD19-specific, 4-1BB/CD3zeta-signaling CAR correlated with the expansion and persistence of the engineered T cells and that pathways such as T cell exhaustion, glycolysis and T cell differentiation segregated responders from non-responders. This Example describes two advanced, chemotherapy-resistant CLL patients with the longest (7 years) follow up on our CART cell trial. Both patients had received five therapies before being treated at the University of Pennsylvania with autologous, murine CART19 (CTL019) cells for their CLL in August and September of 2010, receiving 1.1e9 and 1.4e7 CAR19+ T cells, respectively. Both patients are still in remission with persistence of their CAR-engineered T cells as determined by flow cytometry using a CAR19-specific monoclonal antibody and the more sensitive quantitative PCR assays. Furthermore, both patients are still in remission as determined by flow cytometry (CD19/CD5) and deep sequencing of IgH rearrangements for 5.5-7 years. Thus, the infused CAR T cells have maintained these patients in deep molecular remission of their disease.

To understand the fate of the infused CAR T cell clones the phenotype, function, and clonal nature of the persisting CTL019 cells was determined. Flow cytometric CAR T cell analyses demonstrated that early during the anti-leukemia response, activated, HLA-DR− expressing CD8+ CAR T cells rapidly expanded, trailed by similarly activated CD4+ CAR T cells. With tumor clearance the CAR T cell population contracted, but an activated CD4+ CAR T cell population was maintained and was still detectable at the last follow-up. The CD8+ CAR T cell pool remained present at low frequencies. Both populations had acquired and maintained an effector memory phenotype, a phenotype most consistent with active disease control, plus a sizable central memory pool. Furthermore, the analysis of the classical immune checkpoint inhibitory markers PD1, TIM3, LAG3, and CTLA4 showed that only PD1 was expressed from the earliest to the latest timepoint on >80% of all CAR T cells, whereas LAG3 and TIM3 were expressed only early on but lost after tumor clearance. These data suggest that, in some embodiments, the initial tumor clearance was brought about by CD8+ CAR T cells but, e.g., sustained by a CD4+ CAR T cell population that still actively, effectively engages with target cells.

Finally, to understand the clonal nature of these long-term persisting CAR T cells two, complementary methods were used: a) CAR T cells were sorted from post-infusion aliquots during the first two years for T cell receptor-beta deep sequencing (TCR-seq); b) the CAR integration sites in the genome were sequenced in the infusion product and in circulating CAR T cells. TCR-seq analysis of post-infusion time points demonstrated that the circulating CAR T cell populations consisted of hundreds to thousands distinct clones.

In patient 1 this repertoire was fairly evenly distributed with few dominant clones. However, the make-up of this repertoire shifted towards a higher degree of clonality with few clones starting to dominate after the first year, with some representing 4.4% up to 12% of the CAR T cell repertoire. Patient 2, who had received the low CAR T cell dose, demonstrated a higher degree of clonality already 1 month after infusion, with a single clone present at 48% of all CAR T cell clones. The frequency of this clone diminished over time with other CAR T cell clones emanating at later time points, after 5 months. The analysis of clonotype sharing at the various time points via Morisita's overlap index analysis showed that the T cell repertoire in patient 2 was stable over the 2 years whereas patient 1 experienced a greater degree of overlap in the second year after infusion, similarly suggesting that the CAR T cell repertoire had stabilized. Lastly, fate mapping of the infused CAR T cells via CAR integration site analysis in the infusion product until the latest time point indicated that the infusion products for both patients had a very diverse, non-clonal make-up, containing over 8,000 and 3,700 integration sites in patients 1 and 2, respectively. The higher degree of clonality in patient 2 CAR T cells, but not in patient 1 CAR T cells, as seen by TCR-seq was confirmed by integration site analysis, as was the sharing of CAR T cell clones over time. While the CAR integration site repertoire in patient 1 was diverse in the first two years, it stabilized and trended towards oligoclonality 21 months after infusion. The CAR T cell repertoire in patient 2 displayed waves of stability with the first running from month 1 to month 15, followed by a second period from 1.5 years to year 4, and after 5.5 years, all remaining oligoclonal throughout.

Lastly, it is demonstrated herein that CAR integration site analysis revealed a high degree of clonal persistence, suggesting that in some embodiments tumor control and B cell aplasia were, e.g., maintained by few, functional, e.g., highly functional, CAR T cell clones. In summary, this Example demonstrates that in both patients with the longest persistence of CAR T cells reported thus far, early phases of the anti-CLL response are e.g., controlled, e.g., dominated by, activated CD8+ CAR T cells, and late phases of the anti-CLL response are, e.g., controlled, e.g., dominated by, few, persistent clones.

### Example 6: Characterization of Relapse and Non-Response to Tisagenlecleucel Treatment of Pediatric and Young Adult Patients with Relapsed/Refractory B-Cell Acute Lymphoblastic Leukemia (B-ALL)

### Background

Tisagenlecleucel, an anti-CD19 chimeric antigen receptor (CAR)-T cell therapy, has demonstrated efficacy in relapsed/refractory (r/r) pediatric and young adult B-ALL, and was recently approved by FDA in this indication. However, about 30% of patients either fail to respond or relapse after initial remission on tisagenlecleucel treatment. To understand the mechanisms for non-response (NR) and relapse, biomarker analyses was performed to examine if pre-treatment CD19 intensity and immune gene expression patterns are associated with clinical response and relapse in tisagenlecleucel-treated pediatric and young adult r/r B-ALL patients.

### Methods

One hundred and thirty seven pediatric r/r B-ALL patients infused with tisagenlecleucel in two phase II, single arm, multicenter studies (ELIANA, N=79 [NCT02435849], were analyzed in this study. Morphological disease was defined as ≥5% bone marrow blasts, ≥1% peripheral blood blasts, or extramedullary disease. Patients who maintained morphological disease were defined as non-responders (NR). Patients who had re-appearance of morphological disease, and/or patients who became measurable/minimal residual disease (MRD) positive after remission were analyzed as relapse patients. MRD was measured on fresh peripheral blood and bone marrow samples by flow cytometry with MRD positivity defined as ≥0.01% leukemic cells, as well as by immunoglobulin next generation sequencing (Ig NGS) using Adaptive Biotechnologies immunoSEQ assay. Next generation sequencing of RNA (RNA-seq) was performed for broad transcriptional profiling of 76 available bone marrow samples collected at baseline at a depth of 50 million paired end reads.

### RESULTS AND CONCLUSIONS

Of the 137 infused patients tested across both studies, the Overall Response Rate (ORR) was 72% (75 complete responders (CR); 24 complete responders with incomplete blood count recovery (CRi)); 19 (14%) patients did not respond (NR), and 19 (14%) had unknown response. Out of 107 patients who achieved initial remission, 45 patients had morphological relapse, and additional 7 patients became MRD positive. MRD via high throughput sequencing of immunoglobulin genes was performed to study clonal evolution in relapsed patients. Dominant malignant clone(s) detected at baseline were maintained in both CD19(+) and CD19(−) relapsed patients at the time of relapse, in both early and late relapse, indicating that tisagenlecleucel was unable to, e.g., completely remove, e.g., eradicate, leukemic clones in relapsed patients.

The tisagenlecleucel expansion profiles were compared between CD19(+) relapse, CD19(−) relapse and ongoing CRs. CD19(−) relapse and ongoing CR patients had similar profiles compared to CD19(+) relapse patients, which had lower expansion. This data suggests that, in some embodiments, a mechanism of CD19(+) relapse can be, e.g., insufficient CAR-T expansion, whereas, in some embodiments, CD19(−) relapse can be due to loss of CD19 antigen. Gene expression analyses were performed in baseline bone marrow samples to examine whether specific gene signatures and immune cell pathways were enriched in NR, ongoing CR/CRi, or relapsed patients. Compared to CD19(−) relapse, T cell activation and T cell-mediated immune response genes were up-regulated in ongoing responders. This finding indicates that, in some embodiments, a functional T cell compartment at baseline correlates with, e.g., successful tisagenlecleucel treatment.

In summary, linking the findings of increased T cell activation in the bone marrow of baseline CR/CRi patients with low tisagenlecleucel expansion in CD19(+) relapse patients, suggests that, in some embodiments, functionally intact T cells may be an important factor for successful tisagenlecleucel treatment.

### EQUIVALENTS

Other embodiments of the invention will be apparent to those skilled in the art from a consideration of the specification or practice of the invention disclosed herein. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

1. A method of evaluating a subject having a cancer, comprising:
acquiring a value of responder status to a therapy comprising a CAR-expressing cell population for the subject, wherein said value of responder status comprises a determination of one, two, three, four, five, six or more (all), of the following:
(i) the level or activity of CD27 immune effector cells in a sample;
(ii) the level or activity of CD45RO immune effector cells in a sample;
(iii) the level or activity of CCR7 immune effector cells in a sample;
(iv) the level or activity of HLA-DR immune effector cells in a sample;
(v) the level or activity of CD95 immune effector cells in a sample;
(vi) the level or activity of CD127 immune effector cells in a sample; or
(vii) the level, e.g., number, of functional and/or activated T cells in a sample, and
wherein, the determination comprises acquiring a measure of one, two, three, four, five, six, seven, eight, nine, or ten or all of:
a. (i), (ii) and (iii) in the immune effector cells, wherein (iii) comprises a measure of the level or activity of CCR7+ immune effector cells;
b. (iii) and (iv) in the immune effector cells, wherein (iii) comprises a measure of the level or activity of CCR7+ immune effector cells;
c. (i)(ii) and (vi) in the immune effector cells, wherein (vi) comprises a measure of the level or activity of CD127+ immune effector cells;
d. (i) (iii) (v) and (vi) in the immune effector cells, wherein (iii) comprises a measure of the level or activity of CCR7− and (vi) comprises a measure of the level or activity of CD127+ immune effector cells;
e. (i)(iii) and (v) in the immune effector cells, wherein (iii) comprises a measure of the level or activity of CCR7− immune effector cells;
f. (i)(ii) and (v) in the immune effector cells;
g. (ii) and (iii) in the immune effector cells, wherein (iii) comprises a measure of the level or activity of CCR7+ immune effector cells;
h. (ii) (iii) and (vi) in the immune effector cells, wherein (iii) comprises a measure of the level or activity of CCR7+ and (vi) comprises a measure of the level or activity of CD127− immune effector cells;
i. (i), (ii), (iii) and (vi) in the immune effector cells, wherein (iii) comprises a measure of the level or activity of CCR7+ and (vi) comprises a measure of the level or activity of CD127+ immune effector cells;
j. (ii) and (vi) in the immune effector cells, wherein (iii) comprises a measure of the level or activity of CD127+ immune effector cells; and/or
k. any one of (i)-(vii),
wherein said value is indicative of the subject's responsiveness status to the CAR-expressing cell therapy, thereby evaluating the subject,
thereby evaluating the subject.

2. (canceled)

3. (canceled)

4. A method of evaluating or predicting the responsiveness of a subject having a cancer to treatment with a CAR-expressing cell therapy, comprising a determination of one, two, three, four, five, six or more (all), of the following:
(i) the level or activity of CD27 immune effector cells in a sample;
(ii) the level or activity of CD45RO immune effector cells in a sample;
(iii) the level or activity of CCR7 immune effector cells in a sample;
(iv) the level or activity of HLA-DR immune effector cells in a sample;
(v) the level or activity of CD95 immune effector cells in a sample;
(vi) the level or activity of CD127 immune effector cells in a sample; or
(vii) the level of functional and/or activated T cells in a sample, and
wherein, the determination comprises acquiring a measure of one, two, three, four, five, six, seven, eight, nine, ten or all of:
a. (i), (ii) and (iii) in the immune effector cells, wherein (iii) comprises a measure of the level or activity of CCR7+ immune effector cells;
b. (iii) and (iv) in the immune effector cells, wherein (iii) comprises a measure of the level or activity of CCR7+ immune effector cells;
c. (i)(ii) and (vi) in the immune effector cells, wherein (vi) comprises a measure of the level or activity of CD127+ immune effector cells;
d. (i) (iii) (v) and (vi) in the immune effector cells, wherein (iii) comprises a measure of the level or activity of CCR7− and (vi) comprises a measure of the level or activity of CD127+ immune effector cells;
e. (i)(iii) and (v) in the immune effector cells, wherein (iii) comprises a measure of the level or activity of CCR7− immune effector cells;
f. (i)(ii) and (v) in the immune effector cells;
g. (ii) and (iii) in the immune effector cells, wherein (iii) comprises a measure of the level or activity of CCR7+ immune effector cells;
h. (ii) (iii) and (vi) in the immune effector cells, wherein (iii) comprises a measure of the level or activity of CCR7+ and (vi) comprises a measure of the level or activity of CD127− immune effector cells;
i. (i), (ii), (iii) and (vi) in the immune effector cells, wherein (iii) comprises a measure of the level or activity of CCR7+ and (vi) comprises a measure of the level or activity of CD127+ immune effector cells;
j. (ii) and (vi) in the immune effector cells, wherein (iii) comprises a measure of the level or activity of CD127+ immune effector cells; and/or
k. any one of (i)-(vii),
thereby evaluating the subject, or predicting the responsiveness of the subject to the CAR-expressing cell.

5. (canceled)

6. A method of treating, or providing anti-tumor immunity, to a subject having a cancer, who has been identified as being responsive to a therapy,
comprising a population of immune effector cells that expresses a CAR molecule (a “CAR-expressing cell” or a “CAR therapy”), comprising administering to the subject an effective amount of the CAR-expressing cell population, wherein said identifying comprises a determination of one, two, three, four, five, six or more (all), of the following:
(i) the level or activity of CD27 immune effector cells in a sample;
(ii) the level or activity of CD45RO immune effector cells in a sample;
(iii) the level or activity of CCR7 immune effector cells in a sample;
(iv) the level or activity of HLA-DR immune effector cells in a sample;
(v) the level or activity of CD95 immune effector cells in a sample;
(vi) the level or activity of CD127 immune effector cells in a sample; or
(vii) the level of functional and/or activated T cells in a sample, and
wherein, the determination comprises acquiring a measure of one, two, three, four, five, six, seven, eight, nine, ten or all of:
a. (i), (ii) and (iii) in the immune effector cells, wherein (iii) comprises a measure of the level or activity of CCR7+ immune effector cells;
b. (iii) and (iv) in the immune effector cells, wherein (iii) comprises a measure of the level or activity of CCR7+ immune effector cells;
c. (i)(ii) and (vi) in the immune effector cells, wherein (vi) comprises a measure of the level or activity of CD127+ immune effector cells;
d. (i) (iii) (v) and (vi) in the immune effector cells, wherein (iii) comprises a measure of the level or activity of CCR7− and (vi) comprises a measure of the level or activity of CD127+ immune effector cells;
e. (i)(iii) and (v) in the immune effector cells, wherein (iii) comprises a measure of the level or activity of CCR7− immune effector cells;
f. (i)(ii) and (v) in the immune effector cells;
g. (ii) and (iii) in the immune effector cells, wherein (iii) comprises a measure of the level or activity of CCR7+ immune effector cells;
h. (ii) (iii) and (vi) in the immune effector cells, wherein (iii) comprises a measure of the level or activity of CCR7+ and (vi) comprises a measure of the level or activity of CD127− immune effector cells;
i. (i), (ii), (iii) and (vi) in the immune effector cells, wherein (iii) comprises a measure of the level or activity of CCR7+ and (vi) comprises a measure of the level or activity of CD127+ immune effector cells;
j. (ii) and (vi) in the immune effector cells, wherein (iii) comprises a measure of the level or activity of CD127+ immune effector cells; and/or
k. any one of (i)-(vii),
thereby treating, or providing anti-tumor immunity to, the subject.

7. A method of treating a cancer in a subject, comprising:
acquiring a value of responder status to a therapy comprising a population of immune effector cells that expresses a CAR molecule (a “CAR-expressing cell” or a “CAR therapy”) for the subject wherein said value of responder status comprises a determination of one, two, three, four, five, six or more (all), of the following:
(i) the level or activity of CD27 immune effector cells in a sample;
(ii) the level or activity of CD45RO immune effector cells in a sample;
(iii) the level or activity of CCR7 immune effector cells in a sample;
(iv) the level or activity of HLA-DR immune effector cells in a sample;
(v) the level or activity of CD95 immune effector cells in a sample;
(vi) the level or activity of CD127 immune effector cells in a sample; or
(vii) the level of functional and/or activated T cells in a sample, and
wherein, the determination comprises acquiring a measure of one, two, three, four, five, six, seven, eight, nine, ten or all of:
a. (i), (ii) and (iii) in the immune effector cells, wherein (iii) comprises a measure of the level or activity of CCR7+ immune effector cells;
b. (iii) and (iv) in the immune effector cells, wherein (iii) comprises a measure of the level or activity of CCR7+ immune effector cells;
c. (i)(ii) and (vi) in the immune effector cells, wherein (vi) comprises a measure of the level or activity of CD127+ immune effector cells;
d. (i) (iii) (v) and (vi) in the immune effector cells, wherein (iii) comprises a measure of the level or activity of CCR7− and (vi) comprises a measure of the level or activity of CD127+ immune effector cells;
e. (i)(iii) and (v) in the immune effector cells, wherein (iii) comprises a measure of the level or activity of CCR7− immune effector cells;
f. (i)(ii) and (v) in the immune effector cells;
g. (ii) and (iii) in the immune effector cells, wherein (iii) comprises a measure of the level or activity of CCR7+ immune effector cells;
h. (ii) (iii) and (vi) in the immune effector cells, wherein (iii) comprises a measure of the level or activity of CCR7+ and (vi) comprises a measure of the level or activity of CD127− immune effector cells;
i. (i), (ii), (iii) and (vi) in the immune effector cells, wherein (iii) comprises a measure of the level or activity of CCR7+ and (vi) comprises a measure of the level or activity of CD127+ immune effector cells;
j. (ii) and (vi) in the immune effector cells, wherein (iii) comprises a measure of the level or activity of CD127+ immune effector cells; and/or
k. any one of (i)-(vii), and
responsive to said value, performing one, two, three, four, five, six, seven, or more of:
identifying the subject as a complete responder, partial responder or non-responder, or a relapser or a non-relapser;
administering to a responder or a non-relapser, a CAR-expressing cell therapy;
administering an altered dosing of a CAR-expressing cell therapy;
altering the schedule or time course of a CAR-expressing cell therapy;
administering to a non-responder or a partial responder, an additional agent in combination with a CAR-expressing cell therapy;
administering to a non-responder or partial responder a therapy that increases the number of younger T cells or naïve T cells in the subject prior to treatment with a CAR-expressing cell therapy;
modifying a manufacturing process of a CAR-expressing cell therapy or increasing the transduction efficiency for a subject identified as a non-responder or a partial responder;
administering an alternative therapy for a non-responder or partial responder or relapser; or
if the subject is, or is identified as, a non-responder or a relapser, decreasing the T_{REG} cell population and/or T_{REG} gene signature, or administration of cyclophosphamide, an anti-GITR antibody, an mTOR inhibitor, or a combination thereof.

8. The method of claim 7, wherein the responder status is indicative of a complete response, a partial response, a non-response, or a relapse to the CAR-expressing cell therapy.

9. The method of claim 7, wherein the immune effector cell comprises T cells, CD4+ cells, or CD8+ T cells.

10. The method of claim 7, wherein:
a) the method further comprises identifying the subject as a responder, a non-responder, a relapser or a non-relapser, based on a measure of one or more of (i)-(vi);
c) the measure of one or more of (i)-(vii) comprises evaluating a profile for one or more of gene expression, flow cytometry or protein expression;
d) the level or activity of one or more of (i)-(vii) in an immune effector cell population is evaluated using a profile or signature indicative of the percentage of one or more of (i)-(vii) in the immune effector cell population in the sample; or
e) a responder has, or is identified as having, a greater percentage of CD27+CD45RO− CCR7+ immune effector cells compared to a reference value.

11-13. (canceled)

14. The method of claim 7, wherein a responder has, or is identified as having:
a) a greater percentage of CCR7+ HLA-DR− immune effector cells compared to a reference value;
b) a greater percentage of CD27+CD45RO− CD127+ immune effector cells compared to a reference value;
c) a greater percentage of CD27+ CCR7− CD95+CD127+ immune effector cells compared to a reference value;
d) a greater percentage of CD27+ CCR7− CD95+ immune effector cells compared to a reference value;
e) a greater percentage of CD27+CD45RO− CD95+ immune effector cells compared to a reference value;
f) a greater percentage of CCR7+CD45RO− immune effector cells compared to a reference value;
g) a greater percentage of CD45RO− CCR7+CD127− immune effector cells compared to a reference value;
h) a greater percentage of CD27+CD45RO− CCR7+CD127+ immune effector cells compared to a reference value;
i) a greater percentage of CD45RO− CD127+ immune effector cells compared to a reference value; or
j) (i) a greater percentage of CCR7+ HLA-DR− immune effector cells compared to a reference value; or
(ii) a greater percentage of HLA-DR+ immune effector cells compared to a reference value.

15-23. (canceled)

24. A method of evaluating the potency of a CAR-expressing cell product comprising immune effector cells, said method comprising a determination of one, two, three, four, five, six or more (all), of the following:
(i) the level or activity of CD27 immune effector cells in a sample;
(ii) the level or activity of CD45RO immune effector cells in a sample;
(iii) the level or activity of CCR7 immune effector cells in a sample;
(iv) the level or activity of HLA-DR immune effector cells in a sample;
(v) the level or activity of CD95 immune effector cells in a sample;
(vi) the level or activity of CD127 immune effector cells in a sample; or
(vii) the level of functional and/or activated T cells in a sample, and
wherein, the determination comprises acquiring a measure of one, two, three, four, five, six, seven, eight, nine, ten or all of:
a. (i), (ii) and (iii) in the immune effector cells, wherein (iii) comprises a measure of the level or activity of CCR7+ immune effector cells;
b. (iii) and (iv) in the immune effector cells, wherein (iii) comprises a measure of the level or activity of CCR7+ immune effector cells;
c. (i)(ii) and (vi) in the immune effector cells, wherein (vi) comprises a measure of the level or activity of CD127+ immune effector cells;
d. (i) (iii) (v) and (vi) in the immune effector cells, wherein (iii) comprises a measure of the level or activity of CCR7− and (vi) comprises a measure of the level or activity of CD127+ immune effector cells;
e. (i)(iii) and (v) in the immune effector cells, wherein (iii) comprises a measure of the level or activity of CCR7− immune effector cells;
f. (i)(ii) and (v) in the immune effector cells;
g. (ii) and (iii) in the immune effector cells, wherein (iii) comprises a measure of the level or activity of CCR7+ immune effector cells;
h. (ii) (iii) and (vi) in the immune effector cells, wherein (iii) comprises a measure of the level or activity of CCR7+ and (vi) comprises a measure of the level or activity of CD127− immune effector cells;
i. (i), (ii), (iii) and (vi) in the immune effector cells, wherein (iii) comprises a measure of the level or activity of CCR7+ and (vi) comprises a measure of the level or activity of CD127+ immune effector cells;
j. (ii) and (vi) in the immune effector cells, wherein (iii) comprises a measure of the level or activity of CD127+ immune effector cells; and/or
k. any one of (i)-(vii),
wherein the sample is acquired from a subject and
wherein an increase in (i), (ii), (iv), (v) or (vii) or any combination thereof; or an increase in CCR7+ of (iii) and (iv), is indicative of increased suitability for manufacturing of the CAR-expressing cell product, thereby evaluating the potency of the CAR-expressing cell product.

25. A method for optimizing manufacturing of a CAR-expressing cell product comprising immune effector cells comprising:
(1) acquiring from a subject a sample comprising CAR-expressing cell;
(2) activating the CAR-expressing cell in vitro; and
(3) evaluating the potency of the potency of the activated CAR-expressing cell by the method of claim 24.

26. The method of claim 24, further comprising a step of enriching for cells, the immune effector cell population.

27. The method of claim 24, wherein an increase in:
a) CD27+CD45RO− CD127+ immune effector cells;
b) CD27+ CCR7− CD95+ immune effector cells;
c) CD27+CD45RO− CCR7+ immune effector cells;
d) CCR7+CD45RO− immune effector cells;
e) CD45RO− CCR7+CD127− immune effector cells;
f) CD27+CD45RO− CCR7+CD127+ immune effector cells;
g) CD45RO− CD127+ immune effector cells;
h) (i) CCR7+ HLA-DR− immune effector cells; or
(ii) HLA-DR+ immune effector cells;
i) CD27+ CCR7− CD95+CD127+ immune effector cells,
in the CAR-expressing cell product compared to an otherwise identical cell population is indicative of increased suitability for manufacturing of the CAR-expressing cell product.

28-35. (canceled)

36. The method of claim 7, wherein the CAR-expressing cell therapy comprises CTL019 or a nucleic acid encoding a CAR.

37. (canceled)

38. The method of any of claim 7, further comprising selecting the subject for the CAR-expressing therapy.

39. The method of claim 7, wherein the subject has a disease associated with expression of a tumor- or cancer associated-antigen.

40. The method of claim 39, wherein the disease associated with expression of a tumor- or cancer associated-antigen is:
a) a hyperproliferative disorder;
b) a cancer;
c) a hematological cancer or a solid tumor; or
d) a B-cell acute lymphocytic leukemia (B-ALL), T-cell acute lymphocytic leukemia (T-ALL), acute lymphocytic leukemia (ALL), chronic myelogenous leukemia (CML), chronic lymphocytic leukemia (CLL), B cell promyelocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin's lymphoma (NHL), Hodgkin's lymphoma (HL), plasmablastic lymphoma, plasmacytoid dendritic cell neoplasm, and Waldenstrom macroglobulinemia.

41-43. (canceled)

44. The method of claim 7, wherein the immune effector cell population is acquired from a subject, or is acquired from a subject prior to, or after administration of a chemotherapy to the subject.

45. The method of claim 44, wherein:
a) the chemotherapy comprises one or more of an induction, a consolidation, an interim maintenance, a delayed intensification, or a maintenance therapy cycle; and/or
b) the immune effector cell population is acquired from the subject before the subject has been administered a lymphodepleting regimen, or cyclophosphamide, fludarabine, bendamustine, or a combination thereof.

46. (canceled)

47. The method of claim 7, wherein the CAR-expressing cell therapy comprises a plurality of CAR-expressing immune effector cells.

48. The method of claim 7, wherein the value of one or more of (i)-(vii) is obtained from an apheresis sample acquired from the subject or a manufactured CAR-expressing cell product sample.

49. The method of claim 48, wherein the apheresis sample or the manufactured CAR-expressing cell product is evaluated prior to infusion or re-infusion, or after infusion.

50. The method of claim 7, wherein the subject is evaluated prior to, during, or after receiving the CAR-expressing cell therapy.

51. The method of claim 7, wherein the immune effector cell population comprises a higher number of less differentiated T cells, or a higher number of one or more of naïve T cells, stem central memory T cells, and/or central memory T cells, compared to a reference value.

52. The method or composition for use of claim 51, wherein:
the naïve T cells are identified based upon an expression pattern of CCR7+, CD62L+, CD45RO−, CD95−;
the stem central memory T cells are identified based upon an expression pattern of CCR7+, CD62L+, CD45RO−, CD95+; and
the central memory T cells are identified based upon an expression pattern of CCR7+, CD62L+, CD45RO+, CD95+.

53. The method of claim 7, wherein the immune effector cell population is selected based upon the expression of one or more of CCR7, CD62L, CD45RO, and CD95, or the population of immune effector cells are CCR7+ and CD62L+.

54. The method of claim 7, further comprising removing T regulatory cells from the acquired immune cell population, to thereby provide a population of T regulatory-depleted cells.

55. The method of claim 1, wherein the immune effector cell population has been selected based upon the expression of one or more of CD3, CD28, CD4, CD8, CD45RA, and CD45RO, or the population of immune effector cells are CD3+ and/or CD28+.

56-69. (canceled)

70. A method of evaluating a subject, or evaluating responsiveness to a CAR therapy in a subject, comprising:
determining if the subject has an early phase response, a late phase response, or both an early phase response and a late phase response, wherein:
(i) a determination of an early phase response or a late phase response is indicative that the subject is less responsive to a CAR therapy;
(ii) a determination of both an early phase response and a late phase response is indicative that the subject is more responsive to a CAR therapy.
